SEQUENCE LISTING (152 sequences)

SEQ ID NO: 1 - DNA, 2952 bp, Measles virus
atgtttgttttcctggtgctgctgcctctggtgtctagccagtgtgtcaacctgactacccgaacccagctgccccctgcgtacaccaactcctttactaggggtgtctactaccccgacaaggtctttcggtcttctgtacttcattctacacaagatctcttcctgcccttcttcagcaacgtgacatggtttcacgccatccatgtgtccgggaccaatggaaccaagaggttcgacaacccagtgttgccattcaacgatggagtctacttcgctagcacagagaaaagcaacattataaggggctggatctttggcaccactctggatagcaagactcagtccctcctcatcgtgaacaatgctaccaacgtggttattaaggtgtgcgaatttcagttttgtaatgatcccttcctcggggtctactatcacaaaaataataagtcctggatggagtccgagttccgagtttattcttccgcaaataattgcacatttgagtatgttagtcagcccttcctcatggacctcgaaggcaaacagggaaattttaagaaccttagagaattcgtcttcaaaaatatcgatgggtatttcaagatttatagcaagcatacacccattaacctggtacgagacctccctcagggcttttccgcacttgagcctctggtcgatctcccgataggcataaacataactaggtttcagactttgcttgccctccataggtcctaccttacgcctggggatagcagcagtggttggactgcgggcgccgccgcttactatgttgggtatcttcaacctcgcacgttcctgcttaagtataatgaaaatgggaccattacggatgccgtcgattgtgcgctggatcctttgtccgagacgaaatgtaccctgaaaagtttcaccgtggagaaagggatttatcagacgagcaatttccgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtgctctttcgggggagtctccgtcatcaccccaggaactaacacgtctaaccaggtggctgttctgtatcaggatgttaattgcacagaagtgcccgttgcgatacacgcggaccagcttactcctacctggagggtctattccaccggctctaacgtctttcagacccgcgccggttgcctcataggcgcagaacatgtaaataatagttatgaatgcgacattcctattggcgccgggatctgtgcgagctaccagactcaaaccaacagcccgaggcgggccaggtccgtggcgagtcagtccattattgcttatacaatgtccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaatga

SEQ ID NO: 2 - protein, 1273 aa, Measles virus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3822 bp, Measles virus
atgtttgttttcctggtgctgctgcctctggtgtctagccagtgtgtcaacctgactacccgaacccagctgccccctgcgtacaccaactcctttactaggggtgtctactaccccgacaaggtctttcggtcttctgtacttcattctacacaagatctcttcctgcccttcttcagcaacgtgacatggtttcacgccatccatgtgtccgggaccaatggaaccaagaggttcgacaacccagtgttgccattcaacgatggagtctacttcgctagcacagagaaaagcaacattataaggggctggatctttggcaccactctggatagcaagactcagtccctcctcatcgtgaacaatgctaccaacgtggttattaaggtgtgcgaatttcagttttgtaatgatcccttcctcggggtctactatcacaaaaataataagtcctggatggagtccgagttccgagtttattcttccgcaaataattgcacatttgagtatgttagtcagcccttcctcatggacctcgaaggcaaacagggaaattttaagaaccttagagaattcgtcttcaaaaatatcgatgggtatttcaagatttatagcaagcatacacccattaacctggtacgagacctccctcagggcttttccgcacttgagcctctggtcgatctcccgataggcataaacataactaggtttcagactttgcttgccctccataggtcctaccttacgcctggggatagcagcagtggttggactgcgggcgccgccgcttactatgttgggtatcttcaacctcgcacgttcctgcttaagtataatgaaaatgggaccattacggatgccgtcgattgtgcgctggatcctttgtccgagacgaaatgtaccctgaaaagtttcaccgtggagaaagggatttatcagacgagcaatttccgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtgctctttcgggggagtctccgtcatcaccccaggaactaacacgtctaaccaggtggctgttctgtatcaggatgttaattgcacagaagtgcccgttgcgatacacgcggaccagcttactcctacctggagggtctattccaccggctctaacgtctttcagacccgcgccggttgcctcataggcgcagaacatgtaaataatagttatgaatgcgacattcctattggcgccgggatctgtgcgagctaccagactcaaaccaacagcccgaggcgggccaggtccgtggcgagtcagtccattattgcttatacaatgtccctgggagcagagaactctgtggcttactccaacaatagcatagcgataccaactaattttactatatccgtgacaacggagatccttcccgtctccatgacaaaaactagtgtggactgcacaatgtatatctgtggcgatagcactgaatgctccaaccttctgctgcagtatgggagcttttgcacacagttgaaccgggccctgaccggcatcgccgtagagcaggataaaaatacacaggaggtgtttgctcaggtgaagcaaatttacaagacccctcctattaaagatttcggcgggtttaatttcagccagatcctgccggacccatccaagccgagtaagaggagctttatcgaagacttgctgttcaacaaagtgactttggctgatgcaggcttcattaagcagtacggtgattgtctcggcgacatcgcagcgcgggatctcatatgtgctcaaaagttcaacggtcttacagtactcccacctctgctgaccgacgagatgattgctcagtacacgtctgccttgctggccgggacgatcaccagcgggtggacctttggcgcaggcgcagctcttcaaatcccctttgcaatgcagatggcttatcgatttaacggaatcggggtgacgcagaacgtgttgtatgagaaccaaaaattgatagctaatcaattcaattcagcgataggtaagattcaggactccctgtccagcaccgcttccgcgttgggaaaattgcaggacgtggtgaatcaaaatgcacaagcactgaatacactcgtgaaacagctgtccagtaacttcggggccatctcctccgtgctcaacgacatcctttctagacttgataaagtggaagccgaagtgcagattgatcggcttatcacaggtcgcctgcagtcactgcagacttacgtcactcaacagcttatccgcgccgcagagatcagagcaagtgccaatcttgccgcaaccaaaatgtccgagtgcgtgttgggacagtctaagcgagttgacttttgtggtaaggggtaccacctgatgagtttcccccagtccgccccacacggcgtggtttttctgcacgtgacttatgtgccagcccaggagaagaattttactaccgctccggccatttgccacgacggtaaggcacacttccctagagagggggttttcgtgagtaacggcactcactggtttgtcacacagcggaatttttacgagcctcagatcatcactacagataacactttcgtgtccggaaactgcgacgtggttatcggcatcgtcaacaataccgtctatgatcctctccagccagaactggactcctttaaggaagaacttgataaatacttcaagaatcacacaagtccagatgttgaccttggggacattagcgggatcaatgcaagcgtagttaatattcagaaggagattgaccgcctcaacgaagtggccaaaaatctcaatgaatctcttattgatcttcaggagctcggcaagtacgagcagtacatcaagtggccctggtacatatggctcggatttatcgccgggttgatcgccatcgtgatggttacaatcatgctgtgttgcatgacatcatgttgttcctgcttgaaaggctgctgtagctgcgggtcttgttgtaaattcgatgaggatgattctgagccagtactgaagggcgtcaagctccactatacatga

SEQ ID NO: 4 - protein, 685 aa, Measles virus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 5 - DNA, 2097 bp, Measles virus
atgtttgttttcctggtgctgctgcctctggtgtctagccagtgtgtcaacctgactacccgaacccagctgccccctgcgtacaccaactcctttactaggggtgtctactaccccgacaaggtctttcggtcttctgtacttcattctacacaagatctcttcctgcccttcttcagcaacgtgacatggtttcacgccatccatgtgtccgggaccaatggaaccaagaggttcgacaacccagtgttgccattcaacgatggagtctacttcgctagcacagagaaaagcaacattataaggggctggatctttggcaccactctggatagcaagactcagtccctcctcatcgtgaacaatgctaccaacgtggttattaaggtgtgcgaatttcagttttgtaatgatcccttcctcggggtctactatcacaaaaataataagtcctggatggagtccgagttccgagtttattcttccgcaaataattgcacatttgagtatgttagtcagcccttcctcatggacctcgaaggcaaacagggaaattttaagaaccttagagaattcgtcttcaaaaatatcgatgggtatttcaagatttatagcaagcatacacccattaacctggtacgagacctccctcagggcttttccgcacttgagcctctggtcgatctcccgataggcataaacataactaggtttcagactttgcttgccctccataggtcctaccttacgcctggggatagcagcagtggttggactgcgggcgccgccgcttactatgttgggtatcttcaacctcgcacgttcctgcttaagtataatgaaaatgggaccattacggatgccgtcgattgtgcgctggatcctttgtccgagacgaaatgtaccctgaaaagtttcaccgtggagaaagggatttatcagacgagcaatttccgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtgctctttcgggggagtctccgtcatcaccccaggaactaacacgtctaaccaggtggctgttctgtatcaggatgttaattgcacagaagtgcccgttgcgatacacgcggaccagcttactcctacctggagggtctattccaccggctctaacgtctttcagacccgcgccggttgcctcataggcgcagaacatgtaaataatagttatgaatgcgacattcctattggcgccgggatctgtgcgagctaccagactcaaaccaacagcccgaggcgggccaggtccgtggcgagtcagtccattattgcttatacaatgtcctga

SEQ ID NO: 6 - protein, 588 aa, Artificial Sequence
  Synthetic Construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 194 aa, Artificial Sequence
  Synthetic Construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 8 - DNA, 693 bp, Artificial Sequence
  Synthetic Construct
atgcccatggggtctctgcaaccgctggccaccttgtacctgctgggaatgctggttgcttcctgcctcggacgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctga

SEQ ID NO: 9 - DNA, 744 bp, Artificial Sequence
  Synthetic Construct
atgcccatggggtctctgcaaccgctggccaccttgtacctgctgggaatgctggttgcttcctgcctcggacgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttctga

SEQ ID NO: 10 - DNA, 888 bp, Artificial Sequence
  Synthetic Construct
atgcccatggggtctctgcaaccgctggccaccttgtacctgctgggaatgctggttgcttcctgcctcggacgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtga

SEQ ID NO: 11 - protein, 1254 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 12 - DNA, 3765 bp, Artificial Sequence
  Synthetic Construct
atgtttgttttcctggtgctgctgcctctggtgtctagccagtgtgtcaacctgactacccgaacccagctgccccctgcgtacaccaactcctttactaggggtgtctactaccccgacaaggtctttcggtcttctgtacttcattctacacaagatctcttcctgcccttcttcagcaacgtgacatggtttcacgccatccatgtgtccgggaccaatggaaccaagaggttcgacaacccagtgttgccattcaacgatggagtctacttcgctagcacagagaaaagcaacattataaggggctggatctttggcaccactctggatagcaagactcagtccctcctcatcgtgaacaatgctaccaacgtggttattaaggtgtgcgaatttcagttttgtaatgatcccttcctcggggtctactatcacaaaaataataagtcctggatggagtccgagttccgagtttattcttccgcaaataattgcacatttgagtatgttagtcagcccttcctcatggacctcgaaggcaaacagggaaattttaagaaccttagagaattcgtcttcaaaaatatcgatgggtatttcaagatttatagcaagcatacacccattaacctggtacgagacctccctcagggcttttccgcacttgagcctctggtcgatctcccgataggcataaacataactaggtttcagactttgcttgccctccataggtcctaccttacgcctggggatagcagcagtggttggactgcgggcgccgccgcttactatgttgggtatcttcaacctcgcacgttcctgcttaagtataatgaaaatgggaccattacggatgccgtcgattgtgcgctggatcctttgtccgagacgaaatgtaccctgaaaagtttcaccgtggagaaagggatttatcagacgagcaatttccgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtgctctttcgggggagtctccgtcatcaccccaggaactaacacgtctaaccaggtggctgttctgtatcaggatgttaattgcacagaagtgcccgttgcgatacacgcggaccagcttactcctacctggagggtctattccaccggctctaacgtctttcagacccgcgccggttgcctcataggcgcagaacatgtaaataatagttatgaatgcgacattcctattggcgccgggatctgtgcgagctaccagactcaaaccaacagcccgaggcgggccaggtccgtggcgagtcagtccattattgcttatacaatgtccctgggagcagagaactctgtggcttactccaacaatagcatagcgataccaactaattttactatatccgtgacaacggagatccttcccgtctccatgacaaaaactagtgtggactgcacaatgtatatctgtggcgatagcactgaatgctccaaccttctgctgcagtatgggagcttttgcacacagttgaaccgggccctgaccggcatcgccgtagagcaggataaaaatacacaggaggtgtttgctcaggtgaagcaaatttacaagacccctcctattaaagatttcggcgggtttaatttcagccagatcctgccggacccatccaagccgagtaagaggagctttatcgaagacttgctgttcaacaaagtgactttggctgatgcaggcttcattaagcagtacggtgattgtctcggcgacatcgcagcgcgggatctcatatgtgctcaaaagttcaacggtcttacagtactcccacctctgctgaccgacgagatgattgctcagtacacgtctgccttgctggccgggacgatcaccagcgggtggacctttggcgcaggcgcagctcttcaaatcccctttgcaatgcagatggcttatcgatttaacggaatcggggtgacgcagaacgtgttgtatgagaaccaaaaattgatagctaatcaattcaattcagcgataggtaagattcaggactccctgtccagcaccgcttccgcgttgggaaaattgcaggacgtggtgaatcaaaatgcacaagcactgaatacactcgtgaaacagctgtccagtaacttcggggccatctcctccgtgctcaacgacatcctttctagacttgataaagtggaagccgaagtgcagattgatcggcttatcacaggtcgcctgcagtcactgcagacttacgtcactcaacagcttatccgcgccgcagagatcagagcaagtgccaatcttgccgcaaccaaaatgtccgagtgcgtgttgggacagtctaagcgagttgacttttgtggtaaggggtaccacctgatgagtttcccccagtccgccccacacggcgtggtttttctgcacgtgacttatgtgccagcccaggagaagaattttactaccgctccggccatttgccacgacggtaaggcacacttccctagagagggggttttcgtgagtaacggcactcactggtttgtcacacagcggaatttttacgagcctcagatcatcactacagataacactttcgtgtccggaaactgcgacgtggttatcggcatcgtcaacaataccgtctatgatcctctccagccagaactggactcctttaaggaagaacttgataaatacttcaagaatcacacaagtccagatgttgaccttggggacattagcgggatcaatgcaagcgtagttaatattcagaaggagattgaccgcctcaacgaagtggccaaaaatctcaatgaatctcttattgatcttcaggagctcggcaagtacgagcagtacatcaagtggccctggtacatatggctcggatttatcgccgggttgatcgccatcgtgatggttacaatcatgctgtgttgcatgacatcatgttgttcctgcttgaaaggctgctgtagctgcgggtcttgttgttga

SEQ ID NO: 13 - protein, 1208 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 14 - DNA, 3627 bp, Artificial Sequence
  Synthetic Construct
atgtttgttttcctggtgctgctgcctctggtgtctagccagtgtgtcaacctgactacccgaacccagctgccccctgcgtacaccaactcctttactaggggtgtctactaccccgacaaggtctttcggtcttctgtacttcattctacacaagatctcttcctgcccttcttcagcaacgtgacatggtttcacgccatccatgtgtccgggaccaatggaaccaagaggttcgacaacccagtgttgccattcaacgatggagtctacttcgctagcacagagaaaagcaacattataaggggctggatctttggcaccactctggatagcaagactcagtccctcctcatcgtgaacaatgctaccaacgtggttattaaggtgtgcgaatttcagttttgtaatgatcccttcctcggggtctactatcacaaaaataataagtcctggatggagtccgagttccgagtttattcttccgcaaataattgcacatttgagtatgttagtcagcccttcctcatggacctcgaaggcaaacagggaaattttaagaaccttagagaattcgtcttcaaaaatatcgatgggtatttcaagatttatagcaagcatacacccattaacctggtacgagacctccctcagggcttttccgcacttgagcctctggtcgatctcccgataggcataaacataactaggtttcagactttgcttgccctccataggtcctaccttacgcctggggatagcagcagtggttggactgcgggcgccgccgcttactatgttgggtatcttcaacctcgcacgttcctgcttaagtataatgaaaatgggaccattacggatgccgtcgattgtgcgctggatcctttgtccgagacgaaatgtaccctgaaaagtttcaccgtggagaaagggatttatcagacgagcaatttccgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtgctctttcgggggagtctccgtcatcaccccaggaactaacacgtctaaccaggtggctgttctgtatcaggatgttaattgcacagaagtgcccgttgcgatacacgcggaccagcttactcctacctggagggtctattccaccggctctaacgtctttcagacccgcgccggttgcctcataggcgcagaacatgtaaataatagttatgaatgcgacattcctattggcgccgggatctgtgcgagctaccagactcaaaccaacagcccgaggcgggccaggtccgtggcgagtcagtccattattgcttatacaatgtccctgggagcagagaactctgtggcttactccaacaatagcatagcgataccaactaattttactatatccgtgacaacggagatccttcccgtctccatgacaaaaactagtgtggactgcacaatgtatatctgtggcgatagcactgaatgctccaaccttctgctgcagtatgggagcttttgcacacagttgaaccgggccctgaccggcatcgccgtagagcaggataaaaatacacaggaggtgtttgctcaggtgaagcaaatttacaagacccctcctattaaagatttcggcgggtttaatttcagccagatcctgccggacccatccaagccgagtaagaggagctttatcgaagacttgctgttcaacaaagtgactttggctgatgcaggcttcattaagcagtacggtgattgtctcggcgacatcgcagcgcgggatctcatatgtgctcaaaagttcaacggtcttacagtactcccacctctgctgaccgacgagatgattgctcagtacacgtctgccttgctggccgggacgatcaccagcgggtggacctttggcgcaggcgcagctcttcaaatcccctttgcaatgcagatggcttatcgatttaacggaatcggggtgacgcagaacgtgttgtatgagaaccaaaaattgatagctaatcaattcaattcagcgataggtaagattcaggactccctgtccagcaccgcttccgcgttgggaaaattgcaggacgtggtgaatcaaaatgcacaagcactgaatacactcgtgaaacagctgtccagtaacttcggggccatctcctccgtgctcaacgacatcctttctagacttgataaagtggaagccgaagtgcagattgatcggcttatcacaggtcgcctgcagtcactgcagacttacgtcactcaacagcttatccgcgccgcagagatcagagcaagtgccaatcttgccgcaaccaaaatgtccgagtgcgtgttgggacagtctaagcgagttgacttttgtggtaaggggtaccacctgatgagtttcccccagtccgccccacacggcgtggtttttctgcacgtgacttatgtgccagcccaggagaagaattttactaccgctccggccatttgccacgacggtaaggcacacttccctagagagggggttttcgtgagtaacggcactcactggtttgtcacacagcggaatttttacgagcctcagatcatcactacagataacactttcgtgtccggaaactgcgacgtggttatcggcatcgtcaacaataccgtctatgatcctctccagccagaactggactcctttaaggaagaacttgataaatacttcaagaatcacacaagtccagatgttgaccttggggacattagcgggatcaatgcaagcgtagttaatattcagaaggagattgaccgcctcaacgaagtggccaaaaatctcaatgaatctcttattgatcttcaggagctcggcaagtacgagcagtga

SEQ ID NO: 15 - protein, 1238 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLG

SEQ ID NO: 16 - DNA, 3717 bp, Artificial Sequence
  Synthetic Construct
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggctga

SEQ ID NO: 17 - DNA, 21 bp, Artificial Sequence
  Synthetic Construct
gtggacatatcactcactctg

SEQ ID NO: 18 - DNA, 19 bp, Artificial Sequence
  Synthetic Construct
ggtgtgtgtctcctcctat

SEQ ID NO: 19 - DNA, 21 bp, Artificial Sequence
  Synthetic Construct
ctcattggaacgttgctggtg

SEQ ID NO: 20 - DNA, 19 bp, Artificial Sequence
  Synthetic Construct
ggtgtgtgtctcctcctat

SEQ ID NO: 21 - DNA, 3822 bp, Artificial Sequence; identical to SEQ ID NO: 3
  Synthetic Construct

SEQ ID NO: 22 - DNA, 47 bp, Artificial Sequence
  Synthetic Construct
cgccagcccatcaacgcgtataatgtttgttttcctggtgctgctgc

SEQ ID NO: 23 - DNA, 50 bp, Artificial Sequence
  Synthetic Construct
tttataatggatttaggttgtatcatgtatagtggagcttgacgcccttc

SEQ ID NO: 24 - DNA, 53 bp, Artificial Sequence
  Synthetic Construct
tttataatggatttaggttgtattatcaggacattgtataagcaataatggac

SEQ ID NO: 25 - DNA, 106 bp, Artificial Sequence
  Synthetic Construct
atactcgagatgcccatggggtctctgcaaccgctggccaccttgtacctgctgggaatgctggttgcttcctgcctcggaaacatcacaaacctgtgtcccttcg

SEQ ID NO: 26 - DNA, 50 bp, Artificial Sequence
  Synthetic Construct
tttataatggatttaggttgtattagacggtagctggagcgtgcagcagc

SEQ ID NO: 27 - DNA, 50 bp, Artificial Sequence
  Synthetic Construct
cgccagcccatcaacgcgtatagagatgcccatggggtctctgcaaccgc

SEQ ID NO: 28 - DNA, 24 bp, Artificial Sequence
  Synthetic Construct
accaaacaaagttgggtaaggata

SEQ ID NO: 29 - DNA, 20 bp, Artificial Sequence
  Synthetic Construct
ggtaggcggatgttgttctg

SEQ ID NO: 30 - DNA, 18 bp, Artificial Sequence
  Synthetic Construct
cttctagactaggtgcga

SEQ ID NO: 31 - DNA, 47 bp, Artificial Sequence
  Synthetic Construct
gcagcagcaccaggaaaacaaacattatacgcgttgatgggctggcg

SEQ ID NO: 32 - DNA, 53 bp, Artificial Sequence
  Synthetic Construct
gcggttgcagagaccccatgggcatgagtatacgcgttgatgggctggcggct

SEQ ID NO: 33 - DNA, 50 bp, Artificial Sequence
  Synthetic Construct
gaagggcgtcaagctccactatacatgatacaacctaaatccattataaa

SEQ ID NO: 34 - DNA, 53 bp, Artificial Sequence
  Synthetic Construct
gtccattattgcttatacaatgtcctgataatacaacctaaatccattataaa

SEQ ID NO: 35 - DNA, 50 bp, Artificial Sequence
  Synthetic Construct
gctgctgcacgctccagctaccgtctaatacaacctaaatccattataaa

SEQ ID NO: 36 - DNA, 20 bp, Artificial Sequence
  Synthetic Construct
catgaatatggcagagacgt

SEQ ID NO: 37 - DNA, 20 bp, Artificial Sequence
  Synthetic Construct
cccgacgacactcaactccc

SEQ ID NO: 38 - DNA, 26 bp, Artificial Sequence
  Synthetic Construct
acgtttttcttaattctgatgtctat

SEQ ID NO: 39 - DNA, 19 bp, Artificial Sequence
  Synthetic Construct
acatcaggcatacccacta

SEQ ID NO: 40 - DNA, 19 bp, Artificial Sequence
  Synthetic Construct
cccacatatggcttcttag

SEQ ID NO: 41 - DNA, 21 bp, Artificial Sequence
  Synthetic Construct
gacaaagagtcatgttcagtg

SEQ ID NO: 42 - DNA, 19 bp, Artificial Sequence
  Synthetic Construct
cagacaaagctgggaatag

SEQ ID NO: 43 - DNA, 31 bp, Artificial Sequence
  Synthetic Construct
gcaatatattaaagaaaactttgaaaatacg

SEQ ID NO: 44 - DNA, 27 bp, Artificial Sequence
  Synthetic Construct
gcactagaagatgatcattgattgaac

SEQ ID NO: 45 - DNA, 25 bp, Artificial Sequence
  Synthetic Construct
cctaagttttaattaactaccgata

SEQ ID NO: 46 - DNA, 18 bp, Artificial Sequence
  Synthetic Construct
tccctctggccgaacaat

SEQ ID NO: 47 - DNA, 25 bp, Artificial Sequence
  Synthetic Construct
gcaaccaaaccagaacccagaccac

SEQ ID NO: 48 - DNA, 30 bp, Artificial Sequence
  Synthetic Construct
ctaggtgaacttcagggtataagatctggt

SEQ ID NO: 49 - DNA, 25 bp, Artificial Sequence
  Synthetic Construct
cgttaccttgcatattccctgaacg

SEQ ID NO: 50 - DNA, 29 bp, Artificial Sequence
  Synthetic Construct
gggagctgggtatcctgggatgatctatc

SEQ ID NO: 51 - DNA, 20 bp, Artificial Sequence
  Synthetic Construct
gtacgcagggggcagctggg

SEQ ID NO: 52 - DNA, 20 bp, Artificial Sequence
  Synthetic Construct
cgaggcgaagtggttaaaag

SEQ ID NO: 53 - DNA, 20 bp, Artificial Sequence
  Synthetic Construct
acggatcaacctcattccac

SEQ ID NO: 54 - DNA, 20 bp, Artificial Sequence
  Synthetic Construct
aattcgccaatgacaagacg

SEQ ID NO: 55 - DNA, 19 bp, Artificial Sequence
  Synthetic Construct
gcctgtgaggagccaccaa

SEQ ID NO: 56 - DNA, 19 bp, Artificial Sequence
  Synthetic Construct
cgtcatctgagacaggtag

SEQ ID NO: 57 - DNA, 47 bp, Artificial Sequence; identical to SEQ ID NO: 22
  Synthetic Construct

SEQ ID NO: 58 - DNA, 50 bp, Artificial Sequence; identical to SEQ ID NO: 23
  Synthetic Construct

SEQ ID NO: 59 - DNA, 53 bp, Artificial Sequence; identical to SEQ ID NO: 24
  Synthetic Construct

SEQ ID NO: 60 - DNA, 55 bp, Artificial Sequence
  Synthetic Construct
tttataatggatttaggttgtattatcactgctcgtacttgccgagctcctgaag

SEQ ID NO: 61 - DNA, 106 bp, Artificial Sequence; identical to SEQ ID NO: 25
  Synthetic Construct

SEQ ID NO: 62 - DNA, 106 bp, Artificial Sequence
  Synthetic Construct
atactcgagatgcccatggggtctctgcaaccgctggccaccttgtacctgctgggaatgctggttgcttcctgcctcggacgcgtccagccaaccgagtccattg

SEQ ID NO: 63 - DNA, 50 bp, Artificial Sequence; identical to SEQ ID NO: 26
  Synthetic Construct

SEQ ID NO: 64 - DNA, 50 bp, Artificial Sequence
  Synthetic Construct
tttataatggatttaggttgtattatcagaaattcacgcatttgttcttg

SEQ ID NO: 65 - DNA, 54 bp, Artificial Sequence
  Synthetic Construct
tttataatggatttaggttgtattatcacggagtaatgtcaaggatttccaatg

SEQ ID NO: 66 - DNA, 50 bp, Artificial Sequence; identical to SEQ ID NO: 27
  Synthetic Construct

SEQ ID NO: 67 - DNA, 47 bp, Artificial Sequence
  Synthetic Construct
cgccagcccatcaacgcgtataatgttcgtgttcctggtgctcctgc

SEQ ID NO: 68 - DNA, 52 bp, Artificial Sequence
  Synthetic Construct
tttataatggatttaggttgtatcattagcccaggaatgtgctcagcagtac

SEQ ID NO: 69 - DNA, 24 bp, Artificial Sequence
  Synthetic Construct
accaaacaaagttgggtaaggata

SEQ ID NO: 70 - DNA, 20 bp, Artificial Sequence
  Synthetic Construct
ggtaggcggatgttgttctg

SEQ ID NO: 71 - DNA, 18 bp, Artificial Sequence
  Synthetic Construct
cttctagactaggtgcga

SEQ ID NO: 72 - DNA, 47 bp, Artificial Sequence; identical to SEQ ID NO: 31
  Synthetic Construct

SEQ ID NO: 73 - DNA, 53 bp, Artificial Sequence; identical to SEQ ID NO: 32
  Synthetic Construct

SEQ ID NO: 74 - DNA, 47 bp, Artificial Sequence
  Synthetic Construct
gcaggagcaccaggaacacgaacattatacgcgttgatgggctggcg

SEQ ID NO: 75 - DNA, 50 bp, Artificial Sequence; identical to SEQ ID NO: 33
  Synthetic Construct

SEQ ID NO: 76 - DNA, 53 bp, Artificial Sequence; identical to SEQ ID NO: 34
  Synthetic Construct

SEQ ID NO: 77 - DNA, 50 bp, Artificial Sequence; identical to SEQ ID NO: 35
  Synthetic Construct

SEQ ID NO: 78 - DNA, 50 bp, Artificial Sequence
  Synthetic Construct
caagaacaaatgcgtgaatttctgataatacaacctaaatccattataaa

SEQ ID NO: 79 - DNA, 54 bp, Artificial Sequence
  Synthetic Construct
cattggaaatccttgacattactccgtgataatacaacctaaatccattataaa

SEQ ID NO: 80 - DNA, 55 bp, Artificial Sequence
  Synthetic Construct
cttcaggagctcggcaagtacgagcagtgataatacaacctaaatccattataaa

SEQ ID NO: 81 - DNA, 52 bp, Artificial Sequence
  Synthetic Construct
gtactgctgagcacattcctgggctaatgatacaacctaaatccattataaa

SEQ ID NO: 82 - DNA, 20 bp, Artificial Sequence
  Synthetic Construct
catgaatatggcagagacgt

SEQ ID NO: 83 - DNA, 20 bp, Artificial Sequence
  Synthetic Construct
cccgacgacactcaactccc

SEQ ID NO: 84 - DNA, 26 bp, Artificial Sequence
  Synthetic Construct
acgtttttcttaattctgatgtctat

SEQ ID NO: 85 - DNA, 19 bp, Artificial Sequence
  Synthetic Construct
acatcaggcatacccacta

SEQ ID NO: 86 - DNA, 19 bp, Artificial Sequence
  Synthetic Construct
cccacatatggcttcttag

SEQ ID NO: 87 - DNA, 21 bp, Artificial Sequence
  Synthetic Construct
gacaaagagtcatgttcagtg

SEQ ID NO: 88 - DNA, 19 bp, Artificial Sequence
  Synthetic Construct
cagacaaagctgggaatag

SEQ ID NO: 89 - DNA, 31 bp, Artificial Sequence; identical to SEQ ID NO: 43
  Synthetic Construct

SEQ ID NO: 90 - DNA, 27 bp, Artificial Sequence
  Synthetic Construct
gcactagaagatgatcattgattgaac

SEQ ID NO: 91 - DNA, 25 bp, Artificial Sequence
  Synthetic Construct
cctaagttttaattaactaccgata

SEQ ID NO: 92 - DNA, 18 bp, Artificial Sequence
  Synthetic Construct
tccctctggccgaacaat

SEQ ID NO: 93 - DNA, 25 bp, Artificial Sequence
  Synthetic Construct
gcaaccaaaccagaacccagaccac

SEQ ID NO: 94 - DNA, 30 bp, Artificial Sequence; identical to SEQ ID NO: 48
  Synthetic Construct

SEQ ID NO: 95 - DNA, 25 bp, Artificial Sequence
  Synthetic Construct
cgttaccttgcatattccctgaacg

SEQ ID NO: 96 - DNA, 29 bp, Artificial Sequence
  Synthetic Construct
gggagctgggtatcctgggatgatctatc

SEQ ID NO: 97 - DNA, 20 bp, Artificial Sequence
  Synthetic Construct
gtacgcagggggcagctggg

SEQ ID NO: 98 - DNA, 20 bp, Artificial Sequence
  Synthetic Construct
gcctctaccagcagctcagt

SEQ ID NO: 99 - DNA, 20 bp, Artificial Sequence
  Synthetic Construct
gcagacagggttctccagac

SEQ ID NO: 100 - DNA, 20 bp, Artificial Sequence
  Synthetic Construct
ccatcaaggcagacctgttt

SEQ ID NO: 101 - DNA, 20 bp, Artificial Sequence
  Synthetic Construct
ttcttgttgggacgatttcc

SEQ ID NO: 102 - DNA, 20 bp, Artificial Sequence
  Synthetic Construct
ggtggtgtcagtcattgtgg

SEQ ID NO: 103 - DNA, 20 bp, Artificial Sequence
  Synthetic Construct
agacagcacgaggcatttct

SEQ ID NO: 104 - DNA, 20 bp, Artificial Sequence
  Synthetic Construct
tgcacccacttcaagctcta

SEQ ID NO: 105 - DNA, 20 bp, Artificial Sequence
  Synthetic Construct
gccttcttgggcatgtaaaa

SEQ ID NO: 106 - DNA, 20 bp, Artificial Sequence
  Synthetic Construct
ctccgcaagagacttccatc

SEQ ID NO: 107 - DNA, 20 bp, Artificial Sequence
  Synthetic Construct
accaaacctccgacttgttg

SEQ ID NO: 108 - DNA, 20 bp, Artificial Sequence
  Synthetic Construct
gacgggctgtacctggttta

SEQ ID NO: 109 - DNA, 20 bp, Artificial Sequence
  Synthetic Construct
gagtcggtcacctttctcca

SEQ ID NO: 110 - DNA, 20 bp, Artificial Sequence
  Synthetic Construct
tgccagtgctatcctccttt

SEQ ID NO: 111 - DNA, 20 bp, Artificial Sequence
  Synthetic Construct
gatggcctcagactctggat

SEQ ID NO: 112 - DNA, 20 bp, Artificial Sequence
  Synthetic Construct
aactttggcattgtggaagg

SEQ ID NO: 113 - DNA, 20 bp, Artificial Sequence
  Synthetic Construct
cgacatgtgagatccacgac

SEQ ID NO: 114 - protein, 10 aa, Artificial Sequence
  Synthetic Construct
MFVFLVLLPL

SEQ ID NO: 115 - protein, 9 aa, Artificial Sequence
  Synthetic Construct
AEHVNNSYE

SEQ ID NO: 116 - protein, 10 aa, Artificial Sequence
  Synthetic Construct
GAEHVNNSYE

SEQ ID NO: 117 - protein, 10 aa, Artificial Sequence
  Synthetic Construct
VLKGVKLHYT

SEQ ID NO: 118 - DNA, 28 bp, Artificial Sequence
  Synthetic Construct
gtcattctcctaagaagctattaaaatc

SEQ ID NO: 119 - DNA, 21 bp, Artificial Sequence
  Synthetic Construct
gtgtctttgatttcgagcaac

SEQ ID NO: 120 - DNA, 20 bp, Artificial Sequence
  Synthetic Construct
cattggcatggaagtcacac

SEQ ID NO: 121 - DNA, 20 bp, Artificial Sequence
  Synthetic Construct
tctgcggtaaggcttgagtt

SEQ ID NO: 122 - DNA, 20 bp, Artificial Sequence
  Synthetic Construct
actgtcgttgacaggacacg

SEQ ID NO: 123 - DNA, 20 bp, Artificial Sequence
  Synthetic Construct
acgtcgcgaacctgtaaaac

SEQ ID NO: 124 - protein, 230 aa, Artificial Sequence
  Synthetic Construct
MPMGSLQPLATLYLLGMLVASCLGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 125 - protein, 247 aa, Artificial Sequence
  Synthetic Construct
MPMGSLQPLATLYLLGMLVASCLGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 126 - protein, 295 aa, Artificial Sequence
  Synthetic Construct
MPMGSLQPLATLYLLGMLVASCLGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITP

SEQ ID NO: 127 - protein, 1239 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGR

SEQ ID NO: 128 - DNA, 3717 bp, Artificial Sequence
  Synthetic Construct
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagccctatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggccccgccctgcagatcccctttcccatgcagatggcctatagattcaacggaatcggcgtgacccagaacgtgctgtatgaaaaccagaagctgatcgctaaccagttcaattctgccatcggcaagatccaggactccctctcctctacccccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggcaga

SEQ ID NO: 129 - protein, 1236 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSHGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGR

SEQ ID NO: 130 - DNA, 3708 bp, Artificial Sequence
  Synthetic Construct
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatcagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctacctacggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgacgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccacgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagccctatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggccccgccctgcagatcccctttcccatgcagatggcctatagattcaacggaatcggcgtgacccagaacgtgctgtatgaaaaccagaagctgatcgctaaccagttcaattctgccatcggcaagatccaggactccctctcctctacccccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggcaga

SEQ ID NO: 131 - protein, 1236 aa, Artificial Sequence
  Synthetic Construct
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGR

SEQ ID NO: 132 - DNA, 3708 bp, Artificial Sequence
  Synthetic Construct
atgttcgtgttcctggtgctcctgcctctggtgagcagccagtgcgtgaacttcaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgccaaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccggggcctccctcagggcttcagcgccctggaacccctggtcgacctgcccataggcatcaacataacgcggttccaaaccctgcatatctcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaacatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtgaagggcttcaactgttacttccctctgcaaagctacggcttccagcctacctacggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagccctatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggccccgccctgcagatcccctttcccatgcagatggcctatagattcaacggaatcggcgtgacccagaacgtgctgtatgaaaaccagaagctgatcgctaaccagttcaattctgccatcggcaagatccaggactccctctcctctacccccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggcaga

SEQ ID NO: 133 - protein, 419 aa, Artificial Sequence
  Synthetic Construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 134 - DNA, 1260 bp, Artificial Sequence
  Synthetic Construct
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 135 - protein, 222 aa, Artificial Sequence
  Synthetic Construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 136 - DNA, 669 bp, Artificial Sequence
  Synthetic Construct
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 137 - protein, 75 aa, Artificial Sequence
  Synthetic Construct
MYSFVSEETGTLIVNSVLLFLAFVVFLLVTLAILTALRLCAYCCNIVNVSLVKPSFYVYSRVKNLNSSRVPDLLV

SEQ ID NO: 138 - DNA, 232 bp, Artificial Sequence
  Synthetic Construct
acttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 139 - protein, 245 aa, Artificial Sequence
  Synthetic Construct
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQ

SEQ ID NO: 140 - DNA, 830 bp, Artificial Sequence
  Synthetic Construct
ttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaa

SEQ ID NO: 141 - protein, 121 aa, Artificial Sequence
  Synthetic Construct
MKFLVFLGIITTVAAFHQECSLQSCTQHQPYVVDDPCPIHFYSKWYIRVGARKSAPLIELCVDEAGSKSPIQYIDIGNYTVSCSPFTINCQEPKLGSLVVRCSFYEDFLEYHDVRVVLDFI

SEQ ID NO: 142 - DNA, 366 bp, Artificial Sequence
  Synthetic Construct
atgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaa

SEQ ID NO: 143 - protein, 290 aa, Artificial Sequence
  Synthetic Construct
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 144 - DNA, 876 bp, Artificial Sequence
  Synthetic Construct
atgagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtaa

SEQ ID NO: 145 - protein, 277 aa, Artificial Sequence
  Synthetic Construct
MPMGSLQPLATLYLLGMLVASCLGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGYIPEAPRDGQAYVRKDGEWVLLSTFLG

SEQ ID NO: 146 - DNA, 834 bp, Artificial Sequence
  Synthetic Construct
atgcccatggggtctctgcaaccgctggccaccttgtacctgctgggaatgctggttgcttcctgcctcggacgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcggatccggatacatccccgaggcccccagagatggccaggcctacgtgcggaaggacggcgagtgggtactgctgagcacattcctgggctga

SEQ ID NO: 147 - protein, 728 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSGSGYIPEAPRDGQAYVRKDGEWVLLSTFLG

SEQ ID NO: 148 - DNA, 2187 bp, Artificial Sequence
  Synthetic Construct
atgtttgttttcctggtgctgctgcctctggtgtctagccagtgtgtcaacctgactacccgaacccagctgccccctgcgtacaccaactcctttactaggggtgtctactaccccgacaaggtctttcggtcttctgtacttcattctacacaagatctcttcctgcccttcttcagcaacgtgacatggtttcacgccatccatgtgtccgggaccaatggaaccaagaggttcgacaacccagtgttgccattcaacgatggagtctacttcgctagcacagagaaaagcaacattataaggggctggatctttggcaccactctggatagcaagactcagtccctcctcatcgtgaacaatgctaccaacgtggttattaaggtgtgcgaatttcagttttgtaatgatcccttcctcggggtctactatcacaaaaataataagtcctggatggagtccgagttccgagtttattcttccgcaaataattgcacatttgagtatgttagtcagcccttcctcatggacctcgaaggcaaacagggaaattttaagaaccttagagaattcgtcttcaaaaatatcgatgggtatttcaagatttatagcaagcatacacccattaacctggtacgagacctccctcagggcttttccgcacttgagcctctggtcgatctcccgataggcataaacataactaggtttcagactttgcttgccctccataggtcctaccttacgcctggggatagcagcagtggttggactgcgggcgccgccgcttactatgttgggtatcttcaacctcgcacgttcctgcttaagtataatgaaaatgggaccattacggatgccgtcgattgtgcgctggatcctttgtccgagacgaaatgtaccctgaaaagtttcaccgtggagaaagggatttatcagacgagcaatttccgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcaatttcaacggtctcaccgggacgggcgtgcttacggaaagcaacaagaaattcctgccattccaacagttcggccgggacatcgccgacaccactgacgcggtgcgcgatcctcagacattggaaatccttgacattactccgtgctctttcgggggagtctccgtcatcaccccaggaactaacacgtctaaccaggtggctgttctgtatcaggatgttaattgcacagaagtgcccgttgcgatacacgcggaccagcttactcctacctggagggtctattccaccggctctaacgtctttcagacccgcgccggttgcctcataggcgcagaacatgtaaataatagttatgaatgcgacattcctattggcgccgggatctgtgcgagctaccagactcaaaccaacagcccgaggcgggccaggtccgtggcgagtcagtccattattgcttatacaatgtccggatccggatacatccccgaggcccccagagatggccaggcctacgtgcggaaggacggcgagtgggtactgctgagcacattcctgggctga

SEQ ID NO: 149 - protein, 531 aa, Artificial Sequence
  Synthetic Construct
MPMGSLQPLATLYLLGMLVASCLGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGTRGVRGPHGQRPARPTLCPESDRCTNLCPTGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVRRPASPAPRALAVARGCLARTPCTYFPGAQHGNK

SEQ ID NO: 150 - DNA, 1599 bp, Artificial Sequence
  Synthetic Construct
atgcccatggggtctctgcaaccgctggccaccttgtacctgctgggaatgctggttgcttcctgcctcggacgcgtccagccaaccgagtccattgtgcggtttccgaacatcacaaacctgtgtcccttcggggaggtgttcaacgccacacggtttgctagtgtttacgcatggaatcgaaagcgaattagtaactgcgtggcagactactcagttctttataacagtgcgagcttctctacttttaagtgctacggtgtgtcccccaccaagcttaacgacctctgttttaccaacgtctacgccgatagcttcgtcattaggggggatgaagtgagacagatcgcacctggccagactgggaaaatcgccgattacaactacaaactgccggatgatttcacaggctgcgttatcgcttggaacagtaataacttggactcaaaagtgggagggaactataattatctctatcgactgttcagaaagtctaacctgaaaccttttgaacgagatattagtaccgagatttaccaagctggcagtacaccgtgcaacggcgtcgaggggttcaattgttattttcctcttcaatcctacggttttcagcctactaacggagtgggataccagccataccgagtggttgtgctgtcattcgagctgctgcacgctccagctaccgtctgtggccctaaaaagtccacgaacctggtcaagaacaaatgcgtgaatttcgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaatga

SEQ ID NO: 151 - protein, 982 aa, Artificial Sequence
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGTRGVRGPHGQRPARPTLCPESDRCTNLCPTGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVRRPASPAPRALAVARGCLARTPCTYFPGAQHGNK

SEQ ID NO: 152 - DNA, 2952 bp, Artificial Sequence; identical to SEQ ID NO: 1
  Synthetic Construct

(19) United States
(12) Patent Application Publication
(10) Pub. No.: US 2023/0310581 A1
(43) Pub. Date: Oct. 5, 2023

(54) A LIVE ATTENUATED MEASLES VIRUS VECTORED VACCINE FOR SARS-COV-2

(71) Applicant: OHIO STATE INNOVATION FOUNDATION, Columbus, OH (US)

(72) Inventors: Jianrong LI, Columbus, OH (US); Stefan NIEWIESK, Columbus, OH (US); Anzhong LI, Columbus, OH (US); Mijia LU, Columbus, OH (US)

(21) Appl. No.: 17/905,962

(22) PCT Filed: Apr. 27, 2021
(86) PCT No.: PCT/US21/29373

§ 371 Date: Sep. 9, 2022

Related U.S. Application Data
(60) Provisional application No. 63/016,184, filed on Apr. 27, 2020.
(60) Provisional application No. 63/134,111, filed on Jan. 5, 2021.

(51) Int. Cl. A61K 39/215; A61K 39/00; A61P 37/04

(52) U.S. Cl. CPC A61K 39/215; A61K 39/0015; A61P 37/04; A61K 2039/5254

(57) ABSTRACT

Disclosed herein is a live attenuated recombinant measles virus (rMeV)-based coronavirus vaccine containing a SARS-CoV-2 spike (S) protein that has at least one mutation to remove a glycosylation site. In some embodiments, the rMeVs-based coronavirus vaccine contains full-length stabilized pre-fusion and native S proteins, S proteins of SARS-CoV-2 variants, truncated S proteins lacking its transmembrane and cytoplasmic domains, S proteins lacking glycosylation sites, the monomeric and trimeric receptor-binding domain (RBD), the monomeric and trimeric S1 protein, Fc-fused RBD, or Fc-fused S1 protein. Also disclosed is a live attenuated recombinant coronavirus vaccine, wherein a stabilized prefusion spike (S) protein is inserted into a viral vector genome.

### CROSS-REFERENCE TO RELATED APPLICATIONS

This application is a National Stage of International Application No. PCT/US2021/029373, filed Apr. 27, 2021, which claims benefit of U.S. Provisional Application No. 63/016,184, filed Apr. 27, 2020, and U.S. Provisional Application No. 63/134,111, filed Jan. 5, 2021, which are hereby incorporated herein by reference in its entirety.

### SEQUENCE LISTING

This application contains a sequence listing filed in electronic form as an ASCII.txt file entitled “321501-2430 Sequence Listing_ST25” created on Feb. 1, 2023 and having 217,259 bytes. The content of the sequence listing is incorporated herein in its entirety.

### BACKGROUND

The current pandemic of severe acute respiratory syndrome coronavirus 2 (SARS-CoV-2) is causing tremendous economical, emotional, and public health burdens. There is an urgent need to develop a safe and efficacious vaccine to protect the populace from this new virus.

### SUMMARY

Disclosed herein is a live attenuated recombinant measles virus (rMeV)-based coronavirus vaccine containing a SARS-CoV-2 spike (S) protein that has at least one mutation to remove a glycosylation site. In some embodiments, the rMeVs-based coronavirus vaccine contains full-length stabilized pre-fusion and native S proteins, S proteins of SARS-CoV-2 variants, truncated S proteins lacking its transmembrane and cytoplasmic domains, S proteins lacking glycosylation sites, the monomeric and trimeric receptor-binding domain (RBD), the monomeric and trimeric S1 protein, Fc-fused RBD, or Fc-fused S1 protein. Also disclosed is a live attenuated recombinant coronavirus vaccine, wherein a stabilized prefusion spike (S) protein is inserted into a viral vector genome.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B show optimization of the insertion location in the MeV genome. FIG. 1A shows recovery of rMeV expressing Zika virus prM-E and prM-E-NS1 at two different genome locations. The prM-E and prM-E-NS1 were inserted at two locations: the gene junction at P and M, and the gene junction at H and L genes. Recombinant viruses were recovered using reverse genetics system. Plaques of each recombinant virus was shown. The diagram of the gene insertion is shown. The organization of negative-sense measles virus genome is shown. Le, leader sequence; N, nucleocapsid gene; P, phosphoprotein gene; M, matrix protein gene; F, fusion protein gene; H, Hemagglutinin protein gene; L, large polymerase gene; Tr, trailer sequence. FIG. 1B shows comparison of E and NS1 protein expression at two different locations. Vero cells were infected by rMeV, rMeV-prM-E^{PM}, rMeV-prM-E^{HL}, rMeV-prM-E^{PM}, or rMeV-prM-E-NS1^{PM} at an MOI of 1.0. At 48 h post-infection, cell lysates were harvested and subjected to Western blot using Zika virus E or NS1 antibody. Results showed that Zika genes inserted at the gene junction between P and M had much higher protein expression than the Zika gene inserted at the gene junction between H and L in MeV genome. Thus, the junction between P and M is an optimal position to insert foreign antigens. β-actin was used as loading control.

FIG. 2 illustrates an example of a rapid method for construction of recombinant measles virus (rMeV) expressing SARS-CoV-2 antigens. Top panel: The SARS-CoV-2 S, S1, RBD1, and RBD2 were amplified from a codon optimized S gene of SARS-CoV-2 by PCR, and inserted at the gene junction between P and M in the genome of measles virus (MeV) Edmonston vaccine strain. Middle panel: The organization of negative-sense measles virus genome is shown. Le, leader sequence; N, nucleocapsid gene; P, phosphoprotein gene; M, matrix protein gene; F, fusion protein gene; H, Hemagglutinin protein gene; L, large polymerase gene; Tr, trailer sequence. The full-length cDNA of MeV with SARS-CoV-2 gene was inserted into pYES2 plasmid using six overlapping fragments (designated A to F). Lower panel: The map of plasmid pYES2 containing yeast origin was shown. After recombination in yeast, DNA was extracted and transformed into Top.10 E. coli. competent cells. Plasmid pYES2 expressing SARS-CoV-2 S, S1, RBD1, and RBD2 was constructed. Same strategy was used for construction of other S, S variants, modified S, and other SARS-CoV-2 structural, accessory, and nonstructural protein genes.

FIGS. 3A to 3C show clone verification for positive plasmids from yeast. FIG. 3A shows a schematic showing the cloning location of the SARS-CoV-2 (S, S1, RBD1, and RBD2) into the measles virus (MeV) genome. N=nucleoprotein; P=phosphoprotein; M=matrix protein; F=fusion protein; H=hemagglutinin; L=large polymerase protein (L divided into L1 and L2 for cloning purpose); T7=T7 RNA polymerase promoter; T7 term=T7 RNA polymerase terminator. FIG. 3B shows positive plasmids from yeast screened by PCR. Eight colonies of each construction were picked and cultured at 30° C. overnight. Plasmids were extracted from overnight yeast culture, first round PCR was conducted by using primers annealing to each insertion gene (S, S1 and RBD), respectively. All colonies are positive for insertion gene. FIG. 3C shows two of eight colonies chosen for the second round PCR screen. Primers annealing to each MeV gene or gene junctions were indicated as arrows in FIG. 3A, product fragments #1-5 were labelled accordingly to FIG. 3A and FIG. 3C. #5: PCR product for MeV contains S and S1, #5*: PCR product for MeV contains RBD.

FIG. 4 is a schematic of SARS-CoV-2 S primary structure. SP, signal peptide; RBM, receptor binding motif; RBD, receptor binding domain; FP, fusion peptide; HR1, heptad repeat 1; HR2; TM, transmembrane; CT, cytoplasmic tail.

FIGS. 5A and 5B show the trimer SARS-CoV-2 Spike protein decorated with 66 N-glycans (22 per monomer). Glycans are present in dynamic and essential regions and were chosen for removal to increase antigenicity of the protein. FIG. 5A shows a top view. RBD must swing up (open) to reveal the viral receptor. FIG. 5B shows a side view.

FIGS. 6A and 6B shows the plaque morphology of recombinant MeV expressing SARS-CoV-2 antigens. Recombinant MeVs were recovered from an infectious cDNA clone of Edmonston strain. Plaque morphology of each recombinant virus was shown. All plaques were developed after 5 days of incubation. The average diameter of a total of 20 plaques for each recombinant virus was shown.

FIG. 7 shows recombinant MeV expressing SARs-CoV-2 antigens grow to high titer in Vero cells. Confluent Vero cells were infected with each recombinant MeV at an MOI of 1.0. When maximum cytopathic effects (CPE) were observed (rMeV at 48 h and all other three recombinant viruses at 60 h), cell supernatants were harvested, and viral titer was determined by plaque assay.

FIGS. 8A to 8C show expression of SARS-CoV-2 antigens by the measles virus vector. FIG. 8A shows analysis of S and S1 protein expression in cell lysate by Western blotting. Vero cells were infected with rMeV, rMeV-S1, or rMeV at an MOI of 0.4. At 28 h post-infection, cells were lysed in 500 μl of lysis buffer, and 10 μl of lysate and cell culture supernatants (Super) was analyzed by SDS-PAGE and were blotted with anti-RBD protein monoclonal antibody. FIG. 8B shows analysis of RBD1 and RBD2 protein expression in cell lysate by Western blotting. Vero cells were infected with rMeV-RBD1, rMeV-RBD2, and rMeV at an MOI of 0.4. At 28 h post-infection, cells were lysed in 500 μl of lysis buffer, and 10 μl of lysate and cell culture supernatants (Super) was analyzed by SDS-PAGE and were blotted with anti-RBD protein monoclonal antibody. FIG. 8C shows expression of S, S1, RBD1, and RBD2 by pCI. 293 T cells in six-well-plate were transfected with 2 μg of pCI-S, pCI-S1, pCI-RBD1, pCI-RBD2, or pCI. Total cell lysates were harvested at 48 h post-transfection, and analyzed by Western blot using RBD antibody.

FIGS. 9A to 9F show recovery and characterization of rMeV expressing SARS-CoV-2 S proteins. FIG. 9A shows a strategy for insertion of SARS-CoV-2 S and its variants to MeV genome. The codon optimized full-length S, preS, S-dTM, S1, RBD1, RBD2, and RBD3 were amplified by PCR and inserted into the same position at the gene junction between P and M in the genome of the MeV Edmonston vaccine strain. The domain structure of S protein is shown: SP, signal peptide; RBD, receptor-binding domain; RBM, receptor-binding motif; FP, fusion peptide; HR, heptad repeat; CH, central helix; TM, transmembrane domain; CT, cytoplasmic tail. The organization of negative-sense MeV genome is shown. Le, leader sequence; N, nucleocapsid gene; P, phosphoprotein gene; M, matrix protein gene; F, fusion protein gene; H, Hemagglutinin protein gene; L, large polymerase gene; Tr, trailer sequence. FIG. 9B shows plaque morphology of rMeV expressing SARS-CoV-2 S antigens. All plaques were developed after 5 days of incubation in Vero CCL-81 cells. FIG. 9C is a multi-step growth curve. Confluent Vero CCL81 cells in 12-well-plates were infected with each virus at an MOI of 0.01. After 1 h of absorption, fresh DMEM with 2% FBS was added. The cell culture supernatants and cell lysates were harvested and combined, and virus titers were determined by plaque assay. Data are geometric mean titers (GMT)±standard deviation from n=3 biologically independent experiments. FIGS. 9D and 9E show analysis of SARS-CoV-2 S and S1 protein expression in cell lysate and supernatants by Western blot. Vero CCL81 cells in 12-well-plates were infected with each recombinant virus at an MOI of 0.01. At 72 h (FIG. 9D) or 96 h (FIG. 9E) post-infection, cells were lysed in 300 μl of lysis buffer, and 10 μl of lysate or supernatant was analyzed by SDS-PAGE and blotted with anti-SARS-CoV-2 S protein antibody (top), MeV N antibody (middle), or β-actin antibody (bottom). FIG. 9F shows analysis of RBD protein expression by Western blot. 10 μl of lysate or supernatant at 72 and 96 h post-infection was analyzed. Western blots shown are the representatives of three independent experiments.

FIGS. 10A to 10C show immunogenicity of rMeVs expressing SARS-CoV-2 antigens in cotton rats. FIG. 10A is an immunization schedule in cotton rats. Cotton rats (n=5) were inoculated subcutaneously with PBS or 4×10⁵ PFU of each of the rMeV-based vaccine candidate. Four weeks later, cotton rats were boosted with 10⁶ PFU of each virus. Serum samples were collected at weeks, 4, 6, and 8 for antibody detection. FIG. 9B shows measurement of SARS-CoV-2 S-specific antibody by ELISA. Highly purified preS protein was used as the coating antigen for the ELISA. Dot line indicates the detectable level at the lowest dilution. FIG. 9C shows measurement of SARS-CoV-2-specific neutralizing antibody. Antibody titer was determined by a plaque reduction neutralization assay. Dot line indicates the detectable level at the lowest dilution. Data are expressed as the geometric mean titers (GMT) of five cotton rats±standard deviation. Data were analyzed using Student's t-test (*P<0.05; **P<0.01; ***P<0.001; ****P<0.0001).

FIGS. 11A to 11E show rMeV-preS is highly immunogenic in IFNAR−/−-hCD46 mice and induces strong Th1-biased T cell immune responses. FIG. 11A shows immunization schedule. IFNAR^{-/-}hCD46 mice (n=5 or 6) were inoculated with 8×10⁵ PFU of rMeV, rMeV-preS, or rMeV-S1. Two weeks later, mice were boosted with 6×10⁵ PFU of each virus. Half dose was delivered subcutaneously and other half was delivered intranasally. Serum samples were collected at week 3 for antibody detection. Mice were terminated at week 3 for the T cell assays. FIG. 11B shows measurement of SARS-CoV-2 S-specific antibody by ELISA. Highly purified preS protein was used as the coating antigen for ELISA. Dot line indicates the detectable level at the lowest dilution. FIG. 9C shows ELISPOT quantification of IFNγ-producing T cells. Spot forming cells (SFC) were quantified after the cells were stimulated by peptides representing N (S1 peptides, red color) and C (S2 peptides, green color) termini of SARS-CoV-2 spike protein. Data are means of five mice±standard deviation. *P<0.05 as determined by unpaired t-test. FIGS. 11D and 11E show cytokine expression in CD8+ (FIG. 11D) and CD4+ (FIG. 11E) splenocytes. Splenocytes of four rMeV-preS vaccinated mice with highest SFC were stimulated ex vivo for 5 h with pools of S1 peptides representing the N-terminal of SARS-CoV-2 S protein (5 μg/ml each) in an intracellular cytokine staining assay. Frequencies of CD4+ T cells expressing cytokines represent CD4+ T cells expressing IFN-γ, TNF-α or IL-2. FIG. 11F shows flow plots of cytokine production. Antigen-stimulated CD8+ T cells in one rMeV vector immunized and four rMeV-preS immunized mice. CD8+ T cells expressing CD107a and IFN-γ are shown as red dots and cells also expressing TNF-α are shown as green dots.

FIGS. 12A to 12C show rMeV-preS is highly immunogenic in Golden Syrian hamsters. FIG. 12A shows immunization schedule in hamsters. 4-week-old female Golden Syrian hamsters (n=10) were immunized with 8×10⁵ PFU (half subcutaneous and half intranasal) of rMeV-preS, rMeV-S1, parental rMeV, or PBS. Hamsters were boosted 3 weeks later. At weeks 2, 4, and 6, sera were collected for antibody detection. At week 7, hamsters were challenged with 10⁵ PFU of SARS-CoV-2. Unimmunized unchallenged controls were inoculated with DMEM. FIG. 12B shows measurement of SARS-CoV-2 S-specific antibody. Highly purified preS protein was used as coating antigen for ELISA. Dot line indicates the detectable level at the lowest dilution. FIG. 12C shows measurement of SARS-CoV-2-specific neutralizing antibody. Antibody titer was determined by a plaque reduction neutralization assay. Human convalescent sera from acute infection (V1) and recovered COVID-19 patients (V2) were used as side-by-side controls. Data are expressed as the geometric mean titers (GMT) of 10 hamsters. Dot line indicates the detectable level at the lowest dilution. Data were analyzed using two-way ANOVA and Student's t-test (*P<0.05; **P<0.01; ***P<0.001; ****P<0.0001).

FIGS. 13A to 13M show rMeV-preS provides complete protection against SARS-CoV-2 challenge in Golden Syrian hamsters. FIG. 13A shows dynamics of hamster body weight changes after SARS-CoV-2 challenge. The body weight for each hamster was measured daily and expressed as percentage of body weight at the challenge day. From days 0-4, the average body weight of 10 hamsters (n=10) in each group was shown. From days 5-12, the average body weight of 5 hamsters (n=5) in each group was shown. SARS-CoV-2 titer in lungs (FIG. 13B) and nasal turbinate (FIG. 13C). At day 4 after challenge, 5 hamsters from each group were sacrificed, and lungs and nasal turbinates were collected for virus titration by plaque assay. At day 12, the remaining 5 hamsters in each group were terminated. Viral titers are the geometric mean titer (GMT) of 5 animals±standard deviation. The limit of detection (LoD) is 2.7-2.8 Log 10 PFU per gram of tissue (dotted line). SARS-CoV-2 genome RNA copies in lungs (FIG. 13D), nasal turbinate (FIG. 13E), brain (FIG. 13F), liver (FIG. 13G), and spleen (FIG. 13H). Total RNA was extracted from the homogenized tissue using Trizol reagent. SARS-CoV-2 genome copies were quantified by real-time RT-PCR using primers annealing to the 5′-end of genome. SARS-CoV-2 subgenomic RNA copies in lungs (FIG. 13I), nasal turbinate (FIG. 13J), brain (FIG. 13K), liver (FIG. 13L), and spleen (FIG. 13M). SARS-CoV-2 subgenomic RNA copies were quantified by real-time RT-PCR using primers annealing to the N gene at the 3′ end of the genome. Black bars were shown as GMT of 5 hamsters in each group. Dot line indicates the detection limit. Data were analyzed using Student's t-test (*P<0.05; **P<0.01; ***P<0.001; ****P<0.0001).

FIGS. 14A to 14G show rMeV-preS immunization prevents a cytokine storm in the lungs. Total RNA was extracted from lungs of hamsters terminated at day 4 after challenge with SARS-CoV-2. Hamster IFN-αl (FIG. 14A), IFN-γ (FIG. 14B), IL-1b (FIG. 14C), IL-2 (FIG. 14D), IL-6 (FIG. 14E), TNF (FIG. 14F), and CXCL10 (FIG. 14G) mRNAs were quantified by real-time RT-PCR. GAPDH mRNA was used as an internal control. Data are shown as fold-change in gene expression compared to normal animals (unimmunized and unchallenged) after normalization. * indicates P<0.05; ** indicates P<0.01; *** indicates P<0.001; **** indicates P<0.0001.

FIG. 15 shows lung pathology score after challenge with SARS-CoV-2. Fixed lung tissues from days 4 and 12 after SARS-CoV-2 challenge were embedded in paraffin, sectioned at 5 μm, deparaffinized, rehydrated, and stained with hematoxylin-eosin (HE) for the examination of histological changes by light microscopy. Each slide was quantified based on the severity of histologic changes including inflammation, interstitial pneumonia, edema, alveolitis, bronchiolitis, alveolar destruction, mononuclear cell infiltration, pulmonary hemorrhage, and peribronchiolar inflammation. Score 4=extremely severe lung pathological changes; score 3=severe lung pathological changes; score 2=moderate lung pathological changes; score 1=mild lung pathological changes; and score 0=no pathological changes.

FIG. 16 shows rMeV-preS immunization protects lung pathology. Hematoxylin-eosin (HE) staining of lung tissue of hamsters euthanized at day 4 after SARS-CoV-2 challenge is shown. Micrographs with ×1, ×2, ×4, and ×10 magnification of representative lung section from each group are shown. Scale bars are indicated at the left corner of each image.

FIG. 17 shows rMeV-preS immunization prevents SARS-CoV-2 antigen expression in lungs. Immunohistochemistry (IHC) staining of lung sections from hamsters euthanized at day 4 after SARS-CoV-2 challenge is shown. Lung sections were stained with SARS-CoV-2 N antibody. Micrographs with ×1, ×2, ×4, and ×10 magnification of representative lung section from each group are shown. Scale bars are indicated at the left corner of each image.

FIG. 18 shows a rapid method for construction of recombinant measles virus (rMeV) expressing SARS-CoV-2 antigens. Top panel: The SARS-CoV-2 S, preS, S-dTM, S1, RBD1, RBD2, and RBD3 were amplified from a codon optimized S gene of SARS-CoV-2 by PCR, and inserted at the gene junction between P and M in the genome of measles virus (MeV) Edmonston vaccine strain. Middle panel: The organization of negative-sense measles virus genome is shown. Le, leader sequence; N, nucleocapsid gene; P, phosphoprotein gene; M, matrix protein gene; F, fusion protein gene; H, hemagglutinin protein gene; L, large polymerase gene; Tr, trailer sequence. The plasmid pYES2 vector was modified to insert a yeast replication origin, a T7 RNA polymerase promoter, a hepatitis delta virus (HDV) ribozyme sequence, and a T7 terminator. The full-length cDNA clone of MeV with SARS-CoV-2 S gene was constructed using six overlapping fragments (designated from a to f) by using DNA recombinase in yeasts. Low panel: The map of plasmid pYES2 containing the yeast origin is shown. After recombination in yeast, DNA was extracted and transformed into Top.10 E. coli. competent cells. Plasmid pYES2 expressing SARS-CoV-2 S, preS, S-dTM, S1, RBD1, RBD2, and RBD3 was constructed.

FIG. 19. Recombinant MeV expressing SARS-CoV-2 antigens exhibit delayed syncytia formation and cytopathic effects (CPE). Confluent Vero CCL81 cells in 12-well-plates were infected with individual virus at an MOI of 0.01. After 1 h of absorption, fresh DMEM with 2% FBS was added. Representative images of syncytia and CPE from each virus-infected cells were captured by light microscope at the indicated time points. Images are the representatives of three independent experiments.

FIGS. 20A and 20B show a single immunization of rMeV-preS induces a strong antibody response in IFNAR^{-/-} mice. FIG. 20A is an immunization schedule. 4-week-old IFNAR^{-/-} mice female IFNAR1^{-/-} mice were randomly divided into 3 groups (n=5, or 6). Mice in groups 1 were immunized with 8×10⁵ PFU of rMeV-preS (half subcutaneous and half intranasal). Mice in group 2 were immunized with 8×10⁵ PFU of rMeV-preS (half subcutaneous and half intranasal) and were boosted at the same dose at the same route 4 weeks later. Mice in group 3 were immunized with 8×10⁵ PFU of rMeV and served as controls. At weeks 7 and 8, blood samples were collected from each mouse by facial vein bleeding, and the serum was isolated for detection of S-specific antibody by ELISA. FIG. 20B shows measurement of SARS-CoV-2 S-specific antibody by ELISA. Highly purified preS protein was used as the coating antigen for the ELISA. Dot line indicates the detectable level at the lowest dilution. Data are expressed as the geometric mean titers (GMT) of 5 or 6 mice±standard deviation. Data were analyzed using Student's t-test (**P<0.01; ns indicates no significant difference).

FIG. 21 shows histology of lung sections at day 12. Hematoxylin-eosin (HE) staining of lung tissue of hamsters euthanized at day 12 after SARS-CoV-2 challenge is shown. Micrographs with ×1, ×2, ×4, and ×10 magnification of representative lung section are shown. Scale bars are indicated at the left corner of each image.

FIG. 22 shows immunohistochemistry (IHC) staining of lung sections at day 12. IHC analysis of lung sections from hamsters euthanized at day 12 after SARS-CoV-2 challenge is shown. Lung sections were stained with SARS-CoV-2 N antibody. Micrographs with ×1, ×2, ×4, and ×10 magnification of representative lung section are shown. Scale bars are indicated at the left corner of each image.

FIGS. 23A to 23E show recovery and characterization of rMeV expressing SARS-CoV-2 preS-HexaPro and its variants. FIG. 23A shows a strategy for insertion of SARS-CoV-2 preS-HexaPro and its variants to MeV genome. The codon optimized preS-HexaPro and its variants were amplified by PCR and inserted into the gene junction between P and M in the genome of the MeV Edmonston vaccine strain. The domain structure of S protein is shown: SP, signal peptide; RBD, receptor-binding domain; RBM, receptor-binding motif; FP, fusion peptide; HR, heptad repeat; CH, central helix; TM, transmembrane domain; CT, cytoplasmic tail. preS-HexaPro contains 6 prolines (K986P, V987P, F817P, A892P, A899P, A942P) whereas preS-2P contains only two prolines (K986P, V987P). The organization of negative-sense MeV genome is shown. Le, leader sequence; N, nucleocapsid gene; P, phosphoprotein gene; M, matrix protein gene; F, fusion protein gene; H, Hemagglutinin protein gene; L, large polymerase gene; Tr, trailer sequence. FIG. 23B shows plaque morphology of rMeV expressing preS-HexaPro and preS (with 2 Prolines). Plaques were developed after 4 days of incubation in Vero CCL-81 cells. FIG. 23C shows the expression of preS-HexaPro by rMeV vector. Vero cells in six-well-plate were infected by rMeV-preS-HexaPro or rMeV at an MOI of 1.0. At 72 h post-infection, 10 μl of cell culture supernatants was used for Western blot analysis using antibody against S protein. preS-HexaPro protein was detected in supernatants of rMeV-preS-HexaPro-infected cells but not rMeV-infected cells. FIG. 23D shows the comparison of immunogenicity of rMeVs expressing preS-HexaPro and preS in IFNAR1 knockout mice. An immunization schedule in mice is shown. Mice (n=5 or 6) were inoculated subcutaneously with high (2×10⁵ PFU) or low (4×10⁴ PFU) dose of the parental rMeV, rMeV-preS-HexaPro, or rMeV-preS vaccine candidate. Three weeks later, mice were boosted with each virus. Serum samples were collected at weeks 5 or 7 for antibody detection. FIG. 23E shows measurement of SARS-CoV-2 S-specific antibody by ELISA. Highly purified preS protein was used as the coating antigen for the ELISA. Data are expressed as the geometric mean titers (GMT) of five or six mice±standard deviation. Data were analyzed using Student's t-test (*P<0.05; **P<0.01; ***P<0.001; ****P<0.0001). Results showed that rMeV-preS-HexaPro triggered a higher SARS-CoV-2 specific antibody response than rMeV-preS at both high and low doses. This result also showed that rMeV-preS-HexaPro is a better vaccine candidate compared to rMeV-preS.

FIGS. 24A to 24C show recovery and characterization of rMeV expressing RBD-trimer, S1-trimer, RBD-Fc, and S1-Fc. FIG. 24A shows a strategy for insertion of RBD-trimer, S1-trimer, RBD-Fc, and S1-Fc to MeV genome. The codon optimized version of each gene were amplified by PCR and inserted into the gene junction between P and M in the genome of the MeV Edmonston vaccine strain. The domain structure of S protein is shown: SP, signal peptide; RBD, receptor-binding domain; RBM, receptor-binding motif. The foldon and Fc fusion peptides were fused to RBD and S1 protein. The organization of negative-sense MeV genome is shown. Le, leader sequence; N, nucleocapsid gene; P, phosphoprotein gene; M, matrix protein gene; F, fusion protein gene; H, Hemagglutinin protein gene; L, large polymerase gene; Tr, trailer sequence. FIG. 24B shows plaque morphology of rMeV expressing RBD trimer and S1 trimer. Plaques were developed after 4 days of incubation in Vero CCL-81 cells. FIG. 24C showed expression of monomeric and trimeric RBD by rMeV. Vero cells in six-well-plate were infected by rMeV-RBD2, rMeV-RBD-trimer, or rMeV at an MOI of 1.0. At 72 h post-infection, 10 μl of cell lysate was used for Western blot analysis using antibody against S protein.

FIGS. 25A to 25E show recovery and characterization of rMeV expressing structural proteins (M, N, and E), accessory proteins (ORF3a and ORFS) and nonstructural protein nsp6. FIG. 25A shows a strategy for insertion of M, N, E, ORF3a, ORF8, and nsp6 to MeV genome. Each gene were amplified by PCR and inserted into the gene junction between P and M in the genome of the MeV Edmonston vaccine strain. The organization of negative-sense MeV genome is shown. Le, leader sequence; N, nucleocapsid gene; P, phosphoprotein gene; M, matrix protein gene; F, fusion protein gene; H, Hemagglutinin protein gene; L, large polymerase gene; Tr, trailer sequence. FIG. 25B shows the strategy to construct MeV co-expressing S and X (X designates one or more structural, accessary, and nonstructural proteins) in the same location. An IRES (or 2A protease) will be inserted between the S and X genes, resulting in translation of an equal amount of S and X proteins. The S_{IRES}X cassette will be inserted at the junction between P and M of MeV genome, and rMeV co-expressing S and X proteins will be recovered. FIG. 25C shows the strategy to construct MeV co-expressing S and X (X designates one or more structural, accessary, and nonstructural proteins) at two locations. the S and X genes can be inserted into two separate gene junctions in MeV vector. FIG. 25D shows plaque morphology of rMeV expressing M, N, or E. Plaques were developed after 4 days of incubation in Vero CCL-81 cells. FIG. 25E showed expression of SARS-CoV-2 N and M by rMeV. Vero cells in six-well-plate were infected by rMeV-M, rMeV-N, rMeV-E, or rMeV at an MOI of 1.0. At 72 h post-infection, 10 μl of cell lysate was used for Western blot analysis using antibody against N or M protein. E protein is a small protein and was detected by ELISA.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein. Unless otherwise noted, the term “vaccine immunogen” is used interchangeably with “protein antigen” or “immunogen polypeptide”.

The term “conservatively modified variant” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “epitope” refers to an antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

The term “effective amount” of a vaccine or other agent refers to an amount is sufficient to generate a desired response, such as reduce or eliminate a sign or symptom of a condition or disease, such as pneumonia. For instance, this can be the amount necessary to inhibit viral replication, or to measurably alter outward symptoms of the viral infection. In general, this amount will be sufficient to measurably inhibit virus (for example, SARS-CoV-2) replication or infectivity. When administered to a subject, a dosage will generally be used that will achieve target tissue concentrations that has been shown to achieve in vitro inhibition of viral replication. In some embodiments, an “effective amount” is one that treats (including prophylaxis) one or more symptoms and/or underlying causes of any of a disorder or disease, for example to treat a coronavirus infection. In some embodiments, an effective amount is a therapeutically effective amount. In some embodiments, an effective amount is an amount that prevents one or more signs or symptoms of a particular disease or condition from developing, such as one or more signs or symptoms associated with coronaviral infections.

The term “immunogen” refers to a protein or a portion thereof that is capable of inducing an immune response in a mammal, such as a mammal infected or at risk of infection with a pathogen. Administration of an immunogen can lead to protective immunity and/or proactive immunity against a pathogen of interest.

The term “immunogenic composition” refers to a composition comprising an immunogenic polypeptide that induces a measurable CTL response against virus expressing the immunogenic polypeptide, or induces a measurable B cell response (such as production of antibodies) against the immunogenic polypeptide.

The term “percent (%) sequence identity” or “homology” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “vaccine” refers to a pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In some embodiments of the invention, vaccines or vaccine immunogens or vaccine compositions are expressed from fusion constructs and self-assemble into nanoparticles displaying an immunogen polypeptide or protein on the surface.

Recombinant Measles Virus-Based Coronavirus Vaccine Disclosed herein is a live attenuated recombinant measles virus (rMeV)-based coronavirus vaccine. Therefore, disclosed herein is a recombinant measles virus expressing epitopes of one or more coronavirus antigens, such as a SARS-CoV-2 spike (S) protein, stabilized prefusion S, truncated S, modified S, S variants, RBD, S1, trimerized RBD, trimerized S1, Fc-fused RBD, Fc-fused S1, other structural proteins including M, N, and E, accessary proteins ORF3a and ORFS, and nonstructural protein nsp6.

Recombinant Measles Virus

A recombinant measles viruses for expressing epitopes of antigens of RNA viruses is described in U.S. Pat. No. 9,914,937 to Tangy et al., which is incorporated by reference in its entirety for the teaching of recombinant measles viruses that can be adapted for use in the disclosed compositions and methods.

Measles virus (MeV) is a member of the order mononegavirales, i.e., viruses with a non-segmented negative-strand RNA genome. The non-segmented genome of measles virus has an anti-message polarity which results in a genomic RNA which is not translated either in vivo or in vitro nor infectious when purified. Transcription and replication of non-segmented (−) strand RNA viruses and their assembly as virus particles have been studied and reported especially in Fields virology (3^{rd} edition, vol. 1, 1996, Lippincott—Raven publishers—Fields B N et al). Transcription and replication of measles virus do not involve the nucleus of the infected cells but rather take place in the cytoplasm of said infected cells. The genome of the measles virus comprises genes encoding six major structural proteins from the six genes (designated N, P, M, F, H and L) and an additional two-non structural proteins from the P gene. The gene order is the following: 3′-N-P-M-F-H-L-5′. The genome further comprises non-coding regions in the intergenic region M/F; this non-coding region contains approximately 1000 nucleotides of untranslated RNA. The cited genes respectively encode the proteins of the nucleocapsid of the virus, i.e., the nucleoprotein (N), the phosphoprotein (P), and the large protein (L) which assemble around the genome RNA to provide the nucleocapsid. The other genes encode the proteins of the viral envelope including the hemagglutinin (H), the fusion (F) and the matrix (M) proteins.

The measles virus has been isolated and live attenuated vaccines have been derived from the Edmonston MeV isolated in 1954 (Enders, J. F., et al. Proc. Soc. Exp. Biol. Med. 1954. 86:277-286.), by serial passages on primary human kidney or amnion cells. The used strains were then adapted to chick embryo fibroblasts (CEF) to produce Edmonston A and B seeds (Griffin, D., et al. Measles virus, 1996. p. 1267-1312. In B. Fields, D. Knipe, et al. (ed.), Virology, vol. 2. Lippincott—Raven Publishers, Philadelphia). Edmonston B was licensed in 1963 as the first MV vaccine. Further passages of Edmonston A and B on CEF produced the more attenuated Schwarz and Moraten viruses (Griffin, D., et al.) whose sequences have recently been shown to be identical (Parks, C. L., et al. J Virol. 2001 75:921-933; Parks, C. L., et al. J Virol. 2001 75:910-920). Because Edmonston B vaccine was reactogenic, it was abandoned in 1975 and replaced by the Schwarz/Moraten vaccine which is currently the most widely used measles vaccine in the world (Hilleman, M. Vaccine. 2002 20:651-665). Several other vaccine strains are also used: AIK-C, Schwarz F88, CAM70, TD97 in Japan, Leningrad-16 in Russia, and Edmonston Zagreb. The CAM70 and TD97 Chinese strains were not derived from Edmonston. Schwarz/Moraten and AIK-C vaccines are produced on CEF. Zagreg vaccine is produced on human diploid cells (WI-38).

In some embodiments, the genome sequence of measles virus Edmonston vaccine strain is:

(SEQ ID NO: 1)
ACCAAACAAAGTTGGGTAAGGATAGTTCAATCAATGATCATCTTCTAGTGC
ACTTAGGATTCAAGATCCTATTATCAGGGACAAGAGCAGGATTAGGGATAT
CCGAGATGGCCACACTTTTAAGGAGCTTAGCATTGTTCAAAAGAAACAAGG
ACAAACCACCCATTACATCAGGATCCGGTGGAGCCATCAGAGGAATCAAAC
ACATTATTATAGTACCAATCCCTGGAGATTCCTCAATTACCACTCGATCCA
GACTTCTGGACCGGTTGGTCAGGTTAATTGGAAACCCGGATGTGAGCGGGC
CCAAACTAACAGGGGCACTAATAGGTATATTATCCTTATTTGTGGAGTCTC
CAGGTCAATTGATTCAGAGGATCACCGATGACCCTGACGTTAGCATAAGGC
TGTTAGAGGTTGTCCAGAGTGACCAGTCACAATCTGGCCTTACCTTCGCAT
CAAGAGGTACCAACATGGAGGATGAGGCGGACCAATACTTTTCACATGATG
ATCCAATTAGTAGTGATCAATCCAGGTTCGGATGGTTCGAGAACAAGGAAA
TCTCAGATATTGAAGTGCAAGACCCTGAGGGATTCAACATGATTCTGGGTA
CCATCCTAGCCCAAATTTGGGTCTTGCTCGCAAAGGCGGTTACGGCCCCAG
ACACGGCAGCTGATTCGGAGCTAAGAAGGTGGATAAAGTACACCCAACAAA
GAAGGGTAGTTGGTGAATTTAGATTGGAGAGAAAATGGTTGGATGTGGTGA
GGAACAGGATTGCCGAGGACCTCTCCTTACGCCGATTCATGGTCGCTCTAA
TCCTGGATATCAAGAGAACACCCGGAAACAAACCCAGGATTGCTGAAATGA
TATGTGACATTGATACATATATCGTAGAGGCAGGATTAGCCAGTTTTATCC
TGACTATTAAGTTTGGGATAGAAACTATGTATCCTGCTCTTGGACTGCATG
AATTTGCTGGTGAGTTATCCACACTTGAGTCCTTGATGAACCTTTACCAGC
AAATGGGGGAAACTGCACCCTACATGGTAATCCTGGAGAACTCAATTCAGA
ACAAGTTCAGTGCAGGATCATACCCTCTGCTCTGGAGCTATGCCATGGGAG
TAGGAGTGGAACTTGAAAACTCCATGGGAGGTTTGAACTTTGGCCGATCTT
ACTTTGATCCAGCATATTTTAGATTAGGGCAAGAGATGGTAAGGAGGTCAG
CTGGAAAGGTCAGTTCCACATTGGCATCTGAACTCGGTATCACTGCCGAGG
ATGCAAGGCTTGTTTCAGAGATTGCAATGCATACTACTGAGGACAAGATCA
GTAGAGCGGTTGGACCCAGACAAGCCCAAGTATCATTTCTACACGGTGATC
AAAGTGAGAATGAGCTACCGAGATTGGGGGGCAAGGAAGATAGGAGGGTCA
AACAGAGTCGAGGAGAAGCCAGGGAGAGCTACAGAGAAACCGGGCCCAGCA
GAGCAAGTGATGCGAGAGCTGCCCATCTTCCAACCGGCACACCCCTAGACA
TTGACACTGCATCGGAGTCCAGCCAAGATCCGCAGGACAGTCGAAGGTCAG
CTGACGCCCTGCTTAGGCTGCAAGCCATGGCAGGAATCTCGGAAGAACAAG
GCTCAGACACGGACACCCCTATAGTGTACAATGACAGAAATCTTCTAGACT
AGGTGCGAGAGGCCGAGGACCAGAACAACATCCGCCTACCCTCCATCATTG
TTATAAAAAACTTAGGAACCAGGTCCACACAGCCGCCAGCCCATCAACCAT
CCACTCCCACGATTGGAGCAGATGGCAGAAGAGCAGGCACGCCATGTCAAA
AACGGACTGGAATGCATCCGGGCTCTCAAGGCCGAGCCCATCGGCTCACTG
GCCATCGAGGAAGCTATGGCAGCATGGTCAGAAATATCAGACAACCCAGGA
CAGGAGCGAGCCACCTGCAGGGAAGAGAAGGCAGGCAGTTCGGGTCTCAGC
AAACCATGCCTCTCAGCAATTGGATCAACTGAAGGCGGTGCACCTCGCATC
CGCGGTCAGGGACCTGGAGAGAGCGATGACGACGCTGAAACTTTGGGAATC
CCCCCAAGAAATCTCCAGGCATCAAGCACTGGGCTACAGTGTCATTATGTT
TATGATCACAGCGGTGAAGCGGTTAAGGGAATCCAAGATGCTGACTCTATC
ATGGTTCAATCAGGCCTTGATGGTGATAGCACCCTCTCAGGAGGAGACAAT
GAATCTGAAAACAGCGATGTGGATATTGGCGAACCTGATACCGAGGGATAT
GCTATCACTGACCGGGGATCTGCTCCCATCTCTATGGGGTTCAGGGCTTCT
GATGTTGAAACTGCAGAAGGAGGGGAGATCCACGAGCTCCTGAGACTCCAA
TCCAGAGGCAACAACTTTCCGAAGCTTGGGAAAACTCTCAATGTTCCTCCG
CCCCCGGACCCCGGTAGGGCCAGCACTTCCGGGACACCCATTAAAAAGGGC
ACAGACGCGAGATTAGCCTCATTTGGAACGGAGATCGCGTCTTCATTGACA
GGTGGTGCAACCCAATGTGCTCGAAAGTCACCCTCGGAACCATCAGGGCCA
GGTGCACCTGCGGGGAACGTCCCCGAGTGTGTGAGCAATGCCGCACTGATA
CAGGAGTGGACACCCGAATCTGGCACCACAATCTCCCCGAGATCCCAGAAT
AATGAAGAAGGGGGAGACCATTATGATGATGAGCTGTTCTCTGATGTCCAA
GATATTAAAACAGCCTTGGCCAAAATACACGAGGATAATCAGAAGATAATC
TCCAAGCTAGAATCACTGCTGTTATTGAAGGGAGAAGTTGAGTCAATTAAG
AAGCAGATCAACAGGCAAAATATCAGCATATCCACCCTGGAAGGACACCTC
TCAAGCATCATGATCGCCATTCCTGGACTTGGGAAGGATCCCAACGACCCC
ACTGCAGATGTCGAAATCAATCCCGACTTGAAACCCATCATAGGCAGAGAT
TCAGGCCGAGCACTGGCCGAAGTTCTCAAGAAACCCGTTGCCAGCCGACAA
CTCCAAGGAATGACAAATGGACGGACCAGTTCCAGAGGACAGCTGCTGAAG
GAATTTCAGCTAAAGCCGATCGGGAAAAAGATGAGCTCAGCCGTCGGGTTT
GTTCCTGACACCGGCCCTGCATCACGCAGTGTAATCCGCTCCATTATAAAA
TCCAGCCGGCTAGAGGAGGATCGGAAGCGTTACCTGATGACTCTCCTTGAT
GATATCAAAGGAGCCAATGATCTTGCCAAGTTCCACCAGATGCTGATGAAG
ATAATAATGAAGTAGCTACAGCTCAACTTACCTGCCAACCCCATGCCAGTC
GACCCATCTAGTACAACCTAAATCCATTATAAAAAACTTAGGAGCAAAGTG
ATTGCCTCCCAAGTTCCACAATGACAGAGATCTACGACTTCGACAAGTCGG
CATGGGACATCAAAGGGTCGATCGCTCCGATACAACCCACCACCTACAGTG
ATGGCAGGCTGGTGCCCCAGGTCAGAGTCATAGATCCTGGTCTAGGCGACA
GGAAGGATGAATGCTTTATGTACATGTTTCTGCTGGGGGTTGTTGAGGACA
GCGATCCCCTAGGGCCTCCAATCGGGCGAGCATTTGGGTCCCTGCCCTTAG
GTGTTGGCAGATCCACAGCAAAGCCCGAAAAACTCCTCAAAGAGGCCACTG
AGCTTGACATAGTTGTTAGACGTACAGCAGGGCTCAATGAAAAACTGGTGT
TCTACAACAACACCCCACTAACTCTCCTCACACCTTGGAGAAAGGTCCTAA
CAACAGGGAGTGTCTTCAACGCAAACCAAGTGTGCAATGCGGTTAATCTGA
TACCGCTCGATACCCCGCAGAGGTTCCGTGTTGTTTATATGAGCATCACCC
GTCTTTCGGATAACGGGTATTACACCGTTCCTGGAAGAATGCTGGAATTCA
GATCGGTCAATGCAGTGGCCTTCAACCTGCTGGTGACCCTTAGGATTGACA
AGGCGATAGGCCCTGGGAAGATCATCGACAATACAGAGCAACTTCCTGAGG
CAACATTTATGGTCCACATCGGGAACTTCAGGAGAAAGAAGAGTGAAGTCT
ACTCTGCCGATTATTGCAAAATGAAAATCGAAAAGATGGGCCTGGTTTTTG
CACTTGGTGGGATAGGGGGCACCAGTCTTCACATTAGAAGCACAGGCAAAA
TGAGCAAGACTCTCCATGCACAACTCGGGTTCAAGAAGACCTTATGTTACC
CGCTGATGGATATCAATGAAGACCTTAATCGATTACTCTGGAGGAGCAGAT
GCAAGATAGTAAGAATCCAGGCAGTTTTGCAGCCATCAGTTCCTCAAGAAT
TCCGCATTTACGACGACGTGATCATAAATGATGACCAAGGACTATTCAAAG
TTCTGTAGACCGTAGTGCCCAGCAATGCCCGAAAACGACCCCCCTCACAAT
GACAGCCAGAAGGCCCGGACAAAAAAGCCCCCTCCGAAAGACTCCACGGAC
CAAGCGAGAGGCCAGCCAGCAGCCGACGGCAAGCGCGAACACCAGGCGGCC
CCAGCACAGAACAGCCCTGACACAAGGCCACCACCAGCCACCCCAATCTGC
ATCCTCCTCGTGGGACCCCCGAGGACCAACCCCCAAGGCTGCCCCCGATCC
AAACCACCAACCGCACCCCCACCACCCCCGGGAAAGAAACCCCCAGCAATT
GGAAGGCCCCTCCCCCTCTTCCTCAACACAAGAACTCCACAACCGAACCGC
ACAAGCGACCGAGGTGACCCAACCGCAGGCATCCGACTCCCTAGACAGATC
CTCTCTCCCCGGCAAACTAAACAAAACTTAGGGCCAAGGAACATACACACC
CAACAGAACCCAGACCCCGGCCCACGGCGCCGCGCCCCCAACCCCCGACAA
CCAGAGGGAGCCCCCAACCAATCCCGCCGGCTCCCCCGGTGCCCACAGGCA
GGGACACCAACCCCCGAACAGACCCAGCACCCAACCATCGACAATCCAAGA
CGGGGGGGCCCCCCCAAAAAAAGGCCCCCAGGGGCCGACAGCCAGCACCGC
GAGGAAGCCCACCCACCCCACACACGACCACGGCAACCAAACCAGAACCCA
GACCACCCTGGGCCACCAGCTCCCAGACTCGGCCATCACCCCGCAGAAAGG
AAAGGCCACAACCCGCGCACCCCAGCCCCGATCCGGCGGGGAGCCACCCAA
CCCGAACCAGCACCCAAGAGCGATCCCCGAAGGACCCCCGAACCGCAAAGG
ACATCAGTATCCCACAGCCTCTCCAAGTCCCCCGGTCTCCTCCTCTTCTCG
AAGGGACCAAAAGATCAATCCACCACACCCGACGACACTCAACTCCCCACC
CCTAAAGGAGACACCGGGAATCCCAGAATCAAGACTCATCCAATGTCCATC
ATGGGTCTCAAGGTGAACGTCTCTGCCATATTCATGGCAGTACTGTTAACT
CTCCAAACACCCACCGGTCAAATCCATTGGGGCAATCTCTCTAAGATAGGG
GTGGTAGGAATAGGAAGTGCAAGCTACAAAGTTATGACTCGTTCCAGCCAT
CAATCATTAGTCATAAAATTAATGCCCAATATAACTCTCCTCAATAACTGC
ACGAGGGTAGAGATTGCAGAATACAGGAGACTACTGAGAACTGTTTTGGAA
CCAATTAGAGATGCACTTAATGCAATGACCCAGAATATAAGACCGGTTCAG
AGTGTAGCTTCAAGTAGGAGACACAAGAGATTTGCGGGAGTAGTCCTGGCA
GGTGCGGCCCTAGGCGTTGCCACAGCTGCTCAGATAACAGCCGGCATTGCA
CTTCACCAGTCCATGCTGAACTCTCAAGCCATCGACAATCTGAGAGCGAGC
CTGGAAACTACTAATCAGGCAATTGAGGCAATCAGACAAGCAGGGCAGGAG
ATGATATTGGCTGTTCAGGGTGTCCAAGACTACATCAATAATGAGCTGATA
CCGTCTATGAACCAACTATCTTGTGATTTAATCGGCCAGAAGCTCGGGCTC
AAATTGCTCAGATACTATACAGAAATCCTGTCATTATTTGGCCCCAGCTTA
CGGGACCCCATATCTGCGGAGATATCTATCCAGGCTTTGAGCTATGCGCTT
GGAGGAGACATCAATAAGGTGTTAGAAAAGCTCGGATACAGTGGAGGTGAT
TTACTGGGCATCTTAGAGAGCAGAGGAATAAAGGCCCGGATAACTCACGTC
GACACAGAGTCCTACTTCATTGTCCTCAGTATAGCCTATCCGACGCTGTCC
GAGATTAAGGGGGTGATTGTCCACCGGCTAGAGGGGGTCTCGTACAACATA
GGCTCTCAAGAGTGGTATACCACTGTGCCCAAGTATGTCGCAACCCAAGGG
TACCTTATCTCGAATTTTGATGAGTCATCGTGTACTTTCATGCCAGAGGGA
ACTGTGTGCAGCCAAAATGCCTTGTACCCGATGAGTCCTCTGCTCCAAGAA
TGCCTCCGGGGGTCCACTAAGTCCTGTGCTCGTACACTCGTATCCGGGTCT
TTTGGGAACCGGTTCATTTTATCACAAGGGAACCTAATAGCCAATTGTGCA
TCAATCCTTTGCAAGTGTTACACAACAGGAACGATCATTAATCAAGACCCT
GACAAGATCCTAACATACATTGCTGCCGATCACTGCCCGGTAGTCGAGGTG
AACGGCGTGACCATCCAAGTCGGGAGCAGGAGGTATCCAGACGCTGTGTAC
TTGCACAGAATTGACCTCGGTCCTCCCATATCATTGGAGAGGTTGGACGTA
GGGACAAATCTGGGGAATGCAATTGCTAAGTTGGAGGATGCCAAGGAATTG
TTGGAGTCATCGGACCAGATATTGAGGAGTATGAAAGGTTTATCGAGCACT
AGCATAGTCTACATCCTGATTGCAGTGTGTCTTGGAGGGTTGATAGGGATC
CCCGCTTTAATATGTTGCTGCAGGGGGCGTTGTAATAAAAAGGGAGAACAA
GTTGGTATGTCAAGACCAGGCCTAAAGCCTGATCTTACGGGAACATCAAAA
TCCTATGTAAGGTCGCTCTGATCCTCTACAACTCTTGAAACACAAATGTCC
CACAAGTCTCCTCTTCGTCATCAAGCAACCACCGCACCCAGCATCAAGCCC
ACCTGAAATTATCTCCGGTTTCCCTCTGGCCGAACAATATCGGTAGTTAAT
TAAAACTTAGGGTGCAAGATCATCCACAATGTCACCACAACGAGACCGGAT
AAATGCCTTCTACAAAGATAACCCCCATCCCAAGGGAAGTAGGATAGTCAT
TAACAGAGAACATCTTATGATTGATAGACCTTATGTTTTGCTGGCTGTTCT
GTTTGTCATGTTTCTGAGCTTGATCGGGTTGCTAGCCATTGCAGGCATTAG
ACTTCATCGGGCAGCCATCTACACCGCAGAGATCCATAAAAGCCTCAGCAC
CAATCTAGATGTAACTAACTCAATCGAGCATCAGGTCAAGGACGTGCTGAC
ACCACTCTTCAAAATCATCGGTGATGAAGTGGGCCTGAGGACACCTCAGAG
ATTCACTGACCTAGTGAAATTCATCTCTGACAAGATTAAATTCCTTAATCC
GGATAGGGAGTACGACTTCAGAGATCTCACTTGGTGTATCAACCCGCCAGA
GAGAATCAAATTGGATTATGATCAATACTGTGCAGATGTGGCTGCTGAAGA
GCTCATGAATGCATTGGTGAACTCAACTCTACTGGAGACCAGAACAACCAA
TCAGTTCCTAGCTGTCTCAAAGGGAAACTGCTCAGGGCCCACTACAATCAG
AGGTCAATTCTCAAACATGTCGCTGTCCCTGTTAGACTTGTATTTAGGTCG
AGGTTACAATGTGTCATCTATAGTCACTATGACATCCCAGGGAATGTATGG
GGGAACTTACCTAGTGGAAAAGCCTAATCTGAGCAGCAAAAGGTCAGAGTT
GTCACAACTGAGCATGTACCGAGTGTTTGAAGTAGGTGTTATCAGAAATCC
GGGTTTGGGGGCTCCGGTGTTCCATATGACAAACTATCTTGAGCAACCAGT
CAGTAATGATCTCAGCAACTGTATGGTGGCTTTGGGGGAGCTCAAACTCGC
AGCCCTTTGTCACGGGGAAGATTCTATCACAATTCCCTATCAGGGATCAGG
GAAAGGTGTCAGCTTCCAGCTCGTCAAGCTAGGTGTCTGGAAATCCCCAAC
CGACATGCAATCCTGGGTCCCCTTATCAACGGATGATCCAGTGATAGACAG
GCTTTACCTCTCATCTCACAGAGGTGTTATCGCTGACAATCAAGCAAAATG
GGCTGTCCCGACAACACGAACAGATGACAAGTTGCGAATGGAGACATGCTT
CCAACAGGCGTGTAAGGGTAAAATCCAAGCACTCTGCGAGAATCCCGAGTG
GGCACCATTGAAGGATAACAGGATTCCTTCATACGGGGTCTTGTCTGTTGA
TCTGAGTCTGACAGTTGAGCTTAAAATCAAAATTGCTTCGGGATTCGGGCC
ATTGATCACACACGGTTCAGGGATGGACCTATACAAATCCAACCACAACAA
TGTGTATTGGCTGACTATCCCGCCAATGAAGAACCTAGCCTTAGGTGTAAT
CAACACATTGGAGTGGATACCGAGATTCAAGGTTAGTCCCTACCTCTTCAA
TGTCCCAATTAAGGAAGCAGGCGAAGACTGCCATGCCCCAACATACCTACC
TGCGGAGGTGGATGGTGATGTCAAACTCAGTTCCAATCTGGTGATTCTACC
TGGTCAAGATCTCCAATATGTTTTGGCAACCTACGATACTTCCAGGGTTGA
ACATGCTGTGGTTTATTACGTTTACAGCCCAAGCCGCTCATTTTCTTACTT
TTATCCTTTTAGGTTGCCTATAAAGGGGGTCCCCATCGAATTACAAGTGGA
ATGCTTCACATGGGACCAAAAACTCTGGTGCCGTCACTTCTGTGTGCTTGC
GGACTCAGAATCTGGTGGACATATCACTCACTCTGGGATGGTGGGCATGGG
AGTCAGCTGCACAGTCACCCGGGAAGATGGAACCAATCGCAGATAGGGCTG
CTAGTGAACCAATCACATGATGTCACCCAGACATCAGGCATACCCACTAGT
GTGAAATAGACATCAGAATTAAGAAAAACGTAGGGTCCAAGTGGTTCCCCG
TTATGGACTCGCTATCTGTCAACCAGATCTTATACCCTGAAGTTCACCTAG
ATAGCCCGATAGTTACCAATAAGATAGTAGCCATCCTGGAGTATGCTCGAG
TCCCTCACGCTTACAGCCTGGAGGACCCTACACTGTGTCAGAACATCAAGC
ACCGCCTAAAAAACGGATTTTCCAACCAAATGATTATAAACAATGTGGAAG
TTGGGAATGTCATCAAGTCCAAGCTTAGGAGTTATCCGGCCCACTCTCATA
TTCCATATCCAAATTGTAATCAGGATTTATTTAACATAGAAGACAAAGAGT
CAACGAGGAAGATCCGTGAACTCCTCAAAAAGGGGAATTCGCTGTACTCCA
AAGTCAGTGATAAGGTTTTCCAATGCTTAAGGGACACTAACTCACGGCTTG
GCCTAGGCTCCGAATTGAGGGAGGACATCAAGGAGAAAGTTATTAACTTGG
GAGTTTACATGCACAGCTCCCAGTGGTTTGAGCCCTTTCTGTTTTGGTTTA
CAGTCAAGACTGAGATGAGGTCAGTGATTAAATCACAAACCCATACTTGCC
ATAGGAGGAGACACACACCTGTATTCTTCACTGGTAGTTCAGTTGAGTTGC
TAATCTCTCGTGACCTTGTTGCTATAATCAGTAAAGAGTCTCAACATGTAT
ATTACCTGACATTTGAACTGGTTTTGATGTATTGTGATGTCATAGAGGGGA
GGTTAATGACAGAGACCGCTATGACTATTGATGCTAGGTATACAGAGCTTC
TAGGAAGAGTCAGATACATGTGGAAACTGATAGATGGTTTCTTCCCTGCAC
TCGGGAATCCAACTTATCAAATTGTAGCCATGCTGGAGCCTCTTTCACTTG
CTTACCTGCAGCTGAGGGATATAACAGTAGAACTCAGAGGTGCTTTCCTTA
ACCACTGCTTTACTGAAATACATGATGTTCTTGACCAAAACGGGTTTTCTG
ATGAAGGTACTTATCATGAGTTAATTGAAGCTCTAGATTACATTTTCATAA
CTGATGACATACATCTGACAGGGGAGATTTTCTCATTTTTCAGAAGTTTCG
GCCACCCCAGACTTGAAGCAGTAACGGCTGCTGAAAATGTTAGGAAATACA
TGAATCAGCCTAAAGTCATTGTGTATGAGACTCTGATGAAAGGTCATGCCA
TATTTTGTGGAATCATAATCAACGGCTATCGTGACAGGCACGGAGGCAGTT
GGCCACCGCTGACCCTCCCCCTGCATGCTGCAGACACAATCCGGAATGCTC
AAGCTTCAGGTGATGGGTTAACACATGAGCAGTGCGTTGATAACTGGAAAT
CTTTTGCTGGAGTGAAATTTGGCTGCTTTATGCCTCTTAGCCTGGATAGTG
ATCTGACAATGTACCTAAAGGACAAGGCACTTGCTGCTCTCCAAAGGGAAT
GGGATTCAGTTTACCCGAAAGAGTTCCTGCGTTACGACCCTCCCAAGGGAA
CCGGGTCACGGAGGCTTGTAGATGTTTTCCTTAATGATTCGAGCTTTGACC
CATATGATGTGATAATGTATGTTGTAAGTGGAGCTTACCTCCATGACCCTG
AGTTCAACCTGTCTTACAGCCTGAAAGAAAAGGAGATCAAGGAAACAGGTA
GACTTTTTGCTAAAATGACTTACAAAATGAGGGCATGCCAAGTGATTGCTG
AAAATCTAATCTCAAACGGGATTGGCAAATATTTTAAGGACAATGGGATGG
CCAAGGATGAGCACGATTTGACTAAGGCACTCCACACTCTAGCTGTCTCAG
GAGTCCCCAAAGATCTCAAAGAAAGTCACAGGGGGGGGCCAGTCTTAAAAA
CCTACTCCCGAAGCCCAGTCCACACAAGTACCAGGAACGTGAGAGCAGCAA
AAGGGTTTATAGGGTTCCCTCAAGTAATTCGGCAGGACCAAGACACTGATC
ATCCGGAGAATATGGAAGCTTACGAGACAGTCAGTGCATTTATCACGACTG
ATCTCAAGAAGTACTGCCTTAATTGGAGATATGAGACCATCAGCTTGTTTG
CACAGAGGCTAAATGAGATTTACGGATTGCCCTCATTTTTCCAGTGGCTGC
ATAAGAGGCTTGAGACCTCTGTCCTGTATGTAAGTGACCCTCATTGCCCCC
CCGACCTTGACGCCCATATCCCGTTATATAAAGTCCCCAATGATCAAATCT
TCATTAAGTACCCTATGGGAGGTATAGAAGGGTATTGTCAGAAGCTGTGGA
CCATCAGCACCATTCCCTATCTATACCTGGCTGCTTATGAGAGCGGAGTAA
GGATTGCTTCGTTAGTGCAAGGGGACAATCAGACCATAGCCGTAACAAAAA
GGGTACCCAGCACATGGCCCTACAACCTTAAGAAACGGGAAGCTGCTAGAG
TAACTAGAGATTACTTTGTAATTCTTAGGCAAAGGCTACATGATATTGGCC
ATCACCTCAAGGCAAATGAGACAATTGTTTCATCACATTTTTTTGTCTATT
CAAAAGGAATATATTATGATGGGCTACTTGTGTCCCAATCACTCAAGAGCA
TCGCAAGATGTGTATTCTGGTCAGAGACTATAGTTGATGAAACAAGGGCAG
CATGCAGTAATATTGCTACAACAATGGCTAAAAGCATCGAGAGAGGTTATG
ACCGTTACCTTGCATATTCCCTGAACGTCCTAAAAGTGATACAGCAAATTC
TGATCTCTCTTGGCTTCACAATCAATTCAACCATGACCCGGGATGTAGTCA
TACCCCTCCTCACAAACAACGACCTCTTAATAAGGATGGCACTGTTGCCCG
CTCCTATTGGGGGGATGAATTATCTGAATATGAGCAGGCTGTTTGTCAGAA
ACATCGGTGATCCAGTAACATCATCAATTGCTGATCTCAAGAGAATGATTC
TCGCCTCACTAATGCCTGAAGAGACCCTCCATCAGGTAATGACACAACAAC
CGGGGGACTCTTCATTCCTAGACTGGGCTAGCGACCCTTACTCAGCAAATC
TTGTATGTGTCCAGAGCATCACTAGACTCCTCAAGAACATAACTGCAAGGT
TTGTCCTGATCCATAGTCCAAACCCAATGTTAAAAGGATTATTCCATGATG
ACAGTAAAGAAGAGGACGAGGGACTGGCGGCATTCCTCATGGACAGGCATA
TTATAGTACCTAGGGCAGCTCATGAAATCCTGGATCATAGTGTCACAGGGG
CAAGAGAGTCTATTGCAGGCATGCTGGATACCACAAAAGGCTTGATTCGAG
CCAGCATGAGGAAGGGGGGTTTAACCTCTCGAGTGATAACCAGATTGTCCA
ATTATGACTATGAACAATTCAGAGCAGGGATGGTGCTATTGACAGGAAGAA
AGAGAAATGTCCTCATTGACAAAGAGTCATGTTCAGTGCAGCTGGCGAGAG
CTCTAAGAAGCCATATGTGGGCGAGGCTAGCTCGAGGACGGCCTATTTACG
GCCTTGAGGTCCCTGATGTACTAGAATCTATGCGAGGCCACCTTATTCGGC
GTCATGAGACATGTGTCATCTGCGAGTGTGGATCAGTCAACTACGGATGGT
TTTTTGTCCCCTCGGGTTGCCAACTGGATGATATTGACAAGGAAACATCAT
CCTTGAGAGTCCCATATATTGGTTCTACCACTGATGAGAGAACAGACATGA
AGCTTGCCTTCGTAAGAGCCCCAAGTCGATCCTTGCGATCTGCTGTTAGAA
TAGCAACAGTGTACTCATGGGCTTACGGTGATGATGATAGCTCTTGGAACG
AAGCCTGGTTGTTGGCTAGGCAAAGGGCCAATGTGAGCCTGGAGGAGCTAA
GGGTGATCACTCCCATCTCAACTTCGACTAATTTAGCGCATAGGTTGAGGG
ATCGTAGCACTCAAGTGAAATACTCAGGTACATCCCTTGTCCGAGTGGCGA
GGTATACCACAATCTCCAACGACAATCTCTCATTTGTCATATCAGATAAGA
AGGTTGATACTAACTTTATATACCAACAAGGAATGCTTCTAGGGTTGGGTG
TTTTAGAAACATTGTTTCGACTCGAGAAAGATACCGGATCATCTAACACGG
TATTACATCTTCACGTCGAAACAGATTGTTGCGTGATCCCGATGATAGATC
ATCCCAGGATACCCAGCTCCCGCAAGCTAGAGCTGAGGGCAGAGCTATGTA
CCAACCCATTGATATATGATAATGCACCTTTAATTGACAGAGATGCAACAA
GGCTATACACCCAGAGCCATAGGAGGCACCTTGTGGAATTTGTTACATGGT
CCACACCCCAACTATATCACATTTTAGCTAAGTCCACAGCACTATCTATGA
TTGACCTGGTAACAAAATTTGAGAAGGACCATATGAATGAAATTTCAGCTC
TCATAGGGGATGACGATATCAATAGTTTCATAACTGAGTTTCTGCTCATAG
AGCCAAGATTATTCACTATCTACTTGGGCCAGTGTGCGGCCATCAATTGGG
CATTTGATGTACATTATCATAGACCATCAGGGAAATATCAGATGGGTGAGC
TGTTGTCATCGTTCCTTTCTAGAATGAGCAAAGGAGTGTTTAAGGTGCTTG
TCAATGCTCTAAGCCACCCAAAGATCTACAAGAAATTCTGGCATTGTGGTA
TTATAGAGCCTATCCATGGTCCTTCACTTGATGCTCAAAACTTGCACACAA
CTGTGTGCAACATGGTTTACACATGCTATATGACCTACCTCGACCTGTTGT
TGAATGAAGAGTTAGAAGAGTTCACATTTCTCTTGTGTGAAAGCGACGAGG
ATGTAGTACCGGACAGATTCGACAACATCCAGGCAAAACACTTATGTGTTC
TGGCAGATTTGTACTGTCAACCAGGGACCTGCCCACCAATTCAAGGTCTAA
GACCGGTAGAGAAATGTGCAGTTCTAACCGACCATATCAAGGCAGAGGCTA
TGTTATCTCCAGCAGGATCTTCGTGGAACATAAATCCAATTATTGTAGACC
ATTACTCATGCTCCCTGACTTATCTCCGGCGAGGATCGATCAAACAGATAA
GATTGAGAGTTGATCCAGGATTCATTTTCGACGCCCTCGCTGAGGTAAATG
TCAGTCAGCCAAAGATCGGCAGCAACAACATCTCAAATATGAGCATCAAGG
CTTTCAGACCCCCACACGATGATGTTGCAAAATTGCTCAAAGATATCAACA
CAAGCAAGCACAATCTTCCCATTTCAGGGGGCAATCTCGCCAATTATGAAA
TCCATGCTTTCCGCAGAATCGGGTTGAACTCATCTGCTTGCTACAAAGCTG
TTGAGATATCAACATTAATTAGGAGATGCCTTGAGCCAGGGGAGGACGGCT
TGTTCTTGGGTGAGGGATCGGGTTCTATGTTGATCACTTATAAGGAGATAC
TTAAACTAAGCAAGTGCTTCTATAATAGTGGGGTTTCCGCCAATTCTAGAT
CTGGTCAAAGGGAATTAGCACCCTATCCCTCCGAAGTTGGCCTTGTCGAAC
ACAGAATGGGAGTAGGTAATATTGTCAAAGTGCTCTTTAACGGGAGGCCCG
AAGTCACGTGGGTAGGCAGTGTAGATTGCTTCAATTTCATAGTTAGTAATA
TCCCTACCTCTAGTGTGGGGTTTATCCATTCAGATATAGAGACCTTGCCTG
ACAAAGATACTATAGAGAAGCTAGAGGAATTGGCAGCCATCTTATCGATGG
CTCTGCTCCTGGGCAAAATAGGATCAATACTGGTGATTAAGCTTATGCCTT
TCAGCGGGGATTTTGTTCAGGGATTTATAAGTTATGTAGGGTCTCATTATA
GAGAAGTGAACCTTGTATACCCTAGATACAGCAACTTCATATCTACTGAAT
CTTATTTGGTTATGACAGATCTCAAGGCTAACCGGCTAATGAATCCTGAAA
AGATTAAGCAGCAGATAATTGAATCATCTGTGAGGACTTCACCTGGACTTA
TAGGTCACATCCTATCCATTAAGCAACTAAGCTGCATACAAGCAATTGTGG
GAGACGCAGTTAGTAGAGGTGATATCAATCCTACTCTGAAAAAACTTACAC
CTATAGAGCAGGTGCTGATCAATTGCGGGTTGGCAATTAACGGACCTAAGC
TGTGCAAAGAATTGATCCACCATGATGTTGCCTCAGGGCAAGATGGATTGC
TTAATTCTATACTCATCCTCTACAGGGAGTTGGCAAGATTCAAAGACAACC
AAAGAAGTCAACAAGGGATGTTCCACGCTTACCCCGTATTGGTAAGTAGCA
GGCAACGAGAACTTATATCTAGGATCACCCGCAAATTTTGGGGGCACATTC
TTCTTTACTCCGGGAACAGAAAGTTGATAAATAAGTTTATCCAGAATCTCA
AGTCCGGCTATCTGATACTAGACTTACACCAGAATATCTTCGTTAAGAATC
TATCCAAGTCAGAGAAACAGATTATTATGACGGGGGGTTTGAAACGTGAGT
GGGTTTTTAAGGTAACAGTCAAGGAGACCAAAGAATGGTATAAGTTAGTCG
GATACAGTGCCCTGATTAAGGACTAATTGGTTGAACTCCGGAACCCTAATC
CTGCCCTAGGTGGTTAGGCATTATTTGCAATATATTAAAGAAAACTTTGAA
AATACGAAGTTTCTATTCCCAGCTTTGTCTGGT.

The live attenuated vaccine derived from the Schwarz strain is commercialized by Aventis Pasteur (Lyon France) under the trademark ROUVAX®.

An infectious cDNA corresponding to the antigenome of Edmonston MeV was cloned and an original and efficient reverse genetics procedure established to rescue the corresponding virus (Radecke, F., et al., EMBO Journal. 1995 14:5773-5784) and WO 97/06270. An Edmonston vector for the expression of foreign genes was developed (Radecke, F., et al. Reviews in Medical Virology. 1997 7:49-63) with a large capacity of insertion (as much as 5 kb) and high stability at expressing transgenes (Singh, M., et al. J. Gen. Virol. 1999 80:101-106; Singh, M., et al. J. Virol. 1999 73:4823-4828; Spielhofer, P., et al. J. Virol. 1998 72:2150-2159; Wang, Z., et al. Vaccine. 2001 19:2329-2336. This vector was cloned from the Edmonston B strain of MeV propagated in HeLa cells (Ballart, I., et al. Embo J. 1990 9:379-384). In addition, recombinant measles virus expressing Hepatitis B virus surface antigen has been produced and shown to induce humoral immune responses in genetically modified mice (Singh M. R. et al, J. Virol. 1999 73:4823-4828).

MV vaccine induces a very efficient, life-long immunity after a single low-dose injection (10⁴ TCID₅₀). Protection is mediated both by antibodies and by CD4+ and CD8+ T cells. The MeV genome is very stable and reversion to pathogenicity has never been observed with this vaccine. MeV replicates exclusively in the cytoplasm, ruling out the possibility of integration in host DNA. Furthermore, an infectious cDNA clone corresponding to the anti-genome of the Edmonston strain of MeV and a procedure to rescue the corresponding virus have been established. This cDNA has been made into a vector to express foreign genes. It can accommodate up to 5 kb of foreign DNA and is genetically very stable.

From the observation that the properties of the measles virus and especially its ability to elicit high titers of neutralizing antibodies in vivo and its property to be a potent inducer of long lasting cellular immune response, it may be a good candidate for the preparation of compositions comprising recombinant infectious viruses expressing antigenic peptides or polypeptides of a coronavirus, including SARS CoV-2.

Therefore, disclosed herein is a recombinant measles virus expressing a heterologous coronavirus amino acid sequence antigen capable of eliciting a humoral and/or a cellular immune response against the heterologous amino acid sequence including in individuals having pre-existing measles virus immunity.

Nucleic acid sequences of Measles viruses have been disclosed in International Patent Application WO 98/13501, which is incorporated by reference herein for the teaching of these sequences. In order to produce recombinant measles viruses, a rescue system was developed for the Edmonston MeV strain and described in International Patent Application WO 97/06270, which is incorporated by reference herein for the teaching of this rescue system and viruses.

The expression “heterologous amino acid sequence” is directed to an amino acid sequence which is not derived from the antigens of measles viruses, said heterologous amino acid sequence being accordingly derived from a coronavirus.

The heterologous amino acid sequence expressed in recombinant measles viruses is one that it is capable of eliciting a humoral and/or cellular immune response in a subject against the coronavirus. Accordingly, this amino acid sequence is one which comprises at least one epitope of an antigen, especially a conserved epitope, which epitope is exposed naturally on the antigen or is obtained or exposed as a result of a mutation or modification or combination of antigens.

In some embodiments, the disclosed recombinant measles virus also elicits a humoral and/or cellular immune response against measles virus. In some embodiments, the disclosed recombinant measles virus is derived from the Edmonston strain of measles virus. In some embodiments, the disclosed recombinant measles virus is derived from the Schwarz strain of measles virus.

Therefore, in some embodiments, the disclosed recombinant measles virus is recovered from helper cells transfected with a cDNA encoding the antigenomic RNA ((+)strand) of the measles virus, said cDNA being recombined with a nucleotide sequence encoding the heterologous coronavirus amino acid sequence.

The expression “encoding” in the above definition encompasses the capacity of the cDNA to allow transcription of a full length antigenomic (+)RNA, said cDNA serving especially as template for transcription. Accordingly, when the cDNA is a double stranded molecule, one of the strands has the same nucleotide sequence as the antigenomic (+) strand RNA of the measles virus, except that “U” nucleotides are substituted by “T” in the cDNA.

The helper cells according to the rescue system can be transfected with a transcription vector comprising the cDNA encoding the full length antigenomic (+)RNA of the measles virus, when said cDNA has been recombined with a nucleotide sequence encoding the heterologous coronavirus amino acid sequence and said helper cells are further transfected with an expression vector or several expression vectors providing the helper functions including those enabling expression of trans-acting proteins of measles virus, i.e., N, P and L proteins and providing expression of an RNA polymerase to enable transcription of the recombinant cDNA and replication of the corresponding viral RNA.

In some embodiments, the disclosed recombinant measles virus is suitable to elicit neutralizing antibodies against the heterologous coronavirus amino acid sequence in a mammalian animal model susceptible to measles virus. In some embodiments, the disclosed recombinant measles virus elicits neutralizing antibodies against the heterologous coronavirus amino acid sequence in a mammal, with a titre of at least 1/40000 when measured in ELISA, and a neutralizing titre of at least 1/20.

Also disclosed herein is a recombinant measles virus nucleotide sequence comprising a replicon comprising (i) a cDNA sequence encoding the full length antigenomic (+)RNA of measles virus operatively linked to (ii) an expression control sequence and (iii) a heterologous DNA sequence coding for a heterologous coronavirus amino acid sequence, said heterologous DNA sequence being cloned in said replicon in conditions allowing its expression and in conditions not interfering with transcription and replication of said cDNA sequence, said replicon having a total number of nucleotides which is a multiple of six.

The “rule of six” is expressed in the fact that the total number of nucleotides present in the recombinant cDNA resulting from recombination of the cDNA sequence derived from reverse transcription of the antigenomic RNA of measles virus, and the heterologous DNA sequence finally amount to a total number of nucleotides which is a multiple of six, a rule which allows efficient replication of genome RNA of the measles virus.

In some embodiments, the heterologous DNA sequence is cloned within an Additional Transcription Unit (ATU) inserted in the cDNA corresponding to the antigenomic RNA of measles virus. The location of the ATU within the cDNA derived from the antigenomic RNA of the measles virus can vary along said cDNA. It is however located in such a site that it will benefit from the expression gradient of the measles virus.

This gradient corresponds to the mRNA abundance according to the position of the gene relative to the 3′ end of the template. Accordingly, when the polymerase operates on the template (either genomic and anti-genomic RNA or corresponding cDNAs), it synthesizes more RNA made from upstream genes than from downstream genes. This gradient of mRNA abundance is however relatively smooth for measles virus. Therefore, the ATU or any insertion site suitable for cloning of the heterologous DNA sequence can be spread along the cDNA, with a preferred embodiment for an insertion site and especially in an ATU, present in the N-terminal portion of the sequence and especially within the region upstream from the L-gene of the measles virus and advantageously upstream from the M gene of said virus and more preferably upstream from the N gene of said virus.

Depending on the expression site and the expression control of the heterologous DNA, the disclosed vector can allow the expression of the heterologous amino acid sequence as a fusion protein with one of the measles virus proteins. Alternatively, the insertion site of the DNA sequence in the cDNA of the measles virus can be chosen in such a way that the heterologous DNA expresses the heterologous amino acid sequence in a form which is not a fusion protein with one of the proteins of the measles virus.

The recombinant measles virus vector can be a plasmid. Example vectors obtained with the nucleotide sequence of the Edmonston B. strain include pMeV2(EdB)gp160[delta]V3HIV89.6P CNCM I-2883; pMeV2(EdB)gp160HIV89.6P CNCM I-2884; pMeV2(EdB)gp140HIV89.6P CNCM I-2885; pMV3(EdB)gp140[delta]V3HIV89.6P CNCM I-2886; pMV2(EdB)-NS1YFV17D CNCM I-2887; and pMV2(EdB)-EnvYFV17D CNCM I-2888. Example vectors obtained with the nucleotide sequence of the Schwarz strain include: pTM-MVSchw2-Es(WNV) CNCM I-3033; pTM-MVSchw2-GFPbis-CNCM I-3034; pTM-MVSchw2-p17p24[delta]myr(HIVB) CNCM I-3035; pTM-MVSchw3-Tat(HIV89-6p) CNCM I-3036; pTM-MVschw3-GFP CNCM I-3037; pTM-MVSchw2-Es (YFV) CNCM I-3038; pTM-MVSchw2-gp140 [delta] V1 V2 V3 (HIV89-6) CNCM I-3054; pTM-MVSchw2-gp140 [delta] V3 (HIV89-6) CNCM I-3055; pTM-MVSchw2-gp160 [delta] V1 V2 V3 (HIV89-6) CNCM I-3056; pTM-MVSchw2-gp160 [delta] V1 V2 (HIV89-6) CNCM I-3057; and pTM-MVSchw2-Gag SIV239 p17-p24 [delta] myr-3-gp140 (HIV89-6) CNCM I-3058.

I-2883 (pMV2(EdB)gp160[delta]V3HIV89.6P) is a plasmid derived from Bluescript containing the complete sequence of the measles virus (Edmonston strain B), under the control of the T7 RNA polymerase promoter and containing the gene of the gp160ΔV3+ELDKWAS of the virus SVIH strain 89.6P inserted in an ATU at position 2 (between the N and P genes of measles virus). The size of the plasmid is 21264 nt.

I-2884 (pMV2(EdB)gp160HIV89.6P) is a plasmid derived from Bluescript containing the complete sequence of the measles virus (Edmonston strain B), under the control of the T7 RNA polymerase promoter and containing the gene of the gp160 of the SVIH virus strain 89.6P inserted in an ATU at position 2 (between the N and P genes of measles virus). The size of the plasmid is 21658 nt.

I-2885 (pMV2(EdB)gp140HIV89.6P) is a plasmid derived from Bluescript containing the complete sequence of the measles virus (Edmonston strain B), under the control of the T7 RNA polymerase promoter and containing the gene of the gp140 of the SVIH virus strain 89.6P inserted in an ATU at position 2 (between the N and P genes of measles virus). The size of the plasmid is 21094 nt.

I-2886 (pMV3(EdB)gp140[delta]V3HIV89.6P) is a plasmid derived from Bluescript containing the complete sequence of the measles virus (Edmonston strain B), under the control of the T7 RNA polymerase promoter and containing the gene of the gp140ΔV3 (ELDKWAS; residues 3-9 of SEQ ID NO: 8) of the SVIH virus strain 89.6P inserted in an ATU at position 2 (between the N and P genes of measles virus). The size of the plasmid is 21058 nt.

I-2887 (pMV2(EdB)-NS1YFV17D) is a plasmid derived from Bluescript containing the complete sequence of the measles virus (Edmonston strain B), under the control of the T7 RNA polymerase promoter and containing the NS1 gene of the Yellow Fever virus (YFV 17D) inserted in an ATU at position 2 (between the N and P genes of measles virus). The size of the plasmid is 20163 nt.

I-2888 (pMV2(EdB)-EnvYFV17D) is a plasmid derived from Bluescript containing the complete sequence of the measles virus (Edmonston strain B), under the control of the T7 RNA polymerase promoter and containing the Env gene of the Yellow Fever virus (YFV 17D) inserted in an ATU at position 2 (between the N and P genes of measles virus). The size of the plasmid is 20505 nt.

I-3033 (pTM-MVSchw2-Es(WNV) is a plasmid derived from Bluescript containing a cDNA sequence of the complete infectious genome of the measles virus (Schwarz strain), under the control of the T7 RNA polymerase promoter and expressing the gene of the secreted envelope, (E) of the West Nile virus (WNV), inserted in an ATU.

I-3034 (pTM-MVSchw2-GFPbis) is a plasmid derived from Bluescript containing a cDNA sequence of the complete infectious genome of the measles virus (Schwarz strain), under the control of the T7 RNA polymerase promoter and expressing the gene of the GFP inserted in an ATU.

I-3035 (pTM-MVSchw2-p17p24[delta]myr(HIVB) is a plasmid derived from Bluescript containing a cDNA sequence of the complete infectious genome of the measles virus (Schwarz strain), under the control of the T7 RNA polymerase promoter and expressing the gene of the gag gene encoding p17p24Δmyrproteins of the HIVB virus inserted in an ATU.

I-3036 (pTMVSchw3-Tat(HIV89-6p) is a plasmid derived from Bluescript containing a cDNA sequence of the complete infectious genome of the measles virus (Schwarz strain), under the control of the T7 RNA polymerase promoter and expressing the gene of the Tat gene of the virus strain 89.6P inserted in an ATU.

I-3037 (pTM-MVSchw3-GFP) is a plasmid derived from Bluescript containing a cDNA sequence of the complete infectious genome of the measles virus (Schwarz strain) under the control of the T7 RNA polymerase promoter and expressing the gene of the GFP gene inserted in an ATU having a deletion of one nucleotide.

I-3038 (pTM-MVSchw2-Es) (YFV) is a plasmid derived from Bluescript containing a cDNA sequence of the complete infectious genome of the measles virus (Schwarz strain) under the control of the T7 RNA polymerase promoter and expressing the gene of the secreted protein of the Fever virus (YFV) inserted in an ATU.

I-3054 (pTM-MVSchw2-gp140 [delta] V1 V2 V3 (HIV89-6)) is a plasmid derived from Bluescript containing a cDNA sequence of the complete infectious genome of the measles virus (Schwarz strain), under the control of the T7 RNA polymerase promoter and expressing the gene encoding gp140 [delta] V1 V2 (HIV 89-6) inserted in an ATU.

I-3055 (pTM-MVSchw2-gp140 [delta] V3 (HIV89-6)) is a plasmid derived from Bluescript containing a cDNA sequence of the complete infectious genome of the measles virus (Schwarz strain), under the control of the T7 RNA polymerase promoter and expressing the gene encoding gp14 [delta] V3 (HIV 89-6) inserted in an ATU.

I-3056 (pTM-MVSchw2-gp160 [delta] V1 V2 V3 (HIV89-6)) is a plasmid derived from Bluescript containing a cDNA sequence of the complete infectious genome of the measles virus (Schwarz strain), under the control of the T7 RNA polymerase promoter and expressing the gene encoding gp160 [delta] V1 V2 V3 (HIV 89-6) inserted in an ATU.

I-3057 (pTM-MVSchw2-gp160 [delta] V1 V2 (HIV89-6)) is a plasmid derived from Bluescript containing a cDNA sequence of the complete infectious genome of the measles virus (Schwarz strain), under the control of the T7 RNA polymerase promoter and expressing the gene encoding gp160 [delta] V1 V2 (HIV 89-6) inserted in an ATU.

I-3058 (pTM-MVSchw2-Gag SIV239 p17-p24 [delta] myr-3-gp140 (HIV89-6)) is a plasmid derived from Bluescript containing a cDNA sequence of the complete infectious genome of the measles virus (Schwarz strain), under the control of the T7 RNA polymerase promoter and expressing the gene encoding Gag SIV239 p17-p24 [delta] myr-3-gp140 (HIV89-6) inserted in an ATU.

Also disclosed herein is a rescue system for the assembly of recombinant measles virus expressing a heterologous coronavirus amino acid sequence, which comprises a determined helper cell recombined with at least one vector suitable for expression of T7 RNA polymerase and expression of the N, P and L proteins of the measles virus transfected with a recombinant measles virus vector according to anyone of the definitions provided above. The disclosed recombinant viruses can also be produced in vivo by a live attenuated vaccine like MeV.

The disclosed recombinant virus can be associated with any appropriate adjuvant, or vehicle which may be useful for the preparation of immunogenic compositions.

Coronavirus Antigen

In some embodiments, the heterologous coronavirus amino acid (coronavirus antigen) used in the rMeV-based vaccine is a SARS-CoV-1, MERS-CoV, or SARS-CoV-2 protein. For SARS-CoV-1, MERS-CoV, and SARS-CoV-2, the viral genome encodes spike (S), envelope (E), membrane (M), and nucleocapsid (N) structural proteins, among which the S glycoprotein is responsible for binding the host receptor via the receptor-binding domain (RBD) in its S1 subunit, as well as the subsequent membrane fusion and viral entry driven by its S2 subunit.

As the S glycoprotein is surface-exposed and mediates entry into host cells, it is the main target of neutralizing antibodies (NAbs) upon infection and the focus of vaccine design. S trimers are extensively decorated with N-linked glycans that are important for proper folding and for modulating accessibility to NAbs.

Therefore, disclosed herein are engineered immunogen polypeptides that are derived or modified from the spike (S) glycoprotein of a coronavirus, such as SARS-CoV-1, MERS-CoV, or SARS-CoV-2.

In some embodiments, the disclosed rMeV-based vaccine contains a full-length S protein, i.e. both the S1 and S2 proteins. In some embodiments, the disclosed rMeV-based vaccine contains stabilized prefusion S with 2 Prolines or 6 Prolines. In some embodiments, the disclosed rMeV-based vaccine contains S proteins of SARS-CoV-2 variants. In some embodiments, the disclosed rMeV-based vaccine contains the S1 protein. In some embodiments, the disclosed rMeV-based vaccine contains a Receptor Binding Domain (RBD) of an S protein. In some embodiments, the disclosed rMeV-based vaccine contains truncated S proteins lacking its transmembrane and cytoplasmic domains. In some embodiments, the disclosed rMeV-based vaccine contains S proteins lacking glycosylation sites. In some embodiments, the disclosed rMeV-based vaccine contains Fc-fused or trimeric RBD and S1 proteins. In some embodiments, the disclosed rMeV-based vaccine contains structural proteins N, M and E proteins, accessory protein ORF3a and ORFS, and nonstructural protein nsp6. In some embodiments, the disclosed rMeV-based vaccine contains a combination of S and other structural proteins, accessory protein and nonstructural protein.

In some embodiments, the wildtype soluble S sequence of SARS-CoV-2 can have the amino acid sequence:

(SEQ ID NO: 2)
MFVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSS
SGWTAGAAAYYVGYLQPRTFLLKYNENGTITDAVDCALDPLSETKCTLKSF
TVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECD
IPIGAGICASYQTQTNSPRRARSVASQSIIAYTMSLGAENSVAYSNNSIAI
PTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPLLTD
EMIAQYTSALLAGTITSGWTFGAGAALQIPFAMQMAYRFNGIGVTQNVLYE
NQKLIANQFNSAIGKIQDSLSSTASALGKLQDVVNQNAQALNTLVKQLSSN
FGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQ
EKNFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISGIN
ASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT.

In some embodiments, the wildtype soluble S sequence of SARS-CoV-2 can be encoded by the nucleic acid sequence:

(SEQ ID NO: 3)
ATGTTTGTTTTCCTGGTGCTGCTGCCTCTGGTGTCTAGCCAGTGTGTCAAC
CTGACTACCCGAACCCAGCTGCCCCCTGCGTACACCAACTCCTTTACTAGG
GGTGTCTACTACCCCGACAAGGTCTTTCGGTCTTCTGTACTTCATTCTACA
CAAGATCTCTTCCTGCCCTTCTTCAGCAACGTGACATGGTTTCACGCCATC
CATGTGTCCGGGACCAATGGAACCAAGAGGTTCGACAACCCAGTGTTGCCA
TTCAACGATGGAGTCTACTTCGCTAGCACAGAGAAAAGCAACATTATAAGG
GGCTGGATCTTTGGCACCACTCTGGATAGCAAGACTCAGTCCCTCCTCATC
GTGAACAATGCTACCAACGTGGTTATTAAGGTGTGCGAATTTCAGTTTTGT
AATGATCCCTTCCTCGGGGTCTACTATCACAAAAATAATAAGTCCTGGATG
GAGTCCGAGTTCCGAGTTTATTCTTCCGCAAATAATTGCACATTTGAGTAT
GTTAGTCAGCCCTTCCTCATGGACCTCGAAGGCAAACAGGGAAATTTTAAG
AACCTTAGAGAATTCGTCTTCAAAAATATCGATGGGTATTTCAAGATTTAT
AGCAAGCATACACCCATTAACCTGGTACGAGACCTCCCTCAGGGCTTTTCC
GCACTTGAGCCTCTGGTCGATCTCCCGATAGGCATAAACATAACTAGGTTT
CAGACTTTGCTTGCCCTCCATAGGTCCTACCTTACGCCTGGGGATAGCAGC
AGTGGTTGGACTGCGGGCGCCGCCGCTTACTATGTTGGGTATCTTCAACCT
CGCACGTTCCTGCTTAAGTATAATGAAAATGGGACCATTACGGATGCCGTC
GATTGTGCGCTGGATCCTTTGTCCGAGACGAAATGTACCCTGAAAAGTTTC
ACCGTGGAGAAAGGGATTTATCAGACGAGCAATTTCCGCGTCCAGCCAACC
GAGTCCATTGTGCGGTTTCCGAACATCACAAACCTGTGTCCCTTCGGGGAG
GTGTTCAACGCCACACGGTTTGCTAGTGTTTACGCATGGAATCGAAAGCGA
ATTAGTAACTGCGTGGCAGACTACTCAGTTCTTTATAACAGTGCGAGCTTC
TCTACTTTTAAGTGCTACGGTGTGTCCCCCACCAAGCTTAACGACCTCTGT
TTTACCAACGTCTACGCCGATAGCTTCGTCATTAGGGGGGATGAAGTGAGA
CAGATCGCACCTGGCCAGACTGGGAAAATCGCCGATTACAACTACAAACTG
CCGGATGATTTCACAGGCTGCGTTATCGCTTGGAACAGTAATAACTTGGAC
TCAAAAGTGGGAGGGAACTATAATTATCTCTATCGACTGTTCAGAAAGTCT
AACCTGAAACCTTTTGAACGAGATATTAGTACCGAGATTTACCAAGCTGGC
AGTACACCGTGCAACGGCGTCGAGGGGTTCAATTGTTATTTTCCTCTTCAA
TCCTACGGTTTTCAGCCTACTAACGGAGTGGGATACCAGCCATACCGAGTG
GTTGTGCTGTCATTCGAGCTGCTGCACGCTCCAGCTACCGTCTGTGGCCCT
AAAAAGTCCACGAACCTGGTCAAGAACAAATGCGTGAATTTCAATTTCAAC
GGTCTCACCGGGACGGGCGTGCTTACGGAAAGCAACAAGAAATTCCTGCCA
TTCCAACAGTTCGGCCGGGACATCGCCGACACCACTGACGCGGTGCGCGAT
CCTCAGACATTGGAAATCCTTGACATTACTCCGTGCTCTTTCGGGGGAGTC
TCCGTCATCACCCCAGGAACTAACACGTCTAACCAGGTGGCTGTTCTGTAT
CAGGATGTTAATTGCACAGAAGTGCCCGTTGCGATACACGCGGACCAGCTT
ACTCCTACCTGGAGGGTCTATTCCACCGGCTCTAACGTCTTTCAGACCCGC
GCCGGTTGCCTCATAGGCGCAGAACATGTAAATAATAGTTATGAATGCGAC
ATTCCTATTGGCGCCGGGATCTGTGCGAGCTACCAGACTCAAACCAACAGC
CCGAGGCGGGCCAGGTCCGTGGCGAGTCAGTCCATTATTGCTTATACAATG
TCCCTGGGAGCAGAGAACTCTGTGGCTTACTCCAACAATAGCATAGCGATA
CCAACTAATTTTACTATATCCGTGACAACGGAGATCCTTCCCGTCTCCATG
ACAAAAACTAGTGTGGACTGCACAATGTATATCTGTGGCGATAGCACTGAA
TGCTCCAACCTTCTGCTGCAGTATGGGAGCTTTTGCACACAGTTGAACCGG
GCCCTGACCGGCATCGCCGTAGAGCAGGATAAAAATACACAGGAGGTGTTT
GCTCAGGTGAAGCAAATTTACAAGACCCCTCCTATTAAAGATTTCGGCGGG
TTTAATTTCAGCCAGATCCTGCCGGACCCATCCAAGCCGAGTAAGAGGAGC
TTTATCGAAGACTTGCTGTTCAACAAAGTGACTTTGGCTGATGCAGGCTTC
ATTAAGCAGTACGGTGATTGTCTCGGCGACATCGCAGCGCGGGATCTCATA
TGTGCTCAAAAGTTCAACGGTCTTACAGTACTCCCACCTCTGCTGACCGAC
GAGATGATTGCTCAGTACACGTCTGCCTTGCTGGCCGGGACGATCACCAGC
GGGTGGACCTTTGGCGCAGGCGCAGCTCTTCAAATCCCCTTTGCAATGCAG
ATGGCTTATCGATTTAACGGAATCGGGGTGACGCAGAACGTGTTGTATGAG
AACCAAAAATTGATAGCTAATCAATTCAATTCAGCGATAGGTAAGATTCAG
GACTCCCTGTCCAGCACCGCTTCCGCGTTGGGAAAATTGCAGGACGTGGTG
AATCAAAATGCACAAGCACTGAATACACTCGTGAAACAGCTGTCCAGTAAC
TTCGGGGCCATCTCCTCCGTGCTCAACGACATCCTTTCTAGACTTGATAAA
GTGGAAGCCGAAGTGCAGATTGATCGGCTTATCACAGGTCGCCTGCAGTCA
CTGCAGACTTACGTCACTCAACAGCTTATCCGCGCCGCAGAGATCAGAGCA
AGTGCCAATCTTGCCGCAACCAAAATGTCCGAGTGCGTGTTGGGACAGTCT
AAGCGAGTTGACTTTTGTGGTAAGGGGTACCACCTGATGAGTTTCCCCCAG
TCCGCCCCACACGGCGTGGTTTTTCTGCACGTGACTTATGTGCCAGCCCAG
GAGAAGAATTTTACTACCGCTCCGGCCATTTGCCACGACGGTAAGGCACAC
TTCCCTAGAGAGGGGGTTTTCGTGAGTAACGGCACTCACTGGTTTGTCACA
CAGCGGAATTTTTACGAGCCTCAGATCATCACTACAGATAACACTTTCGTG
TCCGGAAACTGCGACGTGGTTATCGGCATCGTCAACAATACCGTCTATGAT
CCTCTCCAGCCAGAACTGGACTCCTTTAAGGAAGAACTTGATAAATACTTC
AAGAATCACACAAGTCCAGATGTTGACCTTGGGGACATTAGCGGGATCAAT
GCAAGCGTAGTTAATATTCAGAAGGAGATTGACCGCCTCAACGAAGTGGCC
AAAAATCTCAATGAATCTCTTATTGATCTTCAGGAGCTCGGCAAGTACGAG
CAGTACATCAAGTGGCCCTGGTACATATGGCTCGGATTTATCGCCGGGTTG
ATCGCCATCGTGATGGTTACAATCATGCTGTGTTGCATGACATCATGTTGT
TCCTGCTTGAAAGGCTGCTGTAGCTGCGGGTCTTGTTGTAAATTCGATGAG
GATGATTCTGAGCCAGTACTGAAGGGCGTCAAGCTCCACTATACATGA.

In some embodiments, the SARS-CoV-2 S1 protein has the amino acid sequence:

(SEQ ID NO: 4)
MFVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSS
SGWTAGAAAYYVGYLQPRTFLLKYNENGTITDAVDCALDPLSETKCTLKSF
TVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECD
IPIGAGICASYQTQTNSPRRAR.

In some embodiments, the SARS-CoV-2 S1 protein is encoded by the nucleic acid sequence:

(SEQ ID NO: 5)
ATGTTTGTTTTCCTGGTGCTGCTGCCTCTGGTGTCTAGCCAGTGTGTCA
ACCTGACTACCCGAACCCAGCTGCCCCCTGCGTACACCAACTCCTTTAC
TAGGGGTGTCTACTACCCCGACAAGGTCTTTCGGTCTTCTGTACTTCAT
TCTACACAAGATCTCTTCCTGCCCTTCTTCAGCAACGTGACATGGTTTC
ACGCCATCCATGTGTCCGGGACCAATGGAACCAAGAGGTTCGACAACCC
AGTGTTGCCATTCAACGATGGAGTCTACTTCGCTAGCACAGAGAAAAGC
AACATTATAAGGGGCTGGATCTTTGGCACCACTCTGGATAGCAAGACTC
AGTCCCTCCTCATCGTGAACAATGCTACCAACGTGGTTATTAAGGTGTG
CGAATTTCAGTTTTGTAATGATCCCTTCCTCGGGGTCTACTATCACAAA
AATAATAAGTCCTGGATGGAGTCCGAGTTCCGAGTTTATTCTTCCGCAA
ATAATTGCACATTTGAGTATGTTAGTCAGCCCTTCCTCATGGACCTCGA
AGGCAAACAGGGAAATTTTAAGAACCTTAGAGAATTCGTCTTCAAAAAT
ATCGATGGGTATTTCAAGATTTATAGCAAGCATACACCCATTAACCTGG
TACGAGACCTCCCTCAGGGCTTTTCCGCACTTGAGCCTCTGGTCGATCT
CCCGATAGGCATAAACATAACTAGGTTTCAGACTTTGCTTGCCCTCCAT
AGGTCCTACCTTACGCCTGGGGATAGCAGCAGTGGTTGGACTGCGGGCG
CCGCCGCTTACTATGTTGGGTATCTTCAACCTCGCACGTTCCTGCTTAA
GTATAATGAAAATGGGACCATTACGGATGCCGTCGATTGTGCGCTGGAT
CCTTTGTCCGAGACGAAATGTACCCTGAAAAGTTTCACCGTGGAGAAAG
GGATTTATCAGACGAGCAATTTCCGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGTGGCCCTAAAAAGTCCACGAACCTGGTCAAGAACAAATGCGTG
AATTTCAATTTCAACGGTCTCACCGGGACGGGCGTGCTTACGGAAAGCA
ACAAGAAATTCCTGCCATTCCAACAGTTCGGCCGGGACATCGCCGACAC
CACTGACGCGGTGCGCGATCCTCAGACATTGGAAATCCTTGACATTACT
CCGTGCTCTTTCGGGGGAGTCTCCGTCATCACCCCAGGAACTAACACGT
CTAACCAGGTGGCTGTTCTGTATCAGGATGTTAATTGCACAGAAGTGCC
CGTTGCGATACACGCGGACCAGCTTACTCCTACCTGGAGGGTCTATTCC
ACCGGCTCTAACGTCTTTCAGACCCGCGCCGGTTGCCTCATAGGCGCAG
AACATGTAAATAATAGTTATGAATGCGACATTCCTATTGGCGCCGGGAT
CTGTGCGAGCTACCAGACTCAAACCAACAGCCCGAGGCGGGCCAGGTCC
GTGGCGAGTCAGTCCATTATTGCTTATACAATGTCCTGA.

In some embodiments, the SARS-CoV-2 S2 protein has the amino acid sequence:

(SEQ ID NO: 6)
SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRFNGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHY
T.

In some embodiments, the RBD of SARS-CoV-2 S protein has the amino acid sequence:

(SEQ ID NO: 7)
NITNLCPFGEVFNATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATV.

In some embodiments, the RBD comprises the amino acid sequence:

(SEQ ID NO: 124, RBD1)
MPMGSLQPLATLYLLGMLVASCLGRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATV.

Therefore, in some embodiments, the RBD is encoded by the nucleic acid sequence:

(SEQ ID NO: 8, RBD1)
atgcccatggggtctctgcaaccgctggccaccttgtacctgctgggaa
tgctggttgcttcctgcctcggaCGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGA.

In some embodiments, the RBD comprises the amino acid sequence:

(SEQ ID NO: 125, RBD2)
MPMGSLQPLATLYLLGMLVASCLGRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NF.

Therefore, some embodiments, the RBD is encoded by the nucleic acid sequence:

(SEQ ID NO: 9, RBD2)
atgcccatggggtctctgcaaccgctggccaccttgtacctgctgggaa
tgctggttgcttcctgcctcggaCGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGTGGCCCTAAAAAGTCCACGAACCTGGTCAAGAACAAATGCGTG
AATTTCTGA.

In some embodiments, the RBD comprises the amino acid sequence:

(SEQ ID NO: 126, RBD3)
MPMGSLQPLATLYLLGMLVASCLGRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
P.

In some embodiments, the RBD is encoded by the nucleic acid sequence:

(SEQ ID NO: 10, RBD3)
atgcccatggggtctctgcaaccgctggccaccttgtacctgctgggaa
tgctggttgcttcctgcctcggaCGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGTGGCCCTAAAAAGTCCACGAACCTGGTCAAGAACAAATGCGTG
AATTTCAATTTCAACGGTCTCACCGGGACGGGCGTGCTTACGGAAAGCA
ACAAGAAATTCCTGCCATTCCAACAGTTCGGCCGGGACATCGCCGACAC
CACTGACGCGGTGCGCGATCCTCAGACATTGGAAATCCTTGACATTACT
CCGTGA.

In some embodiments, the SARS-CoV-2 S protein has the amino acid sequence:

(SEQ ID NO: 11, S-d19C)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCC.

Therefore, in some embodiments, the SARS-CoV-2 S protein is encoded by the nucleic acid sequence:

(SEQ ID NO: 12, S-d19C)
ATGTTTGTTTTCCTGGTGCTGCTGCCTCTGGTGTCTAGCCAGTGTGTCA
ACCTGACTACCCGAACCCAGCTGCCCCCTGCGTACACCAACTCCTTTAC
TAGGGGTGTCTACTACCCCGACAAGGTCTTTCGGTCTTCTGTACTTCAT
TCTACACAAGATCTCTTCCTGCCCTTCTTCAGCAACGTGACATGGTTTC
ACGCCATCCATGTGTCCGGGACCAATGGAACCAAGAGGTTCGACAACCC
AGTGTTGCCATTCAACGATGGAGTCTACTTCGCTAGCACAGAGAAAAGC
AACATTATAAGGGGCTGGATCTTTGGCACCACTCTGGATAGCAAGACTC
AGTCCCTCCTCATCGTGAACAATGCTACCAACGTGGTTATTAAGGTGTG
CGAATTTCAGTTTTGTAATGATCCCTTCCTCGGGGTCTACTATCACAAA
AATAATAAGTCCTGGATGGAGTCCGAGTTCCGAGTTTATTCTTCCGCAA
ATAATTGCACATTTGAGTATGTTAGTCAGCCCTTCCTCATGGACCTCGA
AGGCAAACAGGGAAATTTTAAGAACCTTAGAGAATTCGTCTTCAAAAAT
ATCGATGGGTATTTCAAGATTTATAGCAAGCATACACCCATTAACCTGG
TACGAGACCTCCCTCAGGGCTTTTCCGCACTTGAGCCTCTGGTCGATCT
CCCGATAGGCATAAACATAACTAGGTTTCAGACTTTGCTTGCCCTCCAT
AGGTCCTACCTTACGCCTGGGGATAGCAGCAGTGGTTGGACTGCGGGCG
CCGCCGCTTACTATGTTGGGTATCTTCAACCTCGCACGTTCCTGCTTAA
GTATAATGAAAATGGGACCATTACGGATGCCGTCGATTGTGCGCTGGAT
CCTTTGTCCGAGACGAAATGTACCCTGAAAAGTTTCACCGTGGAGAAAG
GGATTTATCAGACGAGCAATTTCCGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGTGGCCCTAAAAAGTCCACGAACCTGGTCAAGAACAAATGCGTG
AATTTCAATTTCAACGGTCTCACCGGGACGGGCGTGCTTACGGAAAGCA
ACAAGAAATTCCTGCCATTCCAACAGTTCGGCCGGGACATCGCCGACAC
CACTGACGCGGTGCGCGATCCTCAGACATTGGAAATCCTTGACATTACT
CCGTGCTCTTTCGGGGGAGTCTCCGTCATCACCCCAGGAACTAACACGT
CTAACCAGGTGGCTGTTCTGTATCAGGATGTTAATTGCACAGAAGTGCC
CGTTGCGATACACGCGGACCAGCTTACTCCTACCTGGAGGGTCTATTCC
ACCGGCTCTAACGTCTTTCAGACCCGCGCCGGTTGCCTCATAGGCGCAG
AACATGTAAATAATAGTTATGAATGCGACATTCCTATTGGCGCCGGGAT
CTGTGCGAGCTACCAGACTCAAACCAACAGCCCGAGGCGGGCCAGGTCC
GTGGCGAGTCAGTCCATTATTGCTTATACAATGTCCCTGGGAGCAGAGA
ACTCTGTGGCTTACTCCAACAATAGCATAGCGATACCAACTAATTTTAC
TATATCCGTGACAACGGAGATCCTTCCCGTCTCCATGACAAAAACTAGT
GTGGACTGCACAATGTATATCTGTGGCGATAGCACTGAATGCTCCAACC
TTCTGCTGCAGTATGGGAGCTTTTGCACACAGTTGAACCGGGCCCTGAC
CGGCATCGCCGTAGAGCAGGATAAAAATACACAGGAGGTGTTTGCTCAG
GTGAAGCAAATTTACAAGACCCCTCCTATTAAAGATTTCGGCGGGTTTA
ATTTCAGCCAGATCCTGCCGGACCCATCCAAGCCGAGTAAGAGGAGCTT
TATCGAAGACTTGCTGTTCAACAAAGTGACTTTGGCTGATGCAGGCTTC
ATTAAGCAGTACGGTGATTGTCTCGGCGACATCGCAGCGCGGGATCTCA
TATGTGCTCAAAAGTTCAACGGTCTTACAGTACTCCCACCTCTGCTGAC
CGACGAGATGATTGCTCAGTACACGTCTGCCTTGCTGGCCGGGACGATC
ACCAGCGGGTGGACCTTTGGCGCAGGCGCAGCTCTTCAAATCCCCTTTG
CAATGCAGATGGCTTATCGATTTAACGGAATCGGGGTGACGCAGAACGT
GTTGTATGAGAACCAAAAATTGATAGCTAATCAATTCAATTCAGCGATA
GGTAAGATTCAGGACTCCCTGTCCAGCACCGCTTCCGCGTTGGGAAAAT
TGCAGGACGTGGTGAATCAAAATGCACAAGCACTGAATACACTCGTGAA
ACAGCTGTCCAGTAACTTCGGGGCCATCTCCTCCGTGCTCAACGACATC
CTTTCTAGACTTGATAAAGTGGAAGCCGAAGTGCAGATTGATCGGCTTA
TCACAGGTCGCCTGCAGTCACTGCAGACTTACGTCACTCAACAGCTTAT
CCGCGCCGCAGAGATCAGAGCAAGTGCCAATCTTGCCGCAACCAAAATG
TCCGAGTGCGTGTTGGGACAGTCTAAGCGAGTTGACTTTTGTGGTAAGG
GGTACCACCTGATGAGTTTCCCCCAGTCCGCCCCACACGGCGTGGTTTT
TCTGCACGTGACTTATGTGCCAGCCCAGGAGAAGAATTTTACTACCGCT
CCGGCCATTTGCCACGACGGTAAGGCACACTTCCCTAGAGAGGGGGTTT
TCGTGAGTAACGGCACTCACTGGTTTGTCACACAGCGGAATTTTTACGA
GCCTCAGATCATCACTACAGATAACACTTTCGTGTCCGGAAACTGCGAC
GTGGTTATCGGCATCGTCAACAATACCGTCTATGATCCTCTCCAGCCAG
AACTGGACTCCTTTAAGGAAGAACTTGATAAATACTTCAAGAATCACAC
AAGTCCAGATGTTGACCTTGGGGACATTAGCGGGATCAATGCAAGCGTA
GTTAATATTCAGAAGGAGATTGACCGCCTCAACGAAGTGGCCAAAAATC
TCAATGAATCTCTTATTGATCTTCAGGAGCTCGGCAAGTACGAGCAGTA
CATCAAGTGGCCCTGGTACATATGGCTCGGATTTATCGCCGGGTTGATC
GCCATCGTGATGGTTACAATCATGCTGTGTTGCATGACATCATGTTGTT
CCTGCTTGAAAGGCTGCTGTAGCTGCGGGTCTTGTTGTTGA.

In some embodiments, the SARS-CoV-2 S protein has the amino acid sequence:

(SEQ ID NO: 13, S-dTM)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQ.

Therefore, in some embodiments, the SARS-CoV-2 S protein is encoded by the nucleic acid sequence:

(SEQ ID NO:14, S-dTM)
ATGTTTGTTTTCCTGGTGCTGCTGCCTCTGGTGTCTAGCCAGTGTGTCA
ACCTGACTACCCGAACCCAGCTGCCCCCTGCGTACACCAACTCCTTTAC
TAGGGGTGTCTACTACCCCGACAAGGTCTTTCGGTCTTCTGTACTTCAT
TCTACACAAGATCTCTTCCTGCCCTTCTTCAGCAACGTGACATGGTTTC
ACGCCATCCATGTGTCCGGGACCAATGGAACCAAGAGGTTCGACAACCC
AGTGTTGCCATTCAACGATGGAGTCTACTTCGCTAGCACAGAGAAAAGC
AACATTATAAGGGGCTGGATCTTTGGCACCACTCTGGATAGCAAGACTC
AGTCCCTCCTCATCGTGAACAATGCTACCAACGTGGTTATTAAGGTGTG
CGAATTTCAGTTTTGTAATGATCCCTTCCTCGGGGTCTACTATCACAAA
AATAATAAGTCCTGGATGGAGTCCGAGTTCCGAGTTTATTCTTCCGCAA
ATAATTGCACATTTGAGTATGTTAGTCAGCCCTTCCTCATGGACCTCGA
AGGCAAACAGGGAAATTTTAAGAACCTTAGAGAATTCGTCTTCAAAAAT
ATCGATGGGTATTTCAAGATTTATAGCAAGCATACACCCATTAACCTGG
TACGAGACCTCCCTCAGGGCTTTTCCGCACTTGAGCCTCTGGTCGATCT
CCCGATAGGCATAAACATAACTAGGTTTCAGACTTTGCTTGCCCTCCAT
AGGTCCTACCTTACGCCTGGGGATAGCAGCAGTGGTTGGACTGCGGGCG
CCGCCGCTTACTATGTTGGGTATCTTCAACCTCGCACGTTCCTGCTTAA
GTATAATGAAAATGGGACCATTACGGATGCCGTCGATTGTGCGCTGGAT
CCTTTGTCCGAGACGAAATGTACCCTGAAAAGTTTCACCGTGGAGAAAG
GGATTTATCAGACGAGCAATTTCCGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGTGGCCCTAAAAAGTCCACGAACCTGGTCAAGAACAAATGCGTG
AATTTCAATTTCAACGGTCTCACCGGGACGGGCGTGCTTACGGAAAGCA
ACAAGAAATTCCTGCCATTCCAACAGTTCGGCCGGGACATCGCCGACAC
CACTGACGCGGTGCGCGATCCTCAGACATTGGAAATCCTTGACATTACT
CCGTGCTCTTTCGGGGGAGTCTCCGTCATCACCCCAGGAACTAACACGT
CTAACCAGGTGGCTGTTCTGTATCAGGATGTTAATTGCACAGAAGTGCC
CGTTGCGATACACGCGGACCAGCTTACTCCTACCTGGAGGGTCTATTCC
ACCGGCTCTAACGTCTTTCAGACCCGCGCCGGTTGCCTCATAGGCGCAG
AACATGTAAATAATAGTTATGAATGCGACATTCCTATTGGCGCCGGGAT
CTGTGCGAGCTACCAGACTCAAACCAACAGCCCGAGGCGGGCCAGGTCC
GTGGCGAGTCAGTCCATTATTGCTTATACAATGTCCCTGGGAGCAGAGA
ACTCTGTGGCTTACTCCAACAATAGCATAGCGATACCAACTAATTTTAC
TATATCCGTGACAACGGAGATCCTTCCCGTCTCCATGACAAAAACTAGT
GTGGACTGCACAATGTATATCTGTGGCGATAGCACTGAATGCTCCAACC
TTCTGCTGCAGTATGGGAGCTTTTGCACACAGTTGAACCGGGCCCTGAC
CGGCATCGCCGTAGAGCAGGATAAAAATACACAGGAGGTGTTTGCTCAG
GTGAAGCAAATTTACAAGACCCCTCCTATTAAAGATTTCGGCGGGTTTA
ATTTCAGCCAGATCCTGCCGGACCCATCCAAGCCGAGTAAGAGGAGCTT
TATCGAAGACTTGCTGTTCAACAAAGTGACTTTGGCTGATGCAGGCTTC
ATTAAGCAGTACGGTGATTGTCTCGGCGACATCGCAGCGCGGGATCTCA
TATGTGCTCAAAAGTTCAACGGTCTTACAGTACTCCCACCTCTGCTGAC
CGACGAGATGATTGCTCAGTACACGTCTGCCTTGCTGGCCGGGACGATC
ACCAGCGGGTGGACCTTTGGCGCAGGCGCAGCTCTTCAAATCCCCTTTG
CAATGCAGATGGCTTATCGATTTAACGGAATCGGGGTGACGCAGAACGT
GTTGTATGAGAACCAAAAATTGATAGCTAATCAATTCAATTCAGCGATA
GGTAAGATTCAGGACTCCCTGTCCAGCACCGCTTCCGCGTTGGGAAAAT
TGCAGGACGTGGTGAATCAAAATGCACAAGCACTGAATACACTCGTGAA
ACAGCTGTCCAGTAACTTCGGGGCCATCTCCTCCGTGCTCAACGACATC
CTTTCTAGACTTGATAAAGTGGAAGCCGAAGTGCAGATTGATCGGCTTA
TCACAGGTCGCCTGCAGTCACTGCAGACTTACGTCACTCAACAGCTTAT
CCGCGCCGCAGAGATCAGAGCAAGTGCCAATCTTGCCGCAACCAAAATG
TCCGAGTGCGTGTTGGGACAGTCTAAGCGAGTTGACTTTTGTGGTAAGG
GGTACCACCTGATGAGTTTCCCCCAGTCCGCCCCACACGGCGTGGTTTT
TCTGCACGTGACTTATGTGCCAGCCCAGGAGAAGAATTTTACTACCGCT
CCGGCCATTTGCCACGACGGTAAGGCACACTTCCCTAGAGAGGGGGTTT
TCGTGAGTAACGGCACTCACTGGTTTGTCACACAGCGGAATTTTTACGA
GCCTCAGATCATCACTACAGATAACACTTTCGTGTCCGGAAACTGCGAC
GTGGTTATCGGCATCGTCAACAATACCGTCTATGATCCTCTCCAGCCAG
AACTGGACTCCTTTAAGGAAGAACTTGATAAATACTTCAAGAATCACAC
AAGTCCAGATGTTGACCTTGGGGACATTAGCGGGATCAATGCAAGCGTA
GTTAATATTCAGAAGGAGATTGACCGCCTCAACGAAGTGGCCAAAAATC
TCAATGAATCTCTTATTGATCTTCAGGAGCTCGGCAAGTACGAGCAGTG
A.

In some embodiments, the SARS-CoV-2 S protein has the amino acid sequence:

(SEQ ID NO: 15, preS)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDPPEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQGSGYIPEAPRDGQAYVR
KDGEWLLSTFLG.

Therefore, in some embodiments, the SARS-CoV-2 S protein is encoded by the nucleic acid sequence:

(SEQ ID NO: 16, preS)
ATGTTCGTGTTCCTGGTGCTCCTGCCTCTGGTGAGCAGCCAGTGCGTGA
ACCTGACCACCCGAACCCAGCTCCCACCAGCCTACACCAACAGCTTTAC
ACGGGGCGTGTACTACCCTGACAAGGTGTTCAGATCTAGCGTCCTGCAC
AGCACTCAGGACCTCTTCCTGCCGTTCTTCAGCAACGTGACATGGTTCC
ACGCCATCCACGTGAGCGGCACAAACGGAACCAAGCGGTTTGATAACCC
CGTCCTGCCATTCAATGATGGAGTTTACTTCGCCAGTACCGAGAAGAGT
AACATCATCCGGGGCTGGATCTTCGGCACCACCCTGGATAGCAAAACAC
AGAGCCTCCTGATCGTGAACAATGCCACGAACGTCGTGATCAAGGTGTG
CGAGTTCCAGTTTTGCAATGATCCTTTCCTGGGTGTGTACTACCACAAG
AACAACAAGAGCTGGATGGAAAGCGAGTTCAGAGTCTACAGCAGCGCCA
ACAACTGCACATTCGAGTACGTCTCTCAGCCTTTTCTGATGGACCTTGA
GGGGAAACAAGGCAACTTCAAGAACCTGAGAGAATTCGTGTTCAAGAAC
ATCGACGGCTACTTCAAAATCTACTCCAAGCACACACCCATCAACCTGG
TCCGGGACCTCCCTCAGGGCTTCAGCGCCCTGGAACCCCTGGTCGACCT
GCCCATAGGCATCAACATAACGCGGTTCCAAACCCTGCTGGCCCTGCAT
AGATCCTACCTGACTCCTGGCGACAGCAGCAGCGGATGGACCGCCGGAG
CTGCAGCCTACTATGTGGGCTACCTGCAACCTAGAACCTTCCTGCTGAA
GTACAACGAGAACGGCACAATCACAGACGCCGTCGACTGCGCCCTGGAC
CCTCTCTCTGAGACAAAGTGCACCCTGAAGTCCTTCACCGTGGAAAAGG
GCATCTACCAGACCAGCAACTTCCGGGTGCAGCCTACAGAGAGCATCGT
GCGATTTCCAAACATTACCAACCTCTGCCCCTTCGGCGAGGTGTTTAAC
GCCACAAGATTTGCCTCCGTTTACGCCTGGAATAGAAAGAGAATCAGCA
ATTGTGTGGCCGACTACTCCGTGCTGTATAACAGCGCCTCTTTCAGCAC
CTTCAAGTGCTACGGCGTTTCCCCAACAAAGCTGAATGACCTGTGCTTC
ACCAACGTGTACGCCGACTCCTTCGTAATTAGAGGCGATGAGGTGCGGC
AGATCGCACCAGGCCAGACCGGTAAGATCGCTGACTACAACTATAAGCT
GCCTGATGATTTTACAGGCTGCGTGATCGCCTGGAACTCTAACAACCTG
GATAGCAAGGTGGGCGGCAACTACAACTACCTGTACCGGCTGTTTCGCA
AGTCTAACCTGAAACCTTTCGAGAGAGACATCTCCACAGAGATCTACCA
GGCCGGTTCTACACCTTGTAACGGGGTGGAAGGCTTCAACTGTTACTTC
CCTCTGCAAAGCTACGGCTTCCAGCCTACCAATGGAGTCGGCTACCAGC
CATACCGGGTGGTCGTGCTGTCCTTCGAGTTACTCCACGCCCCCGCCAC
CGTCTGCGGTCCTAAGAAGTCCACCAATCTGGTTAAGAACAAATGCGTG
AACTTCAACTTCAACGGCCTGACCGGGACCGGCGTGCTGACCGAAAGCA
ACAAAAAGTTCCTCCCCTTCCAGCAGTTCGGCCGTGATATCGCTGACAC
CACAGATGCCGTCAGAGATCCACAGACCCTGGAAATCCTGGATATTACA
CCCTGCTCCTTCGGAGGAGTTTCTGTGATCACCCCCGGGACCAATACCA
GCAACCAGGTGGCTGTGCTGTACCAAGATGTTAACTGCACCGAGGTTCC
TGTGGCCATCCACGCCGATCAGCTGACACCTACTTGGAGAGTGTACTCC
ACTGGCTCCAATGTGTTCCAGACCAGGGCCGGATGTCTGATCGGCGCCG
AGCACGTGAATAACAGTTACGAGTGCGACATCCCTATCGGCGCCGGCAT
CTGTGCCAGCTACCAGACCCAGACAAACAGCCCTGGGTCTGCTTCCTCT
GTAGCTAGCCAGAGCATCATCGCCTACACCATGAGCCTGGGCGCAGAGA
ACAGCGTGGCCTATTCCAACAACTCTATCGCCATTCCCACCAACTTTAC
AATTAGCGTCACAACAGAGATCCTGCCCGTGAGCATGACCAAGACCAGC
GTGGACTGTACAATGTACATCTGTGGCGACAGCACTGAATGCAGCAACC
TGCTGCTGCAATACGGCTCCTTTTGCACCCAACTGAACCGGGCGCTGAC
CGGAATCGCCGTGGAACAGGACAAAAATACCCAGGAGGTGTTCGCCCAA
GTGAAGCAGATCTACAAGACCCCACCTATCAAGGACTTCGGCGGCTTTA
ACTTTAGCCAGATTCTCCCTGATCCTTCTAAGCCTAGCAAGCGGAGCTT
TATCGAGGATCTGCTGTTCAACAAGGTCACCCTGGCCGATGCCGGCTTT
ATCAAACAGTATGGCGATTGCCTGGGCGACATAGCCGCCAGAGATCTGA
TCTGCGCCCAGAAATTCAACGGCCTGACAGTTCTCCCACCTCTGCTGAC
CGACGAGATGATCGCTCAGTACACCTCTGCCCTGCTGGCTGGCACCATC
ACATCTGGGTGGACATTTGGCGCCGGCGCCGCCCTGCAGATCCCCTTTG
CCATGCAGATGGCCTATAGATTCAACGGAATCGGCGTGACCCAGAACGT
GCTGTATGAAAACCAGAAGCTGATCGCTAACCAGTTCAATTCTGCCATC
GGCAAGATCCAGGACTCCCTCTCCTCTACCGCCAGCGCCCTGGGCAAAC
TGCAGGACGTGGTGAATCAGAACGCCCAAGCCCTGAACACCCTGGTGAA
GCAGCTCAGCAGCAATTTTGGCGCCATCAGCTCTGTGCTGAACGATATC
CTGTCTAGACTGGACCCTCCAGAAGCCGAAGTCCAGATCGATAGACTGA
TCACAGGCAGACTGCAGTCCCTGCAAACCTACGTGACCCAACAGCTGAT
CAGGGCCGCTGAAATAAGAGCCAGCGCCAATCTCGCCGCTACCAAGATG
TCCGAGTGTGTGCTGGGACAGTCTAAACGCGTTGACTTCTGCGGCAAAG
GCTATCACCTGATGAGCTTCCCCCAGAGCGCGCCGCACGGCGTGGTGTT
CCTGCATGTGACATACGTGCCTGCCCAAGAGAAGAATTTCACAACCGCC
CCTGCCATCTGCCACGACGGCAAGGCCCACTTCCCAAGAGAGGGCGTTT
TCGTTTCCAATGGCACACACTGGTTCGTGACACAAAGAAACTTCTACGA
ACCCCAGATTATCACCACCGACAACACCTTCGTGAGTGGCAATTGTGAC
GTGGTCATCGGAATCGTGAACAACACAGTGTACGACCCTCTGCAACCTG
AGCTGGACTCTTTTAAGGAAGAGCTGGACAAGTACTTTAAAAACCACAC
CAGCCCCGATGTGGACCTGGGCGACATCAGTGGCATTAACGCCAGCGTG
GTGAACATCCAAAAGGAAATCGACAGACTGAACGAGGTGGCCAAGAACC
TGAACGAGTCCCTGATCGACCTGCAGGAGCTCGGCAAATACGAGCAGGG
ATCCGGATACATCCCCGAGGCCCCCAGAGATGGCCAGGCCTACGTGCGG
AAGGACGGCGAGTGGGTACTGCTGAGCACATTCCTGGGCTGA.

In some embodiments, the preS-HexaPro protein has the amino acid sequence:

(SEQ ID NO: 127)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLI
CAQKFNGLTVLPPLLTDEMIAQYTSALLAGTITSGWTFGAGPALQIPFPM
QMAYRFNGIGVTQNVLYENQKLIANQFNSAIGKIQDSLSSTPSALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQGSGYIPEAPRDGQAYVRKDGEWVLLSTFLGR.

Therefore, in some embodiments, the preS-HexaPro protein is encoded by the nucleic acid sequence:

(SEQ ID NO: 128)
ATGTTCGTGTTCCTGGTGCTCCTGCCTCTGGTGAGCAGCCAGTGCGTGAA
CCTGACCACCCGAACCCAGCTCCCACCAGCCTACACCAACAGCTTTACAC
GGGGCGTGTACTACCCTGACAAGGTGTTCAGATCTAGCGTCCTGCACAGC
ACTCAGGACCTCTTCCTGCCGTTCTTCAGCAACGTGACATGGTTCCACGC
CATCCACGTGAGCGGCACAAACGGAACCAAGCGGTTTGATAACCCCGTCC
TGCCATTCAATGATGGAGTTTACTTCGCCAGTACCGAGAAGAGTAACATC
ATCCGGGGCTGGATCTTCGGCACCACCCTGGATAGCAAAACACAGAGCCT
CCTGATCGTGAACAATGCCACGAACGTCGTGATCAAGGTGTGCGAGTTCC
AGTTTTGCAATGATCCTTTCCTGGGTGTGTACTACCACAAGAACAACAAG
AGCTGGATGGAAAGCGAGTTCAGAGTCTACAGCAGCGCCAACAACTGCAC
ATTCGAGTACGTCTCTCAGCCTTTTCTGATGGACCTTGAGGGGAAACAAG
GCAACTTCAAGAACCTGAGAGAATTCGTGTTCAAGAACATCGACGGCTAC
TTCAAAATCTACTCCAAGCACACACCCATCAACCTGGTCCGGGACCTCCC
TCAGGGCTTCAGCGCCCTGGAACCCCTGGTCGACCTGCCCATAGGCATCA
ACATAACGCGGTTCCAAACCCTGCTGGCCCTGCATAGATCCTACCTGACT
CCTGGCGACAGCAGCAGCGGATGGACCGCCGGAGCTGCAGCCTACTATGT
GGGCTACCTGCAACCTAGAACCTTCCTGCTGAAGTACAACGAGAACGGCA
CAATCACAGACGCCGTCGACTGCGCCCTGGACCCTCTCTCTGAGACAAAG
TGCACCCTGAAGTCCTTCACCGTGGAAAAGGGCATCTACCAGACCAGCAA
CTTCCGGGTGCAGCCTACAGAGAGCATCGTGCGATTTCCAAACATTACCA
ACCTCTGCCCCTTCGGCGAGGTGTTTAACGCCACAAGATTTGCCTCCGTT
TACGCCTGGAATAGAAAGAGAATCAGCAATTGTGTGGCCGACTACTCCGT
GCTGTATAACAGCGCCTCTTTCAGCACCTTCAAGTGCTACGGCGTTTCCC
CAACAAAGCTGAATGACCTGTGCTTCACCAACGTGTACGCCGACTCCTTC
GTAATTAGAGGCGATGAGGTGCGGCAGATCGCACCAGGCCAGACCGGTAA
GATCGCTGACTACAACTATAAGCTGCCTGATGATTTTACAGGCTGCGTGA
TCGCCTGGAACTCTAACAACCTGGATAGCAAGGTGGGCGGCAACTACAAC
TACCTGTACCGGCTGTTTCGCAAGTCTAACCTGAAACCTTTCGAGAGAGA
CATCTCCACAGAGATCTACCAGGCCGGTTCTACACCTTGTAACGGGGTGG
AAGGCTTCAACTGTTACTTCCCTCTGCAAAGCTACGGCTTCCAGCCTACC
AATGGAGTCGGCTACCAGCCATACCGGGTGGTCGTGCTGTCCTTCGAGTT
ACTCCACGCCCCCGCCACCGTCTGCGGTCCTAAGAAGTCCACCAATCTGG
TTAAGAACAAATGCGTGAACTTCAACTTCAACGGCCTGACCGGGACCGGC
GTGCTGACCGAAAGCAACAAAAAGTTCCTCCCCTTCCAGCAGTTCGGCCG
TGATATCGCTGACACCACAGATGCCGTCAGAGATCCACAGACCCTGGAAA
TCCTGGATATTACACCCTGCTCCTTCGGAGGAGTTTCTGTGATCACCCCC
GGGACCAATACCAGCAACCAGGTGGCTGTGCTGTACCAAGATGTTAACTG
CACCGAGGTTCCTGTGGCCATCCACGCCGATCAGCTGACACCTACTTGGA
GAGTGTACTCCACTGGCTCCAATGTGTTCCAGACCAGGGCCGGATGTCTG
ATCGGCGCCGAGCACGTGAATAACAGTTACGAGTGCGACATCCCTATCGG
CGCCGGCATCTGTGCCAGCTACCAGACCCAGACAAACAGCCCTGGGTCTG
CTTCCTCTGTAGCTAGCCAGAGCATCATCGCCTACACCATGAGCCTGGGC
GCAGAGAACAGCGTGGCCTATTCCAACAACTCTATCGCCATTCCCACCAA
CTTTACAATTAGCGTCACAACAGAGATCCTGCCCGTGAGCATGACCAAGA
CCAGCGTGGACTGTACAATGTACATCTGTGGCGACAGCACTGAATGCAGC
AACCTGCTGCTGCAATACGGCTCCTTTTGCACCCAACTGAACCGGGCGCT
GACCGGAATCGCCGTGGAACAGGACAAAAATACCCAGGAGGTGTTCGCCC
AAGTGAAGCAGATCTACAAGACCCCACCTATCAAGGACTTCGGCGGCTTT
AACTTTAGCCAGATTCTCCCTGATCCTTCTAAGCCTAGCAAGCGGAGCCC
TATCGAGGATCTGCTGTTCAACAAGGTCACCCTGGCCGATGCCGGCTTTA
TCAAACAGTATGGCGATTGCCTGGGCGACATAGCCGCCAGAGATCTGATC
TGCGCCCAGAAATTCAACGGCCTGACAGTTCTCCCACCTCTGCTGACCGA
CGAGATGATCGCTCAGTACACCTCTGCCCTGCTGGCTGGCACCATCACAT
CTGGGTGGACATTTGGCGCCGGCCCCGCCCTGCAGATCCCCTTTCCCATG
CAGATGGCCTATAGATTCAACGGAATCGGCGTGACCCAGAACGTGCTGTA
TGAAAACCAGAAGCTGATCGCTAACCAGTTCAATTCTGCCATCGGCAAGA
TCCAGGACTCCCTCTCCTCTACCCCCAGCGCCCTGGGCAAACTGCAGGAC
GTGGTGAATCAGAACGCCCAAGCCCTGAACACCCTGGTGAAGCAGCTCAG
CAGCAATTTTGGCGCCATCAGCTCTGTGCTGAACGATATCCTGTCTAGAC
TGGACCCTCCAGAAGCCGAAGTCCAGATCGATAGACTGATCACAGGCAGA
CTGCAGTCCCTGCAAACCTACGTGACCCAACAGCTGATCAGGGCCGCTGA
AATAAGAGCCAGCGCCAATCTCGCCGCTACCAAGATGTCCGAGTGTGTGC
TGGGACAGTCTAAACGCGTTGACTTCTGCGGCAAAGGCTATCACCTGATG
AGCTTCCCCCAGAGCGCGCCGCACGGCGTGGTGTTCCTGCATGTGACATA
CGTGCCTGCCCAAGAGAAGAATTTCACAACCGCCCCTGCCATCTGCCACG
ACGGCAAGGCCCACTTCCCAAGAGAGGGCGTTTTCGTTTCCAATGGCACA
CACTGGTTCGTGACACAAAGAAACTTCTACGAACCCCAGATTATCACCAC
CGACAACACCTTCGTGAGTGGCAATTGTGACGTGGTCATCGGAATCGTGA
ACAACACAGTGTACGACCCTCTGCAACCTGAGCTGGACTCTTTTAAGGAA
GAGCTGGACAAGTACTTTAAAAACCACACCAGCCCCGATGTGGACCTGGG
CGACATCAGTGGCATTAACGCCAGCGTGGTGAACATCCAAAAGGAAATCG
ACAGACTGAACGAGGTGGCCAAGAACCTGAACGAGTCCCTGATCGACCTG
CAGGAGCTCGGCAAATACGAGCAGGGATCCGGATACATCCCCGAGGCCCC
CAGAGATGGCCAGGCCTACGTGCGGAAGGACGGCGAGTGGGTACTGCTGA
GCACATTCCTGGGCAGA.

In some embodiments, the preS-HexaPro protein UK variant has the amino acid sequence:

(SEQ ID NO: 129)
MFVFLVLLPLVSSQCVNLTTRTQLPPAYTNSFTRGVYYPDKVFRSSVLHS
TQDLFLPFFSNVTWFHAISGTNGTKRFDNPVLPFNDGVYFASTEKSNIIR
GWIFGTTLDSKTQSLLIVNNATNVVIKVCEFQFCNDPFLGVYHKNNKSWM
ESEFRVYSSANNCTFEYVSQPFLMDLEGKQGNFKNLREFVFKNIDGYFKI
YSKHTPINLVRDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTYGV
GYQPYRVVVLSFELLHAPATVCGPKKSTNLVKNKCVNFNFNGLTGTGVLT
ESNKKFLPFQQFGRDIDDTTDAVRDPQTLEILDITPCSFGGVSVITPGTN
TSNQVAVLYQDVNCTEVPVAIHADQLTPTWRVYSTGSNVFQTRAGCLIGA
EHVNNSYECDIPIGAGICASYQTQTNSHGSASSVASQSIIAYTMSLGAEN
SVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSPIEDLLFNKVTLADAGFIKQYGDCLGDIAARDLICAQ
KFNGLTVLPPLLTDEMIAQYTSALLAGTITSGWTFGAGPALQIPFPMQMA
YRFNGIGVTQNVLYENQKLIANQFNSAIGKIQDSLSSTPSALGKLQDVVN
QNAQALNTLVKQLSSNFGAISSVLNDILSRLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQGSGYIPEAPRDGQAYVRKDGEWVLLSTFLGR.

Therefore, in some embodiments, the preS-HexaPro protein UK variant protein is encoded by the nucleic acid sequence:

(SEQ ID NO: 130)
ATGTTCGTGTTCCTGGTGCTCCTGCCTCTGGTGAGCAGCCAGTGCGTGAA
CCTGACCACCCGAACCCAGCTCCCACCAGCCTACACCAACAGCTTTACAC
GGGGCGTGTACTACCCTGACAAGGTGTTCAGATCTAGCGTCCTGCACAGC
ACTCAGGACCTCTTCCTGCCGTTCTTCAGCAACGTGACATGGTTCCACGC
CATCAGCGGCACAAACGGAACCAAGCGGTTTGATAACCCCGTCCTGCCAT
TCAATGATGGAGTTTACTTCGCCAGTACCGAGAAGAGTAACATCATCCGG
GGCTGGATCTTCGGCACCACCCTGGATAGCAAAACACAGAGCCTCCTGAT
CGTGAACAATGCCACGAACGTCGTGATCAAGGTGTGCGAGTTCCAGTTTT
GCAATGATCCTTTCCTGGGTGTGTACCACAAGAACAACAAGAGCTGGATG
GAAAGCGAGTTCAGAGTCTACAGCAGCGCCAACAACTGCACATTCGAGTA
CGTCTCTCAGCCTTTTCTGATGGACCTTGAGGGGAAACAAGGCAACTTCA
AGAACCTGAGAGAATTCGTGTTCAAGAACATCGACGGCTACTTCAAAATC
TACTCCAAGCACACACCCATCAACCTGGTCCGGGACCTCCCTCAGGGCTT
CAGCGCCCTGGAACCCCTGGTCGACCTGCCCATAGGCATCAACATAACGC
GGTTCCAAACCCTGCTGGCCCTGCATAGATCCTACCTGACTCCTGGCGAC
AGCAGCAGCGGATGGACCGCCGGAGCTGCAGCCTACTATGTGGGCTACCT
GCAACCTAGAACCTTCCTGCTGAAGTACAACGAGAACGGCACAATCACAG
ACGCCGTCGACTGCGCCCTGGACCCTCTCTCTGAGACAAAGTGCACCCTG
AAGTCCTTCACCGTGGAAAAGGGCATCTACCAGACCAGCAACTTCCGGGT
GCAGCCTACAGAGAGCATCGTGCGATTTCCAAACATTACCAACCTCTGCC
CCTTCGGCGAGGTGTTTAACGCCACAAGATTTGCCTCCGTTTACGCCTGG
AATAGAAAGAGAATCAGCAATTGTGTGGCCGACTACTCCGTGCTGTATAA
CAGCGCCTCTTTCAGCACCTTCAAGTGCTACGGCGTTTCCCCAACAAAGC
TGAATGACCTGTGCTTCACCAACGTGTACGCCGACTCCTTCGTAATTAGA
GGCGATGAGGTGCGGCAGATCGCACCAGGCCAGACCGGTAAGATCGCTGA
CTACAACTATAAGCTGCCTGATGATTTTACAGGCTGCGTGATCGCCTGGA
ACTCTAACAACCTGGATAGCAAGGTGGGCGGCAACTACAACTACCTGTAC
CGGCTGTTTCGCAAGTCTAACCTGAAACCTTTCGAGAGAGACATCTCCAC
AGAGATCTACCAGGCCGGTTCTACACCTTGTAACGGGGTGGAAGGCTTCA
ACTGTTACTTCCCTCTGCAAAGCTACGGCTTCCAGCCTACCTACGGAGTC
GGCTACCAGCCATACCGGGTGGTCGTGCTGTCCTTCGAGTTACTCCACGC
CCCCGCCACCGTCTGCGGTCCTAAGAAGTCCACCAATCTGGTTAAGAACA
AATGCGTGAACTTCAACTTCAACGGCCTGACCGGGACCGGCGTGCTGACC
GAAAGCAACAAAAAGTTCCTCCCCTTCCAGCAGTTCGGCCGTGATATCGA
CGACACCACAGATGCCGTCAGAGATCCACAGACCCTGGAAATCCTGGATA
TTACACCCTGCTCCTTCGGAGGAGTTTCTGTGATCACCCCCGGGACCAAT
ACCAGCAACCAGGTGGCTGTGCTGTACCAAGATGTTAACTGCACCGAGGT
TCCTGTGGCCATCCACGCCGATCAGCTGACACCTACTTGGAGAGTGTACT
CCACTGGCTCCAATGTGTTCCAGACCAGGGCCGGATGTCTGATCGGCGCC
GAGCACGTGAATAACAGTTACGAGTGCGACATCCCTATCGGCGCCGGCAT
CTGTGCCAGCTACCAGACCCAGACAAACAGCCACGGGTCTGCTTCCTCTG
TAGCTAGCCAGAGCATCATCGCCTACACCATGAGCCTGGGCGCAGAGAAC
AGCGTGGCCTATTCCAACAACTCTATCGCCATTCCCACCAACTTTACAAT
TAGCGTCACAACAGAGATCCTGCCCGTGAGCATGACCAAGACCAGCGTGG
ACTGTACAATGTACATCTGTGGCGACAGCACTGAATGCAGCAACCTGCTG
CTGCAATACGGCTCCTTTTGCACCCAACTGAACCGGGCGCTGACCGGAAT
CGCCGTGGAACAGGACAAAAATACCCAGGAGGTGTTCGCCCAAGTGAAGC
AGATCTACAAGACCCCACCTATCAAGGACTTCGGCGGCTTTAACTTTAGC
CAGATTCTCCCTGATCCTTCTAAGCCTAGCAAGCGGAGCCCTATCGAGGA
TCTGCTGTTCAACAAGGTCACCCTGGCCGATGCCGGCTTTATCAAACAGT
ATGGCGATTGCCTGGGCGACATAGCCGCCAGAGATCTGATCTGCGCCCAG
AAATTCAACGGCCTGACAGTTCTCCCACCTCTGCTGACCGACGAGATGAT
CGCTCAGTACACCTCTGCCCTGCTGGCTGGCACCATCACATCTGGGTGGA
CATTTGGCGCCGGCCCCGCCCTGCAGATCCCCTTTCCCATGCAGATGGCC
TATAGATTCAACGGAATCGGCGTGACCCAGAACGTGCTGTATGAAAACCA
GAAGCTGATCGCTAACCAGTTCAATTCTGCCATCGGCAAGATCCAGGACT
CCCTCTCCTCTACCCCCAGCGCCCTGGGCAAACTGCAGGACGTGGTGAAT
CAGAACGCCCAAGCCCTGAACACCCTGGTGAAGCAGCTCAGCAGCAATTT
TGGCGCCATCAGCTCTGTGCTGAACGATATCCTGTCTAGACTGGACCCTC
CAGAAGCCGAAGTCCAGATCGATAGACTGATCACAGGCAGACTGCAGTCC
CTGCAAACCTACGTGACCCAACAGCTGATCAGGGCCGCTGAAATAAGAGC
CAGCGCCAATCTCGCCGCTACCAAGATGTCCGAGTGTGTGCTGGGACAGT
CTAAACGCGTTGACTTCTGCGGCAAAGGCTATCACCTGATGAGCTTCCCC
CAGAGCGCGCCGCACGGCGTGGTGTTCCTGCATGTGACATACGTGCCTGC
CCAAGAGAAGAATTTCACAACCGCCCCTGCCATCTGCCACGACGGCAAGG
CCCACTTCCCAAGAGAGGGCGTTTTCGTTTCCAATGGCACACACTGGTTC
GTGACACAAAGAAACTTCTACGAACCCCAGATTATCACCACCGACAACAC
CTTCGTGAGTGGCAATTGTGACGTGGTCATCGGAATCGTGAACAACACAG
TGTACGACCCTCTGCAACCTGAGCTGGACTCTTTTAAGGAAGAGCTGGAC
AAGTACTTTAAAAACCACACCAGCCCCGATGTGGACCTGGGCGACATCAG
TGGCATTAACGCCAGCGTGGTGAACATCCAAAAGGAAATCGACAGACTGA
ACGAGGTGGCCAAGAACCTGAACGAGTCCCTGATCGACCTGCAGGAGCTC
GGCAAATACGAGCAGGGATCCGGATACATCCCCGAGGCCCCCAGAGATGG
CCAGGCCTACGTGCGGAAGGACGGCGAGTGGGTACTGCTGAGCACATTCC
TGGGCAGA.

In some embodiments, the preS-HexaPro protein South African variant has the amino acid sequence:

(SEQ ID NO: 131)
MFVFLVLLPLVSSQCVNFTTRTQLPPAYTNSFTRGVYYPDKVFRSSVLHS
TQDLFLPFFSNVTWFHAIHVSGTNGTKRFANPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRGLPQGFSALEPLVDLPIGINITRFQTLHISYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAW
NRKRISNCVADYSVLYNSASFSTFKCYGVSPTKLNDLCFTNVYADSFVIR
GDEVRQIAPGQTGNIADYNYKLPDDFTGCVIAWNSNNLDSKVGGNYNYLY
RLFRKSNLKPFERDISTEIYQAGSTPCNGVKGFNCYFPLQSYGFQPTYGV
GYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTN
TSNQVAVLYQDVNCTEVPVAIHADQLTPTWRVYSTGSNVFQTRAGCLIGA
EHVNNSYECDIPIGAGICASYQTQTNSPGSASSVASQSIIAYTMSLGAEN
SVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSPIEDLLFNKVTLADAGFIKQYGDCLGDIAARDLICAQ
KFNGLTVLPPLLTDEMIAQYTSALLAGTITSGWTFGAGPALQIPFPMQMA
YRFNGIGVTQNVLYENQKLIANQFNSAIGKIQDSLSSTPSALGKLQDVVN
QNAQALNTLVKQLSSNFGAISSVLNDILSRLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQGSGYIPEAPRDGQAYVRKDGEWVLLSTFLGR.

Therefore, in some embodiments, the preS-HexaPro protein is encoded by the nucleic acid sequence:

(SEQ ID NO: 132)
ATGTTCGTGTTCCTGGTGCTCCTGCCTCTGGTGAGCAGCCAGTGCGTGA
ACTTCACCACCCGAACCCAGCTCCCACCAGCCTACACCAACAGCTTTAC
ACGGGGCGTGTACTACCCTGACAAGGTGTTCAGATCTAGCGTCCTGCAC
AGCACTCAGGACCTCTTCCTGCCGTTCTTCAGCAACGTGACATGGTTCC
ACGCCATCCACGTGAGCGGCACAAACGGAACCAAGCGGTTTGCCAACCC
CGTCCTGCCATTCAATGATGGAGTTTACTTCGCCAGTACCGAGAAGAGT
AACATCATCCGGGGCTGGATCTTCGGCACCACCCTGGATAGCAAAACAC
AGAGCCTCCTGATCGTGAACAATGCCACGAACGTCGTGATCAAGGTGTG
CGAGTTCCAGTTTTGCAATGATCCTTTCCTGGGTGTGTACTACCACAAG
AACAACAAGAGCTGGATGGAAAGCGAGTTCAGAGTCTACAGCAGCGCCA
ACAACTGCACATTCGAGTACGTCTCTCAGCCTTTTCTGATGGACCTTGA
GGGGAAACAAGGCAACTTCAAGAACCTGAGAGAATTCGTGTTCAAGAAC
ATCGACGGCTACTTCAAAATCTACTCCAAGCACACACCCATCAACCTGG
TCCGGGGCCTCCCTCAGGGCTTCAGCGCCCTGGAACCCCTGGTCGACCT
GCCCATAGGCATCAACATAACGCGGTTCCAAACCCTGCATATCTCCTAC
CTGACTCCTGGCGACAGCAGCAGCGGATGGACCGCCGGAGCTGCAGCCT
ACTATGTGGGCTACCTGCAACCTAGAACCTTCCTGCTGAAGTACAACGA
GAACGGCACAATCACAGACGCCGTCGACTGCGCCCTGGACCCTCTCTCT
GAGACAAAGTGCACCCTGAAGTCCTTCACCGTGGAAAAGGGCATCTACC
AGACCAGCAACTTCCGGGTGCAGCCTACAGAGAGCATCGTGCGATTTCC
AAACATTACCAACCTCTGCCCCTTCGGCGAGGTGTTTAACGCCACAAGA
TTTGCCTCCGTTTACGCCTGGAATAGAAAGAGAATCAGCAATTGTGTGG
CCGACTACTCCGTGCTGTATAACAGCGCCTCTTTCAGCACCTTCAAGTG
CTACGGCGTTTCCCCAACAAAGCTGAATGACCTGTGCTTCACCAACGTG
TACGCCGACTCCTTCGTAATTAGAGGCGATGAGGTGCGGCAGATCGCAC
CAGGCCAGACCGGTAACATCGCTGACTACAACTATAAGCTGCCTGATGA
TTTTACAGGCTGCGTGATCGCCTGGAACTCTAACAACCTGGATAGCAAG
GTGGGCGGCAACTACAACTACCTGTACCGGCTGTTTCGCAAGTCTAACC
TGAAACCTTTCGAGAGAGACATCTCCACAGAGATCTACCAGGCCGGTTC
TACACCTTGTAACGGGGTGAAGGGCTTCAACTGTTACTTCCCTCTGCAA
AGCTACGGCTTCCAGCCTACCTACGGAGTCGGCTACCAGCCATACCGGG
TGGTCGTGCTGTCCTTCGAGTTACTCCACGCCCCCGCCACCGTCTGCGG
TCCTAAGAAGTCCACCAATCTGGTTAAGAACAAATGCGTGAACTTCAAC
TTCAACGGCCTGACCGGGACCGGCGTGCTGACCGAAAGCAACAAAAAGT
TCCTCCCCTTCCAGCAGTTCGGCCGTGATATCGCTGACACCACAGATGC
CGTCAGAGATCCACAGACCCTGGAAATCCTGGATATTACACCCTGCTCC
TTCGGAGGAGTTTCTGTGATCACCCCCGGGACCAATACCAGCAACCAGG
TGGCTGTGCTGTACCAAGATGTTAACTGCACCGAGGTTCCTGTGGCCAT
CCACGCCGATCAGCTGACACCTACTTGGAGAGTGTACTCCACTGGCTCC
AATGTGTTCCAGACCAGGGCCGGATGTCTGATCGGCGCCGAGCACGTGA
ATAACAGTTACGAGTGCGACATCCCTATCGGCGCCGGCATCTGTGCCAG
CTACCAGACCCAGACAAACAGCCCTGGGTCTGCTTCCTCTGTAGCTAGC
CAGAGCATCATCGCCTACACCATGAGCCTGGGCGCAGAGAACAGCGTGG
CCTATTCCAACAACTCTATCGCCATTCCCACCAACTTTACAATTAGCGT
CACAACAGAGATCCTGCCCGTGAGCATGACCAAGACCAGCGTGGACTGT
ACAATGTACATCTGTGGCGACAGCACTGAATGCAGCAACCTGCTGCTGC
AATACGGCTCCTTTTGCACCCAACTGAACCGGGCGCTGACCGGAATCGC
CGTGGAACAGGACAAAAATACCCAGGAGGTGTTCGCCCAAGTGAAGCAG
ATCTACAAGACCCCACCTATCAAGGACTTCGGCGGCTTTAACTTTAGCC
AGATTCTCCCTGATCCTTCTAAGCCTAGCAAGCGGAGCCCTATCGAGGA
TCTGCTGTTCAACAAGGTCACCCTGGCCGATGCCGGCTTTATCAAACAG
TATGGCGATTGCCTGGGCGACATAGCCGCCAGAGATCTGATCTGCGCCC
AGAAATTCAACGGCCTGACAGTTCTCCCACCTCTGCTGACCGACGAGAT
GATCGCTCAGTACACCTCTGCCCTGCTGGCTGGCACCATCACATCTGGG
TGGACATTTGGCGCCGGCCCCGCCCTGCAGATCCCCTTTCCCATGCAGA
TGGCCTATAGATTCAACGGAATCGGCGTGACCCAGAACGTGCTGTATGA
AAACCAGAAGCTGATCGCTAACCAGTTCAATTCTGCCATCGGCAAGATC
CAGGACTCCCTCTCCTCTACCCCCAGCGCCCTGGGCAAACTGCAGGACG
TGGTGAATCAGAACGCCCAAGCCCTGAACACCCTGGTGAAGCAGCTCAG
CAGCAATTTTGGCGCCATCAGCTCTGTGCTGAACGATATCCTGTCTAGA
CTGGACCCTCCAGAAGCCGAAGTCCAGATCGATAGACTGATCACAGGCA
GACTGCAGTCCCTGCAAACCTACGTGACCCAACAGCTGATCAGGGCCGC
TGAAATAAGAGCCAGCGCCAATCTCGCCGCTACCAAGATGTCCGAGTGT
GTGCTGGGACAGTCTAAACGCGTTGACTTCTGCGGCAAAGGCTATCACC
TGATGAGCTTCCCCCAGAGCGCGCCGCACGGCGTGGTGTTCCTGCATGT
GACATACGTGCCTGCCCAAGAGAAGAATTTCACAACCGCCCCTGCCATC
TGCCACGACGGCAAGGCCCACTTCCCAAGAGAGGGCGTTTTCGTTTCCA
ATGGCACACACTGGTTCGTGACACAAAGAAACTTCTACGAACCCCAGAT
TATCACCACCGACAACACCTTCGTGAGTGGCAATTGTGACGTGGTCATC
GGAATCGTGAACAACACAGTGTACGACCCTCTGCAACCTGAGCTGGACT
CTTTTAAGGAAGAGCTGGACAAGTACTTTAAAAACCACACCAGCCCCGA
TGTGGACCTGGGCGACATCAGTGGCATTAACGCCAGCGTGGTGAACATC
CAAAAGGAAATCGACAGACTGAACGAGGTGGCCAAGAACCTGAACGAGT
CCCTGATCGACCTGCAGGAGCTCGGCAAATACGAGCAGGGATCCGGATA
CATCCCCGAGGCCCCCAGAGATGGCCAGGCCTACGTGCGGAAGGACGGC
GAGTGGGTACTGCTGAGCACATTCCTGGGCAGA.

In some embodiments, the SARS-CoV-2 N protein has the amino acid sequence:

(SEQ ID NO: 133)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA.

Therefore, in some embodiments, the SARS-CoV-2 N protein is encoded by the nucleic acid sequence:

(SEQ ID NO: 134)
ATGTCTGATAATGGACCCCAAAATCAGCGAAATGCACCCCGCATTACGTT
TGGTGGACCCTCAGATTCAACTGGCAGTAACCAGAATGGAGAACGCAGTG
GGGCGCGATCAAAACAACGTCGGCCCCAAGGTTTACCCAATAATACTGCG
TCTTGGTTCACCGCTCTCACTCAACATGGCAAGGAAGACCTTAAATTCCC
TCGAGGACAAGGCGTTCCAATTAACACCAATAGCAGTCCAGATGACCAAA
TTGGCTACTACCGAAGAGCTACCAGACGAATTCGTGGTGGTGACGGTAAA
ATGAAAGATCTCAGTCCAAGATGGTATTTCTACTACCTAGGAACTGGGCC
AGAAGCTGGACTTCCCTATGGTGCTAACAAAGACGGCATCATATGGGTTG
CAACTGAGGGAGCCTTGAATACACCAAAAGATCACATTGGCACCCGCAAT
CCTGCTAACAATGCTGCAATCGTGCTACAACTTCCTCAAGGAACAACATT
GCCAAAAGGCTTCTACGCAGAAGGGAGCAGAGGCGGCAGTCAAGCCTCTT
CTCGTTCCTCATCACGTAGTCGCAACAGTTCAAGAAATTCAACTCCAGGC
AGCAGTAGGGGAACTTCTCCTGCTAGAATGGCTGGCAATGGCGGTGATGC
TGCTCTTGCTTTGCTGCTGCTTGACAGATTGAACCAGCTTGAGAGCAAAA
TGTCTGGTAAAGGCCAACAACAACAAGGCCAAACTGTCACTAAGAAATCT
GCTGCTGAGGCTTCTAAGAAGCCTCGGCAAAAACGTACTGCCACTAAAGC
ATACAATGTAACACAAGCTTTCGGCAGACGTGGTCCAGAACAAACCCAAG
GAAATTTTGGGGACCAGGAACTAATCAGACAAGGAACTGATTACAAACAT
TGGCCGCAAATTGCACAATTTGCCCCCAGCGCTTCAGCGTTCTTCGGAAT
GTCGCGCATTGGCATGGAAGTCACACCTTCGGGAACGTGGTTGACCTACA
CAGGTGCCATCAAATTGGATGACAAAGATCCAAATTTCAAAGATCAAGTC
ATTTTGCTGAATAAGCATATTGACGCATACAAAACATTCCCACCAACAGA
GCCTAAAAAGGACAAAAAGAAGAAGGCTGATGAAACTCAAGCCTTACCGC
AGAGACAGAAGAAACAGCAAACTGTGACTCTTCTTCCTGCTGCAGATTTG
GATGATTTCTCCAAACAATTGCAACAATCCATGAGCAGTGCTGACTCAAC
TCAGGCCTAA.

In some embodiments, the SARS-CoV-2 M protein has the amino acid sequence:

(SEQ ID NO: 135)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ.

Therefore, in some embodiments, the SARS-CoV-2 M is encoded by the nucleic acid sequence:

(SEQ ID NO: 136)
ATGGCAGATTCCAACGGTACTATTACCGTTGAAGAGCTTAAAAAGCTCCT
TGAACAATGGAACCTAGTAATAGGTTTCCTATTCCTTACATGGATTTGTC
TTCTACAATTTGCCTATGCCAACAGGAATAGGTTTTTGTATATAATTAAG
TTAATTTTCCTCTGGCTGTTATGGCCAGTAACTTTAGCTTGTTTTGTGCT
TGCTGCTGTTTACAGAATAAATTGGATCACCGGTGGAATTGCTATCGCAA
TGGCTTGTCTTGTAGGCTTGATGTGGCTCAGCTACTTCATTGCTTCTTTC
AGACTGTTTGCGCGTACGCGTTCCATGTGGTCATTCAATCCAGAAACTAA
CATTCTTCTCAACGTGCCACTCCATGGCACTATTCTGACCAGACCGCTTC
TAGAAAGTGAACTCGTAATCGGAGCTGTGATCCTTCGTGGACATCTTCGT
ATTGCTGGACACCATCTAGGACGCTGTGACATCAAGGACCTGCCTAAAGA
AATCACTGTTGCTACATCACGAACGCTTTCTTATTACAAATTGGGAGCTT
CGCAGCGTGTAGCAGGTGACTCAGGTTTTGCTGCATACAGTCGCTACAGG
ATTGGCAACTATAAATTAAACACAGACCATTCCAGTAGCAGTGACAATAT
TGCTTTGCTTGTACAGTAA.

In some embodiments, the SARS-CoV-2 E protein has the amino acid sequence:

(SEQ ID NO: 137)
MYSFVSEETGTLIVNSVLLFLAFVVFLLVTLAILTALRLCAYCCNIVNVS
LVKPSFYVYSRVKNLNSSRVPDLLV.

Therefore, in some embodiments, the SARS-CoV-2 E protein is encoded by the nucleic acid sequence:

(SEQ ID NO: 138)
ACTTATGTACTCATTCGTTTCGGAAGAGACAGGTACGTTAATAGTTAATA
GCGTACTTCTTTTTCTTGCTTTCGTGGTATTCTTGCTAGTTACACTAGCC
ATCCTTACTGCGCTTCGATTGTGTGCGTACTGCTGCAATATTGTTAACGT
GAGTCTTGTAAAACCTTCTTTTTACGTTTACTCTCGTGTTAAAAATCTGA
ATTCTTCTAGAGTTCCTGATCTTCTGGTCTAA.

In some embodiments, the SARS-CoV-2 ORF3a protein has the amino acid sequence:

(SEQ ID NO: 139)
MDLFMRIFTIGTVTLKQGEIKDATPSDFVRATATIPIQASLPFGWLIVGV
ALLAVFQSASKIITLKKRWQLALSKGVHFVCNLLLLFVTVYSHLLLVAAG
LEAPFLYLYALVYFLQSINFVRIIMRLWLCWKCRSKNPLLYDANYFLCWH
TNCYDYCIPYNSVTSSIVITSGDGTTSPISEHDYQIGGYTEKWESGVKDC
VVLHSYFTSDYYQLYSTQLSTDTGVEHVTFFIYNKIVDEPEEHVQ.

Therefore, in some embodiments, the SARS-CoV-2 ORF3a protein is encoded by the nucleic acid sequence:

(SEQ ID NO: 140)
TTATGGATTTGTTTATGAGAATCTTCACAATTGGAACTGTAACTTTGAAG
CAAGGTGAAATCAAGGATGCTACTCCTTCAGATTTTGTTCGCGCTACTGC
AACGATACCGATACAAGCCTCACTCCCTTTCGGATGGCTTATTGTTGGCG
TTGCACTTCTTGCTGTTTTTCAGAGCGCTTCCAAAATCATAACCCTCAAA
AAGAGATGGCAACTAGCACTCTCCAAGGGTGTTCACTTTGTTTGCAACTT
GCTGTTGTTGTTTGTAACAGTTTACTCACACCTTTTGCTCGTTGCTGCTG
GCCTTGAAGCCCCTTTTCTCTATCTTTATGCTTTAGTCTACTTCTTGCAG
AGTATAAACTTTGTAAGAATAATAATGAGGCTTTGGCTTTGCTGGAAATG
CCGTTCCAAAAACCCATTACTTTATGATGCCAACTATTTTCTTTGCTGGC
ATACTAATTGTTACGACTATTGTATACCTTACAATAGTGTAACTTCTTCA
ATTGTCATTACTTCAGGTGATGGCACAACAAGTCCTATTTCTGAACATGA
CTACCAGATTGGTGGTTATACTGAAAAATGGGAATCTGGAGTAAAAGACT
GTGTTGTATTACACAGTTACTTCACTTCAGACTATTACCAGCTGTACTCA
ACTCAATTGAGTACAGACACTGGTGTTGAACATGTTACCTTCTTCATCTA
CAATAAAATTGTTGATGAGCCTGAAGAACATGTCCAAATTCACACAATCG
ACGGTTCATCCGGAGTTGTTAATCCAGTAATGGAACCAATTTATGATGAA
CCGACGACGACTACTAGCGTGCCTTTGTAA.

In some embodiments, the SARS-CoV-2 ORF8 protein has the amino acid sequence:

(SEQ ID NO: 141)
MKFLVFLGIITTVAAFHQECSLQSCTQHQPYVVDDPCPIHFYSKWYIRVG
ARKSAPLIELCVDEAGSKSPIQYIDIGNYTVSCSPFTINCQEPKLGSLVV
RCSFYEDFLEYHDVRVVLDFI.

Therefore, in some embodiments, the SARS-CoV-2 ORF8 protein is encoded by the nucleic acid sequence:

(SEQ ID NO: 142)
ATGAAATTTCTTGTTTTCTTAGGAATCATCACAACTGTAGCTGCATTTC
ACCAAGAATGTAGTTTACAGTCATGTACTCAACATCAACCATATGTAGT
TGATGACCCGTGTCCTATTCACTTCTATTCTAAATGGTATATTAGAGTA
GGAGCTAGAAAATCAGCACCTTTAATTGAATTGTGCGTGGATGAGGCTG
GTTCTAAATCACCCATTCAGTACATCGATATCGGTAATTATACAGTTTC
CTGTTCACCTTTTACAATTAATTGCCAGGAACCTAAATTGGGTAGTCTT
GTAGTGCGTTGTTCGTTCTATGAAGACTTTTTAGAGTATCATGACGTTC
GTGTTGTTTTAGATTTCATCTAA.

In some embodiments, the SARS-CoV-2 nsp6 protein has the amino acid sequence:

(SEQ ID NO: 143)
SAVKRTIKGTHHWLLLTILTSLLVLVQSTQWSLFFFLYENAFLPFAMGI
IAMSAFAMMFVKHKHAFLCLFLLPSLATVAYFNMVYMPASWVMRIMTWL
DMVDTSLSGFKLKDCVMYASAVVLLILMTARTVYDDGARRVWTLMNVLT
LVYKVYYGNALDQAISMWALIISVTSNYSGVVTTVMFLARGIVFMCVEY
CPIFFITGNTLQCIMLVYCFLGYFCTCYFGLFCLLNRYFRLTLGVYDYL
VSTQEFRYMNSQGLLPPKNSIDAFKLNIKLLGVGGKPCIKVATVQ.

Therefore, in some embodiments, the SARS-CoV-2 nsp6 protein is encoded by the nucleic acid sequence:

(SEQ ID NO: 144)
ATGAGTGCAGTGAAAAGAACAATCAAGGGTACACACCACTGGTTGTTAC
TCACAATTTTGACTTCACTTTTAGTTTTAGTCCAGAGTACTCAATGGTC
TTTGTTCTTTTTTTTGTATGAAAATGCCTTTTTACCTTTTGCTATGGGT
ATTATTGCTATGTCTGCTTTTGCAATGATGTTTGTCAAACATAAGCATG
CATTTCTCTGTTTGTTTTTGTTACCTTCTCTTGCCACTGTAGCTTATTT
TAATATGGTCTATATGCCTGCTAGTTGGGTGATGCGTATTATGACATGG
TTGGATATGGTTGATACTAGTTTGTCTGGTTTTAAGCTAAAAGACTGTG
TTATGTATGCATCAGCTGTAGTGTTACTAATCCTTATGACAGCAAGAAC
TGTGTATGATGATGGTGCTAGGAGAGTGTGGACACTTATGAATGTCTTG
ACACTCGTTTATAAAGTTTATTATGGTAATGCTTTAGATCAAGCCATTT
CCATGTGGGCTCTTATAATCTCTGTTACTTCTAACTACTCAGGTGTAGT
TACAACTGTCATGTTTTTGGCCAGAGGTATTGTTTTTATGTGTGTTGAG
TATTGCCCTATTTTCTTCATAACTGGTAATACACTTCAGTGTATAATGC
TAGTTTATTGTTTCTTAGGCTATTTTTGTACTTGTTACTTTGGCCTCTT
TTGTTTACTCAACCGCTACTTTAGACTGACTCTTGGTGTTTATGATTAC
TTAGTTTCTACACAGGAGTTTAGATATATGAATTCACAGGGACTACTCC
CACCCAAGAATAGCATAGATGCCTTCAAACTCAACATTAAATTGTTGGG
TGTTGGTGGCAAACCTTGTATCAAAGTAGCCACTGTACAGTAA.

In some embodiments, the antigen is an RBD trimer protein, e.g. having the amino acid sequence:

(SEQ ID NO: 145)
MPMGSLQPLATLYLLGMLVASCLGRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFGSGYIPEAPRDGQAYVRKDGEWVLLSTFLG.

Therefore, in some embodiments, the RBD trimer protein is encoded by the nucleic acid sequence:

(SEQ ID NO: 146)
ATGCCCATGGGGTCTCTGCAACCGCTGGCCACCTTGTACCTGCTGGGAA
TGCTGGTTGCTTCCTGCCTCGGACGCGTCCAGCCAACCGAGTCCATTGT
GCGGTTTCCGAACATCACAAACCTGTGTCCCTTCGGGGAGGTGTTCAAC
GCCACACGGTTTGCTAGTGTTTACGCATGGAATCGAAAGCGAATTAGTA
ACTGCGTGGCAGACTACTCAGTTCTTTATAACAGTGCGAGCTTCTCTAC
TTTTAAGTGCTACGGTGTGTCCCCCACCAAGCTTAACGACCTCTGTTTT
ACCAACGTCTACGCCGATAGCTTCGTCATTAGGGGGGATGAAGTGAGAC
AGATCGCACCTGGCCAGACTGGGAAAATCGCCGATTACAACTACAAACT
GCCGGATGATTTCACAGGCTGCGTTATCGCTTGGAACAGTAATAACTTG
GACTCAAAAGTGGGAGGGAACTATAATTATCTCTATCGACTGTTCAGAA
AGTCTAACCTGAAACCTTTTGAACGAGATATTAGTACCGAGATTTACCA
AGCTGGCAGTACACCGTGCAACGGCGTCGAGGGGTTCAATTGTTATTTT
CCTCTTCAATCCTACGGTTTTCAGCCTACTAACGGAGTGGGATACCAGC
CATACCGAGTGGTTGTGCTGTCATTCGAGCTGCTGCACGCTCCAGCTAC
CGTCTGTGGCCCTAAAAAGTCCACGAACCTGGTCAAGAACAAATGCGTG
AATTTCGGATCCGGATACATCCCCGAGGCCCCCAGAGATGGCCAGGCCT
ACGTGCGGAAGGACGGCGAGTGGGTACTGCTGAGCACATTCCTGGGCTG
A.

In some embodiments, the antigen is an S1 trimer protein, e.g. having the amino acid sequence:

(SEQ ID NO: 147)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSGSGYIPEAPRDGQAYVRKDGEWVLLSTFLG.

Therefore, in some embodiments, the S1 trimer protein is encoded by the nucleic acid sequence:

(SEQ ID NO: 148)
ATGTTTGTTTTCCTGGTGCTGCTGCCTCTGGTGTCTAGCCAGTGTGTCAACCTGACT
ACCCGAACCCAGCTGCCCCCTGCGTACACCAACTCCTTTACTAGGGGTGTCTACTA
CCCCGACAAGGTCTTTCGGTCTTCTGTACTTCATTCTACACAAGATCTCTTCCTGCC
CTTCTTCAGCAACGTGACATGGTTTCACGCCATCCATGTGTCCGGGACCAATGGAA
CCAAGAGGTTCGACAACCCAGTGTTGCCATTCAACGATGGAGTCTACTTCGCTAGC
ACAGAGAAAAGCAACATTATAAGGGGCTGGATCTTTGGCACCACTCTGGATAGCAA
GACTCAGTCCCTCCTCATCGTGAACAATGCTACCAACGTGGTTATTAAGGTGTGCG
AATTTCAGTTTTGTAATGATCCCTTCCTCGGGGTCTACTATCACAAAAATAATAAGTC
CTGGATGGAGTCCGAGTTCCGAGTTTATTCTTCCGCAAATAATTGCACATTTGAGTA
TGTTAGTCAGCCCTTCCTCATGGACCTCGAAGGCAAACAGGGAAATTTTAAGAACC
TTAGAGAATTCGTCTTCAAAAATATCGATGGGTATTTCAAGATTTATAGCAAGCATAC
ACCCATTAACCTGGTACGAGACCTCCCTCAGGGCTTTTCCGCACTTGAGCCTCTGG
TCGATCTCCCGATAGGCATAAACATAACTAGGTTTCAGACTTTGCTTGCCCTCCATA
GGTCCTACCTTACGCCTGGGGATAGCAGCAGTGGTTGGACTGCGGGCGCCGCCG
CTTACTATGTTGGGTATCTTCAACCTCGCACGTTCCTGCTTAAGTATAATGAAAATG
GGACCATTACGGATGCCGTCGATTGTGCGCTGGATCCTTTGTCCGAGACGAAATGT
ACCCTGAAAAGTTTCACCGTGGAGAAAGGGATTTATCAGACGAGCAATTTCCGCGT
CCAGCCAACCGAGTCCATTGTGCGGTTTCCGAACATCACAAACCTGTGTCCCTTCG
GGGAGGTGTTCAACGCCACACGGTTTGCTAGTGTTTACGCATGGAATCGAAAGCGA
ATTAGTAACTGCGTGGCAGACTACTCAGTTCTTTATAACAGTGCGAGCTTCTCTACT
TTTAAGTGCTACGGTGTGTCCCCCACCAAGCTTAACGACCTCTGTTTTACCAACGTC
TACGCCGATAGCTTCGTCATTAGGGGGGATGAAGTGAGACAGATCGCACCTGGCC
AGACTGGGAAAATCGCCGATTACAACTACAAACTGCCGGATGATTTCACAGGCTGC
GTTATCGCTTGGAACAGTAATAACTTGGACTCAAAAGTGGGAGGGAACTATAATTAT
CTCTATCGACTGTTCAGAAAGTCTAACCTGAAACCTTTTGAACGAGATATTAGTACC
GAGATTTACCAAGCTGGCAGTACACCGTGCAACGGCGTCGAGGGGTTCAATTGTTA
TTTTCCTCTTCAATCCTACGGTTTTCAGCCTACTAACGGAGTGGGATACCAGCCATA
CCGAGTGGTTGTGCTGTCATTCGAGCTGCTGCACGCTCCAGCTACCGTCTGTGGC
CCTAAAAAGTCCACGAACCTGGTCAAGAACAAATGCGTGAATTTCAATTTCAACGGT
CTCACCGGGACGGGCGTGCTTACGGAAAGCAACAAGAAATTCCTGCCATTCCAACA
GTTCGGCCGGGACATCGCCGACACCACTGACGCGGTGCGCGATCCTCAGACATTG
GAAATCCTTGACATTACTCCGTGCTCTTTCGGGGGAGTCTCCGTCATCACCCCAGG
AACTAACACGTCTAACCAGGTGGCTGTTCTGTATCAGGATGTTAATTGCACAGAAGT
GCCCGTTGCGATACACGCGGACCAGCTTACTCCTACCTGGAGGGTCTATTCCACC
GGCTCTAACGTCTTTCAGACCCGCGCCGGTTGCCTCATAGGCGCAGAACATGTAAA
TAATAGTTATGAATGCGACATTCCTATTGGCGCCGGGATCTGTGCGAGCTACCAGA
CTCAAACCAACAGCCCGAGGCGGGCCAGGTCCGTGGCGAGTCAGTCCATTATTGC
TTATACAATGTCCGGATCCGGATACATCCCCGAGGCCCCCAGAGATGGCCAGGCC
TACGTGCGGAAGGACGGCGAGTGGGTACTGCTGAGCACATTCCTGGGCTGA.

In some embodiments, the antigen is an Fc-fused RBD protein, e.g. having the amino acid sequence:

(SEQ ID NO: 149)
MPMGSLQPLATLYLLGMLVASCLGRVQPTESIVRFPNITNLCPFGEVFNAT
RFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQ
PTNGVGYQPYRVVVLSFELLHAPATVCGPKKSTNLVKNKCVNFAPELLGGP
SVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
GGTRGVRGPHGQRPARPTLCPESDRCTNLCPTGQPREPQVYTLPPSRDELT
KNQVSLTCLVKGFYPSDIAVEWESNGQPENNYKTTPPVLDSDGSFFLYSKL
TVDKSRWQQGNVFSCSVMHEALHNHYTQKSLSLSPGK*VRRPASPAPRALA
VARGCLARTPCTYFPGAQHGNK.

Therefore, in some embodiments, the Fc-fused RBD protein is encoded by the nucleic acid sequence:

(SEQ ID NO: 150)
ATGCCCATGGGGTCTCTGCAACCGCTGGCCACCTTGTACCTGCTGGGAATG
CTGGTTGCTTCCTGCCTCGGACGCGTCCAGCCAACCGAGTCCATTGTGCGG
TTTCCGAACATCACAAACCTGTGTCCCTTCGGGGAGGTGTTCAACGCCACA
CGGTTTGCTAGTGTTTACGCATGGAATCGAAAGCGAATTAGTAACTGCGTG
GCAGACTACTCAGTTCTTTATAACAGTGCGAGCTTCTCTACTTTTAAGTGC
TACGGTGTGTCCCCCACCAAGCTTAACGACCTCTGTTTTACCAACGTCTAC
GCCGATAGCTTCGTCATTAGGGGGGATGAAGTGAGACAGATCGCACCTGGC
CAGACTGGGAAAATCGCCGATTACAACTACAAACTGCCGGATGATTTCACA
GGCTGCGTTATCGCTTGGAACAGTAATAACTTGGACTCAAAAGTGGGAGGG
AACTATAATTATCTCTATCGACTGTTCAGAAAGTCTAACCTGAAACCTTTT
GAACGAGATATTAGTACCGAGATTTACCAAGCTGGCAGTACACCGTGCAAC
GGCGTCGAGGGGTTCAATTGTTATTTTCCTCTTCAATCCTACGGTTTTCAG
CCTACTAACGGAGTGGGATACCAGCCATACCGAGTGGTTGTGCTGTCATTC
GAGCTGCTGCACGCTCCAGCTACCGTCTGTGGCCCTAAAAAGTCCACGAAC
CTGGTCAAGAACAAATGCGTGAATTTCGCACCTGAACTCCTGGGGGGACCG
TCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACA
AAGCCGCGGGAGGAGCAGTACAACAGCACGTACCGGGTGGTCAGCGTCCTC
ACCGTCCTGCACCAGGACTGGCTGAATGGCAAGGAGTACAAGTGCAAGGTC
TCCAACAAAGCCCTCCCAGCCCCCATCGAGAAAACCATCTCCAAAGCCAAA
GGTGGGACCCGTGGGGTGCGAGGGCCACATGGACAGAGGCCGGCTCGGCCC
ACCCTCTGCCCTGAGAGTGACCGCTGTACCAACCTCTGTCCTACAGGGCAG
CCCCGAGAACCACAGGTGTACACCCTGCCCCCATCCCGGGATGAGCTGACC
AAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACC
ACGCCTCCCGTGCTGGACTCCGACGGCTCCTTCTTCCTCTACAGCAAGCTC
ACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCT
CCGGGTAAATGAGTGCGACGGCCGGCAAGCCCCGCTCCCCGGGCTCTCGCG
GTCGCACGAGGATGCTTGGCACGTACCCCCTGTACATACTTCCCGGGCGCC
CAGCATGGAAATAAATGA.

In some embodiments, the antigen is an Fc-fused S1 protein, e.g. having the amino acid sequence:

(SEQ ID NO: 151)
MFVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSS
SGWTAGAAAYYVGYLQPRTFLLKYNENGTITDAVDCALDPLSETKCTLKSF
TVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECD
IPIGAGICASYQTQTNSPRRARSVASQSIIAYTMSAPELLGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGGTRGVRG
PHGQRPARPTLCPESDRCTNLCPTGQPREPQVYTLPPSRDELTKNQVSLTC
LVKGFYPSDIAVEWESNGQPENNYKTTPPVLDSDGSFFLYSKLTVDKSRWQ
QGNVFSCSVMHEALHNHYTQKSLSLSPGK*VRRPASPAPRALAVARGCLAR
TPCTYFPGAQHGNK.

Therefore, in some embodiments, the Fc-fused S1 protein is encoded by the nucleic acid sequence:

(SEQ ID NO: 152)
ATGTTTGTTTTCCTGGTGCTGCTGCCTCTGGTGTCTAGCCAGTGTGTCAACCTGACT
ACCCGAACCCAGCTGCCCCCTGCGTACACCAACTCCTTTACTAGGGGTGTCTACTA
CCCCGACAAGGTCTTTCGGTCTTCTGTACTTCATTCTACACAAGATCTCTTCCTGCC
CTTCTTCAGCAACGTGACATGGTTTCACGCCATCCATGTGTCCGGGACCAATGGAA
CCAAGAGGTTCGACAACCCAGTGTTGCCATTCAACGATGGAGTCTACTTCGCTAGC
ACAGAGAAAAGCAACATTATAAGGGGCTGGATCTTTGGCACCACTCTGGATAGCAA
GACTCAGTCCCTCCTCATCGTGAACAATGCTACCAACGTGGTTATTAAGGTGTGCG
AATTTCAGTTTTGTAATGATCCCTTCCTCGGGGTCTACTATCACAAAAATAATAAGTC
CTGGATGGAGTCCGAGTTCCGAGTTTATTCTTCCGCAAATAATTGCACATTTGAGTA
TGTTAGTCAGCCCTTCCTCATGGACCTCGAAGGCAAACAGGGAAATTTTAAGAACC
TTAGAGAATTCGTCTTCAAAAATATCGATGGGTATTTCAAGATTTATAGCAAGCATAC
ACCCATTAACCTGGTACGAGACCTCCCTCAGGGCTTTTCCGCACTTGAGCCTCTGG
TCGATCTCCCGATAGGCATAAACATAACTAGGTTTCAGACTTTGCTTGCCCTCCATA
GGTCCTACCTTACGCCTGGGGATAGCAGCAGTGGTTGGACTGCGGGCGCCGCCG
CTTACTATGTTGGGTATCTTCAACCTCGCACGTTCCTGCTTAAGTATAATGAAAATG
GGACCATTACGGATGCCGTCGATTGTGCGCTGGATCCTTTGTCCGAGACGAAATGT
ACCCTGAAAAGTTTCACCGTGGAGAAAGGGATTTATCAGACGAGCAATTTCCGCGT
CCAGCCAACCGAGTCCATTGTGCGGTTTCCGAACATCACAAACCTGTGTCCCTTCG
GGGAGGTGTTCAACGCCACACGGTTTGCTAGTGTTTACGCATGGAATCGAAAGCGA
ATTAGTAACTGCGTGGCAGACTACTCAGTTCTTTATAACAGTGCGAGCTTCTCTACT
TTTAAGTGCTACGGTGTGTCCCCCACCAAGCTTAACGACCTCTGTTTTACCAACGTC
TACGCCGATAGCTTCGTCATTAGGGGGGATGAAGTGAGACAGATCGCACCTGGCC
AGACTGGGAAAATCGCCGATTACAACTACAAACTGCCGGATGATTTCACAGGCTGC
GTTATCGCTTGGAACAGTAATAACTTGGACTCAAAAGTGGGAGGGAACTATAATTAT
CTCTATCGACTGTTCAGAAAGTCTAACCTGAAACCTTTTGAACGAGATATTAGTACC
GAGATTTACCAAGCTGGCAGTACACCGTGCAACGGCGTCGAGGGGTTCAATTGTTA
TTTTCCTCTTCAATCCTACGGTTTTCAGCCTACTAACGGAGTGGGATACCAGCCATA
CCGAGTGGTTGTGCTGTCATTCGAGCTGCTGCACGCTCCAGCTACCGTCTGTGGC
CCTAAAAAGTCCACGAACCTGGTCAAGAACAAATGCGTGAATTTCAATTTCAACGGT
CTCACCGGGACGGGCGTGCTTACGGAAAGCAACAAGAAATTCCTGCCATTCCAACA
GTTCGGCCGGGACATCGCCGACACCACTGACGCGGTGCGCGATCCTCAGACATTG
GAAATCCTTGACATTACTCCGTGCTCTTTCGGGGGAGTCTCCGTCATCACCCCAGG
AACTAACACGTCTAACCAGGTGGCTGTTCTGTATCAGGATGTTAATTGCACAGAAGT
GCCCGTTGCGATACACGCGGACCAGCTTACTCCTACCTGGAGGGTCTATTCCACC
GGCTCTAACGTCTTTCAGACCCGCGCCGGTTGCCTCATAGGCGCAGAACATGTAAA
TAATAGTTATGAATGCGACATTCCTATTGGCGCCGGGATCTGTGCGAGCTACCAGA
CTCAAACCAACAGCCCGAGGCGGGCCAGGTCCGTGGCGAGTCAGTCCATTATTGC
TTATACAATGTCCGCACCTGAACTCCTGGGGGGACCGTCAGTCTTCCTCTTCCCCC
CAAAACCCAAGGACACCCTCATGATCTCCCGGACCCCTGAGGTCACATGCGTGGT
GGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGT
ACCGGGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCT
CCAAAGCCAAAGGTGGGACCCGTGGGGTGCGAGGGCCACATGGACAGAGGCCGG
CTCGGCCCACCCTCTGCCCTGAGAGTGACCGCTGTACCAACCTCTGTCCTACAGG
GCAGCCCCGAGAACCACAGGTGTACACCCTGCCCCCATCCCGGGATGAGCTGACC
AAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTC
CCGTGCTGGACTCCGACGGCTCCTTCTTCCTCTACAGCAAGCTCACCGTGGACAA
GAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAATGAGTGCGAC
GGCCGGCAAGCCCCGCTCCCCGGGCTCTCGCGGTCGCACGAGGATGCTTGGCAC
GTACCCCCTGTACATACTTCCCGGGCGCCCAGCATGGAAATAAATGA.

Pharmaceutical Compositions and Therapeutic Applications

Also disclosed herein are pharmaceutical compositions and related therapeutic methods of using the rMeV-based coronavirus vaccine disclosed herein. In various embodiments, the rMeV-based coronavirus vaccine disclosed herein can be used for preventing and treating coronavirus infections. Some embodiments relate to use of the rMeV-based coronavirus vaccine disclosed herein for preventing or treating SARS-CoV-1, MERS-CoV, or SARS-CoV-2 infections in human subjects.

In some embodiments, the disclosed rMeV-based coronavirus vaccine is in a pharmaceutical composition. The pharmaceutical composition can be either a therapeutic formulation or a prophylactic formulation. Typically, the composition can additionally include one or more pharmaceutically acceptable vehicles and, optionally, other therapeutic ingredients (for example, antiviral drugs). Various pharmaceutically acceptable additives can also be used in the compositions.

For vaccine compositions, appropriate adjuvants can be additionally included. Examples of suitable adjuvants include, e.g., aluminum hydroxide, lecithin, Freund's adjuvant, MPL™ and IL-12. In some embodiments, the rMeV-based coronavirus vaccine can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art. See, e.g., Remington's Pharmaceutical Sciences, 19th Ed., Mack Publishing Company, Easton, Pa., 1995; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

The disclosed pharmaceutical compositions can be readily employed in a variety of therapeutic or prophylactic applications, e.g., for treating SARS-CoV-2 infection or eliciting an immune response to SARS-CoV-2 in a subject. As exemplification, a rMeV-based SARS-CoV-2 vaccine composition can be administered to a subject to induce an immune response to SARS-CoV-2, e.g., to induce production of broadly neutralizing antibodies to the virus. For subjects at risk of developing an SARS-CoV-2 infection, a rMeV-based SARS-CoV-2 vaccine can be administered to provide prophylactic protection against viral infection. Therapeutic and prophylactic applications of vaccines derived from the other immunogens described herein can be similarly performed. Depending on the specific subject and conditions, pharmaceutical compositions can be administered to subjects by a variety of administration modes known to the person of ordinary skill in the art, for example, intramuscular, subcutaneous, intravenous, intra-arterial, intra-articular, intraperitoneal, or parenteral routes.

In general, the pharmaceutical composition is administered to a subject in need of such treatment for a time and under conditions sufficient to prevent, inhibit, and/or ameliorate a selected disease or condition or one or more symptom(s) thereof. In various embodiments, the therapeutic methods of the invention relate to methods of blocking the entry of a coronavirus (e.g., SARS-CoV, SARS-CoV-2, or MERS-CoV) into a host cell, e.g., a human host cell, methods of preventing the S protein of a coronavirus from binding the host receptor, and methods of treating acute respiratory distress that is often associated with coronavirus infections. In some embodiments, the therapeutic methods and compositions described herein can be employed in combination with other known therapeutic agents and/or modalities useful for treating or preventing coronavirus infections. The known therapeutic agents and/or modalities include, e.g., a nuclease analog or a protease inhibitor (e.g., remdesivir), monoclonal antibodies directed against one or more coronaviruses, an immunosuppressant or anti-inflammatory drug (e.g., sarilumab or tocilizumab), ACE inhibitors, vasodilators, or any combination thereof.

For therapeutic applications, the compositions should contain a therapeutically effective amount of the nanoparticle immunogen described herein. For prophylactic applications, the compositions should contain a prophylactically effective amount of the nanoparticle immunogen described herein. The appropriate amount of the immunogen can be determined based on the specific disease or condition to be treated or prevented, severity, age of the subject, and other personal attributes of the specific subject (e.g., the general state of the subject's health and the robustness of the subject's immune system). Determination of effective dosages is additionally guided with animal model studies followed up by human clinical trials and is guided by administration protocols that significantly reduce the occurrence or severity of targeted disease symptoms or conditions in the subject.

For prophylactic applications, the immunogenic composition is provided in advance of any symptom, for example in advance of infection. The prophylactic administration of the immunogenic compositions serves to prevent or ameliorate any subsequent infection. Thus, in some embodiments, a subject to be treated is one who has, or is at risk for developing, an infection (e.g., SARS-CoV-2 infection), for example because of exposure or the possibility of exposure to the virus (e.g., SARS-CoV-2). Following administration of a therapeutically effective amount of the disclosed therapeutic compositions, the subject can be monitored for an infection (e.g., SARS-CoV-2 infection), symptoms associated with an infection (e.g., SARS-CoV-2 infection), or both.

For therapeutic applications, the immunogenic composition is provided at or after the onset of a symptom of disease or infection, for example after development of a symptom of infection (e.g., SARS-CoV-2 infection), or after diagnosis of the infection. The immunogenic composition can thus be provided prior to the anticipated exposure to the virus so as to attenuate the anticipated severity, duration or extent of an infection and/or associated disease symptoms, after exposure or suspected exposure to the virus, or after the actual initiation of an infection. The pharmaceutical composition of the invention can be combined with other agents known in the art for treating or preventing infections by a relevant pathogen (e.g., SARS-CoV-2 infection).

The nanoparticle vaccine compositions containing novel structural components as described in the invention (e.g., SARS-CoV-2 vaccine) or pharmaceutical compositions of the invention can be provided as components of a kit. Optionally, such a kit includes additional components including packaging, instructions and various other reagents, such as buffers, substrates, antibodies or ligands, such as control antibodies or ligands, and detection reagents. An optional instruction sheet can be additionally provided in the kits.

Aspects of the Disclosure

Aspect 1. A live attenuated recombinant measles virus (rMeV)-based coronavirus vaccine comprising a SARS-CoV-2 spike (S) protein inserted between the P and M genes of the rMeV genome, wherein the S protein comprises at least one mutation to remove a glycosylation site.

Aspect 2. The vaccine of aspect 1, wherein the S protein is a soluble stabilized prefusion S protein.

Aspect 3. The vaccine of aspect 2, wherein the soluble stabilized prefusion S protein comprises the amino acid sequence SEQ ID NO:15, SEQ ID NO:127, SEQ ID NO:129, or SEQ ID NO:131.

Aspect 4. The vaccine of aspect 1, wherein the S protein is S1 protein.

Aspect 5. The vaccine of aspect 4, wherein the S1 comprises the amino acid sequence SEQ ID NO:4.

Aspect 6. The vaccine of aspect 1, wherein the S protein is S2 protein.

Aspect 7. The vaccine of aspect 6, wherein the S2 comprises the amino acid sequence SEQ ID NO:6.

Aspect 8. The vaccine of aspect 1, wherein the S protein lacks the transmembrane domain.

Aspect 9. The vaccine of aspect 8, wherein the S protein comprises the amino acid sequence SEQ ID NO:13.

Aspect 10. The vaccine of aspect 1, wherein the S protein is an S protein fragment comprising at least the receptor-binding domain (RBD).

Aspect 11. The vaccine of aspect 10, wherein the RBD comprises the amino acid sequence SEQ ID NO:124, 125, or 126.

Aspect 12. The vaccine of aspect 1, wherein the S protein is a trimeric S1 protein.

Aspect 13. The vaccine of aspect 12, wherein the trimeric S1 comprises the amino acid sequence SEQ ID NO:147.

Aspect 14. The vaccine of aspect 1, wherein the S protein is an Fc-fused S1 protein.

Aspect 15. The vaccine of aspect 14, wherein the Fc-fused S1 protein comprises the amino acid sequence SEQ ID NO:151.

Aspect 16. The vaccine of aspect 1, wherein the S protein is a trimeric RBD protein.

Aspect 17. The vaccine of aspect 16, wherein the trimeric RBD comprises the amino acid sequence SEQ ID NO:147.

Aspect 18. The vaccine of aspect 1, wherein the S protein is an Fc fused RBD protein.

Aspect 19. The vaccine of aspect 18, wherein the Fc fused RBD comprises the amino acid sequence SEQ ID NO:149.

Aspect 20. The vaccine of any one of aspects 1 to 19, wherein the rMeV comprises the Edmonston strain, Schwarz strain, or Shanghai strain.

Aspect 21. A live attenuated recombinant coronavirus vaccine, wherein a stabilized prefusion spike (S) protein is inserted into a viral vector genome.

Aspect 22. The vaccine of aspect 21, wherein the viral vector is a live attenuated recombinant measles virus (rMeV).

Aspect 23. The vaccine of aspect 21 or 22, wherein the coronavirus is SARS-CoV-2.

Aspect 24. The vaccine of any one of aspects 21 to 23, wherein the stabilized prefusion S protein comprises the amino acid sequence SEQ ID NO:15, SEQ ID NO:127, SEQ ID NO:129, or SEQ ID NO:131.

Aspect 25. The vaccine of any one of aspects 21 to 24, further comprising at least one coronavirus structural protein, accessory protein, nonstructural protein, or a combination thereof.

Aspect 26. The vaccine of aspect 25, wherein the structural protein comprises an M, N, or E protein.

Aspect 27. The vaccine of aspect 25 or 26, wherein the accessory protein comprises ORF3a or ORFS.

Aspect 28. The vaccine of any one of aspects 25 to 27, wherein the nonstructural protein comprises nsp6.

Aspect 29. A recombinant measles virus (rMeV) system, comprising a yeast expression vector comprising a yeast replication origin, a T7 RNA polymerase, a hepatitis delta virus (HDV) ribozyme sequence, a T7 promoter, and a cDNA clone of measles virus (MeV) genome.

Aspect 30. The system of aspect 29, further comprising a coronavirus antigen inserted between the P and M genes of the MeV genome.

Aspect 31. The system of aspect 29 or 30, wherein the yeast expression vector comprises a pYES2 vector.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Summary

On Mar. 11, 2020, the World Health Organization (WHO) declared the novel coronavirus outbreak (COVID-19) a global pandemic. The causative agent, named severe acute respiratory syndrome coronavirus 2 (SARS-CoV-2), was first identified in December 2019 in Wuhan City, Hubei Province, China. It spread rapidly within China and swept into at least 200 countries within 3 months. Symptoms are primarily pneumonia as with other human coronaviruses, such as SARS-CoV and MERS-CoV. As of Apr. 22, 2020, more than 2,603,147 cases had been reported worldwide, with 180,784 deaths (˜6.9% mortality). However, mortality has varied among countries. In Italy, mortality reached 13.4%. In the US, the total cases reached 837,136, which has resulted in 46,997 deaths (˜5.6% mortality). There is an urgent need to develop a safe and efficacious vaccine to protect the populace from this new virus. In the US, the first clinical trial of an mRNA vaccine candidate (called mRNA-1273) developed by Moderna was initiated on March 16. On the next day, China approved the first homegrown COVID-19 vaccine clinical trial. This vaccine candidate, known as Ad5-nCoV, is a replication-defective adenovirus type 5 (a DNA virus)-vectored vaccine. As of Apr. 22, 2020, the safety and efficacy of these two vaccine candidates are currently being investigated.

The family Coronaviridae includes many important human and animal pathogens, which can be classified into Coronavirinae and Torovirinae subfamily. The Coronavirinae subfamily can be further subdivided into four genera, Alphacoronavirus, Betacoronavirus, Gammacoronavirus, and Deltacoronavirus. The genus Alphacoronavirus includes several economically important pig CoVs such as human coronavirus NL63 (HCoV-NL63), porcine epidemic diarrhea virus (PEDV), transmissible gastroenteritis virus (TGEV), and swine enteric alphacoronavirus (SeACoV). The genus Betacoronavirus includes many important human pathogens such as SARS-CoV, Middle East respiratory syndrome coronavirus (MERS-CoV), and the 2019 newly emerged SARS-CoV-2. Example of Gammacoronavirus includes avian infectious bronchitis virus (IBV). The genus Deltacoronavirus includes porcine deltacoronavirus (PDCoV) and avian deltacoronavirus. Currently, there is no FDA-approved vaccine for most of CoVs.

CoV entry is mediated by its spike (S) protein, a “class 1” fusion protein that possesses both receptor binding and fusion activity. As such, the S protein is the main target for neutralizing antibodies that protect from future CoV infection. Thus, the S protein is the main focus for CoV vaccine development. The ectodomain includes an S1 subunit, which includes the receptor-binding domain (RBD), and the S2 subunit, which includes the membrane-fusing mechanism.

Live attenuated measles virus (MeV) vaccine is one of the safest and most efficient human vaccines that has been used in children since the 1960s. The vaccination campaigns in industrialized world have been very successful in controlling measles. MeV is an enveloped non-segmented negative-sense RNA virus that belongs to the genus Morbillivirus within the Paramyxoviridae family. In 1997, a reverse genetics system, which allows us to recover recombinant MeV (rMeV) from the cloned full-length MeV genomic cDNA, was established. With this technology, an exogenous foreign gene can be inserted into the MeV genome and recombinant MeV expressing this antigen can be generated. Once the recombinant viruses are inoculated into animals or humans, the exogenous antigen is expressed continuously in vivo thus trigger specific immune responses. This vaccine is termed “live vectored vaccine”. MeV is an excellent vector to deliver vaccines for other pathogens. First, live attenuated MeV vaccine has been widely used and has excellent track record of high safety and efficacy in human population. Second, MeV is an RNA virus and it does not undergo either recombination or integration into host cell DNA. Third, MeV has an excellent genetic stability. The genome of MeV is relatively simple that can accommodate up to 6 kb of foreign genes. The foreign gene remains genetically stable in MeV genome. Fourth, the inserted foreign antigens are highly expressed by MeV vector, which in turn generate long-lasting humoral, cellular, and mucosal immunities. Fifth, MeV grows to a high titer in Vero cells, a WHO approved cell line for vaccine production, facilitating vaccine manufacture. Sixth, large numbers of vaccination doses can be easily produced, making vaccine production economically feasible, and thus MeV vectored vaccine can be affordable in low or moderate income countries. Seventh, MeV vectored vaccines are highly efficacious even in the presence of pre-existing MeV immunity. A large number of preclinical, non-human primate, and human clinical trials demonstrated that MeV vectored vaccine is capable of replicating and expressing foreign proteins efficiently in vivo and generating high level of immune responses despite the pre-existing anti-MeV immunity. rMeV has been shown to be a highly efficacious vaccine vector for a number of viral disease such as human immunodeficiency virus (HIV), respiratory syncytial virus (RSV), hepatitis B and C viruses, influenza virus, and flaviviruses (WNV, DENV, YFV, and CHIKV). Particularly, recent human clinical trials showed that rMeV-based CHIKV vaccine was safe and highly immunogenic in healthy adults, even in the presence of pre-existing anti-MeV vector immunity. Thus, MeV-vectored vaccine is highly promising for future use in humans.

Herein, a rMeV-based vaccine platform for SARS-CoV-2 is described. The rMeV-based SARS-CoV-2 candidate vaccine is a live attenuated recombinant viral vectored vaccine based on the Edmonston strain of measles vaccine, which has been widely used in the US and many other countries since 1960. rMeV expressing 1) full-length pre-fusion and post-fusion S proteins; 2) truncated S proteins lacking its transmembrane and cytoplasmic domains; 3) S proteins lacking glycosylation sites, and 4) the receptor-binding domain (RBD) of S protein was generated. These recombinant viruses grew to a high titer in Vero cells and SARS-CoV-2 S and RBD antigens were highly expressed by MeV vector. These findings demonstrated that rMeV-based vaccine candidate is highly promising for SARS-CoV-2. These vaccine candidates can directly lead to clinical trials in nonhuman primate and humans in the future.

Materials and Methods

Cells culture: Vero CCL81 cells (African green monkey, ATCC-CCL-81) and HEp-2 cells (ATCC CCL-23) were grown in Dulbecco's modified Eagle's medium (DMEM; Life Technologies) supplemented with 10% fetal bovine serum (FBS).

Insertion of Zika virus prM-E and prM-E-NS1 gene at different locations in the MeV genome. The full-length anchor C (signal peptide, sp)-premembrane-envelope (prM-E), and sp-premembrane-envelope-nonstructural protein 1 (prM-E-NS1) genes were amplified from an infectious cDNA clones of ZIKV Cambodian strain by high fidelity PCR with the upstream and downstream primers containing measles virus gene start and gene end sequences. These DNA fragments were digested with MluI and NruI and cloned into the H and L gene junction in an infectious cDNA clone of attenuated measles virus Edmonston vaccine strain (pMeV) at the same sites by standard cloning procedure. The resulting plasmids were designated pMeV(+)-prM-E^{HL}, and pMeV(+)-prM-E-NS1^{HL} respectively. Using a similar strategy, the prM-E and prM-E-NS1 were inserted into the P and M gene junction in MeV genome, which resulted in the construction of pMeV(+)-prM-E^{pM}, and pMeV(+)-prM-E-NS1^{PM} respectively. All of the constructs were confirmed by sequencing.

Recovery of recombinant MeV expressing ZIKV antigens. Recovery of recombinant MeV from the infectious clone was carried out as described previously. Briefly, recombinant MeV was recovered by cotransfection of the plasmid encoding genome of the MeV Edmonston strain, and support plasmids encoding the MeV nucleocapsid complex (pN, pP, and pL) into Vero cells infected with a recombinant vaccinia virus (MVA-T7) expressing T7 RNA polymerase (kindly provided by Dr. Bernard Moss). At 96 h post-transfection, cell culture fluids were collected, and the recombinant virus was further amplified in Vero cells. Subsequently, the viruses were plaque purified as described previously. Individual plaques were isolated, and seed stocks were amplified in Vero cells. The viral titer was determined by a plaque assay performed in Vero cells. The recovered recombinant viruses were named rMeV(+)-prM-E^{HL}, rMeV(+)-prM-E-NS1^{HL}, rMeV(+)-prM-E^{PM}, and rMeV(+)-prM-E-NS1^{PM}.

Verification of Zika genes by RT-PCR. To characterize the insertion of ZIKV genes, viral RNA was extracted from recombinant MeVs by using an RNeasy minikit (Qiagen, Valencia, CA) according to the manufacturer's instructions. Zika virus E or prM-E gene were amplified by a One Step RT-PCR kit (Qiagen) using primers annealing to the MeV H gene at position 9042 (5′-GTGGACATATCACTCACTCTG-3′, SEQ ID NO:17) and the MeV L gene at position 9811 (5′-GGTGTGTGTCTCCTCCTAT-3′, SEQ ID NO:18). In addition, the ZIKV NS1 gene was amplified using primers annealing to the ZIKV E gene at position 1957 (5′-CTCATTGGAACGTTGCTGGTG-3′, SEQ ID NO:19) and the MeV L gene at position 9811 (5′-GGTGTGTGTCTCCTCCTAT-3′, SEQ ID NO:20). The amplified products were analyzed on 1% agarose gel electrophoresis and sequenced.

Comparison of ZIKV antigen expression at different location of MeV geneome by Western blot. Vero cells were infected with each rMeV expressing ZIKV antigen at an MOI of 1.0 as described above. At the indicated times post-infection, cell culture medium was harvested and clarified at 5,000 g for 15 min and further concentrated at 40,000 g for 1.5 h. In the meantime, cells were lysed in lysis buffer containing 5% β-mercaptoethanol, 0.01% NP-40, and 2% SDS. Proteins were separated by 12% SDS-PAGE and transferred to a Hybond enhanced chemiluminescence nitrocellulose membrane (Amersham) in a Mini Trans-Blot electrophoretic transfer cell (Bio-Rad). The blot was probed with rabbit anti-ZIKV E or NS1 antibody at a dilution of 1:2,000, followed by horseradish peroxidase-conjugated goat anti-rabbit IgG secondary antibody (Santa Cruz) at a dilution of 1:5,000. The blot was developed with SuperSignal West Pico chemiluminescent substrate (Thermo Scientific) and exposed to Kodak BioMax MR film.

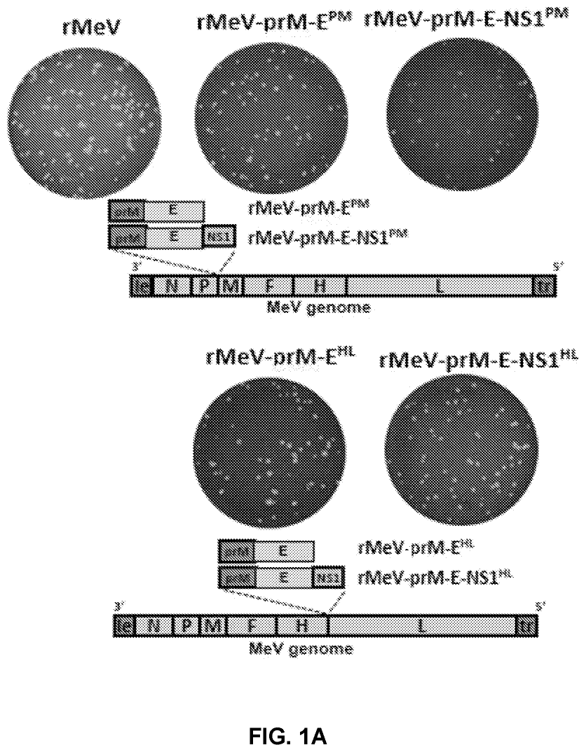

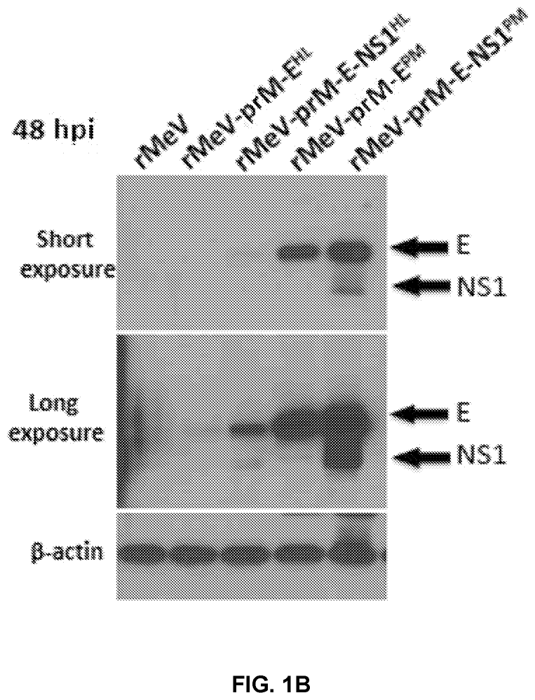

Rapid assembly of the full-length genomic cDNA of MeV by yeast-based recombination system. The full-length genomic cDNA of Edmonston strain of measles vaccine was assembled into pYES2 vector. The pYES vector was modified to insert a yeast replication origin from the plasmid pYES1L (Invitrogen), a T7 RNA polymerase promoter, a hepatitis delta virus ribozyme (HDVRz) sequence, and a T7 terminator. The full-length cDNA clone of MeV was constructed using six overlapping fragments (designated from A to F) by using yeast recombination system. Briefly, 100 ng of pYES2 vector was mixed with 200 ng of each MeV DNA fragment in PEG/LiAc solution, and the ligation products were transformed into MaV 203 competent yeast cells by electroporation and plated on SD/Ura⁻ agar plates. After incubation for 2 days at 30° C., individual colony was picked for yeast colony PCR analysis. For initial screening, the connection regions between fragments were amplified by RT-PCR and sequenced. The positive plasmid was then transformed into TOP10B competent cells, and plasmid DNA was verified by restriction enzyme digestion, PCR analysis, and sequenced to confirm that no additional mutations were introduced during the assembly. The final plasmid was designated as pYES2-SARS-CoV-2 (FIG. 1). Primers used in this study was listed in Table 1. Using this method, SARS-CoV-2 S, S1, RBD1, and RBD2 containing MeV gene start and gene end sequences were inserting into the gene junction between P and M genes in the MeV genome. These plasmids were named pYES2-S, S1, S1, RBD1, and RBD2. All of the constructs were confirmed by sequencing.

Prediction 3D structure model of SARS-CoV-2. Crystal structure of SARS-CoV S was chosen as the template to generate 3D model. Protein structure predictions were carried via MODELLER, and using publicly available service for fold recognition: mGenTHREADER. Structural figures were drawn with Chimera. Structural based sequence alignments were displayed with ESPRIPT. The N-glycan in S protein was predicted by PyMol software.

Recovery of recombinant MeV expressing SARS-CoV-2 antigens. Recovery of recombinant MeV from the infectious clone was carried out as described previously. Briefly, recombinant MeV was recovered by cotransfection of plasmid encoding genome of MeV Edmonston strain with S gene, truncated S, and RBDs of S, and support plasmids encoding MeV nucleocapsid complex (pN, pP, and pL) into Vero cells infected with a recombinant vaccinia virus (MVA-T7) expressing T7 RNA polymerase. At 96 h post-transfection, cell culture fluids were collected, and the recombinant virus was further amplified in Vero cells. Subsequently, the viruses were plaque purified as described previously. Individual plaques were isolated, and seed stocks were amplified in Vero cells. The viral titer was determined by a plaque assay performed in Vero cells.

RT-PCR verification of SARS-CoV-2 gene. To characterize the insertion of SARS-CoV-2 genes, viral RNA was extracted from recombinant MeVs by using an RNeasy minikit (Qiagen, Valencia, CA) according to the manufacturer's instructions. SARS-CoV-2 S, S1, RBD1, and RBD2 gene were amplified by a One Step RT-PCR kit (Qiagen) using primers annealing to MeV P gene and MeV M gene. The amplified products were analyzed on 1% agarose gel electrophoresis and sequenced. Primers used for RT-PCR and sequencing of pVBS-SARS-CoV-2 are listed in Table 1.

Single-cycle growth curves. Confluent Vero cells were infected with individual viruses at a multiplicity of infection (MOI) of 0.1. After 1 h of absorption, the inoculum was removed, the cells were washed twice with Dulbecco's modified Eagle's medium (DMEM), fresh DMEM (supplemented with 2% fetal bovine serum) was added, and the infected cells were incubated at 37° C. Aliquots of the cell culture fluid were removed at the indicated intervals, and virus titers were determined by plaque assay in Vero cells.

Plaque assays. Confluent Vero CC81 cells in 6-well plates were infected with serial dilutions of rMeV or rMeV expressing SARS-CoV-2 antigen in DMEM. After absorption for 1 h at 37° C., cells were washed three times with DMEM and overlaid with 2 ml of DMEM containing low-melting agarose (1% w/v). After incubation at 37° C. for 4-5 days, cells were fixed with 4% paraformaldehyde for 2 h. The overlays were removed, and the plaques were visualized after staining by crystal violet. The diameter of plaques for each virus were measured using Image J Software.

Detection of SARS-CoV-2 S antigen by Western blot. Vero cells were infected with each rMeV expressing SARS-CoV-2 antigen as described above. At the indicated times post-infection, cell culture medium was harvested and clarified at 5,000 g for 15 min and further concentrated at 40,000 g for 1.5 h. In the meantime, cells were lysed in lysis buffer containing 5% β-mercaptoethanol, 0.01% NP-40, and 2% SDS. Proteins were separated by 12% SDS-PAGE and transferred to a Hybond enhanced chemiluminescence nitrocellulose membrane (Amersham) in a Mini Trans-Blot electrophoretic transfer cell (Bio-Rad). The blot was probed with rabbit RBD or S antibody at a dilution of 1:2,000, followed by horseradish peroxidase-conjugated goat anti-rabbit IgG secondary antibody (Santa Cruz) at a dilution of 1:5,000. The blot was developed with SuperSignal West Pico chemiluminescent substrate (Thermo Scientific) and exposed to Kodak BioMax MR film. Similar protocol was used for determine the expression of the Zika virus protein expression using antibodies against Zika virus E or NS1 protein.

Statistical analysis. Quantitative analysis was performed by either densitometric scanning of autoradiographs or by using a phosphorimager (Typhoon; GE Healthcare, Piscataway, NJ) and ImageQuant TL software (GE Healthcare, Piscataway, NJ) or Image J software (NIH, Bethesda, MD). Statistical analysis was performed by one-way multiple comparisons using SPSS (version 8.0) statistical analysis software (SPSS Inc., Chicago, IL). A P value of <0.05 was considered statistically significant.

Results

Identify an Ideal Location at MeV Genome for Insertion of Foreign Gene.

Edmonston strain of measles vaccine, which is one of the most safest and efficient human vaccines, was used as the vector to deliver SARS-CoV-2 vaccine. MeV is a non-segmented negative-sense (NNS) RNA virus, belonging to the family Paramyxoviridae in the order Mononegavirales. The MV genome is typically 15,894 nucleotides (nt) in length, and it encodes 6 structural proteins arranged in the order of 3′-leader-NP-P-M-F-H-L-trailer 5′. Theoretically, foreign gene can be inserted into each of gene junction. One unique feature of MeV gene expression strategy is that the abundance of gene expression decreases with distance from the 3′ end to the 5′end of the MeV genome. Thus, the foreign gene inserted at the gene junction at the 3′ of MeV genome will be more abundantly expressed than those inserted at the 5′ end. Ideally, the SARS-CoV-2 S gene should be inserted at the first gene junction (between leader sequence and NP gene) or the second gene junction (between NP and P genes) at the 3′ end of MeV genome. However, the S gene of SARS-CoV-2 is large (approximately 4 kb) and will likely interfere gene expression of MeV which may be lethal to MeV. Thus, it is critical to identify an optimal location in MeV genome that will not affect the recovery of viable recombinant virus but achieve a maximum expression of the inserted antigen. Before the COVID-19 outbreaks, prM-E (approximately 3 kb) and prM-E-NS1 (approximately 4 kb) of Zika virus was inserted to each gene junction in MeV genome, and attempted to recover recombinant MeVs from cDNA clone. In both cases, prM-E-NS1 inserted at the first junction between leader and NP, and the second junction between NP and P were lethal to MeV. However, prM-E-NS1 was moved to the third gene junction between P and M genes, rMeV-expressing prM-E-NS1 (rMeV-prM-E-NS1^{PM}) and rMeV-expressing prM-E (rMeV-prM-E^{PM}) were successfully recovered (FIG. 1A). For comparison of gene expression, two additional viruses, rMeV-expressing prM-E-NS1 (rMeV-prM-E-NS1^{HL}) and rMeV-expressing prM-E (rMeV-prM-E^{HL}), were recovered in which prM-E-NS1 and prM-E were inserted at the junction between H and L genes at the 5′ end of MeV genome (FIG. 1A). Next, the expression level of prM-E-NS1 was compared between rMeV-prM-E-NS1^{PM} and rMeV-prM-E-NS1^{HL}, and the expression level of prM-E between rMeV-prM-E^{PM} and rMeV-prM-E^{HL}. In both cases, Vero cells infected by rMeV-prM-E^{PM} or rMeV-prM-E-NS1^{PM} had approximately 50 times more prM-E or prM-E-NS1 expression compared to Vero cells infected by rMeV-prM-E^{HL} or rMeV-prM-E-NS1^{HL} (FIG. 1B). Thus, it was concluded that gene junction between P and M genes is an ideal location for inserting foreign antigen. This location not only ensures the success of the recovery of the recombinant MeV but also results in maximal expression of the inserted foreign antigen.

Optimize the Codon Usage of S Protein of SARS-CoV.

The S gene of 2019-nCoV/USA-WA1/2020 strain (isolated in Washington State, GenBank accession no. MN985325) was synthesized by IDT (Coralville, Iowa). To achieve maximal protein expression, the codon usage the S gene was optimized. The optimized sequence is show below as SEQ ID NO:21. In an alternative embodiment, the optimized codon S gene may comprise 60%, 70%, 80%, 90%, or 95% homology to SEQ ID NO:21.

The codon optimized S protein of SARS-CoV-2:

(SEQ ID NO: 21)
ATGTTTGTTTTCCTGGTGCTGCTGCCTCTGGTGTCTAGCCAGTGTGTCAACCTGACT
ACCCGAACCCAGCTGCCCCCTGCGTACACCAACTCCTTTACTAGGGGTGTCTACTA
CCCCGACAAGGTCTTTCGGTCTTCTGTACTTCATTCTACACAAGATCTCTTCCTGCC
CTTCTTCAGCAACGTGACATGGTTTCACGCCATCCATGTGTCCGGGACCAATGGAA
CCAAGAGGTTCGACAACCCAGTGTTGCCATTCAACGATGGAGTCTACTTCGCTAGC
ACAGAGAAAAGCAACATTATAAGGGGCTGGATCTTTGGCACCACTCTGGATAGCAA
GACTCAGTCCCTCCTCATCGTGAACAATGCTACCAACGTGGTTATTAAGGTGTGCG
AATTTCAGTTTTGTAATGATCCCTTCCTCGGGGTCTACTATCACAAAAATAATAAGTC
CTGGATGGAGTCCGAGTTCCGAGTTTATTCTTCCGCAAATAATTGCACATTTGAGTA
TGTTAGTCAGCCCTTCCTCATGGACCTCGAAGGCAAACAGGGAAATTTTAAGAACC
TTAGAGAATTCGTCTTCAAAAATATCGATGGGTATTTCAAGATTTATAGCAAGCATAC
ACCCATTAACCTGGTACGAGACCTCCCTCAGGGCTTTTCCGCACTTGAGCCTCTGG
TCGATCTCCCGATAGGCATAAACATAACTAGGTTTCAGACTTTGCTTGCCCTCCATA
GGTCCTACCTTACGCCTGGGGATAGCAGCAGTGGTTGGACTGCGGGCGCCGCCG
CTTACTATGTTGGGTATCTTCAACCTCGCACGTTCCTGCTTAAGTATAATGAAAATG
GGACCATTACGGATGCCGTCGATTGTGCGCTGGATCCTTTGTCCGAGACGAAATGT
ACCCTGAAAAGTTTCACCGTGGAGAAAGGGATTTATCAGACGAGCAATTTCCGCGT
CCAGCCAACCGAGTCCATTGTGCGGTTTCCGAACATCACAAACCTGTGTCCCTTCG
GGGAGGTGTTCAACGCCACACGGTTTGCTAGTGTTTACGCATGGAATCGAAAGCGA
ATTAGTAACTGCGTGGCAGACTACTCAGTTCTTTATAACAGTGCGAGCTTCTCTACT
TTTAAGTGCTACGGTGTGTCCCCCACCAAGCTTAACGACCTCTGTTTTACCAACGTC
TACGCCGATAGCTTCGTCATTAGGGGGGATGAAGTGAGACAGATCGCACCTGGCC
AGACTGGGAAAATCGCCGATTACAACTACAAACTGCCGGATGATTTCACAGGCTGC
GTTATCGCTTGGAACAGTAATAACTTGGACTCAAAAGTGGGAGGGAACTATAATTAT
CTCTATCGACTGTTCAGAAAGTCTAACCTGAAACCTTTTGAACGAGATATTAGTACC
GAGATTTACCAAGCTGGCAGTACACCGTGCAACGGCGTCGAGGGGTTCAATTGTTA
TTTTCCTCTTCAATCCTACGGTTTTCAGCCTACTAACGGAGTGGGATACCAGCCATA
CCGAGTGGTTGTGCTGTCATTCGAGCTGCTGCACGCTCCAGCTACCGTCTGTGGC
CCTAAAAAGTCCACGAACCTGGTCAAGAACAAATGCGTGAATTTCAATTTCAACGGT
CTCACCGGGACGGGCGTGCTTACGGAAAGCAACAAGAAATTCCTGCCATTCCAACA
GTTCGGCCGGGACATCGCCGACACCACTGACGCGGTGCGCGATCCTCAGACATTG
GAAATCCTTGACATTACTCCGTGCTCTTTCGGGGGAGTCTCCGTCATCACCCCAGG
AACTAACACGTCTAACCAGGTGGCTGTTCTGTATCAGGATGTTAATTGCACAGAAGT
GCCCGTTGCGATACACGCGGACCAGCTTACTCCTACCTGGAGGGTCTATTCCACC
GGCTCTAACGTCTTTCAGACCCGCGCCGGTTGCCTCATAGGCGCAGAACATGTAAA
TAATAGTTATGAATGCGACATTCCTATTGGCGCCGGGATCTGTGCGAGCTACCAGA
CTCAAACCAACAGCCCGAGGCGGGCCAGGTCCGTGGCGAGTCAGTCCATTATTGC
TTATACAATGTCCCTGGGAGCAGAGAACTCTGTGGCTTACTCCAACAATAGCATAG
CGATACCAACTAATTTTACTATATCCGTGACAACGGAGATCCTTCCCGTCTCCATGA
CAAAAACTAGTGTGGACTGCACAATGTATATCTGTGGCGATAGCACTGAATGCTCC
AACCTTCTGCTGCAGTATGGGAGCTTTTGCACACAGTTGAACCGGGCCCTGACCG
GCATCGCCGTAGAGCAGGATAAAAATACACAGGAGGTGTTTGCTCAGGTGAAGCAA
ATTTACAAGACCCCTCCTATTAAAGATTTCGGCGGGTTTAATTTCAGCCAGATCCTG
CCGGACCCATCCAAGCCGAGTAAGAGGAGCTTTATCGAAGACTTGCTGTTCAACAA
AGTGACTTTGGCTGATGCAGGCTTCATTAAGCAGTACGGTGATTGTCTCGGCGACA
TCGCAGCGCGGGATCTCATATGTGCTCAAAAGTTCAACGGTCTTACAGTACTCCCA
CCTCTGCTGACCGACGAGATGATTGCTCAGTACACGTCTGCCTTGCTGGCCGGGA
CGATCACCAGCGGGTGGACCTTTGGCGCAGGCGCAGCTCTTCAAATCCCCTTTGC
AATGCAGATGGCTTATCGATTTAACGGAATCGGGGTGACGCAGAACGTGTTGTATG
AGAACCAAAAATTGATAGCTAATCAATTCAATTCAGCGATAGGTAAGATTCAGGACT
CCCTGTCCAGCACCGCTTCCGCGTTGGGAAAATTGCAGGACGTGGTGAATCAAAAT
GCACAAGCACTGAATACACTCGTGAAACAGCTGTCCAGTAACTTCGGGGCCATCTC
CTCCGTGCTCAACGACATCCTTTCTAGACTTGATAAAGTGGAAGCCGAAGTGCAGA
TTGATCGGCTTATCACAGGTCGCCTGCAGTCACTGCAGACTTACGTCACTCAACAG
CTTATCCGCGCCGCAGAGATCAGAGCAAGTGCCAATCTTGCCGCAACCAAAATGTC
CGAGTGCGTGTTGGGACAGTCTAAGCGAGTTGACTTTTGTGGTAAGGGGTACCAC
CTGATGAGTTTCCCCCAGTCCGCCCCACACGGCGTGGTTTTTCTGCACGTGACTTA
TGTGCCAGCCCAGGAGAAGAATTTTACTACCGCTCCGGCCATTTGCCACGACGGTA
AGGCACACTTCCCTAGAGAGGGGGTTTTCGTGAGTAACGGCACTCACTGGTTTGTC
ACACAGCGGAATTTTTACGAGCCTCAGATCATCACTACAGATAACACTTTCGTGTCC
GGAAACTGCGACGTGGTTATCGGCATCGTCAACAATACCGTCTATGATCCTCTCCA
GCCAGAACTGGACTCCTTTAAGGAAGAACTTGATAAATACTTCAAGAATCACACAAG
TCCAGATGTTGACCTTGGGGACATTAGCGGGATCAATGCAAGCGTAGTTAATATTC
AGAAGGAGATTGACCGCCTCAACGAAGTGGCCAAAAATCTCAATGAATCTCTTATT
GATCTTCAGGAGCTCGGCAAGTACGAGCAGTACATCAAGTGGCCCTGGTACATATG
GCTCGGATTTATCGCCGGGTTGATCGCCATCGTGATGGTTACAATCATGCTGTGTT
GCATGACATCATGTTGTTCCTGCTTGAAAGGCTGCTGTAGCTGCGGGTCTTGTTGT
AAATTCGATGAGGATGATTCTGAGCCAGTACTGAAGGGCGTCAAGCTCCACTATAC
ATGA.

Develop a Yeast-Based Recombination System for Rapid Construction of cDNA Clone of rMeV Expressing SARS-CoV-2.

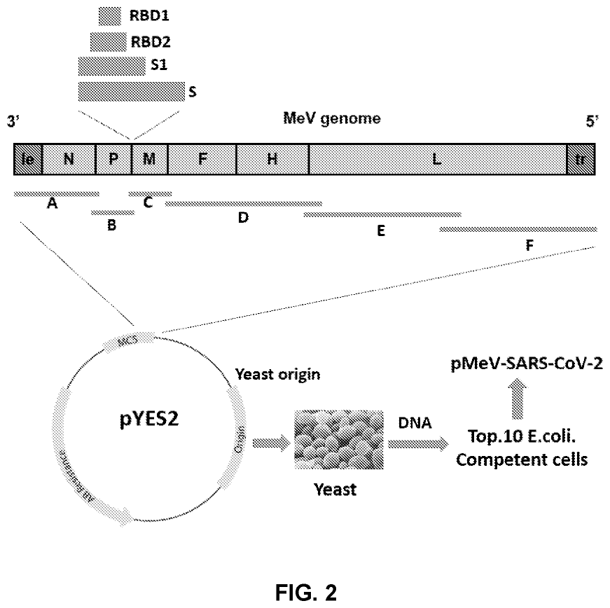

Having determined that the junction between P and M genes is the ideal location to insert a foreign gene, a rapid and convenient yeast-based strategy was next developed to clone the SARS-CoV-2 S genes into the junction between P and M genes in MeV genome. The traditional method for assembly of an infectious cDNA requires multiple cloning steps involved in restriction enzyme digestion and ligation, which are time consuming, labor extensive, and technically challenging. The traditional cloning strategy also often leads to some unexpected deletions, insertions, and mutations in the viral genome, which hamper the subsequent virus rescue. Similarly, it has been a challenge to insert a foreign gene using traditional restriction enzyme digestion and ligation due to the large size of MeV genome. To overcome this problem, a novel, rapid, and highly efficient assembly strategy was developed, allowing full-length cDNA clones with inserted SARS-CoV-2 gene in a single step (FIG. 2).

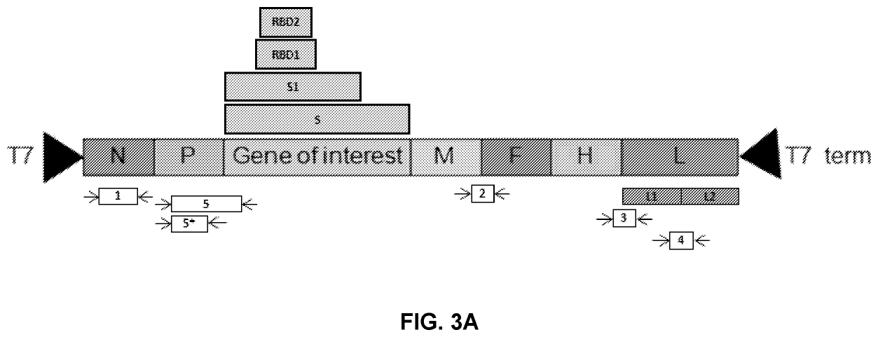

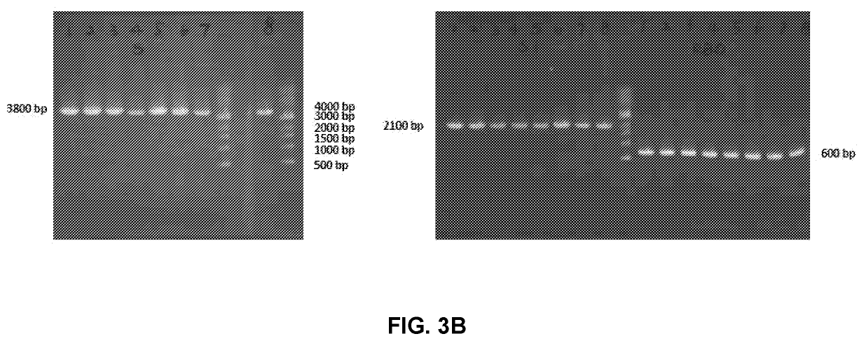

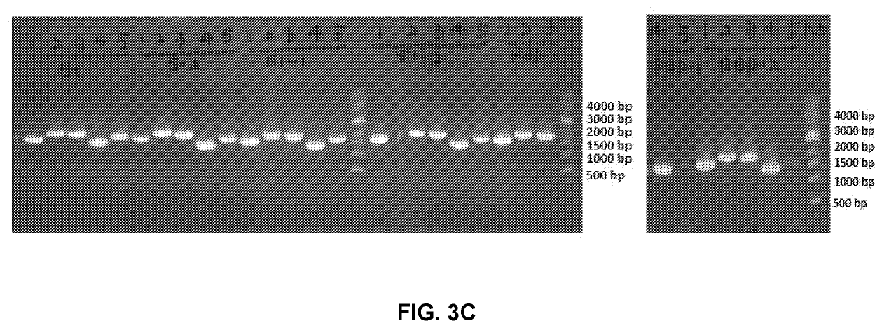

The plasmid pYES2 vector was modified to insert a yeast replication origin, a T7 RNA polymerase promoter, a hepatitis delta virus (HDV) ribozyme sequence, and a T7 terminator. The full-length cDNA clone of MeV was constructed using six overlapping fragments (designated from A to F) by using DNA recombinase in yeasts (FIG. 2). Briefly, 100 ng of pYES2 vector was mixed with 200 ng of each MeV DNA fragment in PEG/LiAc solution, and the ligation products were transformed into competent yeast cells by electroporation and plated on SD/Ura⁻ agar plates. After incubation for 2 days at 30° C., individual colony was picked for yeast colony PCR analysis. For initial screening, the connection regions between fragments were amplified by RT-PCR and sequenced. The positive plasmid was then transformed into TOP10B competent cells, and plasmid DNA was verified by restriction enzyme digestion, PCR analysis, and sequenced to confirm that no additional mutations were introduced during the assembly. The final plasmid was designated as pYES2-SARS-CoV-2 (FIG. 2). This method is very convenient to insert foreign gene, in this case, SARS-CoV-2 S genes, S truncations, and RBDs, to MeV genome. Examples of the construction and verification of pYES2-S, pYES2-S1, pYES2-RBD1, pYES2-RBD2, and pYES2-RBD3 were shown in FIG. 3. Primers used in this study was listed in Table 1.

TABLE 1
Primers used in construction of infectious cDNA clone expressing
SARS-CoV-2 S, S1, RBD1, and RBD2 genes

Primers for cloning:
SARS-COV-2 S: S opti F/S opti R
SARS-COV-2 S1: S opti F/S1 opti R
SARS-COV-2 RBD: Step 1. RBD opti F: (CD5 SP)/RBD opti R
Step 2. (use product of step 1 as template) RBD opti-P F/RBD opti R
MeV-N: MeV-5end-F/MV-N-R
MeV-P (for S/S1): MeV-P-F/MeV-P (S opti) R
MeV-P (for RBD): MeV-P-F/MeV-P (CD5) R
MeV-M (for S): MeV-M (S opti) F/MeV-M-R
MeV-M (for S1): MeV-M (S1 opti) F/MeV-M-R
MeV-M (for RBD): MeV-M (RBD opti) F/MeV-M-R
MeV-FH: MeV-F-F/MeV-H-R
MeV-L1: MeV-L1-F/MeV-L1-R
MeV-L2: MeV-L2-F/MeV-3end-R
pYES2 vector: pYES2-F/MeV-5end-R
Primers for clone verification:
1. MeV-N: MV-5end-F/ MeV-N-R (1723)
2. Junction of M-FH: M7281F/MeV-F-R (2121)
3. Junction of FH-L1: MeV-H-F/L1R11430 (2071)
4. Junction of L1-L2: L1F14034/L2R15531 (1498)
5. Junction of P-S: MeV-P-F/S opti sq R93 (1869)
Primers sequences:
For SARS-COV-2 S/S1/RBD:
S opti F: CGCCAGCCCATCAACGCGTATAATGTTTGTTTTCCTGGTGCTGCTGC
(SEQ ID NO: 22)
S opti R: TTTATAATGGATTTAGGTTGTATCATGTATAGTGGAGCTTGACGCCCTTC
(SEQ ID NO: 23)
S1 opti R:
TTTATAATGGATTTAGGTTGTATTATCAGGACATTGTATAAGCAATAATGGAC
(SEQ ID NO: 24)
RBD opti F: (CD5 SP)
ATACTCGAGATGCCCATGGGGTCTCTGCAACCGCTGGCCACCTTGTACCTGCTG
GGAATGCTGGTTGCTTCCTGCCTCGGAAACATCACAAACCTGTGTCCCTTCG
(SEQ ID NO: 25)
RBD opti R:
TTTATAATGGATTTAGGTTGTATTAGACGGTAGCTGGAGCGTGCAGCAGC (SEQ
ID NO: 26)
RBD opti-P F:
CGCCAGCCCATCAACGCGTATAGAGATGCCCATGGGGTCTCTGCAACCGC (SEQ
ID NO: 27)
For pYES2-MeV:
MeV-5end-F: ACCAAACAAAGTTGGGTAAGGATA (SEQ ID NO: 28)
MeV-N-R: GGTAGGCGGATGTTGTTCTG (SEQ ID NO: 29)
MeV-P-F: CTTCTAGACTAGGTGCGA (SEQ ID NO: 30)
MeV-P (S opti) R:
GCAGCAGCACCAGGAAAACAAACATTATACGCGTTGATGGGCTGGCG (SEQ ID
NO: 31)
MeV-P (CD5) R:
GCGGTTGCAGAGACCCCATGGGCATGAGTATACGCGTTGATGGGCTGGCGGCT
(SEQ ID NO: 32)
MeV-M (S opti) F:
GAAGGGCGTCAAGCTCCACTATACATGATACAACCTAAATCCATTATAAA (SEQ ID
NO: 33)
MeV-M (S1 opti) F:
GTCCATTATTGCTTATACAATGTCCTGATAATACAACCTAAATCCATTATAAA (SEQ
ID NO: 34)
MeV-M (RBD opti) F:
GCTGCTGCACGCTCCAGCTACCGTCTAATACAACCTAAATCCATTATAAA (SEQ ID
NO: 35)
MeV-M-R: CATGAATATGGCAGAGACGT (SEQ ID NO: 36)
MeV-F-F: CCCGACGACACTCAACTCCC (SEQ ID NO: 37)
MeV-H-R: ACGTTTTTCTTAATTCTGATGTCTAT (SEQ ID NO: 38)
MeV-L1-F: ACATCAGGCATACCCACTA (SEQ ID NO: 39)
MeV-L1-R: CCCACATATGGCTTCTTAG (SEQ ID NO: 40)
MeV-L2-F: GACAAAGAGTCATGTTCAGTG (SEQ ID NO: 41)
MeV-3end-R: CAGACAAAGCTGGGAATAG (SEQ ID NO: 42)
pYES2-F: GCAATATATTAAAGAAAACTTTGAAAATACG (SEQ ID NO: 43)
MeV-5end-R: GCACTAGAAGATGATCATTGATTGAAC (SEQ ID NO: 44)
For junction PCR:
MeV-F-R: CCTAAGTTTTAATTAACTACCGATA (SEQ ID NO: 45)
MeV-H-F: TCCCTCTGGCCGAACAAT (SEQ ID NO: 46)
M7281F: GCAACCAAACCAGAACCCAGACCAC (SEQ ID NO: 47)
L1R11430: CTAGGTGAACTTCAGGGTATAAGATCTGGT (SEQ ID NO: 48)
L1F14034: CGTTACCTTGCATATTCCCTGAACG (SEQ ID NO: 49)
L2R15531: GGGAGCTGGGTATCCTGGGATGATCTATC (SEQ ID NO: 50)
S opti sq R93: GTACGCAGGGGGCAGCTGGG (SEQ ID NO: 51)

Develop strategies to optimize the S antigens of SARS-CoV-2. The ultimate goal of this project is to identify a safe and immunogenic rMeV-based SARS-CoV-2 vaccine. Although it is known that the S protein is the major target for inducing neutralizing antibody, it is unknown which form of S antigen is the safest and most immunogenic. In addition, it has been reported that DNA vaccine expressing full-length S of SARS-CoV-1 and virus-like particles (VLP) containing full-length S protein can induce Antibody-Dependent Enhancement (ADE) of infection upon re-infection with SARS-CoV-1. ADE has been one of major hurdle to develop vaccines for respiratory viruses including human respiratory syncytial virus (RSV), human metapneumovirus (hMPV), SARS-CoV-1, and SARS-CoV-2. However, a Venezuelan Equine Encephalitis Virus (VEE)-vectored vaccine expressing S gene protected animals against SARS-CoV-1 infection with no ADE. In addition, ADE has not been reported for RBD-based subunit vaccines for SARS-CoV-1 or MERS-CoV. Therefore, it is necessary to construct a panel of rMeVs expressing full-length S, S truncations, and RBDs, to identify the most immunogenic S antigen that do not induce ADE.

Generate rMeV expressing different RBD regions. The receptor-binding domain (RBD) of S protein contains major neutralizing epitopes that can protect from CoV infection. However, it is unknown the exact boundary of the RBD. Thus, rMeV expressing different length of RBDs (named RBD1, RBD2, and RBD3) were constructed.

Generate rMeV expressing soluble stabilized prefusion S protein. The prefusion CoV S protein is a trimeric class I fusion protein in virions. It is cleaved between S1 and S2 by furin as it leaves the producer cell. At the target cell surface or in the lysosome, depending on if the cell represents in vivo or immortalized cells, respectively, S2 is cleaved TMPRSS2 or cathepsin L/B at the S2′, releasing its highly hydrophobic N-terminal fusion peptide [6, 8, 9]. Upon triggering, the S1 domain is released, exposing the S2 fusion peptide which inserts into the target cell membrane to initiate fusion.

Because the “prefusion” form of the of paramyxoviruses, pneumoviruses, and HIV fusion proteins induce antibodies with significantly higher neutralizing activity than antibodies to the postfusion form [51-53], a stabilized version of the prefusion S protein as an immunogen is expressed. The same approach is used for production of the soluble, stabilized S protein that was used for cryo-EM structure determination, replacing the TM/CT region with a self-trimerizing T4 fibritin trimerization motif, adding two proline mutations (aa 986 and 987) and disrupting the S2′ protease site. This stabilized soluble S protein is produced from rMeV as a vaccine candidate.

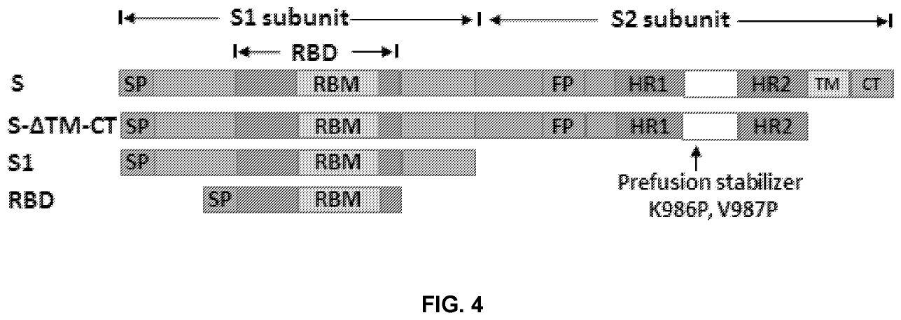

Generate rMeV expressing stabilized full-length prefusion S protein. The same modifications described in the previous section are made but retaining the natural C-terminal TM/CT domains so that the full-length S protein remain in the prefusion-stabilized form that is cell-associated (FIG. 4).

Generate rMeV expressing stabilized full-length prefusion S protein with the CT domain from MeV. During coronavirus infection, the S protein collects in the endoplasmic reticulum-Golgi intermediate compartment (ERGIC) due to an ER retention signal located in its cytoplasmic tail (CT). As a result, virions assemble on, and bud into, the lumen of the ERGIC. To produce a form of the S protein that are expressed on the plasma membrane, increasing its exposure to the immune system, the construct produced is modified by replacing the S protein CT domain with the analogous region of the VSV G protein or the MeV fusion (F) protein. The S proteins with CT corresponding to VSV or MeV are inserted as an extra gene into the relevant virus. Both these S proteins should accumulate at the cell surface, instead of the ERGIC. In addition, these modified S proteins are packaged into the virions, because of their CT. Virion incorporation has been shown to greatly enhance immunogenicity of RSV glycoproteins.

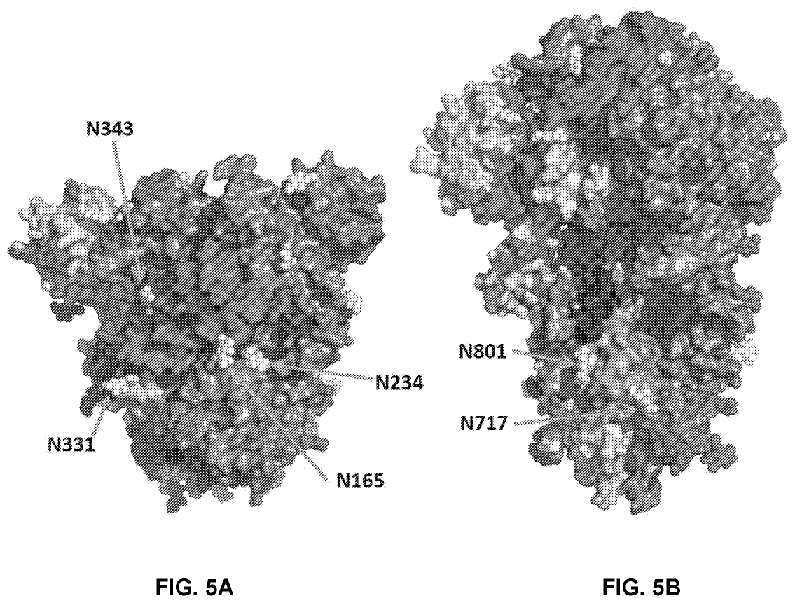

Generate rMeV expressing stabilized S protein lacking critical glycosylation sites. Removal of N-glycosylation sites in many viral glycoproteins (HIV, RSV, and influenza virus) have been shown to enhance their induction of neutralizing antibodies. The surface of the SARS-CoV-2 S protein is decorated with 66 N-glycans (22/monomer), 4 of which are located near the RBD where bound antibodies might prevent the RBD from swinging open (N165, N234, N331 and N343) (FIG. 5A). A 5^{th} N-glycan, N801, is near the S2′ cleavage site where a host cell protease (TMPRSS2 or cathepsin L/B) cleavage is essential for releasing its fusion peptide (FIG. 5B). Antibodies binding in this region could block that essential cleavage. A 6^{th} N-glycan site, N717, is on a β-sheet parallel to the HR1 α-helix that must move dramatically to initiate fusion. Antibody binding in this region could prevent HR1 from refolding, thereby preventing fusion.

All 6 of these glycosylation sites are removed by mutating the Ser or Thr in the Asn-X-Ser/Thr N-glycosylation signal, to expose these critical sites to B cells. High affinity antibodies to these sites may be able to bind to the S protein in the virion despite partial interference from these N-glycans, thereby enhancing neutralization.

Recovery of Recombinant Measles Virus (rMeV) Expressing SARS-CoV-2 Antigens.

Some of the rMeVs expressing S antigens were recovered. To recover infectious MV, HEp-2 cells were first infected by vaccinia virus MVA-T7 expressing T7 RNA polymerase, followed by co-transfected with full-length cDNA clone pVBS-MV(+)-S1, RBD1, or RBD2, and the support plasmids expressing ribonucleoprotein (pN, pP, and pL). After three days, cell monolayers were trypsinized and co-cultured with Vero-CCL81 cells. Typically, cell-to-cell fusion or syncytia were observed at days 2 or 3 co-culture. At day 4 or 5, cell culture supernatants were harvested and used to infect new Vero cells. When extensive syncytia were observed, the supernatants were used for further passage in Vero cells. Subsequently, plaque assay was performed and individual plaques were picked from each virus. Each plaque was inoculated into Vero cells, and recombinant virus was harvested for further characterization when extensive syncytia were observed.

Characterization of rMeV Expressing SARS-CoV-2 Antigens.

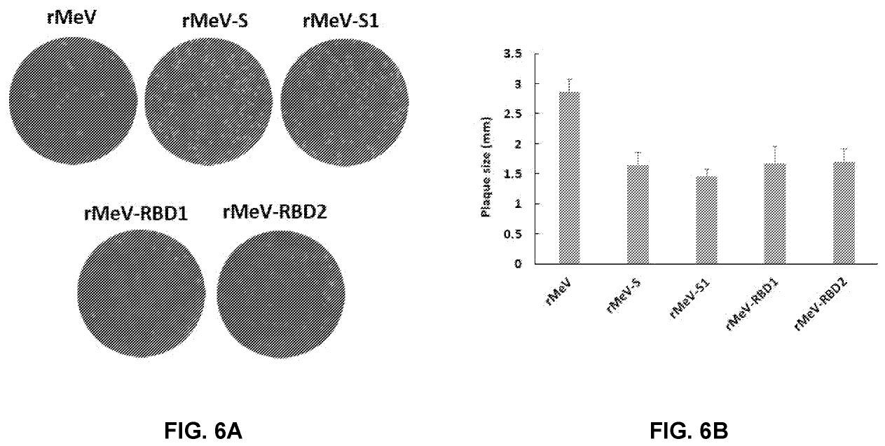

All recombinant viruses (rMeV-S1, rMeV-RBD1, and rMeV-RBD2) were plaque purified. To confirm that the recovered virus indeed contained the target gene, viral genomic RNA was extracted followed by RT-PCR using two primers annealing to the MeV P or M gene. The cDNA was purified and sequenced, confirming that S1, RBD1, and RBD2 were indeed inserted into the MeV genome at the gene junction between P and M genes. Finally, the entire genome of each recombinant virus was sequenced to confirm that no additional mutation was introduced. FIG. 6A showed plaque morphology of three recombinant viruses in Vero cells. After 5 day of incubation, rMeV formed plaques that were 2.86±0.22 mm (mean±standard deviation) in diameter. The average plaque size for rMeV-S1, rMeV-RBD1 and rMeV-RBD2 was 1.45±0.13 mm, 1.67±0.28 mm, and 1.70±0.21 mm respectively, which was significantly smaller than parental rMeV (p<0.05) (FIG. 6B). This suggests that these recombinant viruses may have a defect in viral growth and/or cell-to-cell spread, and the inserted S antigens of SARS-CoV-2 further attenuate the MeV.

Syncytium formation of each recombinant virus was next monitored in virus-infected cells. Briefly, confluent Vero cells were infected with each recombinant virus at an MOI of 1.0. Parental rMeV started to develop small syncytia at 12 h post-infection and formed large syncytia at 24 h post-infection; and most cells were fused at 36 h post-infection and cells were lysed at 48 h. All three recombinant viruses had a delay in the development of cell-cell fusion. Syncytia were observed at 24 h post-infection, extensive cell-cell fusion was observed at 36-48 h, and cells were lysed at 60 h post-infection.

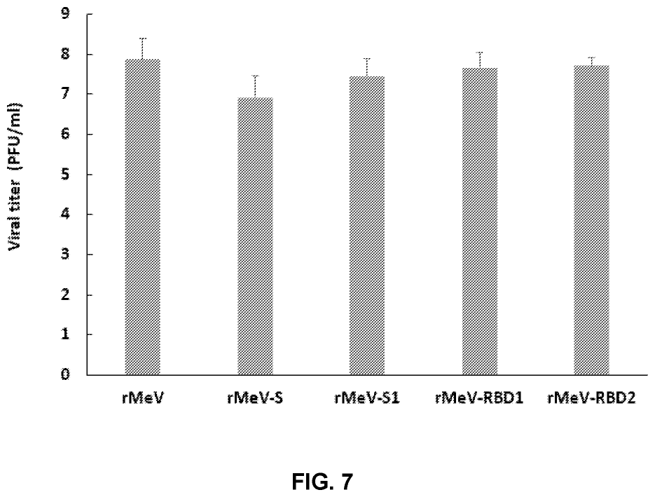

Next determined was whether these recombinant viruses grew to high titer in cell culture. Briefly, confluent Vero cells were infected with each recombinant virus at an MOI of 0.1. The parental rMeV reached maximum cytopathic effects (CPE) at 48 h post-infection and all other three recombinant reached maximum CPE approximately at 60 h post-infection. Cell supernatants were harvested and viral titer was determined by plaque assay. As shown in FIG. 7, all recombinant viruses grew to high titer and there was no significant difference among these viruses (P>0.05).

High-Level Expression of SARS-CoV-2 S Proteins by the MeV Vector.

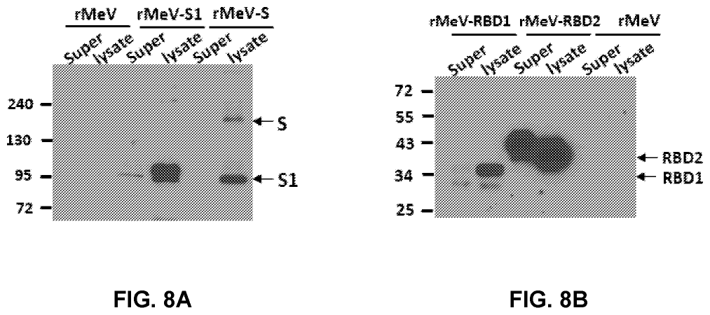

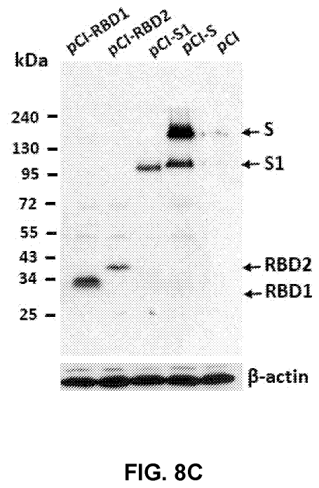

Next determined was whether SARS-CoV-2 S antigens can be expressed by rMeV. To do this, confluent Vero cells were infected by each recombinant virus at MOI of 0.4, cell lysates were harvested at 28 h post-infection, and proteins were analyzed by SDS-PAGE followed by Western blot using antibody against RBD protein. As shown in FIG. 8A, a 95 kDa protein band was detected in rMeV-S1-infected Vero cells, but not rMeV-infected cells. This is consistent with the predicted size of S1 protein of SARS-CoV-2. Two protein bands with molecular weight of 190 and 95 kDa protein band were detected in rMeV-S-infected Vero cells, which are consistent with the predicted size of full-length S protein and the cleaved product S1 protein, respectively. Similarly, 34 kDa and 43 kDa protein bands were detected in rMeV-RBD1 and rMeV-RBD2 infected cells, respectively, which is consistent with the predicted size of RBD1 and RBD2 of SARS-CoV-2 S protein (FIG. 8B). To further confirm the S protein expression, the S, S1, RBD1, and RBD2 genes were cloned into pCI vector under the control of CMV promoter, which resulted in the construction of pCI-S, pCI-S1, pCI-RBD1, and pCI-RBD2 respectively. 293T cells were transfected with each of these plasmid, total cell lysates were harvested at 72 h, and subjected to Western blot using RBD antibody. A 230, 95, 34 and 43 kDa protein band was detected in pCI-S, pCI-S1, pCI-RBD1, and pCI-RBD2-transfected cells but not pCI-transfected cells (FIG. 8C). Collectively, these results demonstrated that S, S1, RBD1, and RBD2 proteins of SARS-CoV-2 were highly expressed by rMeV vector.

### Example 2

Introduction

In December 2019, a novel coronavirus disease (COVID-19) was first identified in Wuhan City, Hubei Province, P. R. China. The causative agent was named severe acute respiratory syndrome coronavirus 2 (SARS-CoV-2). On Mar. 11, 2020, the World Health Organization (WHO) declared COVID-19 a global pandemic (Li Q, et al. N Engl J Med 2020 382:1199-1207; Huang C, et al. Lancet 2020 395:497-506; Zhu N, et al. N Engl J Med 2020 382:727-733). It spread rapidly within China and swept into at least 200 countries within 3 months. Symptoms are primarily pneumonia as with two other important human coronaviruses (CoVs), SARS-CoV-1 and MERS-CoV. As of Feb. 1, 2021, more than 102,399,513 cases had been reported worldwide, with 2,217,005 deaths (˜2.2% mortality). There is an urgent need to develop a safe and efficacious vaccine to protect the populace from this new virus. Globally, more than 300 SARS-CoV-2 vaccine candidates are in preclinical development (Sharpe H R, et al. Immunology 2020; Thanh Le T, et al. Nat Rev Drug Discov 2020 19:305-306; Hotez, P J, et al. Nat Rev Immunol 2020 20:347-348) and at least 30 vaccine candidates have entered human clinical trials (Sharpe H R, et al. Immunology 2020; Thanh Le T, et al. Nat Rev Drug Discov 2020 19:305-306; Sahin U, et al. Nature 2020 586:594-599; Krammer, F. Nature 2020 586:516-527). Among them, vaccines based on mRNA, inactivated virus, and adenovirus vectors (Ad5-nCoV and ChAdOx1) are now in phase III clinical trials. Excitingly, preliminary results indicate that these vaccines are highly efficacious, reaching 90 to 95% effectiveness against SARS-CoV-2 infection in some cases. The durability of the protection conferred by these vaccine candidates is unknown. Although these vaccine candidates are highly promising, exploration of other vaccine platforms is needed.

The CoV spike (S) protein is the main target for neutralizing antibodies that inhibit infection and prevent disease. As such, the S protein is the primary focus for CoV vaccine development (Wrapp D, et al. Science 2020 367:1260-1263; Walls A C, et al. Cell 2020). The CoV S protein is a class I fusion protein trimer that is incorporated into virions as they bud into the endoplasmic reticulum-Golgi intermediate compartment. For SARS-CoV-2, S is cleaved into S1 and S2 subunits by furin before the virion is released. The S1 subunit contains the receptor-binding domain (RBD) that attaches to the hACE2 receptor on the surface of a target cell. The S2 subunit is further cleaved by TMPRSS2 (or cathepsin L/B) and possesses the membrane-fusing activity (Wrapp D, et al. Science 2020 367:1260-1263; Li F, et al. Science 2005 309:1864-1868; Shang J, et al. Nature 2020). Both S and its RBD domain have been shown to be immunogenic for many CoVs (Tai W, et al. Cell Mol Immunol 2020 17:613-620; Zhou Y, et al. Expert Rev Vaccines 2018 17:677-686; Chen W H, et al. Hum Vacc Immunother 2020). The native S in the virion is in its “prefusion” form. Upon triggering, the prefusion S (preS) undergoes significant conformational changes to insert its fusion peptide into the target cell membrane and bring the virion and cell membranes together, arriving at its postfusion S form as it causes the membranes to fuse. For paramyxoviruses, pneumoviruses, and HIV, it has been shown that prefusion forms of glycoprotein are more potent in inducing neutralizing antibodies that their post-fusion forms (Crank M C, et al., Science 2019 365:505-509; McLellan J S, et al. Science 2013 340:1113-1117; McLellan J S. et al. Science 2013 342:592-598; Kwong P D, et al. Immunity 2018 48:855-871; Stewart-Jones G B E, et al. Proc Natl Acad Sci USA 2018 115:12265-12270). Currently, whether the SARS-CoV-2 prefusion S protein is more immunogenic than the postfusion S protein is unknown.

Live attenuated measles virus (MeV) vaccine has been one of the safest and most efficient human vaccines and has been used in children since the 1960s (Lin W H W, et al. Sci Transl Med 2020 12; Griffin D E. Viral Immunol 2018 31:86-95). Worldwide MeV vaccination campaigns have been very successful in controlling measles. MeV is an enveloped non-segmented negative sense RNA virus that belongs to the genus Morbillivirus within the Paramyxoviridae family. MeV is an excellent vector to deliver vaccines for human pathogens primarily because of its high safety, efficacy, and long-lived immunity (Griffin D E. Viral Immunol 2018 31:86-95; Frantz P N, et al. Microbes Infect 2018 20:493-500). MeV has previously been shown to be a highly efficacious vaccine vector for many viral diseases such as human immunodeficiency virus (HIV) (Lorin C, et al. J Virol 2004 78:146-157; Wang Z, et al. Vaccine 2001 19:2329-2336), SARS-CoV-1 (Escriou N., et al. Virology 2014 452-453:32-41; Liniger M, et al. Vaccine 2008 26:2164-2174), MERS-CoV (Malczyk A H, et al. J Virol 2015 89:11654-11667; Bodmer B S, et al. Virology 2018 521:99-107), respiratory syncytial virus (RSV) (Swett-Tapia C, et al. J Gen Virol 2016 97:2117-2128), hepatitis B and C viruses (Reyes-del Valle J, et al. J Virol 2012 86:11558-11566), influenza virus (Swett-Tapia C, et al. J Gen Virol 2016 97:2117-2128; Ito T, et al. Vaccines (Basel) 2020 8), chikungunya virus (CHIKV) (33), and flaviviruses [Zika virus (ZIKV), dengue virus (DENV), West Nile virus (WNV), and yellow fever virus (YFV)] (Nurnberger C, et al. J Virol 2019 93; Brandler S, et al. J Infect Dis 2012 206:212-219; Brandler S, et al. PLoS Negl Trop Dis 2007 1:e96). Recent human clinical trials have demonstrated that rMeV-based CHIKV vaccine is safe and highly immunogenic in healthy adults, even in the presence of pre-existing anti-MeV vector immunity (Reisinger E C, et al. Lancet 2018 392:2718-2727).

In this study, a series of MeV-based vaccine candidates expressing different forms of the SARS-CoV-2 S protein were developed and evaluated in cotton rats, IFNAR^{-/-}-hCD46 mice, and Golden Syrian hamsters. All SARS-CoV-2 S antigens are highly expressed by the MeV vector. Among these vaccine candidates, rMeV expressing stabilized prefusion S (rMeV-preS) and full-length S (rMeV-S) proteins were the most potent in triggering SARS-CoV-2-specific antibody. Animals immunized with rMeV-preS induced the highest level of neutralizing antibodies that were higher than convalescent sera of patients recovered from COVID-19, and the highest Th1-biased T cell immune response. Furthermore, hamsters immunized with rMeV-preS provided complete protection against SARS-CoV-2 challenge and lung pathology.

Materials and Methods

Biosafety. All experiments with infectious SARS-CoV-2 were conducted under biosafety level 3 (BSL3).

Cell cultures. Vero CCL81 cells (African green monkey, ATCC no. CCL81), Vero E6 cells (ATCC CRL-1586), and HEp-2 cells (ATCC no. CCL-23) were grown in Dulbecco's modified Eagle's medium (DMEM; Life Technologies) supplemented with 10% fetal bovine serum (FBS). FreeStyle293F cells (Thermo Fisher) were grown in protein-free medium in suspension culture.

Virus strain. The SARS-CoV-2 USA-WA1/2020 natural isolate (GenBank accession no. MN985325) was obtained from BEI Resources (NR-52281) and amplified on Vero E6 cells. This strain was originally isolated from an oropharyngeal swab from a patient with respiratory illness.

Animals. Specific-pathogen-free (SPF) IFNAR1^{-/-} and C57BL/6J-hCD46 mice were purchased from Jackson Laboratories (Bar Harbor, ME). Golden Syrian hamsters and cotton rats (Sigmodon hispidus) were purchased from Envigo (Indianapolis, IN). IFNAR1^{-/-}-hCD46/mice were generated by hybridization of IFNAR1^{-/-} mice (Jackson laboratory) with C57BL/6J-hCD46 mice (Jackson laboratory). IFNAR1 knockout homozygous with hCD46 knock-in mice are derived by sib mating of the first filial generation. Genotype of IFNAR1^{-/-} and hCD46 was determined by PCR from alkaline lysed ear tissue of each mouse. Sequences of PCR primers are: IFNAR1 common forward: 5′-CGA GGC GAA GTG GTT AAA AG (SEQ ID NO:52); IFNAR1 wild type reverse: 5′-ACG GAT CAA CCT CAT TCC AC (SEQ ID NO:53); IFNAR1 mutant reverse: 5′-AAT TCG CCA ATG ACA AGA CG (SEQ ID NO:54); CD46 forward: 5′-GCC TGT GAG GAG CCA CCA A (SEQ ID NO:55); CD46 reverse: 5′-CGT CAT CTG AGA CAG GTA G (SEQ ID NO:56). For PCR reaction, 2 μl of mouse DNA was mixed with primers and 2×KAPA2G Fast HotStart Genotyping Mix with dye [KAPABIOSYSTEMS, KK5621 07961316001 (6.25 ml)].

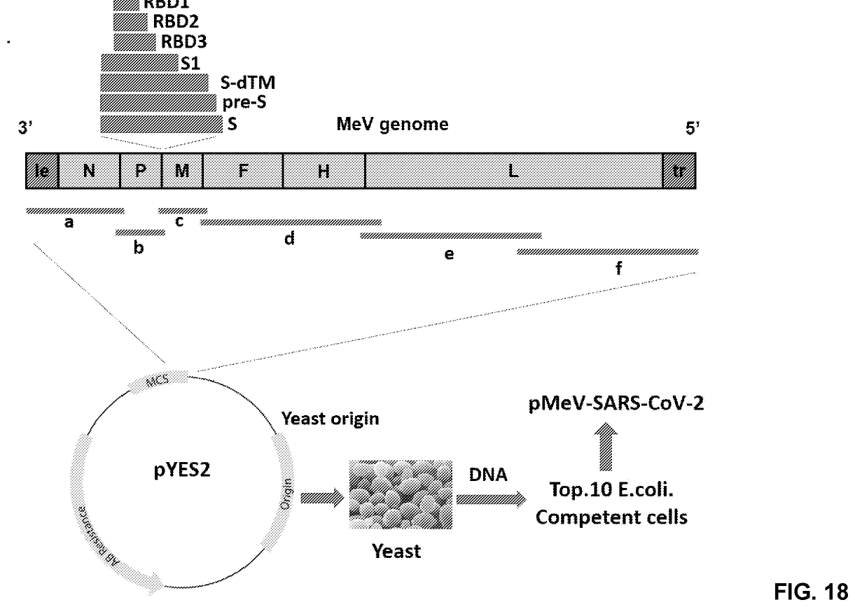

Rapid assembly of the full-length genomic cDNA of MeV by yeast-based recombination system. The full-length genomic cDNA of Edmonston strain of measles vaccine was assembled into pYES2 vector. The pYES2 vector was modified to insert a yeast replication origin from the plasmid pYES1L (Invitrogen), a T7 RNA polymerase promoter, a hepatitis delta virus ribozyme (HDVRz) sequence, and a T7 terminator. The full-length cDNA clone of MeV was constructed using six overlapping fragments (designated from A to F) by using yeast recombination system. Briefly, 100 ng of pYES2 vector was mixed with 200 ng of each MeV DNA fragment in PEG/LiAc solution, and the ligation products were transformed into MaV 203 competent yeast cells by heat-shock and plated on SD/Ura⁻ agar plates. After incubation for 3 days at 30° C., individual colony was picked, cultured in SD/Ura⁻ broth at 30° C. overnight for plasmid mini-prep. For initial screening, the connection regions between fragments were amplified by PCR and sequenced. The positive plasmid was then transformed into TOP10 competent cells, and plasmid DNA was verified by restriction enzyme digestion, PCR analysis, and sequenced to confirm that no additional mutations were introduced during the assembly. The final plasmid was designated as pMeV-SARS-CoV-2 (FIG. 18). Primers used in this study was listed in Table 2. Using this method, SARS-CoV-2 full-length S (S), a stabilized prefusion S (preS) with deletion of the furin cleavage site, two proline mutations, and a foldon trimerization domain (Wrapp D, et al. Science 2020 367:1260-1263), S with deletion of the transmembrane domain and cytoplasmic tail (S-dTM), S1 subunit, and three different length of RBDs (RBD1, RBD2, and RBD3) containing MeV gene start and gene end sequences were inserting into the gene junction between P and M genes in the MeV genome. These plasmids were named pYES2-S, preS, S-dTM, S1, RBD1, RBD2, and RBD3. All the constructs were confirmed by sequencing. All the S genes and S truncations used in this study were codon optimized for mammalian cells expression.

TABLE 2
Primers used in construction of infectious cDNA clones expressing
SARS-CoV-2 S, preS, S-dTM, S1, RBD1, RBD2, and RBD3 genes

| | |
|---|---|
| Name | Sequences (5' to 3') |

Primers used for cloning SARS-C0V-2 S genes

| | |
|---|---|
| S/S1/S-dTM opti F | CGCCAGCCCATCAACGCGTATAATGTTTGTTTTCCTGGTGCTGCTGC (SEQ ID NO: 57) |
| S opti R | TTTATAATGGATTTAGGTTGTATCATGTATAGTGGAGCTTGACGCCCTTC (SEQ ID NO: 58) |
| S1 opti R | TTTATAATGGATTTAGGTTGTATTATCAGGACATTGTATAAGCAATAATGGAC (SEQ ID NO: 59) |
| S opti dTM (1208) R: | TTTATAATGGATTTAGGTTGTAttatcaCTGCTCGTACTTGCCGAGCTCCTGAAG (SEQ ID NO: 60) |
| RBD opti short F: (CD5 | ATACTCGAGA TGCCCATGGG GTCTCTGCAA CCGCTGGCCA CCTTGTACCT GCTGGGAATG |
| SP) | CTGGTTGCTT CCTGCCTCGG AAACATCACA AACCTGTGTC CCTTCG (SEQ ID NO: 61) |
| RBD opti medium/long F: | ATACTCGAGA TGCCCATGGG GTCTCTGCAA CCGCTGGCCA CCTTGTACCT GCTGGGAATG |
| (CD5 SP) | CTGGTTGCTT CCTGCCTCGG ACGCGTCCAG CCAACCGAGT CCATTG (SEQ ID NO: 62) |
| RBD opti short R: | TTTATAATGGATTTAGGTTGTATTAGACGGTAGCTGGAGCGTGCAGCAGC (SEQ ID NO: 63) |
| RBD opti medium R: | TTTATAATGGATTTAGGTTGTATTATCAGAAATTCACGCATTTGTTCTTG (SEQ ID NO: 64) |
| RBD opti long R: | TTTATAATGGATTTAGGTTGTAttatcaCGGAGTAATGTCAAGGATTTCCAATG (SEQ ID NO: 65) |
| RBD opti-P F: | CGCCAGCCCATCAACGCGTATAGAGATGCCCATGGGGTCTCTGCAACCGC (SEQ ID NO: 66) |
| preS-F | CGCCAGCCCATCAACGCGTATAATGTTCGTGTTCCTGGTGCTCCTGC (SEQ ID NO: 67) |
| preS-R | TTTATAATGGATTTAGGTTGTATCATTAGCCCAGGAATGTGCTCAGCAGTAC (SEQ ID NO: 68) |

Primers used for construction of infectious cDNA clone of MeV

| | |
|---|---|
| MeV-5end-F | ACCAAACAAAGTTGGGTAAGGATA (SEQ ID NO: 69) |
| MeV-N-R | GGTAGGCGGATGTTGTTCTG (SEQ ID NO: 70) |
| MeV-P-F | CTTCTAGACTAGGTGCGA (SEQ ID NO: 71) |
| MeV-P (S opti) R | GCAGCAGCACCAGGAAAACAAACATTATACGCGTTGATGGGCTGGCG (SEQ ID NO: 72) |
| MeV-P (CD5) R: | GCGGTTGCAGAGACCCCATGGGCATGAGTATACGCGTTGATGGGCTGGCGGCT (SEQ ID |
| | NO: 73) |
| MeV-P (preS) R: | GCAGGAGCACCAGGAACACGAACATTATACGCGTTGATGGGCTGGCG (SEQ ID NO: 74) |
| MeV-M (S opti) F | GAAGGGCGTCAAGCTCCACTATACATGATACAACCTAAATCCATTATAAA (SEQ ID NO: 75) |
| MeV-M (S1 opti) F | GTCCATTATTGCTTATACAATGTCCTGATAATACAACCTAAATCCATTATAAA (SEQ ID NO: 76) |
| MeV-M (RBD opti short) F | GCTGCTGCACGCTCCAGCTACCGTCTAATACAACCTAAATCCATTATAAA (SEQ ID NO: 77) |
| MeV-M (RBD opti | CAAGAACAAATGCGTGAATTTCTGATAATACAACCTAAATCCATTATAAA (SEQ ID NO: 78) |
| medium) F | |
| MeV-M (RBD opti long) F | CATTGGAAATCCTTGACATTACTCCGtgataaTACAACCTAAATCCATTATAAA (SEQ ID NO: 79) |
| MeV-M (S opit dTM) F | CTTCAGGAGCTCGGCAAGTACGAGCAGtgataaTACAACCTAAATCCATTATAAA (SEQ ID NO: 80) |
| MeV-M (preS) F | GTACTGCTGAGCACATTCCTGGGCTAATGATACAACCTAAATCCATTATAAA (SEQ ID NO: 81) |
| MeV-M-R | CATGAATATGGCAGAGACGT (SEQ ID NO: 82) |
| MeV-F-F | CCCGACGACACTCAACTCCC (SEQ ID NO: 83) |
| MeV-H-R | ACGTTTTTCTTAATTCTGATGTCTAT (SEQ ID NO: 84) |
| MeV-L1-F | ACATCAGGCATACCCACTA (SEQ ID NO: 85) |
| MeV-L1-R | CCCACATATGGCTTCTTAG (SEQ ID NO: 86) |
| MeV-L2-F | GACAAAGAGTCATGTTCAGTG (SEQ ID NO: 87) |
| MeV-3end-R | CAGACAAAGCTGGGAATAG (SEQ ID NO: 88) |
| pYES2-F | GCAATATATTAAAGAAAACTTTGAAAATACG (SEQ ID NO: 89) |
| MeV-5end-R | GCACTAGAAGATGATCATTGATTGAAC (SEQ ID NO: 90) |

Primers used for junction PCR:

| | |
|---|---|
| MeV-F-R | CCTAAGTTTTAATTAACTACCGATA (SEQ ID NO: 91) |
| MeV-H-F | TCCCTCTGGCCGAACAAT (SEQ ID NO: 92) |
| M7281F | GCAACCAAACCAGAACCCAGACCAC (SEQ ID NO: 93) |
| L1R11430 | CTAGGTGAACTTCAGGGTATAAGATCTGGT (SEQ ID NO: 94) |
| L1F14034 | CGTTACCTTGCATATTCCCTGAACG (SEQ ID NO: 95) |
| L2R15531 | GGGAGCTGGGTATCCTGGGATGATCTATC (SEQ ID NO: 96) |
| S opti sq R93 | GTACGCAGGGGGCAGCTGGG (SEQ ID NO: 97) |

Recovery of recombinant MeV (rMeV) expressing SARS-CoV-2 S antigens.

Recovery of rMeV from the infectious clone was carried out as described previously (Wang Y, et al. Virology 2018 518:210-220; Radecke F, et al. Embo J 1995 14:5773-5784). Briefly, plasmid encoding the full-length genome of MeV Edmonston strain with S, preS, S-dTM, S1, or RBDs, and support plasmids encoding MeV ribonucleocapsid complex (pN, pP, and pL) were co-transfected into HEp-2 cells infected with a recombinant modified vaccinia Ankara virus (MVA-T7) expressing T7 RNA polymerase (Fuerst, T R, et al. P Natl Acad Sci USA 1986 83:8122-8126). At day 4 post-transfection, cells and supernatants were collected, and co-cultured with 90% confluent Vero CCL81 cells. At day 4, the recovered recombinant virus was further amplified in Vero CCL81 cells. Subsequently, the viruses were plaque purified as described previously (Li, J R, et al. P Natl Acad Sci USA 2006 103:8493-8498; Li, J R, et al. J Virol 2005 79:13373-13384). Individual plaques were isolated, and seed stocks were amplified in Vero CCL81 cells. The viral titer was determined by a plaque assay performed in Vero CCL81 cells.

RT-PCR verification of SARS-CoV-2 gene. To characterize the insertion of SARS-CoV-2 genes, viral RNA was extracted from rMeVs by using a RNeasy minikit (Qiagen, Valencia, CA) according to the manufacturer's instructions. SARS-CoV-2 S, preS, S-dTM, S1, RBD1, RBD2, and RBD3 genes were amplified by a One Step RT-PCR kit (Qiagen) using primers annealing to MeV P gene and MeV M gene. The amplified products were analyzed on 1% agarose gel electrophoresis and sequenced. Primers used for RT-PCR and sequencing of pYES2-SARS-CoV-2 are listed in Table 3.

Multi-step growth curves. Confluent monolayers of Vero CCL81 cells in 12-well-plates were infected with individual viruses at a multiplicity of infection (MOI) of 0.01. After 1 h of absorption, the inoculum was removed, the cells were washed twice with Dulbecco's modified Eagle's medium (DMEM), fresh DMEM (supplemented with 2% fetal bovine serum) was added, and the infected cells were incubated at 37° C. The cell culture fluid and cell lysates were harvested and combined at the indicated intervals, and virus titers were determined by plaque assay in Vero CCL81 cells.

MeV and SARS-CoV-2 plaque assays. MeV and SARS-CoV-2 plaque assay was performed on Vero CCL81 and Vero-E6 cells in 12-well plates, respectively. For MeV, confluent Vero CCL81 cells in 12-well plates were infected with serial dilutions of rMeV or rMeV expressing SARS-CoV-2 antigen in DMEM. Similar procedure was used for SARS-CoV-2 plaque assay. After absorption for 1 h at 37° C., cells were washed three times with DMEM and overlaid with 2 ml of DMEM containing low-melting agarose (0.25% w/v). After incubation at 37° C. for 4-5 days (MeV) or 2 days (SARS-CoV-2), cells were fixed with 4% paraformaldehyde for 2 h. The overlays were removed, and the plaques were visualized after staining by crystal violet. The diameter of plaques for each virus were measured using Image J Software.

Preparation of large stock of rMeVs. T150 flasks of Vero CCL81 cells were infected with individual rMeV at a MOI of 0.1. When extensive CPEs were observed at day 3 or 4, the supernatants were harvested and kept on ice. Cell pellets were subjected to three freeze-thaw cycles in 0.5 ml of fresh DMEM with 10% trehalose (Xue M G. et al. J Virol 20209 4). The two portions of supernatants were combined and the virus titers were determined by plaque assay in Vero CCL81 cells.

Detection of SARS-CoV-2 S antigen by Western blot. Vero CCL-81 cells were infected with parental rMeV or rMeV expressing SARS-CoV-2 S antigens as described above. At the indicated times post-infection, cell culture medium was harvested and clarified at 5,000 g for 15 min. In the meantime, cells were lysed in RIPA buffer (Abcam, ab156034). Proteins were separated by 12% SDS-PAGE and transferred to a Hybond enhanced chemiluminescence nitrocellulose membrane (Amersham) in a Mini Trans-Blot electrophoretic transfer cell (Bio-Rad). The blot was probed with rabbit anti-SARS-CoV-2 S or RBD antibody at a dilution of 1:2,000, followed by horseradish peroxidase-conjugated goat anti-rabbit IgG secondary antibody (Santa Cruz) at a dilution of 1:5,000. The blot was developed with SuperSignal West Pico chemiluminescent substrate (Thermo Scientific) and exposed to Kodak BioMax MR film.

Human sera. Human serum samples were collected from six SARS-CoV-2 positive individuals once diagnosis of SARS-CoV-2 was confirmed (V1) and 30 days later (V2). All human studies were conducted in compliance with all relevant local, state, and federal regulations.

Animal experiments: All animals were housed within ULAR facilities of The Ohio State University under approved Institutional Animal Care and Use Committee (IACUC) guidelines (protocol no. 2009A0183 and 2020A00000053). Each inoculation group was separately housed in rodent cages under animal biosafety level 2 (BSL-2 for rMeV) or BSL3 (for SARS-CoV-2) conditions.

Immunogenicity in cotton rats. Cotton rats (Sigmodon hispidus) are susceptible to MeV infection (Green M G, et al. Lab Animal 2013 42:170-176; Niewiesk S. Curr Top Microbiol Immunol 2009 330:89-110). Forty-five 4-week-old specific-pathogen-free (SPF) cotton rats (Envigo, Indianapolis, IN) were randomly divided into 9 groups, with 5 cotton rats per group (n=5). Cotton rats in groups 1-9 were inoculated subcutaneously with PBS, 4×10⁵ PFU of each of Edmonston vaccine strain (parental rMeV, rMeV-S, rMeV-preS, rMeV-S1, rMeV-RBD1, rMeV-RBD2, or rMeV-RBD3). Four weeks later, cotton rats were boosted with 2×10⁶ PFU of each virus at the same immunization route. After inoculation, the animals were evaluated twice every day for any possible abnormal reaction. Blood samples were collected from each cotton rat at weeks 4, 6, and 8 by retro-orbital bleeding, and the serum was isolated for antibody detection.

Immunogenicity in IFNAR^{-/-}-hCD46 transgenic mice. IFNAR^{-/-}-hCD46 transgenic mice that are deficient for type I IFN receptor and transgenically express human CD46 (Nurnberger C, et al. J Virol 2019 93; Mura M, et al. Virology 2018 524:151-159) were bred in-house under SPF conditions. Twenty-one four-week-old female IFNAR1^{-/-}-hCD46 mice were randomly divided into 4 groups (n=5, or 6). Mice in groups 1-3 were immunized with 8×10⁵ PFU (half subcutaneous and half intranasal) of parental rMeV, rMeV-preS, or rMeV-S1. Mice in group 4 served as normal controls (unimmunized and unchallenged controls). Two weeks later, mice were boosted with 6×10⁵ PFU of each virus (half subcutaneous and half intranasal). After inoculation, the animals were evaluated twice every day for safety. Blood samples were collected from each mouse at weeks 3 by facial vein bleeding, and the serum was isolated for antibody detection. At week 3 post-immunization, spleens were isolated from each mouse for a T cell assay.

Comparison of single and booster immunization of rMeV-preS in IFNAR^{-/-} mice. 4-week-old IFNAR^{-/-} mice female IFNAR1^{-/-} mice were randomly divided into 3 groups (n=5, or 6). Mice in groups 1 were immunized with 8×10⁵ PFU of rMeV-preS (half subcutaneous and half intranasal). Mice in group 2 were immunized with 8×10⁵ PFU of rMeV-preS (half subcutaneous and half intranasal) and were boosted at the same dose at the same route 4 weeks later. Mice in group 3 were immunized with 8×10⁵ PFU of rMeV and served as controls. At weeks 7 and 8, blood samples were collected from each mouse by facial vein bleeding, and the serum was isolated for detection of S-specific antibody by ELISA.

Immunization and challenge experiment in Golden Syrian hamsters. 2 vaccine candidates (rMeV-preS and rMeV-S1) were selected for immunization and challenge experiments in Golden Syrian hamsters. Forty 4-week-old female Golden Syrian hamsters were initially housed in BSL2 animal facility and randomly divided into 4 groups (n=10). Group 1 received 8×10⁵ PFU of rMeV-preS, Group 2 received 8×10⁵ PFU of rMeV-S1, Group 3 received 8×10⁵ PFU of parental rMeV, and Group 4 received PBS. Three weeks later, hamsters in each group were boosted with the respective rMeV strain. All immunizations were done by combination of subcutaneous and intranasal routes (4×10⁵ PFU for subcutaneous and 4×10⁵ PFU for intranasal inoculation). At weeks 2, 4, and 6 post-immunization, blood was collected from each hamster for antibody detection. At week 4 post-booster immunization, animals of groups 1-3 were transferred into BSL3 facility and challenged intranasally with 10⁵ PFU of SARS-CoV-2. Hamsters in group 4 were inoculated with DMEM and served as unimmunized unchallenged controls. After challenge, clinical sign and body weight of each hamsters were monitored daily. At day 4 post-challenge, 5 hamsters in each group were euthanized, left lung, nasal turbinate, brain, liver, and spleen were collected for detection of SARS-CoV-2 and viral RNA. In addition, the right lung was preserved in 4% (vol/vol) phosphate-buffered formaldehyde for histology and immunohistochemistry (IHC). At day 12 post-challenge, the remaining 5 hamsters were terminated, and tissues were collected and processed as described above.

S protein purification. The stabilized prefusion S protein (amino acids 1-1273) of SARS-CoV-2 was cloned into pCAGGS and transfected into FreeStyle293F cells for protein expression. The secreted preS in cell culture supernatants were then purified via affinity chromatography. The purity of the protein was analyzed by SDS-PAGE and Coomassie blue staining. Protein concentration was measured using Bradford reagent (Sigma Chemical Co., St. Louis, MO).

Peptides. A set of 181 peptides spanning the complete S protein of the USA-WA1/2020 strain of SARS-CoV-2 (GenPept: QH060594) were obtained from BEI resources (National Institute of Allergy and Infectious Diseases) (cat.no. NR-52402). These peptides are 13 to 17 amino acids long, with 10 amino acid overlaps. The Spike 1 (S1) peptide pools contain 93 peptides representing the N terminal half of the S protein (MFVFLVLLPL (SEQ ID NO:114) to AEHVNNSYE (SEQ ID NO:115)) and the Spike 2 (S2) peptide pools contain 88 peptides representing the C terminal half of the S protein (GAEHVNNSYE (SEQ ID NO:116) to VLKGVKLHYT (SEQ ID NO:117)). Peptides were dissolved in sterile water containing 10% DMSO. The final concentration of each peptide in all functional assays was 2 μg/ml.

EL/SPOT assay. Spleens of immunized IFNAR^{-/-}-CD46 transgenic mice were aseptically removed 35 days after immunization and minced by pressing through cell strainers. Red blood cells were removed by incubation in 0.84% ammonium chloride and, following a series of washes in RPMI 1640, cells were resuspended in RPMI 1640 supplemented with 2 mM I-glutamine, 1 mM sodium pyruvate, 10 mM HEPES, 100 U/ml penicillin, 100 μg/ml streptomycin, and 10% fetal calf serum. Antigen-specific T cells secreting IFN-γ were enumerated using anti-mouse IFNγ enzyme linked immunospot (ELISpot) assay (U-Cytech catalogue no, CT317-PB5). Cells were plated in 96 well PVDF plates at 2×10⁵ per well in duplicate, and stimulated separately with the SARS-CoV-2 peptide pools (2 μg/ml), Concanavalin-A (5 μg/ml, Sigma) or media alone, as positive and negative controls, receptively. The plates were incubated for 42-48 h and then developed according to manufacturer's instructions. The number of spot-forming cells (SFC) were measured using an automatic counter (Immunospot). A positive response was considered only when the mean of peptides-stimulated wells was more than the mean of negative wells+3 standard deviation. The total number of spot-forming cells (SFC) were calculated by subtracting the mean number of SFC in negative control wells from that of peptides containing wells.

Quantification of intracellular cytokine production. For detection of SARS-CoV-2-specific intracellular cytokine production, 10⁶ cells were stimulated in 96-well round bottom plates with peptide pool (5 μg/ml), media alone or PMA/Ionomycin (BioLegend) as negative and positive controls, respectively, for 5-h in the presence of GolgiPlug (BD Biosciences). Following incubation, cells were surface stained for CD3, CD4, and CD8 for 30 min at 4° C., fixed and permeabilized using the cytofix/cytoperm kit (BD Biosciences), and intracellularly stained for IFNγ, TNFα, IL-2, Granzyme B, IL-10 & IL-4 for 30 min at room temperature. Dead cells were removed using the LIVE/DEAD fixable Near-IR dead cell stain kit (Invitrogen). A positive response was defined as >3 times the background of the negative control sample. The percentage of cytokine positive cells was then calculated by subtracting the frequency of positive events in negative control samples from that of test samples.

Flow cytometric analysis. The following mouse reactive antibodies (clone, catalog number, dilution) from BioLegend, BD Biosciences, and ThermoFisher Scientific were used for analysis of T cells: CD3-PE/Cyanine7 (145-2C11, 100319, 1:400), IFNγ-PE/Dazzle 594 (XMG1.2, 505845, 1:400), TNFα-Brilliant Violet 785 (MP6-XT22, 506341, 1:400), CD107a-Alexa Fluor 488 (1D4B, 121607, 1:400), granzyme-B-Alexa Fluor 647 (GB11, 515405 1:200), IL-4-Brilliant Violet 711 (11B11, 504133, 1:100), CD4-BUV 496 (GK1.5, 612952, 1:400), CD8-BUV737 (53-6.7, 612759, 1:400), IL-10-Brilliant Violet 510 (JES5-16E3, 563277, 1:100), IL-2-PE (JES6-5H4, 12-7021-82, 1:200). Surface and intracellular staining was performed as described previously (Hartlage A S, et al. Nat Commun 2019 10). Events were collected on a BD LSRFortessa X-20 flow cytometer following compensation with UltraComp eBeads (Invitrogen). Data were analyzed using FlowJo v10 (Tree Star).

Detection of SARS-CoV-2-specific antibody by ELISA. Ninety-six-well plates were first coated with 50 μl of highly purified prefusion SARS-CoV-2 preS protein (8 μg/ml, in 50 mM Na₂ CO3 buffer, pH 9.6) per well at 4° C. overnight, and then blocked with Bovine Serum Albumin (BSA, 1% W/V in PBS, 100 μl/well) at 37° C. for 2 h. Subsequently, individual serum samples were tested for S-specific Ab on antigen-coated plates. Briefly, serum samples were 2-fold serially diluted and added to S protein-coated wells (100 μl/well). After 2 h of incubation at room temperature, the plates were washed three times with phosphate-buffered saline containing 0.05% Tween (PBST), followed by incubation with 100 μl of horseradish peroxidase (HRP)-conjugated secondary Abs (Sigma) at a dilution of 1:15,000 for 1 h. The plates were washed, developed with 100 μl of SureBlue™ TMB 1-Component Microwell Peroxidase Substrate (Fisher Scientific, Catalog No. 50-674-93), and stopped by 100 μl of H₂SO₄ (2 mol/L). Optical densities (OD) at 450 nm were determined by a BioTek microplate reader. Endpoint titers were determined as the reciprocal of the highest dilution that had an absorbance value 2.1 folds greater than the background level (normal control serum). Ab titers are reported as geometric mean titers (GMT).

Detection of SARS-CoV-2 neutralizing antibody by plaque reduction. SARS-CoV-2-specific neutralizing antibody was determined using an endpoint dilution plaque reduction neutralization (PRNT) assay. The serum samples were heat inactivated at 56° C. for 30 min. Two-fold dilutions of the serum samples were mixed with an equal volume of DMEM containing approximately 100 PFU/well SARS-CoV-2 in a 96-well plate, and the plate was incubated at 37° C. for 1 h with constant rotation. The mixtures were then transferred to confluent Vero-E6 cells in a 12-well plate. After 1 h of incubation at 37° C., the virus-serum mixtures were removed and the cell monolayers were covered with 1 ml of Eagle's minimal essential media (MEM) containing 0.25% agarose, 0.12% sodium bicarbonate (NaHCO₃), 2% FBS, 25 mM HEPES, 2 mM L-Glutamine, 100 μg/ml of streptomycin, and 100 U/ml penicillin. Then, the cells were incubated for another 2 days and then fixed with 4% formaldehyde. The plaques were counted; serum dilution with 50% plaque reduction were calculated as the SARS-CoV-2-specific neutralizing antibody titers.

Determination of SARS-CoV-2 titer in hamster tissues. After SARS-CoV-2 challenge, left lung, nasal turbinate, brain, liver, and spleen was collected. Organs were weighed and homogenized by hand with a mortar and pestle (Golden, CO) in 1 mL of sterile PBS. Each sample was subjected to 10-fold serial dilutions. The initial dilution of each tissue sample is 1:10. The presence of infectious SARS-CoV-2 was determined by plaque assay in Vero-E6 cells in 12-well plates. The limit of detection (LoD) is calculated with the following formula: LoD=Log₁₀ [1(1 plaque in a well)/0.2 (0.2 ml tissue sample)×10 (lowest dilution)/average tissue weight].

Measurement of SARS-CoV-2 genomic and subgenomic RNA burden. The total RNA was extracted from homogenized left lung, nasal turbinate, brain, liver, and spleen tissue samples using TRIzol Reagent (Life technologies, Carlsbad, CA). For total viral RNA (genome and subgenome), reverse transcription (RT) was conducted using a primer (GTCATTCTCCTAAGAAGCTATTAAAATC (SEQ ID NO:118)) targeting the 3′-UTR of SARS-CoV-2 and the Superscript III transcriptase kit (Invitrogen, Carlsbad, CA). For genome RNA, the RT primer (GTGTCTTTGATTTCGAGCAAC (SEQ ID NO:119)) was annealing to 5′ of SARS-CoV-2 genome. The RT products were then used to perform real-time PCR using primers specifically targeting the N gene of SARS-CoV-2 (forward, CATTGGCATGGAAGTCACAC (SEQ ID NO:120); reverse, TCTGCGGTAAGGCTTGAGTT (SEQ ID NO:121)) or targeting the 5′-end of SARS-CoV-2 genome (forward, ACTGTCGTTGACAGGACACG (SEQ ID NO:122); reverse, ACGTCGCGAACCTGTAAAAC (SEQ ID NO:123)) in a StepOne real-time PCR system (Applied Biosystems). A standard curve was generated using a plasmid encoding the nucleocapsid (N) gene or full-length genome of SARS-CoV-2 plasmid. Amplification cycles used were 2 min at 95° C., and 40 cycles of 15 s at 95° C., and 1 min at 60° C. The threshold for detection of fluorescence above the background was set within the exponential phase of the amplification curves. For each assay, 10-fold dilutions of standard plasmid or viral RNA were generated, and negative-control samples and double-distilled water (ddH₂O) were included in each assay. After real-time qPCR, the Ct value from each sample was converted into log₁₀ viral RNA copies/mg tissue according to the standard curve. The RNA copies were calculated with the following formula: RNA copies/mg tissue=Log₁₀ [Ct-converted copies/μl×10(2 μl from 20 μl total cDNA)×25 (2 μl from 50 μl total RNA)×10 (100 μl from 1 ml homogenized tissue)/tissue weight (mg)]. The LoD is set as the maximum value of the normal control group. The exact log₁₀ RNA copies/mg was reported for each sample.

Quantification of cytokine in lungs of hamsters. Total RNA was extracted from lungs of Golden Syrian hamsters, and IFN-α1, IFN-γ, IL-1b, IL-2, IL-6, TNF, and CXCL10 mRNAs were quantified by real-time RT-PCR (Zivcec, M, et al. J Immunol Methods 2011 368:24-35; Safronetz D. et al. PLoS Pathog 2011 7:e1002426). GAPDH mRNA was used as internal controls. The cytokine mRNA of each group was expressed as fold-change in gene expression compared to normal animals (unimmunized and unchallenged) after normalization. Primers used for RT-qPCR were listed in Table 3.

TABLE 3
Primers used for RT-qPCR of hamster cytokines

| | GenBank | |
|---|---|---|
| Cytokine | Accession No. | Primer name and sequence |
| IFN-α1 | S78750.1 | Ham-IFN-a1-F: GCCTCTACCAGCAGCTCAGT (SEQ ID NO: 98) |
| | | Ham-IFN-a1-R: GCAGACAGGGTTCTCCAGAC (SEQ ID NO: 99) |
| IFN-γ | AF034482.1 | Ham-IFN-g-F: CCATCAAGGCAGACCTGTTT (SEQ ID NO: 100) |
| | | Ham-IFN-g-R: TTCTTGTTGGGACGATTTCC (SEQ ID NO: 101) |
| IL-1ß | AB028497.1 | Ham-IL-1b-F: GGTGGTGTCAGTCATTGTGG (SEQ ID NO: 102) |
| | | Ham-IL-1b-R: AGACAGCACGAGGCATTTCT (SEQ ID NO: 103) |
| IL-2 | EU729351.1 | Ham-IL-2-F: TGCACCCACTTCAAGCTCTA (SEQ ID NO: 104) |
| | | Ham-IL-2-R: GCCTTCTTGGGCATGTAAAA (SEQ ID NO: 105) |
| IL-6 | AB028635.1 | Ham-IL-6-F: CTCCGCAAGAGACTTCCATC (SEQ ID NO: 106) |
| | | Ham-IL-6-R: ACCAAACCTCCGACTTGTTG (SEQ ID NO: 107) |
| TNF | XM_005086799.3 | Ham-TNF-F: GACGGGCTGTACCTGGTTTA (SEQ ID NO: 108) |
| | | Ham-TNF-R: GAGTCGGTCACCTTTCTCCA (SEQ ID NO: 109) |
| CXCL10 | NM_001281344.1 | Ham-CXCL10-F: TGCCAGTGCTATCCTCCTTT (SEQ ID NO: 110) |
| | | Ham-CXCL10-R: GATGGCCTCAGACTCTGGAT (SEQ ID NO: 111) |
| GAPDH | DQ403055.1 | Ham-GAPDH-F: AACTTTGGCATTGTGGAAGG (SEQ ID NO: 112) |
| | | Ham-GAPDH-R: CGACATGTGAGATCCACGAC (SEQ ID NO: 113) |

Histology. The right lung lobes from each hamster were preserved in 4% (vol/vol) phosphate-buffered paraformaldehyde for 14 days before transferred out of the BSL-3 facility. Fixed tissues were embedded in paraffin, sectioned at 5 μm, deparaffinized, rehydrated, and stained with hematoxylin-eosin (HE) for the examination of histological changes by light microscopy.

Immunohistochemistry (IHC). Five-micron sections of paraffin embedded tissues were placed onto positively charged slides. After deparaffinization, sections were incubated with target retrieval solution (Dako, Carpinteria, CA) for antigen retrieval. After blocking, lung sections were subjected to IHC staining using a rabbit SARS-CoV-2 N protein (NB100-56576, Novus Biologicals). Slides were counter stained with hematoxylin.

Statistical analysis. Quantitative analysis was performed by either densitometric scanning of autoradiographs or by using a phosphorimager (Typhoon; GE Healthcare, Piscataway, NJ) and ImageQuant TL software (GE Healthcare, Piscataway, NJ) or Image J software (NIH, Bethesda, MD). Statistical analysis was performed by one-way or two-way multiple comparisons using SPSS (version 8.0) statistical analysis software (SPSS Inc., Chicago, IL), two-way ANOVA, or Student's t test. A P value of <0.05 was considered statistically significant.

Results

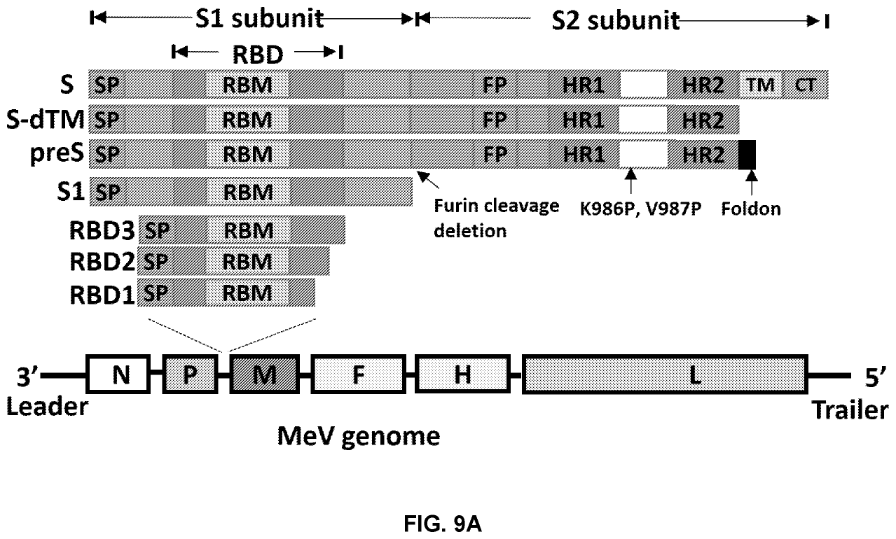

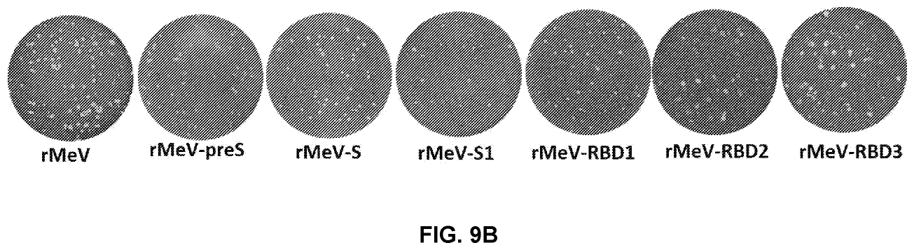

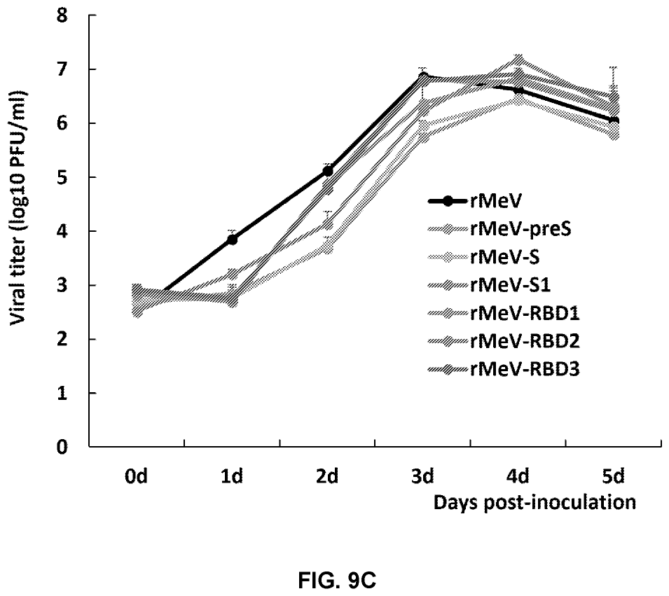

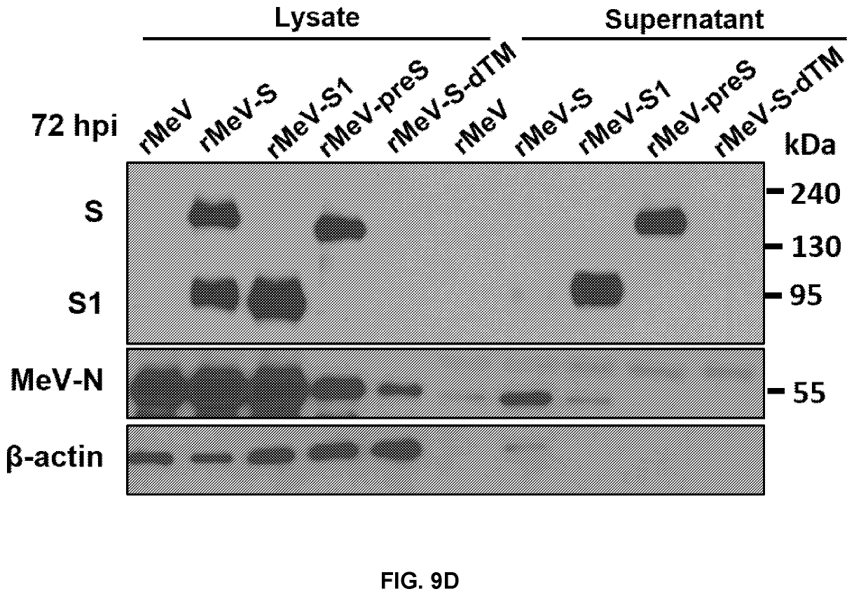

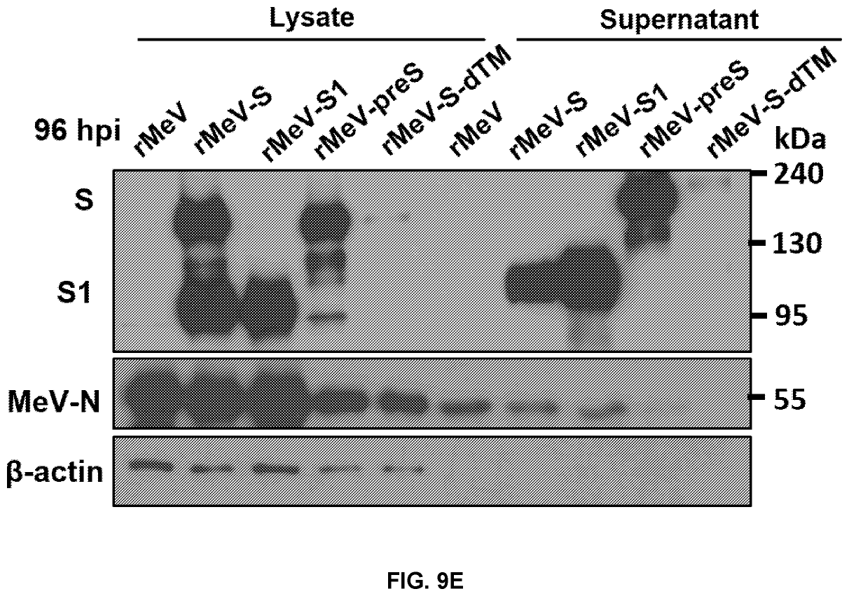

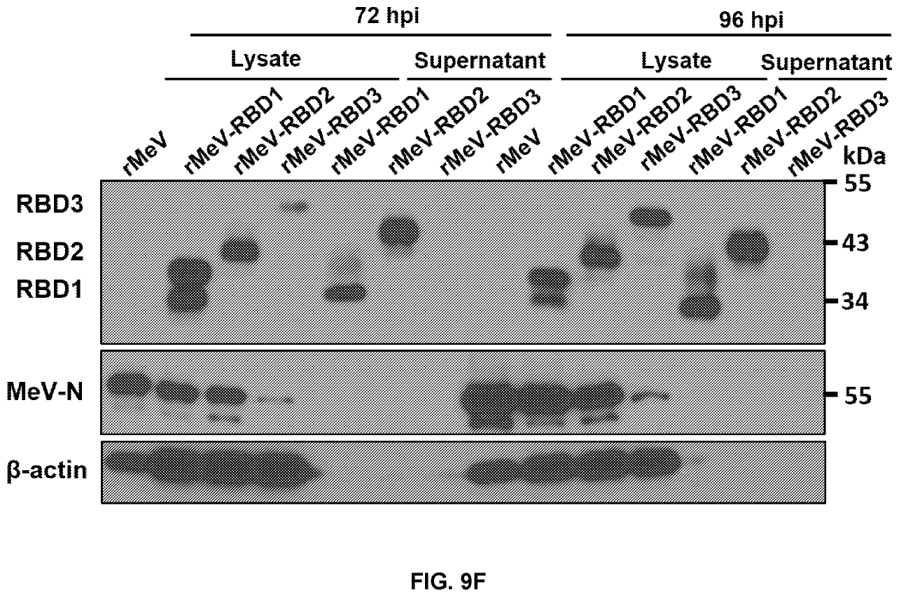

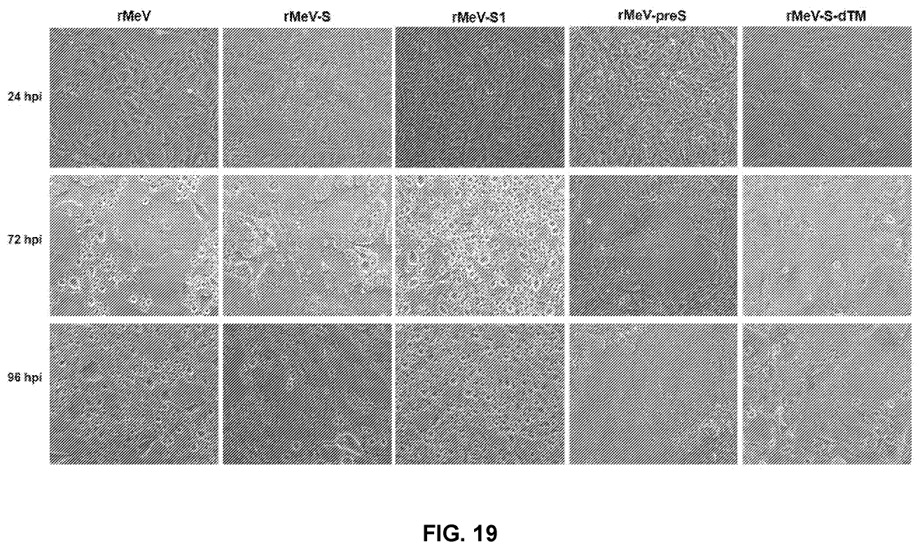

Recovery of recombinant MeV expressing SARS-CoV-2 S antigens. A yeast-based recombination system was developed for rapidly constructing cDNA clones of rMeV expressing foreign genes such as the SARS-CoV-2 S antigens. Six overlapping DNA fragments (designated a to f) spanning the full-length MeV Edmonston vaccine strain and a SARS-CoV-2 gene annealing to the junction between the P and M genes were ligated into the pYES2 vector in a single step mediated by DNA recombinases present in yeast (FIG. 18). Using this strategy, a series of MeV vaccine vectors expressing eight variants of the SARS-CoV-2 S protein were constructed: (i) full length S (S), (ii) deletion of the transmembrane domain and cytoplasmic tail reflecting the soluble ectodomain (S-dTM), (iii) S1 subunit (S1), (v) three different lengths of receptor-binding domain (RBD1, RBD2, and RBD3) of S, and (vi) a prefusion-stabilized soluble ectodomain with deletion of the furin cleavage site, two proline mutations (amino acids 986 and 987), and a self-trimerizing T4 fibritin trimerization motif replacing its transmembrane and cytoplasmic domains (pre-S) (Wrapp D, et al. Science 2020 367:1260-1263) (FIG. 9A). All rMeV viruses were recovered from full-length genome cDNAs using the standard reverse genetics system and plaque purified. To confirm that the recombinant viruses indeed contained the target gene, viral genomic RNA was extracted followed by RT-PCR using primers annealing to the flanking MeV P and M genes. PCR products were sequenced, confirming that S and its variants were inserted into the MeV genome between the P and M genes. Finally, the entire genome of each recombinant virus was sequenced to confirm that no additional mutations had been introduced. Compared to the parental rMeV, all recombinant viruses formed relatively smaller plaques (FIG. 9B) and exhibited delayed syncytia formation and cytopathic effects (CPE) (FIG. 19). Multi-step replication curve showed that these recombinant viruses had delayed replication kinetics in Vero CCL81 cells (FIG. 9C). However, the peak titer of rMeV-S1 (10^{7.2} PFU/ml) was higher than that of the parental rMeV (10^{6.9} PFU/ml). Three recombinant viruses (rMeV-RBD1, RBD2, and RBD3) grew to titers comparable to the parental rMeV in Vero CCL-81 cells whereas rMeV-S and rMeV-preS had 0.3-0.5 log reductions in peak titer. These results suggest that insertion of near full-length SARS-CoV-2 S genes into the MeV genome further attenuates MeV replication.

SARS-CoV-2 S proteins are highly expressed by the rMeV vector. The expression of the SARS-CoV-2 S proteins was examined by rMeV in confluent Vero CCL81 cells inoculated at an MOI of 0.01. Cell culture supernatants and lysates were harvested at 72 and 96 h post-infection and analyzed by Western blot using antibody against SARS-CoV-2 S1 protein or MeV N protein. As expected, two proteins with molecular weights of 190 and 95 kDa were detected in rMeV-S infected cells at 72 h, reflecting the full-length S and cleaved S1 (FIG. 9D). In rMeV-preS infected cells, the 180 kDa uncleaved, stabilized prefusion S (preS) protein was detected, somewhat smaller because it lacks the transmembrane and cytoplasmic domains. In rMeV-S1-infected cells, the 95 kDa S1 protein was detected. The preS and S1 but not the full-length S were also secreted into the culture medium.

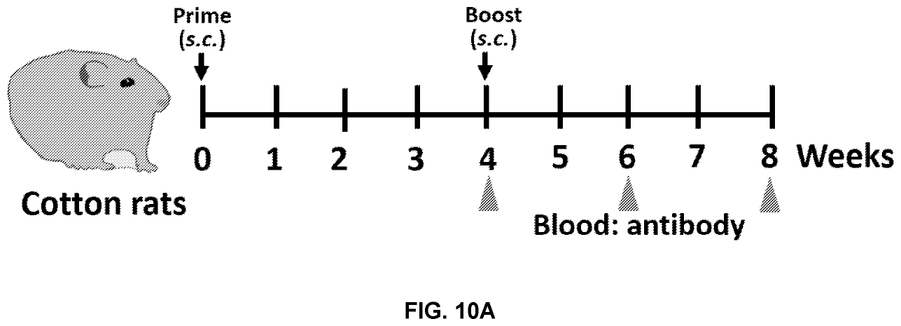

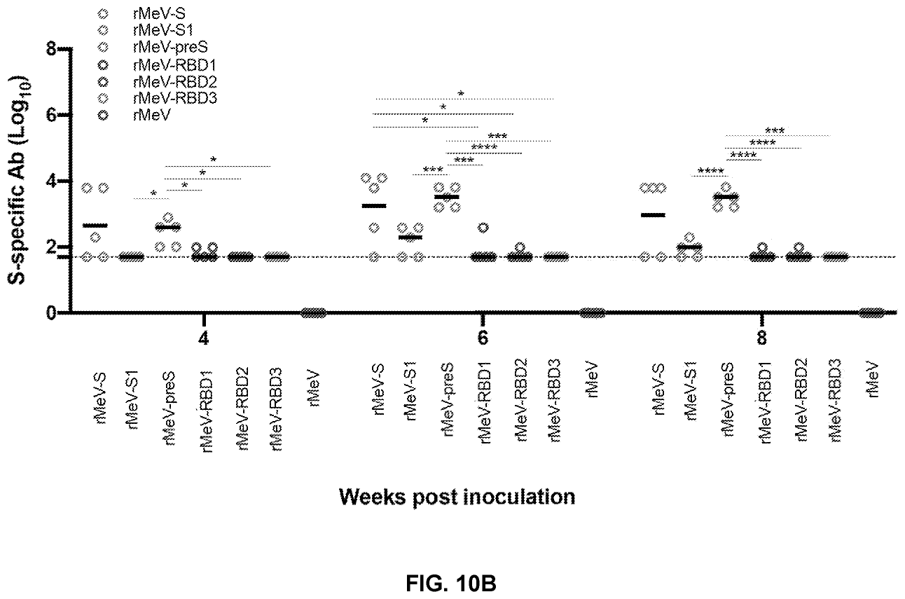

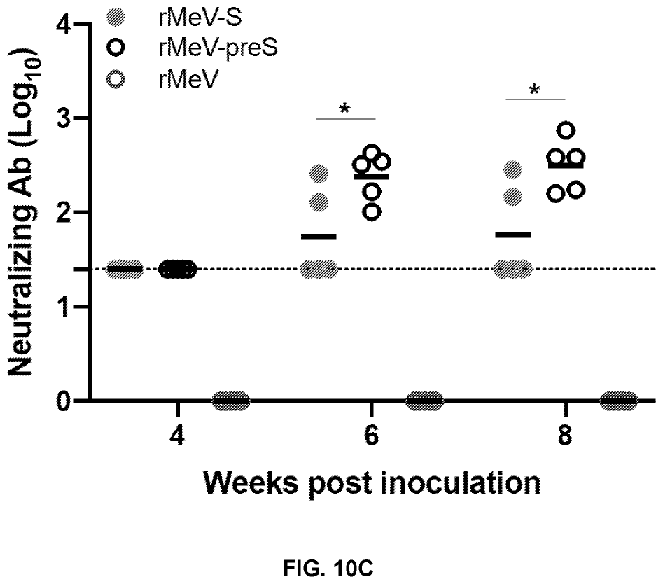

By 96 h post-infection, protein expression had increased (FIG. 9D). Although the S1 subunit from rMeV-S infected cells had not been detected in the supernatant at 72 h, it was at 96 h (FIG. 10E). At both times, much of the S protein had been cleaved to its active form but the release of S1 into the supernatant at the later time suggests that some of the metastable cleaved S protein had either triggered spontaneously or following engagement with its receptor on a neighboring cell. Triggering releases S1 and allows S2 to refold, engaging with the target cell membrane and causing fusion between the two membranes.

RBD1 (34 kDa), RBD2 (40 kDa), and RBD3 (45 kDa) proteins were produced by their respective rMeV vector infected cells (FIG. 10F), consistent with their predicted molecular weights. High levels of RBD1 and RBD2 were secreted into the cell culture supernatant. These results demonstrated that all of these SARS-CoV-2 S antigens were highly expressed by the rMeV vector, with the exception of S-dTM (FIG. 10E) which was not pursued further. The extensive fusion CPE observed at both 72 and 96 h (FIG. 19) is most likely due to MeV which causes this type of CPE. Interestingly, the CPE did not impair the production of most versions of the S protein over the 96 h of the experiment.

rMeV-expressed S and preS are highly immunogenic in cotton rats. Cotton rats (Sigmodon hispidus) are a susceptible model for MeV infection (Green M G, et al. Lab Animal 2013 42:170-176). Thus, the immunogenicity of these rMeV-based SARS-CoV-2 vaccine candidates was first tested in cotton rats (FIG. 10A). Four-week-old SPF cotton rats were immunized subcutaneously with 4×10⁵ PFU of each rMeV-based SARS-CoV-2 vaccine candidate and boosted with 2×10⁶ PFU of the same vaccine candidate 4 weeks later. Sera were collected at weeks 4, 6, and 8, and S-specific antibodies were detected by ELISA using preS protein as the antigen. By week 4, all five cotton rats in the rMeV-preS group had developed S-specific antibodies whereas only 3 out of 5 cotton rats in the rMeV-S group had (FIG. 10B). However, antibodies were detectable at the lowest dilution in most cotton rats in the rMeV-S1 and RBD1-3 groups. After the booster immunization, antibodies in the rMeV-preS group were uniformly high whereas 3 cotton rats in rMeV-S had high antibody titers and 2 had low antibody titers. Despite the booster immunization, antibody titers in rMeV-S1 group remained low and antibody titers in rMeV-RBD1-3 were at the minimum detectable level (FIG. 10B).

The functional activity of the antibodies in sera from the two groups with the most antibody to S, rMeV-S and rMeV-preS, were tested for their ability to neutralize live SARS-CoV-2, in comparison to the rMeV group. Neutralizing antibody titers in the rMeV-preS group were significantly higher than those in the rMeV-S group (P<0.05), on average 5.5-fold higher (FIG. 100). Therefore, in the MeV expression system, preS is the most effective immunogen for inducing neutralizing antibodies in the cotton rat.

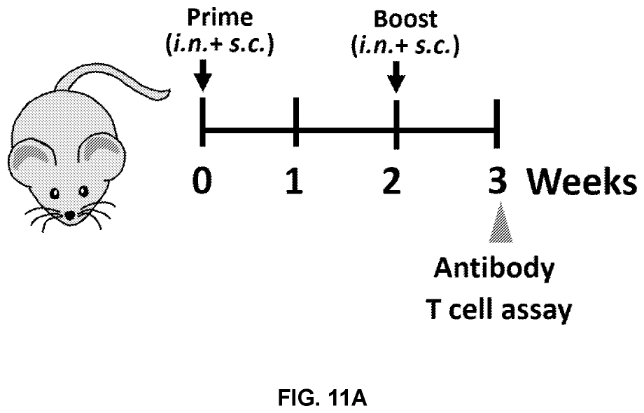

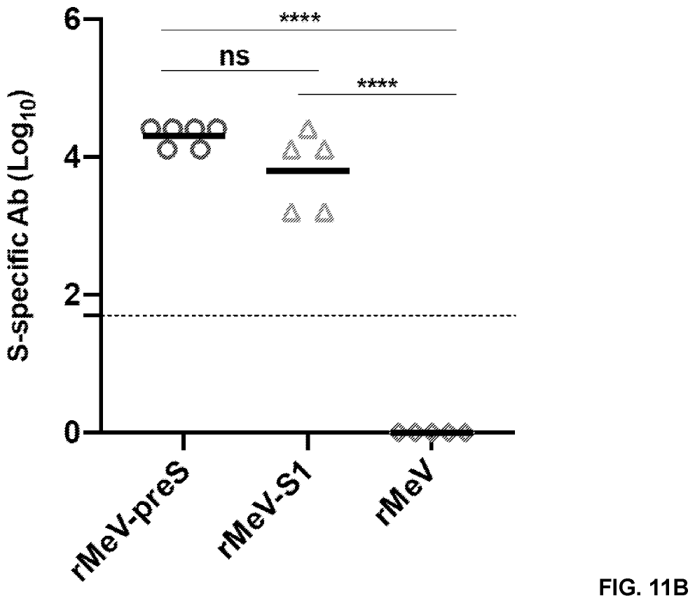

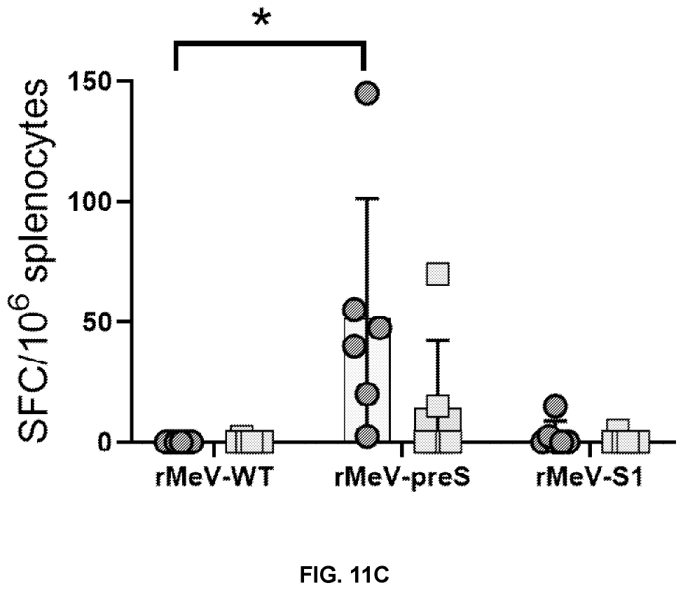

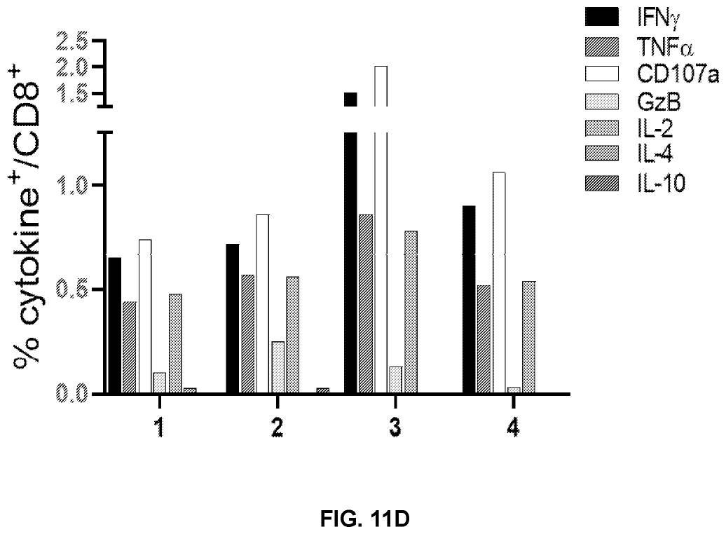

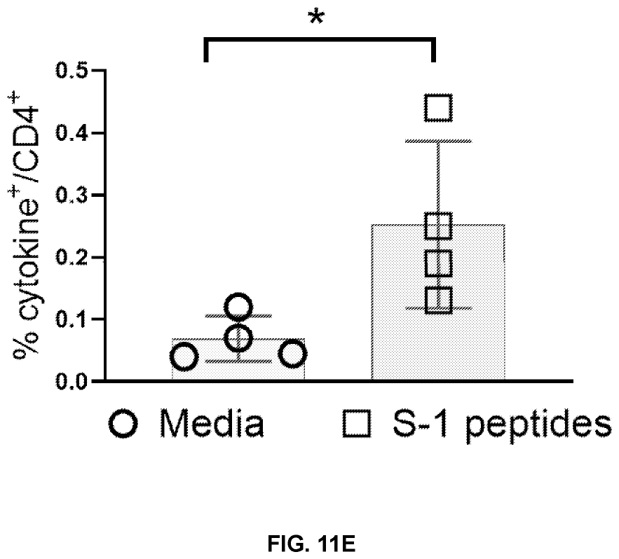

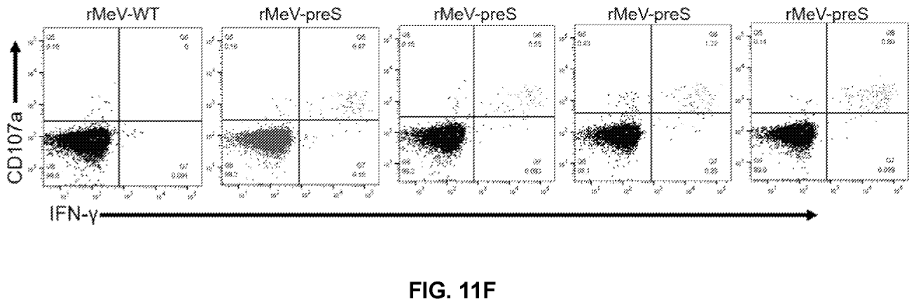

rMeV-preS is highly immunogenic in IFNAR1^{-/-}-hCD46 mice and induces high levels of Th1-biased T cell immune responses. MeV vaccine strains can use several receptors (human CD46, CD150, and Nectin 4) to infect different cell types (Griffin D E. Viral Immunol 2018 31:86-95). Type-I interferon receptor subunit 1 (IFNAR1) knockout, human CD46 transgenic mice (IFNAR1^{-/-}-hCD46) mice can be robustly infected by MeV and have been used as a model to test the efficacy of many rMeV-based vaccine candidates (Mura M, et al. Virology 2018 524:151-159). Thus, rMeV-preS and rMeV-S1 were test in IFNAR1^{-/-}-hCD46 mice to determine if they are immunogenic (FIG. 11A). Six-week-old IFNAR1^{-/-}-hCD46 mice were immunized with 8×10⁵ PFU of each vaccine candidate (half subcutaneous and half intranasal) and at week 2 were boosted with the same vaccine candidate at a dose of 6×10⁵ PFU. Sera were collected at week 3 and antibody to preS was quantified by ELISA. It was observed that rMeV-S1 induced higher antibody in IFNAR1^{-/-}-hCD46 mice than in cotton rats. However, rMeV-preS induced more antibody than rMeV-S1, but the difference was not significant (P>0.05) (FIG. 11B). These results suggest that rMeV-S1 may replicate more robustly in the presence of hCD46 receptor than it did in the cotton rat, where it induced a lower level of antibody.

At week 3, all groups were terminated and their splenocytes were used to characterize vaccine induced T cell immunity. SARS-CoV-2 antigen-specific IFNγ-producing T cells was first quantified by ELISPOT. Mice immunized with rMeV-preS had significantly higher frequencies of S1 peptide-specific IFNγ-producing T cells compared to the control mice vaccinated with rMeV vector (P<0.05) (FIG. 11C). Upon stimulation with peptide pools spanning the S1 subunit, 5 out of 6 mice in rMeV-preS group showed a strong antigen-specific IFNγ-producing T cell response whereas only 2 out of 6 mice in rMeV-S1 group showed a weak T cell response (FIG. 11C). When S2 peptide pools were used for stimulation, only 2 of 6 mice in rMeV-preS group had a strong IFNγ-producing T cell response (FIG. 11C), indicating that the vaccine candidate induced T cells primarily targeting the N-terminus of the SARS-CoV-2 S protein. To further characterize the nature of vaccine induced T cells, 4 mice with the strongest IFNγ-producing T cell responses in the rMeV-preS group were analyzed using flow cytometry and intracellular cytokine staining (FIGS. 11D and 11E). Th1 cells, which produce cytokines such as IFN-γ, TNF-α, and IL-2, play an important role in protection against viral infection (Liew F. Y. Nat Rev Immunol 2002 2:55-60). After peptide stimulation ex vivo, CD8+ T cells producing one or more of the three signature Th1 cytokines, IFNγ, TNFα, and IL-2 were detected in all four mice immunized with rMeV-preS (FIGS. 11D and 11E). Moreover, antigen-specific cytokine-producing CD4⁺ T cells were also detected but at lower frequencies representing 01-0.5% of the total CD4⁺ T cells (FIG. 11D). Together, these data suggest that rMeV-preS vaccine candidate is capable of inducing robust T cell immunity that is predominated by CD8⁺ T cells capable of producing Th1 cytokines.

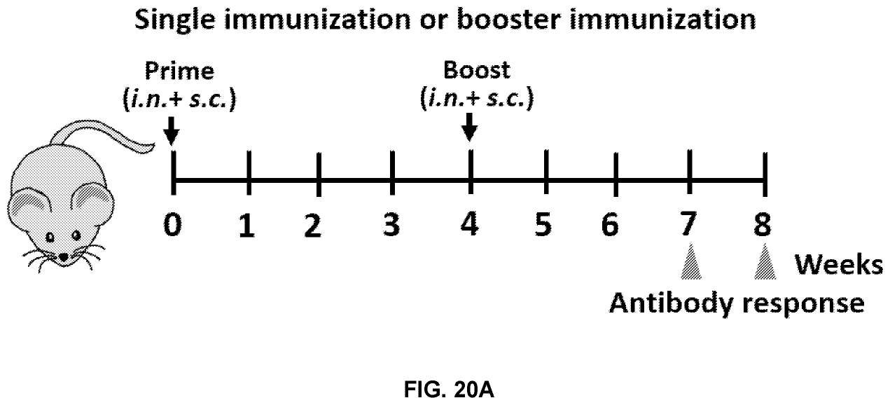

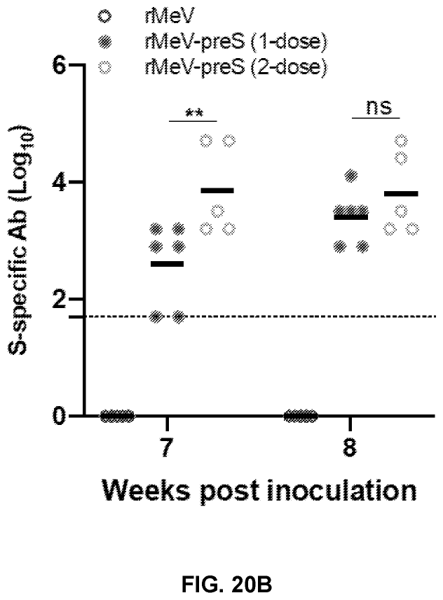

A single immunization of rMeV-preS induces a high level of antibody in IFNAR1^{-/-} mice. Recently, it was shown that typed interferon, but not the hCD46, is the barrier for MeV infection in mice³⁸. IFNAR1^{-/-} mice can be readily infected by MeV. Thus, the effectiveness of single immunization and booster immunization in inducing S-specific antibody in IFNAR1^{-/-} mice was compared. For the single immunization group, IFNAR1^{-/-} mice were immunized with 8×10⁵ PFU of rMeV-preS (half subcutaneous and half intranasal). For the booster immunization group, IFNAR1^{-/-} mice were immunized with 8×10⁵ PFU of rMeV-preS and were boosted at the same dose 4 weeks later (FIG. 20A). At week 7, S-specific antibody in the booster immunization group was significantly higher than the single immunization group (P<0.01) (FIG. 20B). At week 8, there was no significant difference between these two groups (P>0.05) (FIG. 20B). This result suggests that a single immunization of rMeV-preS may be sufficient to induce a high level of SARS-CoV-2 specific antibody.

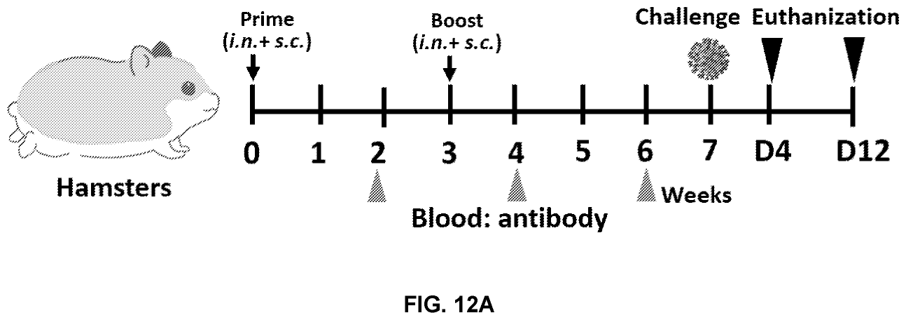

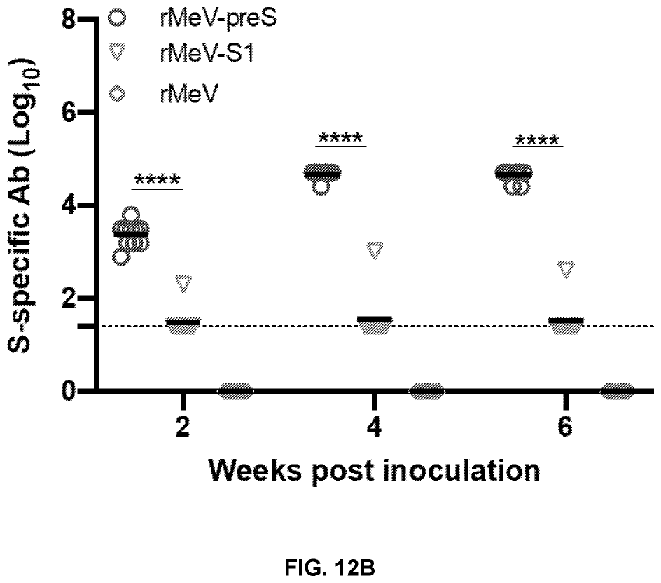

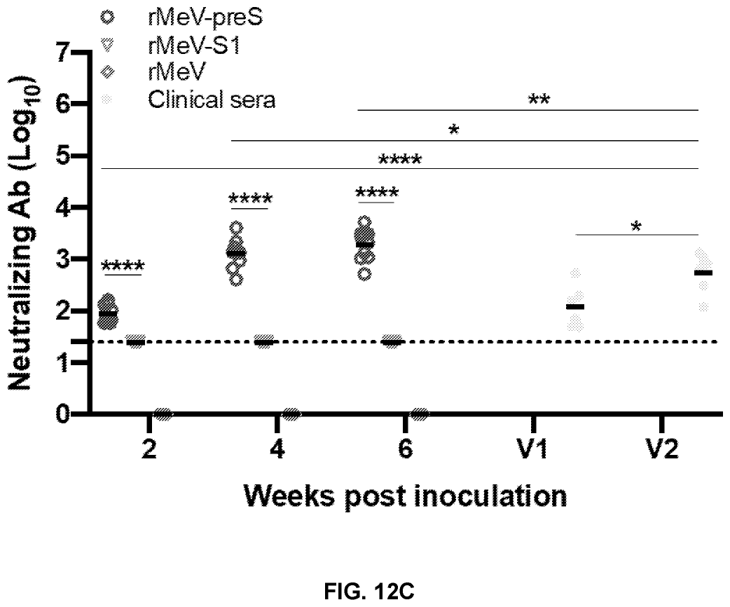

rMeV-preS is highly immunogenic in Golden Syrian hamsters. Golden Syrian hamsters are an excellent animal model to evaluate SARS-CoV-2 pathogenesis and the efficacy of vaccine candidates or antiviral drugs. Early studies also suggest that Golden Syrian hamsters are susceptible to MeV infection (Wear D J, et al. Nature 1970 227:1347-1348; Mirchamsy H, et al. Acta Virol 1972 16:77-79). However, the optimal route for MeV immunization in hamsters is unknown. Thus, the combination of intranasal and subcutaneous route was chosen for immunization in order to achieve maximal levels of immune responses. rMeV-S1 was chosen to compare with rMeV-preS in the hamster study as rMeV-S1 induced good antibody responses in IFNAR1^{-/-}-hCD46 mice (FIG. 11B) and grew to the highest titer in Vero cells (FIG. 9C). Ten 4-week-old Golden Syrian hamsters in each group were first immunized with 8×10⁵ PFU of the parental rMeV, rMeV-preS, or rMeV-S1, and boosted with the same dose three weeks later (FIG. 12A). High antibody titers were detected in all 10 hamsters in the rMeV-preS group at week 2 after a single dose vaccination. After a booster immunization at week 3, antibody titers further increased at weeks 4 and 6 (FIG. 12B). Unfortunately, only 1 out of 10 hamsters in the rMeV-S1 group produced a robust antibody response (FIG. 12B). As expected, neutralizing antibodies in the rMeV-preS group were detectable at week 2, and increased at weeks 4 and 6, whereas neutralizing antibodies in the rMeV-S1 group were at detectable levels (FIG. 12C).

The level of neutralizing antibody induced by rMeV-preS was compared in hamsters with that induced in acute and convalescent sera collected from 6 COVID-19 patients at two time points: during acute infection (V1) and after recovery (V2). As expected, antibody titer of convalescent sera from the recovered COVID-19 patients was significantly higher than the titer of sera collected from the same patients during acute infection (P<0.05) (FIG. 12C). Importantly, neutralizing antibody titers at weeks 4 and 6 in rMeV-preS immunized hamsters were significantly higher than these random human convalescent sera (P<0.05, P<0.01) (FIG. 12C). These results confirm that rMeV-preS is highly immunogenic.

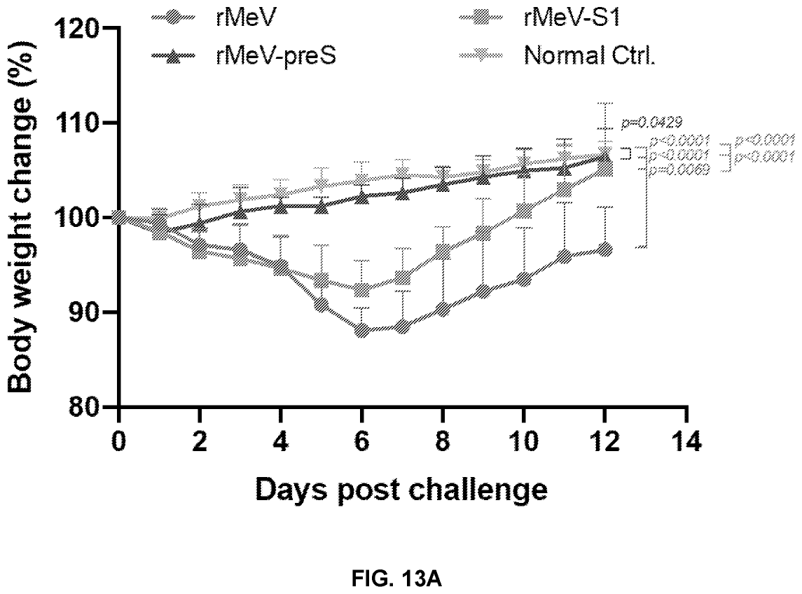

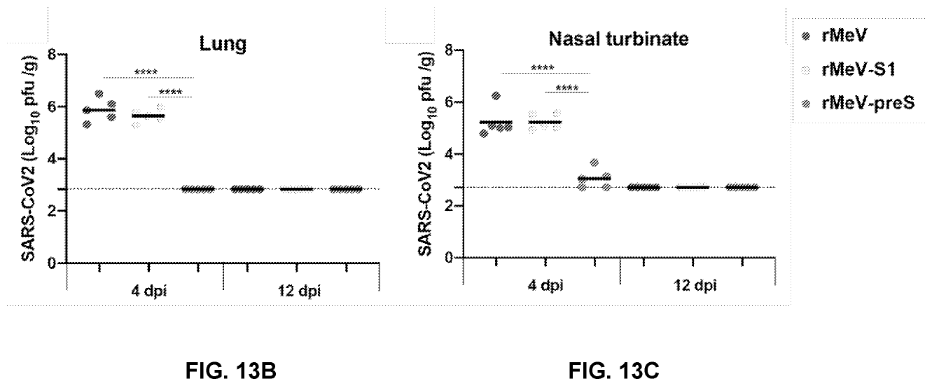

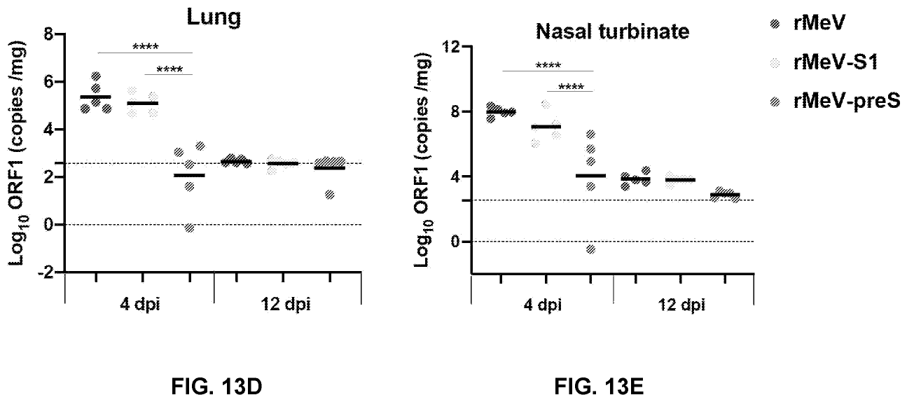

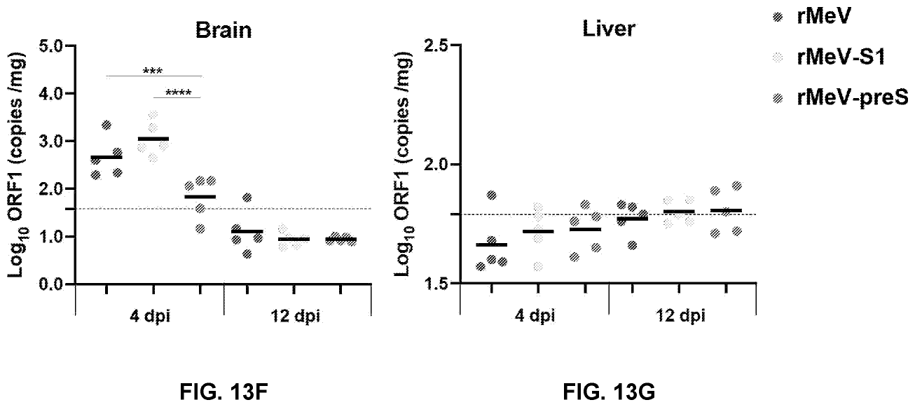

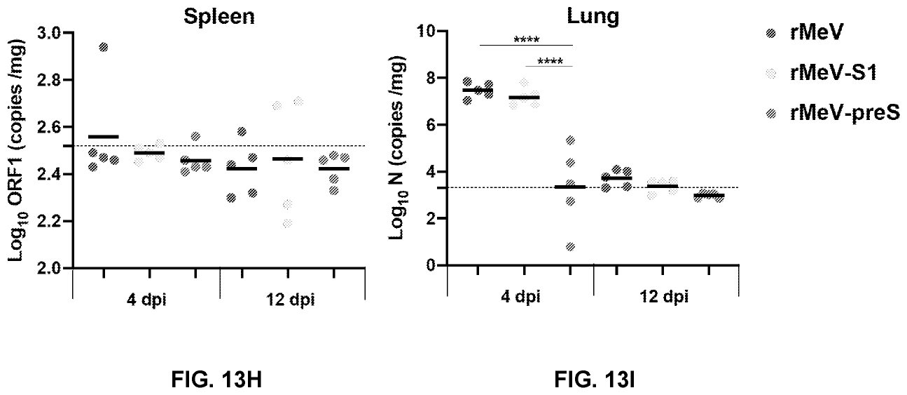

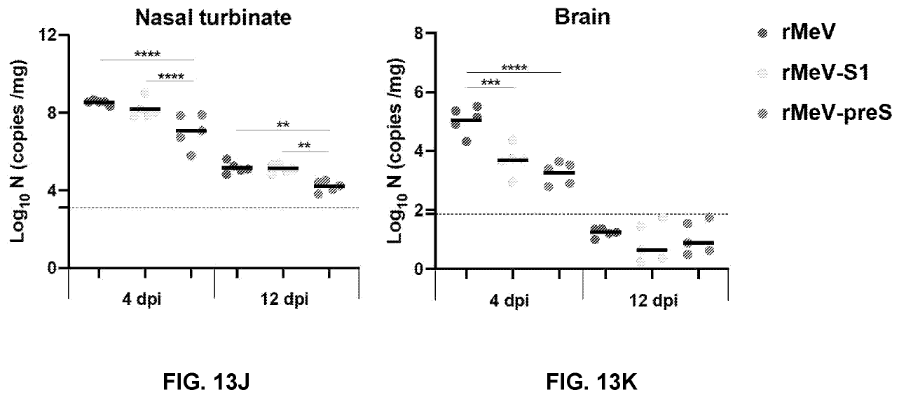

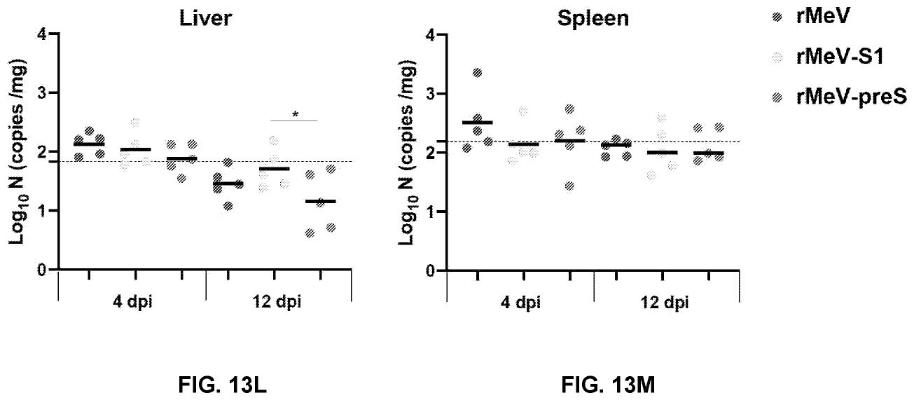

rMeV-preS vaccination provides complete protection against SARS-CoV-2 replication in Golden Syrian hamsters. At week 7, hamsters in rMeV, rMeV-S1, and rMeV-preS groups were moved to a BSL3 animal facility and challenged intranasally with 10⁵ PFU of SARS-CoV-2. The normal control hamsters continued to be housed at BSL2 animal facility and were inoculated with DMEM. At day 4 post-challenge, five animals from each group were euthanized, and the remaining 5 animals were euthanized at day 12 post-challenge. the protection efficacy of rMeV-based vaccine candidates including clinical signs, weight loss, viral replication, RNA replication, cytokine responses in the lung, and lung histology and immunohistochemistry (IHC) was systemically evaluated. Hamsters in the rMeV vector control group that were inoculated with SARS-CoV-2 exhibited clinical symptoms such as ruffled coat and weight loss (FIG. 13A). Hamsters in the rMeV group started to lose weight at day 1 post-challenge and reached approximately 15% weight loss at day 6, and then started to regain weight from day 8 to 12 (FIG. 13A). Hamsters in rMeV-S1 groups had similar weight loss from days 1 to 6 but had a faster weight recovery compared to rMeV group (FIG. 13A). Importantly, hamsters in rMeV-preS group did not have any abnormal reaction or weight loss. The body weight in rMeV-preS group was not significantly different at most time points compared to the normal controls (FIG. 13A).

At day 4, 5 animals from each group were euthanized, and lungs, nasal turbinate, brain, liver, and spleen were collected for virus titration by plaque assay. An average titer of 7.4×10⁵ and 1.7×10⁵ PFU/g of SARS-CoV-2 were detected in lungs (FIG. 13B) and nasal turbinates (FIG. 13C) in the rMeV group, respectively. No infectious virus was detected in brain, liver, or spleen tissues in the rMeV group. Similarly, 4.4×10⁵ and 1.7×10⁵ PFU/g of SARS-CoV-2 were detected in lungs (FIG. 13B) and nasal turbinates (FIG. 13C) in the rMeV-S1 group, respectively, which were not significantly different from the rMeV group (P>0.05). Importantly, infectious SARS-CoV-2 was below the detection limit in the lung in rMeV-preS group (FIG. 13B) and only 3 out 5 animals had low viral titer (1.9×10³ PFU/g) in nasal tissue (FIG. 13C). At day 12, the remaining 5 hamsters in each group were euthanized. No infectious SARS-CoV-2 was detected in lung (FIG. 13B), nasal turbinate (FIG. 13C), or other tissues of any group.

To determine if SARS-CoV-2 genome RNA was present in these tissues primers annealing to the 5′-end of SARS-CoV-2 genome were used. The highest number of background RNA copies detected in an unchallenged control group was set as the detection limit. As expected, high genome RNA copies were detected in both the lung (FIG. 13D) and nasal turbinate (FIG. 13E), moderate levels of viral RNA were detected in brain (FIG. 13F), near detectable levels of viral genome RNA were detected in liver (FIG. 13G) and spleen (FIG. 13H) tissues in rMeV group at day 4. It should be noted that genomic RNA copies in lung, nasal turbinate and brain in the rMeV-preS group were significantly lower than rMeV and rMeV-S1 groups (P<0.001, P<0.0001). Importantly, the average RNA copies in lungs, brain, liver, and spleen from rMeV-preS group were near or below the detection limit whereas nasal turbinate had RNA titers of approximately 10⁴ RNA copies/g tissue. At day 12, low levels of RNA were detected in nasal tissue and little or no RNA was detectable in all other tissues in all groups.

In addition to the full-length genome RNA, SARS-CoV-2 replication generates subgenomic RNA, which is more abundant than genomic RNA. Thus, the levels of total viral RNA including genomic and subgenomic RNA was determined using primers annealing to the N gene located at the 3′ end of genome. Overall, the patterns of total RNA titers in lung (FIG. 13I), nasal turbinate (FIG. 13J), brain (FIG. 13K), liver (FIG. 13L), and spleen (FIG. 13M) were similar to those of genomic RNA in these tissue at days 4 and 12. Collectively, these results demonstrate that rMeV-preS vaccination provided complete protection against SARS-CoV-2 infection in hamsters whereas rMeV-S1 was unable to protect hamsters from SARS-CoV-2 infection.

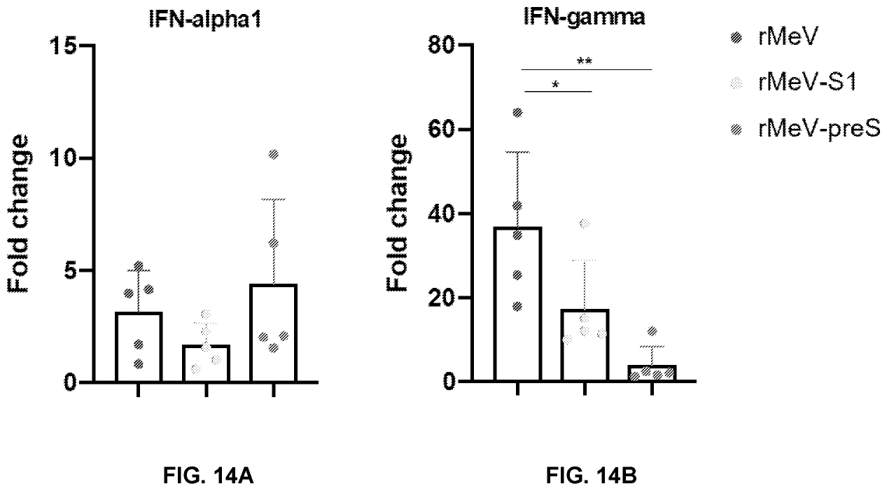

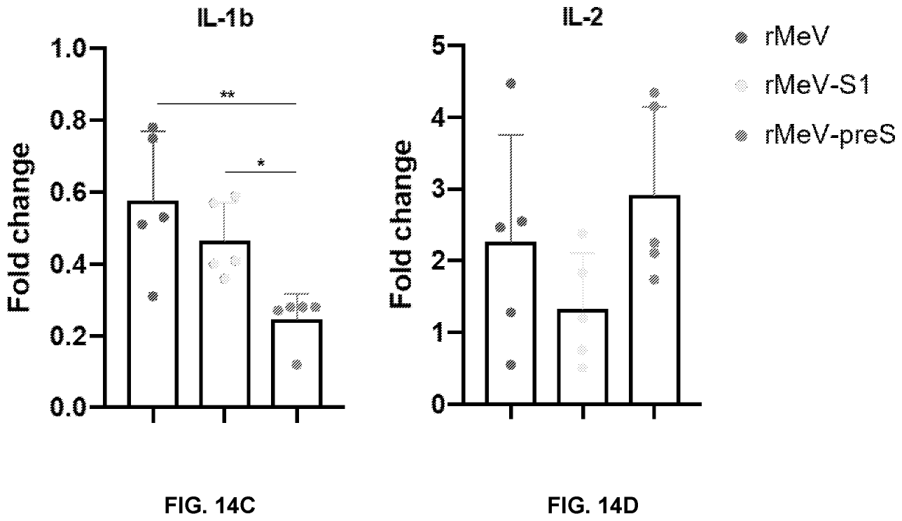

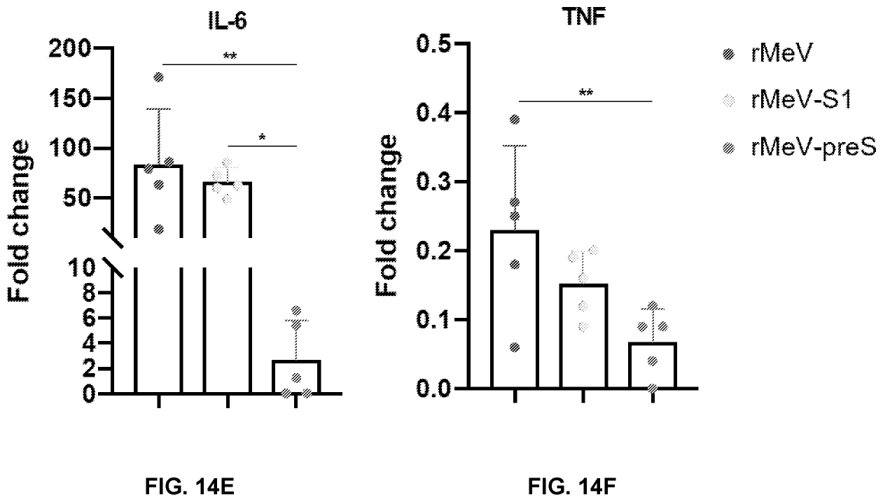

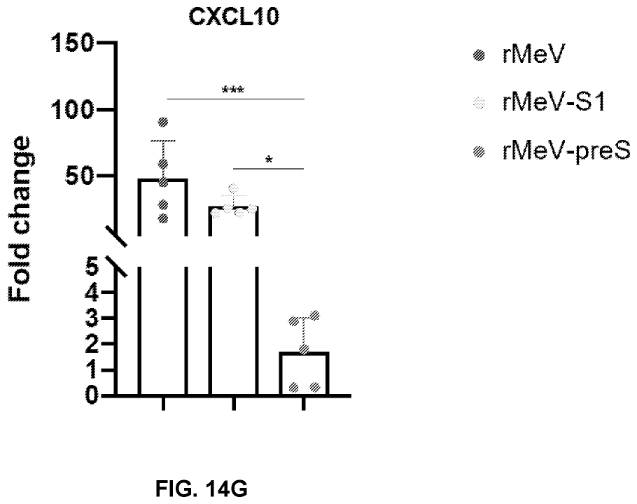

rMeV-preS vaccination prevents the SARS-CoV-2 induced cytokine storm in lungs. Cytokine storms play an important role in the pathogenesis and disease severity of COVID-19 patients (Zhang X et al., Nature 2020 583:437-440). Thus, it was determined whether rMeV-preS vaccination can prevent cytokine storm in the lungs. Briefly, IFN-α1, IFN-γ, IL-1b, IL-2, IL-6, TNF, and CXCL10 in lungs in each group were quantified by real-time RT-PCR and normalized to a control. Lung IFN-γ (FIG. 14B), IL-6 (FIG. 14E), and CXCL10 (FIG. 14G) mRNA had approximately 17-36, 66-84 and 27-48-fold increases in rMeV and rMeV-S1 groups compared to the normal control group, respectively. However, the increases in these three cytokine mRNAs in the rMeV-preS group were minimal (2- to 4-fold increase). Statistically, IFN-γ, IL-6 and CXCL10 were indistinguishable between the rMeV-preS group and the normal control group (P>0.05). In addition, increases in TNF (FIG. 14F) and IL-1b (FIG. 14C) in the rMeV-preS group were significantly less than rMeV and rMeV-S1 groups (P<0.05). IFN-α1 (FIG. 14A) and IL-2 (FIG. 14D) in rMeV, rMeV-S, and rMeV-preS groups were similar (P>0.05). These results suggest that rMeV-preS immunization prevents the cytokine storm in hamster lungs caused by a SARS-CoV-2 challenge.

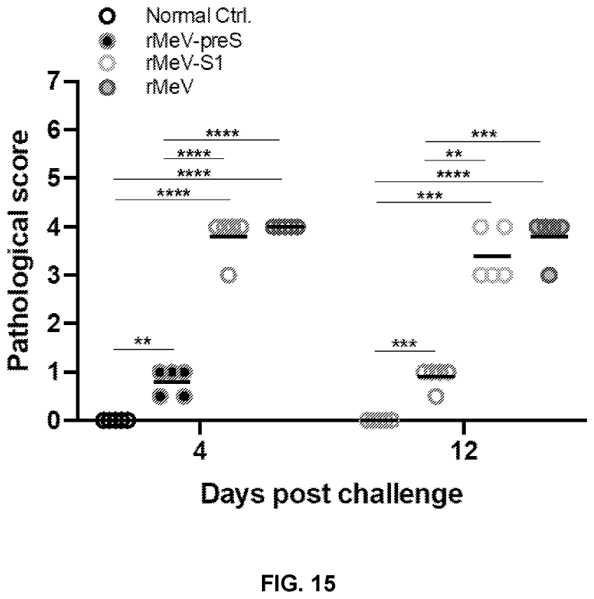

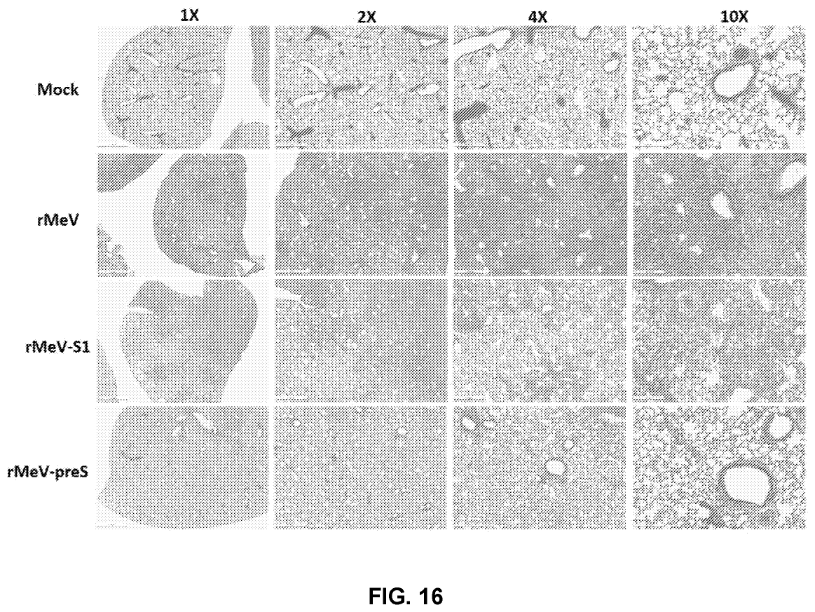

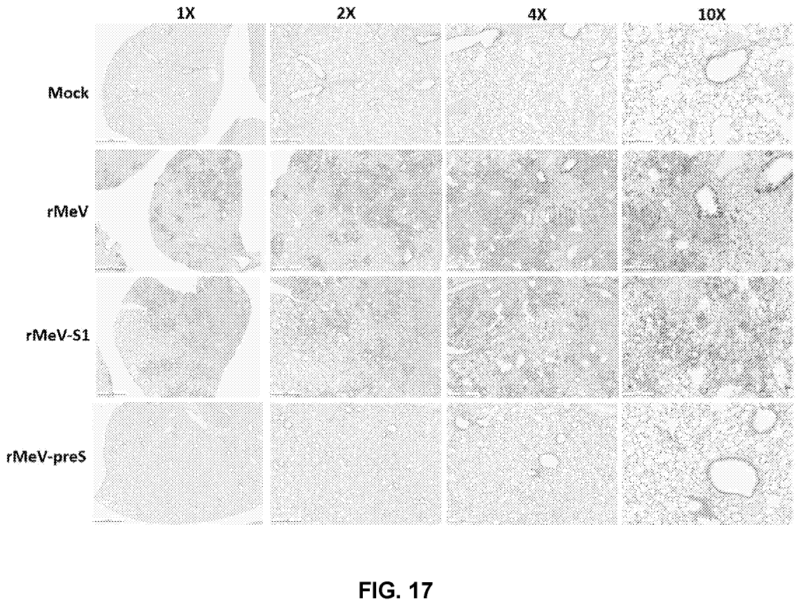

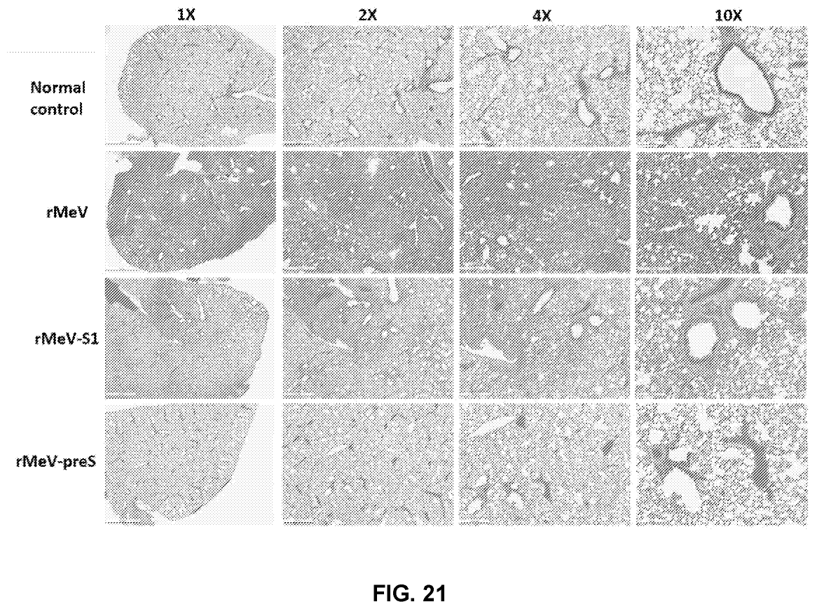

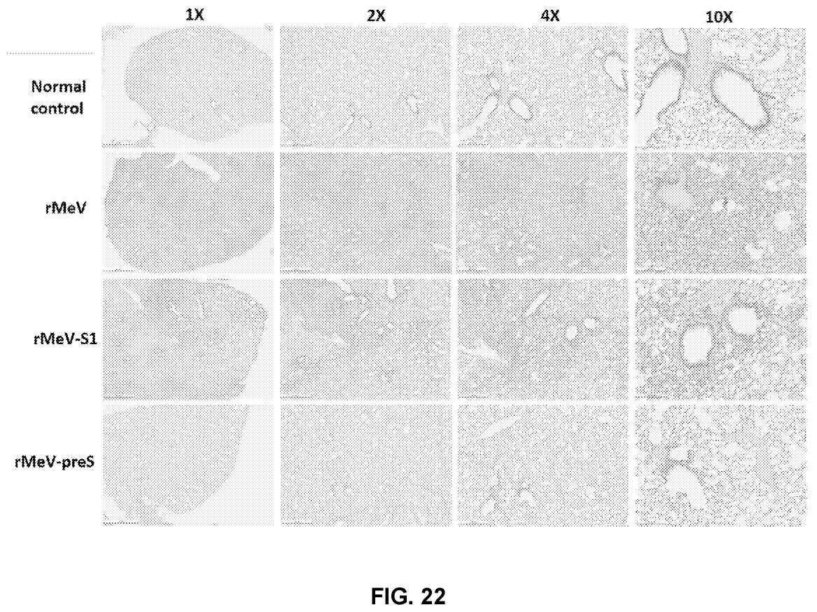

rMeV-preS vaccination protects hamsters from SARS-CoV-2 induced lung pathology. All lungs from hamster challenge study were stained with H&E and the severity of histological changes was scored blindly by a trained veterinary pathologist (FIG. 15). At day 4 post-challenge, all lung tissues from the SARS-CoV-2 inoculated rMeV group had extremely severe lung histopathological changes (average score of 4.0) characterized by extensive inflammation, interstitial pneumonia, edema, alveolitis, bronchiolitis, alveolar destruction, mononuclear cell infiltration, pulmonary hemorrhage, and peribronchiolar inflammation (FIGS. 15 and 16). Lung pathology in the rMeV-S1 group was also very severe (average score of 3.8) but slightly less than the rMeV group (P>0.05) (FIGS. 15 and 16). In contrast, lung tissues from the rMeV-preS group had little to mild pathological changes (average score of 0.8) (FIGS. 15 and 16). No lung pathology was found in the normal control group (score of 0) (FIGS. 15 and 6). At day 12, lung pathology in the rMeV group was still extremely severe (average score of 3.8) (FIGS. 7 and 21). Severe lung pathology (average score of 3.4) was found in the rMeV-S1 group. However, mild lung pathology (score of 0.9) was detected in the rMeV-preS group (FIGS. 15 and 22). Lung sections were also stained with SARS-CoV-2 N antibody by immunohistochemistry. At day 4, large amounts of SARS-CoV-2 N antigen were detected in all lung sections from the rMeV and rMeV-S1 groups (FIG. 17). In contrast, no N antigen was detected in lungs of the rMeV-preS group or the normal control (FIG. 17). At day 12, little N antigen was detected in the rMeV and rMeV-S1 groups and no antigen was detected in the lungs of the rMeV-preS group or normal control (FIG. 22). These results demonstrate that rMeV-preS vaccination protects hamsters from lung pathology and prevents SARS-CoV-2 antigen expression in lungs.

Discussion

In this study, a highly efficacious rMeV-based SARS-CoV-2 vaccine candidate was developed. The rMeV-preS based vaccine candidate is more potent in triggering SARS-CoV-2-specific neutralizing antibody than rMeV-based full-length S vaccine candidate. Antibodies induced by rMeV-preS were uniformly high in all four animal models including cotton rats, IFNAR^{-/-} mice, IFNAR1^{-/-}-hCD46 mice, and Syrian Golden hamsters and were significantly higher than antibody titers of human sera from convalescent COVID-19 patients. A single immunization of rMeV-preS was sufficient to induce a high level of SARS-CoV-2 specific antibody. In addition, rMeV-preS induces high levels of Th1-biased T cell immunity. Syrian Golden hamsters immunized with rMeV-preS were completely protected against SARS-CoV-2 challenge including body weight loss, viral replication, cytokine storm, and lung pathology.

The MMR (Measles, Mumps, and Rubella) vaccine is one of the most successful vaccines in human history (Lin W H W, et al. Sci Transl Med 2020 12; Griffin D E. Viral Immunol 2018 31:86-95). Based on the CDC data, one dose of MMR vaccine is 93% effective against MeV, 78% effective against mumps virus (MuV), and 97% effective against rubella. Two doses of MMR vaccine are 97% effective against MeV and 88% effective against MuV. Both MeV and MuV belong to non-segmented negative-sense (NNS) RNA virus and have potential as vectors to deliver foreign antigens. Particularly, MeV has been widely used as a vaccine vector. To date, more than 100 antigens have been expressed by MeV and more than 20 rMeV-based vaccines have been tested in preclinical trials (Frantz P N, et al. Microbes Infect 2018 20:493-500; Lorin C, et al. J Virol 2004 78:146-157). Animal studies have shown that rMeV-based vaccines are highly effective against infectious diseases. Common immunization routes such as i.m., s.c., i.p. and i.n. were effective to induce a high level of immune responses in cotton rats, IFNAR1^{-/-}-hCD46 mice, and nonhuman primates (Niewiesk S. Curr Top Microbiol Immunol 2009 330:89-110; Nurnberger C, et al. J Virol 2019 93; Gerke C, et al. Expert Rev Vaccines 2019 18:393-403). Currently, phase I clinical trials are being conducted to evaluate MeV-vectored vaccines against Zika virus (NCT02996890, NCT04033068), Lassa virus (NCT04055454), and HIV (NCT01320176). In addition, a phase II clinical trials have demonstrated that a rMeV-vectored chikungunya virus (CHIKV) vaccine was highly effective against CHIKV infection in humans (Reisinger E C, et al. Lancet 2018 392:2718-2727).

The disclosed data demonstrate that MeV is an excellent vaccine platform for delivering a SARS-CoV-2 vaccine. Live attenuated MeV vaccine has been widely used and has excellent track record of high safety and efficacy in the human population since the 1960's (Hughes S L, et al. Vaccine 2020 38:460-469; Reisinger E C, et al. et al. Lancet 2019 392:2718-2727; Strebel P M. N Engl J Med 2019 381:349-357). MeV grows to high titers in Vero cells, a WHO approved cell line for vaccine production, facilitating vaccine manufacturing. Natural immunity to SARS-CoV-2 may not be long-lived (Prompetchara E, et al. Asian Pac J Allergy Immunol 2020 38:1-9; Decaro N, et al. Res Vet Sci 131, 21-23 (2020)). However, MeV vaccine induces long-lasting immunity and protection against MeV infections (Griffin D E. Viral Immunol 2018 31:86-95; Orenstein W A, et al. Vaccine 36 Suppl 1, A35-A42 (2018)). By expressing the S protein from an rMeV vector, it may be possible to also induce long-lasting immunity to the S protein and protect against COVID-19 disease. In areas of the world where MeV vaccination is not complete, such a combination vaccine could protect against both diseases. By incorporating rMeV-preS into the existing MMR vaccine, a quadruple vaccine could be developed against these four important pathogens for children. According to American Academy of Pediatrics, the number of U.S. infants, children and teens diagnosed with COVID-19 had reached more 2.6 million by Jan. 21, 2021, accounting for 12.7% of all cases in the US. Such a quadruple vaccine would be highly attractive for children.

In this study, the efficacy of preS, native full-length S, S1, and three different lengths of RBD antigens in cotton rats were directly compared. The preS protein is the most potent antigen in inducing SARS-CoV-2 specific ELISA, but more importantly, neutralizing antibodies. All five cotton rats immunized with rMeV-preS triggered uniformly high antibody responses whereas antibody titers in the rMeV-S group were variable. Although there was no significant difference in antibody titers (P>0.05), the rMeV-preS induced significantly higher neutralizing antibodies than rMeV-S(P<0.05). Similarly, rMeV-preS induced uniformly high antibodies in other animal models including IFNAR^{-/-}-CD46 mice and Golden Syrian hamsters. In hamsters, the neutralizing antibody induced by rMeV-preS were significantly higher than human COVID-19 convalescent sera (P<0.05). These results suggest that preS is more immunogenic than native full-length S. It is likely that spontaneous triggering of the metastable native full-length S leads to release of the S1 portion of the protein and refolding to the postfusion form, thereby losing the prefusion-specific antigenic sites and reducing its ability to induce neutralizing antibodies. This is similar to many fusion glycoproteins (such as those from paramyxoviruses, pneumoviruses, and HIV) in that the “prefusion” form of proteins are more potent in inducing neutralizing activity than its “postfusion” forms (Crank M C, et al., Science 2019 365:505-509; McLellan J S, et al. Science 2013 340:1113-1117; McLellan J S. et al. Science 2013 342:592-598; Kwong P D, et al. Immunity 2018 48:855-871; Stewart-Jones G B E, et al. Proc Natl Acad Sci USA 2018 115:12265-12270). S1 and the RBDs are poor antigens in the MeV vector, which is probably due to the suboptimal conformation of these monomeric proteins. Interestingly, Pfizer's BNT162b1, a lipid-nanoparticle-formulated, nucleoside-modified mRNA vaccine that encodes the trimerized RBD, was effective in triggering neutralizing antibody in human clinical trials (Sahin U, et al. Nature 2020 586:594-599; Mulligan M J, et al. Nature 2020 586:589-593). Perhaps, the trimerization and/or adjuvants enhance its immunogenicity.

One important advantage of using rMeV-preS-based vaccine candidate is that rMeV-preS induced predominately Th1-biased T-cell response thereby reducing the risk of potential antibody-dependent enhancement (ADE). High frequencies of CD8⁺ T cells capable of producing Th1 cytokines was observed, whereas frequencies of CD4⁺ T cells were low. Similar results were observed in an earlier study in which mice were vaccinated with recombinant adenovirus vector expressing SARS-CoV-2 S protein (Hassan A O, et al. Cell 2020 183:169-184 e113), Consistent with this, hamsters immunized with rMeV-preS were completely protected against SARS-CoV-2 challenge without any enhanced lung immunopathology. These results suggest that rMeV-preS is safe and highly efficacious. Historically, ADE has been a challenge in coronavirus vaccine development (Lee W S, et al. Nat Microbiol 2020 5:1185-1191). It was reported that inactivated MERS-CoV vaccine candidates (Agrawal A S, et al. Hum Vacc Immunother 2016 12:2351-2356) and several SARS-CoV-1 vaccine candidates including an inactivated whole virus vaccine (Bolles M, et al. J Virol 2011 85:12201-12215), virus-like-particle vaccine (Tseng C T, et al. PLoS One 2012 7:e35421), and modified vaccinia virus Ankara-based recombinant vaccine (Czub M, et al. Vaccine 2005 23:2273-2279) induced ADE in various animal models. Mechanistically, the excessive Th2-cytokine-biased responses and inadequate Th1-biased T-cell response contributed to the immunopathology upon SARS-CoV-1 infection (Lee W S, et al. Nat Microbiol 2020 5:1185-1191; Bolles M, et al. J Virol 2011 85:12201-12215; Tseng C T, et al. PLoS One 2012 7:e35421). Thus, an ideal SARS-CoV-2 vaccine should induce a high level of Th1 but not Th2-biased T-cell response. Clearly, the rMeV-preS-based vaccine platform meets this criteria.

During preparation of this manuscript, Hörner et al., reported a rMeV-based SARS-CoV-2 vaccine candidate (Homer C, et al. Proc Natl Acad Sci USA 2020). In their study, the full-length S gene was inserted at the H and L gene junction located at 5′ proximity of the MeV genome. A single immunization of this recombinant virus [MeV_{vac2}-SARS2-S(H)] did not induce any SARS-CoV-2 specific neutralizing antibody. After booster immunization, only 3 out of 7 animals produced detectable neutralizing antibody. After challenge with SARS-CoV-2, the MeV_{vac2}-SARS2-S(H)-immunized hamsters had significant weight loss at PIDs 1-3 but started to gain weight at PID 4. Furthermore, 4-5 log PFU/g tissue of SARS-CoV-2 were still detected in the nasal turbinate in MeV_{vac2}-SARS2-S(H)-immunized hamsters. Thus, MeV_{vac2}-SARS2-S(H) only induced partial protection against SARS-CoV-2 challenge (Homer C, et al. Proc Natl Acad Sci USA 2020). The efficacy of rMeV-based SARS-CoV-2 vaccine was significantly improved by using two novel strategies. First, rMeV expressing a stabilized, prefusion spike (S) (rMeV-preS) and rMeV expressing full-length S protein (rMeV-S) was generated. rMeV-preS was significantly more potent in inducing SARS-CoV-2 specific neutralizing antibodies than rMeV-preS. Second, or preS and S genes were inserted at the P and M gene junction located at 3′ proximity of MeV genome. As a typical non-segmented negative-sense RNA virus, MeV mRNA transcription is sequential and gradient such that 3′ proximal genes are transcribed more abundantly than 5′ distal genes. Thus, the expression of preS and S in the disclosed vaccines is much higher than Homer's vaccine, which further enhance the immunogenicity. rMeV-preS induced uniformly high levels of neutralizing antibody in all animals in all four animal models. A single immunization of rMeV-preS is sufficient to induce a high level of antibody response. Importantly, rMeV-preS induced higher levels of neutralizing antibody than found in convalescent sera from COVID-19 patients. Furthermore, rMeV-preS provides complete protection against SARS-CoV-2 challenge.

In summary, a safe and highly efficacious rMeV-based prefusion S vaccine candidate was developed that can provide complete protection against severe SARS-CoV-2 infection and lung pathology in animal models, supporting its further development as a vaccine.

### Example 3: Generate Measles Viruses Expressing preS with HexaPro and preS of SARS-CoV-2 Variants

SARS-CoV-2 virion is in its “prefusion” form (preS), which is a class I fusion protein trimer. Upon triggering, preS undergoes dramatic structural rearrangement, resulting in the post-fusion S (postS) protein. Antibodies to the prefusion form of the paramyxovirus, pneumovirus, and HIV fusion proteins have significantly higher neutralizing activity than antibodies to their “postfusion” forms. The stabilized prefusion S (preS) induced significantly higher neutralizing antibody than the native full-length S protein (Lu et al., PNAS, 2021). In this preS version, the furin site was deleted to prevent S1/S2 cleavage, two amino acids in the S2 subunit was replaced with prolines (2Pro), and the C-terminal transmembrane/cytoplasmic tail (TM/CT) domain was replaced with a T4 fibritin self-trimerizing domain. Recently, a more stable soluble preS with 6 strategic amino acids replaced with prolines (HexaPro) has been reported (Hsieh et al., Science, 2020). The preS with HexaPro (preS-HexaPro) has a higher protein expression than preS with 2Pro (preS-2Pro). preS-HexaPro is also more resistant to heat stress, storage at room temperature, and three freeze-thaw cycles. Thus, preS-HexaPro may enable better B cell activation over a longer period, which enhance the antibody responses.

Recombinant MeV expressing preS-HexaPro of recent emergent SARS-CoV-2 variants is generated in order to develop MeV-based vaccines for these variants. Genetic variants of SARS-CoV-2 have been emerging and circulating around the world throughout the COVID-19 pandemic. Examples of the most well-known SARS-CoV-2 variants include United Kingdom variant, South Africa Variant, Brazil variant, and New York variant. In the United States, viral mutations and variants are routinely monitored through sequence-based surveillance, laboratory studies, and epidemiological investigations. Recently, US government agency developed a Variant Classification scheme that defines three classes of SARS-CoV-2 variants: Variant of Interest; Variant of Concern; Variant of High Consequence.

Variants of interest: This type of variants including amino acid changes in S proteins that are associated with changes to receptor binding, reduced neutralization by antibodies generated against previous infection or vaccination, reduced efficacy of treatments, or increased in transmissibility or disease severity. Examples of Variants of interest include B.1.526, B.1.526.1, B.1.525, and P.2 variants circulating in the United States. The genetic markers for this type of variants are summarized in Table 4.

TABLE 4

| Name | Changes in spike protein | First detected |
|---|---|---|
| B.1.526 | Spike: (L5F*), T951, D253G, | United States (New |
| | (S477N*), (E484K*), D614G, | York) - November |
| | (A701V*) | 2020 |
| B.1.526.1 | Spike: D80G, Δ144, F157S, L452R, | United States (New |
| | D614G, (T791I*), (T859N*), D950H | York) - October 2020 |
| B.1.525 | Spike: A67V, Δ69/70, Δ144, E484K, | United Kingdom/ |
| | D614G, Q677H, F888L | Nigeria - December |
| | | 2020 |
| P.2 | Spike: E484K, (F565L*), D614G, | Brazil - April 2020 |
| | V1176F | |

Variants of concern: The characteristics of these variants include an increase in transmissibility, more severe disease (e.g., increased hospitalizations or deaths), significant reduction in neutralization by antibodies generated during previous infection or vaccination, reduced effectiveness of treatments or vaccines, or diagnostic detection failures. Examples of these variants include B.1.1.7, B.1.351, P.1, B.1.427, and B.1.429 variants circulating in the United States. The genetic markers for this type of variants are summarized as below:

TABLE 5

| Name | Changes in spike protein | First detected |
|---|---|---|
| B.1.1.7 | Δ69/70, | United Kingdom |
| | Δ144, | |
| | (E484K*), | |
| | (S494P*), | |
| | N501Y, | |
| | A570D, | |
| | D614G, | |
| | P681H, T716I, S982A, D1118H | |
| | (K1191N*) | |
| P.1 | L18F, T20N, P26S, D138Y, R190S, | Japan/ |
| | K417T, E484K, N501Y, D614G, | Brazil |
| | H655Y, T1027I | |
| B.1.351 | D80A, D215G, Δ241/242/243, | South Africa |
| | K417N, E484K, N501Y, D614G, | |
| | A701V | |
| B.1.427 | L452R, D614G | United States-(California) |
| B.1.429 | S13I | United States-(California) |
| | W152C | |
| | L452R | |
| | D614G | |

Variant of High Consequence: This type of variants has clear evidence that prevention measures or medical countermeasures (MCMs) have significantly reduced effectiveness relative to previously circulating variants. To date, no variants of high consequence have been identified in the United States.

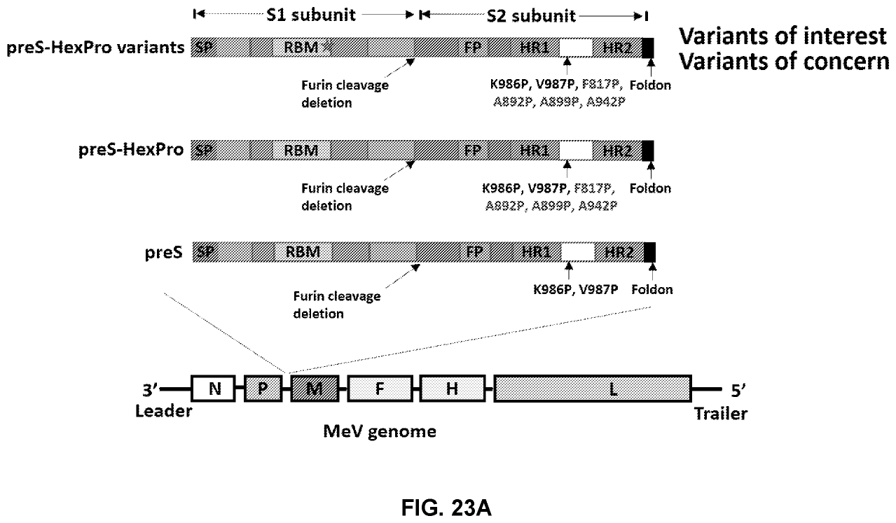

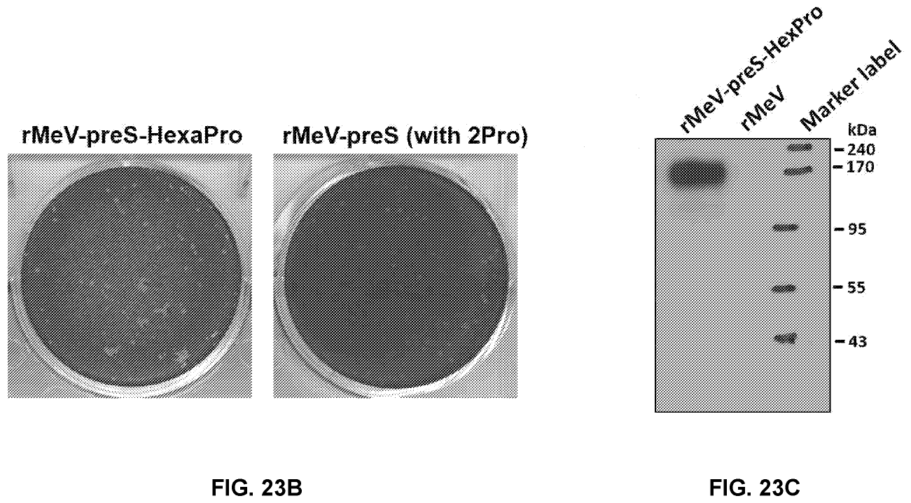

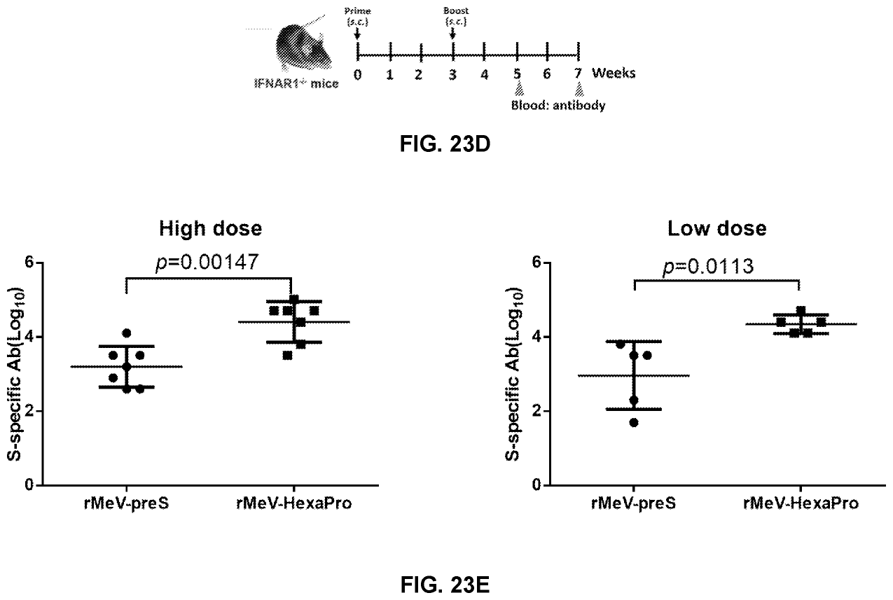

Recombinant MeV expressing preS-HexaPro was constructed. The preS-HexaPro was cloned into the P and M gene junction in the genome of MeV Edmonston strain using the yeast-based recombination system (FIG. 23A). The plasmid was named pMeV-preS-HexaPro. Similarly, preS-HexaPro carrying mutations of SARS-CoV-2 variants will be inserted into the P and M gene junction in MeV genome (FIG. 23A). These plasmids were named pMeV-preS-HexaPro-variants Using the reverse genetics system, we have recovered recombinant MeV expressing preS-HexaPro (rMeV-preS-HexaPro) (FIG. 23B). Next, we determined the expression of preS-HexaPro by rMeV vector. Briefly, Vero CCL81 cells were infected by rMeV-preS-HexaPro or the parental rMeV at an MOI of 1.0. Cell culture supernatants were collected at 72 h post-infection and subjected to Western blot using antibody against S protein. preS-HexaPro protein was detected in supernatants from rMeV-preS-HexaPro-infected cells but not rMeV-infected cells (FIG. 23C), confirming that preS-HexaPro is highly expressed by rMeV vector.

The immunogenicity of rMeV-preS-HexaPro and rMeV-preS (with 2Pro) was compared (FIG. 23D). Briefly, type-1 interferon receptor subunit 1 (IFNAR1) knockout mice (IFNAR1^{-/-}) mice were immunized subcutaneously with 2×10⁵ or 2×10⁴ PFU of rMeV-preS-HexaPro, rMeV-preS, or rMeV, and were boosted with the same dose via the same route at week 3. At week 7 (for high dose) or 5 (for low dose) post-immunization, serum was collected from each mouse to determine the antibody level. As shown in FIG. 23E, rMeV-preS-HexaPro triggered a significantly higher antibody titer compared to rMeV-preS at both high and low doses (P<0.01 and P<0.05).

In summary, these results demonstrate that rMeV-preS-HexaPro is more immunogenic than rMeV-preS in mice. Therefore, the expression of prefusion S can be further optimized by add 6 strategic prolines in S thereby enhancing immunogenicity. Furthermore, rMeV expressing preS-HexaPro variants will provide protection against recent emergent SARS-CoV-2 variants.

### Example 4: Generate Recombinant Measles Virus Expressing Fc-Fused or Trimerized RBD and S1 Protein

Coronavirus S protein is trimeric. The S protein consists of a receptor-binding subunit S1 and a membrane-fusion subunit S2. S1 is further divided into two domains: an N-terminal domain (NTD) and a C-terminal domain (CTD) also called receptor-binding domain (RBD). Thus, the RBD and S1 are trimer forms in the S structure. However, expression of RBD and S1 protein leads to monomer version of protein. Thus, a stabilized trimer version of RBD and S1 may elicit an improved immune response compared to their monomer versions.

The Fc fusion protein have been used as a peptide to promote the correct folding of protein and to enhance binding to antigen-presenting cells (APCs) and cells expressing Fc receptors (FcR). Thus, the Fc fusion protein can increase the immunogenicity of target antigens and enhance the neutralizing antibody response. The fusion of Fc to a target protein can also offer other advantages including an expedient for rapid purification and longer half-life of the targeted protein.

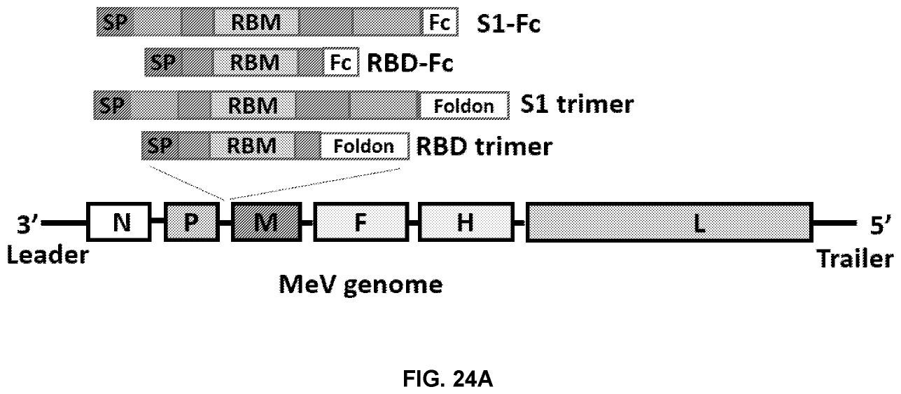

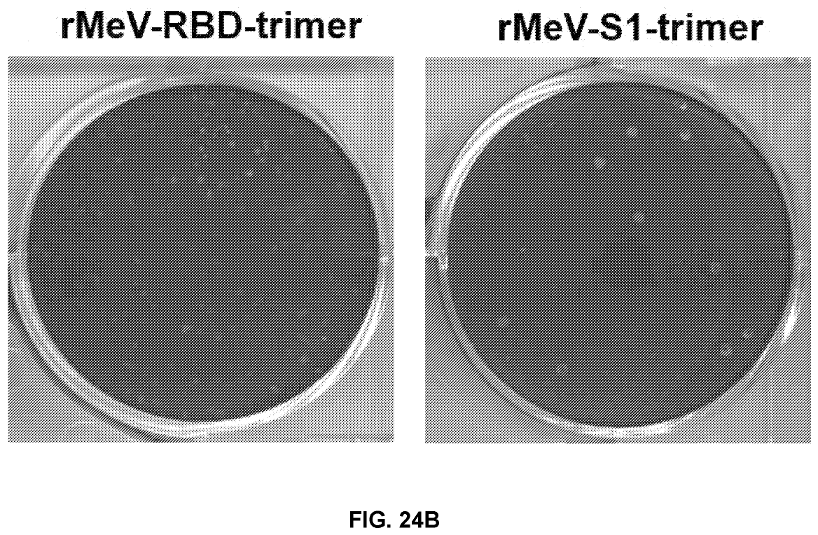

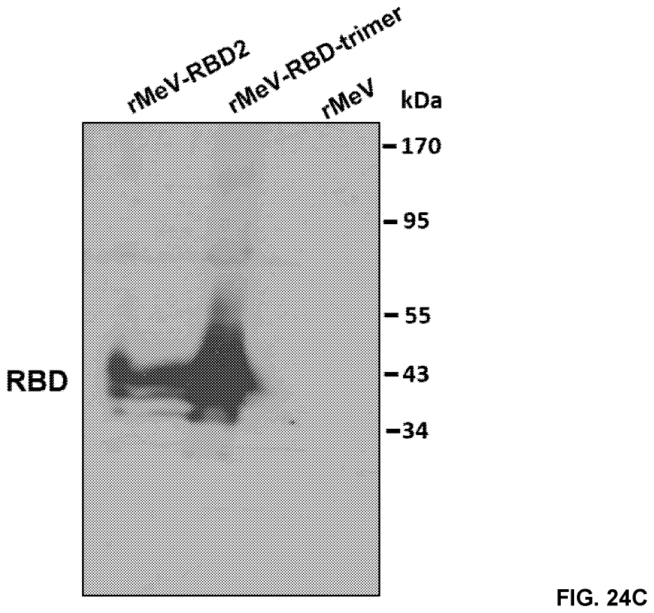

Trimerized RBD and S1 and the Fc-fused RBD and S1 were designed (FIG. 24A). Specifically, a signal peptide (SP) and T4 fibritin-derived foldon trimerization domain was fused to the N-terminal and C-terminal of RBD and S1 proteins, respectively. The resultant RBD and S1 were named as RBD-trimer and S1-trimer respectively. The C-terminus of RBD and S1 genes was fused to the human IgG1 Fc fragment. The RBD-trimer, S1-trimer, RBD-Fc, and S1-Fc were cloned into the P and M gene junction in the genome of MeV Edmonston strain using the yeast-based recombination system (FIG. 24A). The plasmids were named pMeV-RBD-trimer, pMeV-S1-trimer, pMeV-RBD-Fc, and pMeV-S1-Fc. Using the reverse genetics system, recombinant MeV expressing RBD-trimer, S1-trimer, RBD-Fc, and S1-Fc were recovered (FIG. 24B). Example of plaque formation of rMeV-RBD-trimer and rMeV-S1-trimer was shown in FIG. 24B. Next, the expression of RBD-trimer by rMeV vector was determined. Briefly, Vero CCL81 cells were infected by rMeV-RBD2 (monomer), rMeV-RBD-trimer, or the parental rMeV at an MOI of 1.0. Cell lysates were collected at 72 h post-infection and subjected to Western blot using antibody against S protein. RBD-trimer had a higher expression compared to its monomer version by MeV vector (FIG. 24C).

In summary, measles virus expressing trimerized RBD and S1 proteins was constructed. Trimerized RBD and S1 proteins will likely have a better immunogenicity than their monomer versions because trimerized RBD and S1 proteins will have optimal conformations and more protein expression.

### Example 5: Generate Recombinant Measles Virus Expressing Other Structural Proteins, Accessary Protein, and Nonstructural Protein that Containing T Cell Epitopes

To date, SARS-CoV-2 vaccine research has exclusively focused on the major target of neutralizing antibodies, the S protein. However, other structural, nonstructural, and accessory proteins may produce T cell immune responses that may play in a role in protection against CoV infection are unknown. For many viruses, multiple viral proteins collectively contribute to immuno-protection. In addition to glycoproteins or capsid proteins, nonstructural proteins (nsp) of many positive-sense RNA viruses can provide complete protection against infection via T cell-mediated killing of infected cells. Multiple viral proteins induce protection against other viruses by a variety of mechanisms including the induction of T cells or antibodies that do not neutralize but enable NK cell killing of infected cells by antibody-dependent cell-mediated cytotoxicity (ADCC), or cytotoxic T cell killing.

SARS-CoV-2 encodes 4 structural proteins, S, membrane (M), envelope (E), and nucleocapsid (N). S, M, and E are embedded in the membrane of the virion whereas N binds to the viral genome inside the virion. SARS-CoV-2 encodes a large ORF1, whose ORF1a/b is cleaved to produce a total of 16 nonstructural proteins (nsp1-nsp16). In addition, the 3′ end of the SARS-CoV-2 genome encodes several accessory proteins (ORF3 and ORF6-10) with anti-innate immunity or other unknown functions. These viral proteins may possess T cell epitopes that are important for viral clearance. Identification of viral protein(s) that induce protective T cell responses would be novel and could be co-delivered with S to provide synergistic protection from SARS-CoV-2 infection.

In fact, high levels of IgG antibodies against N were detected in sera from SARS patients, and the N protein can induce SARS-specific T cells with cytotoxic activity. Recently, the nature of the T cell immune response in human patients with SARS-CoV-2 infection was analyzed. S, M, and N accounted for 27%, 21%, and 11% of the total CD4+ T cell response, respectively. In addition, nsp3, nsp4, and ORF8 each accounted for 5% of the total CD4+ T cell response. As for CD8+ T cell responses, S, M, N, nsp6, ORF8, and ORF3a accounting for 26%, 22%, 12%, 15%, 10%, and 7%, respectively. These results suggest that T cell immunity may be important for protection against COVID-19.

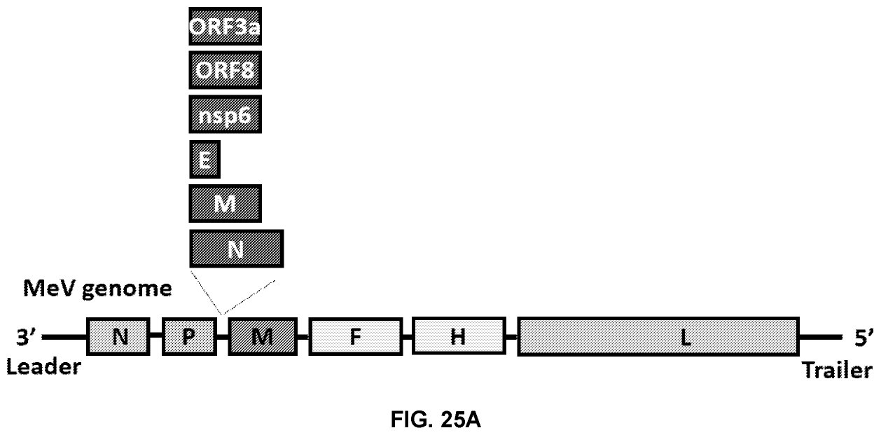

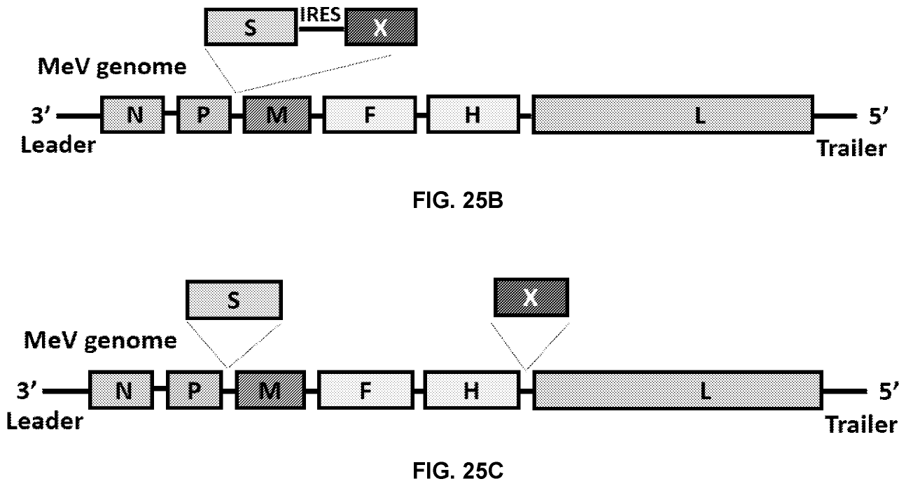

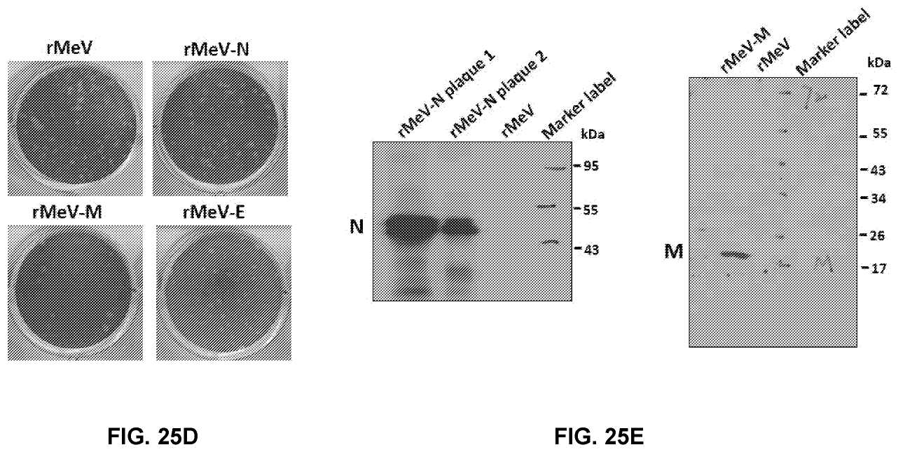

To determine the roles of T cell immune responses in protecting SARS-CoV-2 infection, each viral protein found to induce 10% or more of the CD4⁺ or CD8⁺ T cells in the COVID-19 patient study described above will be inserted individually into the MeV vector: M, N, nsp6, ORF3a and ORF8 (FIG. 25A). MeV co-expressing S and X (here “X” designates one or more structural, accessary, and nonstructural proteins) is also constructed in the same location in MeV genome (FIG. 25B). An IRES (or 2A protease) will be inserted between the S and X genes, resulting in translation of an equal amount of S and X proteins. The S_{IRES}X cassette is inserted at the junction between P and M of MeV genome, and rMeV co-expressing S and X proteins will be recovered (FIG. 25B). Alternatively, MeV co-expressing S and X (X designates one or more structural, accessary, and nonstructural proteins) at two different locations is constructed (FIG. 25C). The S and X genes can be inserted into two separate gene junctions in MeV vector. To this end, we have constructed recombinant measles virus expressing M, N, and E genes of SARS-CoV-2 at the P and M gene junction (FIG. 25D). These recombinant viruses were named rMeV-N, rMeV-M, rMeV-E. Plaque assay showed that these recombinant viruses formed smaller plaques compared to the parental rMeV (FIG. 25D). Next, the expression of M, N, and E proteins by rMeV vector was determined. Briefly, Vero CCL81 cells were infected by rMeV-M, rMeV-N, rMeV-E, or the parental rMeV at an MOI of 1.0. Cell lysate were collected at 72 h post-infection and subjected to Western blot using antibody against N protein. N protein was highly expressed by MeV vector (FIG. 25E).

In summary, recombinant measles virus expressing structural proteins M, N, and E was have constructed. The feasibility of using MeV as the vector to express other nonstructural, accessory, and nonstructural proteins was also demonstrated. These proteins contain T cell epitopes that may protect SARS-CoV-2 infection. Combination of S with one or more structural proteins, accessory proteins, and nonstructural proteins may provide synergistic effects against SARS-CoV-2 because they induce strong neutralizing antibodies and T cell immunity.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A live attenuated recombinant measles virus (rMeV)-based coronavirus vaccine comprising a SARS-CoV-2 spike (S) protein inserted between the P and M genes of the rMeV genome, wherein the S protein comprises at least one mutation to remove a glycosylation site.

2. The vaccine of claim 1, wherein the S protein is a soluble stabilized prefusion S protein.

3. The vaccine of claim 2, wherein the soluble stabilized prefusion S protein comprises the amino acid sequence SEQ ID NO:15, or a variant thereof having K986P, V987P, F817P, A892P, A899P, and A942P mutations.

4. The vaccine of claim 1, wherein the S protein is S1 protein or S2 protein.

5. The vaccine of claim 4, wherein the S1 comprises the amino acid sequence SEQ ID NO:4.

6. (canceled)

7. The vaccine of claim 4, wherein the S2 comprises the amino acid sequence SEQ ID NO:6.

8. The vaccine of claim 1, wherein the S protein lacks the transmembrane domain.

9. The vaccine of claim 8, wherein the S protein comprises the amino acid sequence SEQ ID NO:13.

10. The vaccine of claim 1, wherein the S protein is an S protein fragment comprising at least the receptor-binding domain (RBD).

11. The vaccine of claim 10, wherein the RBD comprises the amino acid sequence SEQ ID NO:124, 125, or 126.

12. The vaccine of claim 1, wherein the S protein is a trimeric S1 protein.

13. The vaccine of claim 12, wherein the trimeric S1 protein comprises the amino acid sequence SEQ ID NO:147.

14. The vaccine of claim 1, wherein the S protein is an Fc-fused S1 protein.

15. The vaccine of claim 14, wherein the Fc-fused S1 protein comprises the amino acid sequence SEQ ID NO:151.

16. The vaccine of claim 1, wherein the S protein is a trimeric RBD protein.

17. The vaccine of claim 16, wherein the trimeric RBD comprises the amino acid sequence SEQ ID NO:147.

18. The vaccine of claim 1, wherein the S protein is an Fc fused RBD protein.

19. The vaccine of claim 18, wherein the Fc fused RBD comprises the amino acid sequence SEQ ID NO:149.

20. (canceled)

21. A live attenuated recombinant coronavirus vaccine, wherein a stabilized prefusion spike (S) protein is inserted into a viral vector genome.

22-28. (canceled)

29. A recombinant measles virus (rMeV) system, comprising a yeast expression vector comprising a yeast replication origin, a T7 RNA polymerase, a hepatitis delta virus (HDV) ribozyme sequence, a T7 promoter, and a cDNA clone of measles virus (MeV) genome.

30-31. (canceled)
